(19) United States
(12) Patent Application Publication
(10) Pub. No.: US 2024/0308988 A1
(43) Pub. Date: Sep. 19, 2024

(54) Method For Controlling Diamide Resistant Pests and Compounds Therefor

(71) Applicant: SYNGENTA CROP PROTECTION AG, Basel (CH)

(72) Inventors: Amandine KOLLETH KRIEGER, Leyman (FR); Fides BENFATTI, Stein (CH); Peter FINKBEINER, Murg (DE); Roger Graham HALL, Stein (CH); Mattia Riccardo MONACO, Stein (CH); Stefano RENDINE, Stein (CH); Andre STOLLER, Blotzheim (FR)

(73) Assignee: SYNGENTA CROP PROTECTION AG, Basel (CH)

(21) Appl. No.: 18/681,238

(22) PCT Filed: Jul. 31, 2022
(86) PCT No.: PCT/EP2022/071489

§ 371 Date: Feb. 5, 2024

(30) Foreign Application Priority Data
Aug. 5, 2021 (EP) 21189831.7
Jan. 27, 2022 (EP) 22153779.8

(51) Int. Cl. C07D 403/14; A01N 43/56; A01N 43/647; A01N 43/713; A01P 7/04; C07D 403/04

(52) U.S. Cl. CPC C07D 403/14; A01N 43/56; A01N 43/647; A01N 43/713; A01P 7/04; C07D 403/04

(57) ABSTRACT

A method for combating and controlling diamide-resistant insects to (i) reduce damage on a plant, which comprises applying to the insect, to a locus of the insect, or to a plant susceptible to attack by the insect, an effective amount of a compound of formula (I); or (ii) protect plant propagation material, which comprises treating the propagation material or the site, where the propagation material is planted, with an effective amount of a compound of formula (I); wherein the compound of formula (I) is wherein the substituents are as defined in claim 1, and the agrochemically acceptable salts, stereoisomers, enantiomers, tautomers and N-oxides of those compounds, can be used as insecticides.

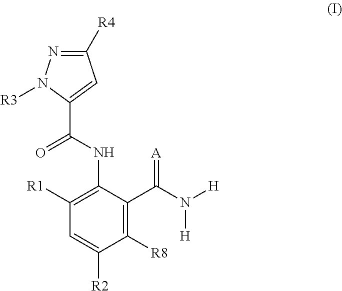

The present invention relates to method of controlling diamide-resistant pests by use of certain pesticidally active, in particular insecticidally active, diamide compounds. Further, present invention also relates to certain pesticidally active, in particular insecticidally active, diamide compounds, to processes for their preparation, to compositions comprising those compounds, and to their use for controlling animal pests, including arthropods and in particular insects or representatives of the order Lepidoptera, in particular diamide resistant Lepidoptera insects.

Diamide (or bisamide) derivatives with insecticidal action are known and described, for example, in WO 2005/085234, WO 2007/093402 and WO2003/015518.

Bisamide insecticidal derivatives have been used widely during more than a decade and some insect populations have developed a level of resistance that renders them not susceptible enough to be sufficiently controlled by compounds of the bisamide class available on the market. The consequence of this evolution is that a higher dose of protectant must be used and/or the protection of the crops might be insufficient.

Diamide insecticides target the ryanodine receptor in insects and lead to a depletion of the intracellular calcium reservoirs (Ebbinghaus-Kintscher et al. 2006; Sattelle, Cordova, and Cheek 2008; Cordova et al. 2006). Commercial diamides can be attributed to two classes, the phthalic diamides with its sole representative being flubendiamide and the anthranilic diamides comprising chlorantraniliprole, cyantraniliprole, cyclaniliprole, and tetraniliprole. Other examples of phthalic diamides and anthranilic diamides are cyhalodiamide, fluchlordiniliprole and tetrachlorantraniliprole. All diamides share the same mode of action and so are grouped in the IRAC MoA Group 28.

The diamides represent a fast-growing class of insecticides introduced to the market since the commercialization of neonicotinoids (Sparks and Nauen 2015; Richardson et al. 2020; Troczka et al. 2017) and are extremely valuable insect control agents not least because they had exhibited little or no cross-resistance to older insecticide classes, which suffer markedly from resistance problems. However, reports of insect resistance to the diamides class of insecticides are on the increase.

The increase in resistance of such insects to diamide insecticides thus poses a significant threat to the cultivation of a number of commercially important crops, fruits and vegetables, and there is thus a need to find alternative insecticides capable of better controlling diamide resistant insects (e.g. to find insecticides that do not exhibit any cross-resistance with the diamide class).

Resistance may be defined as “a heritable change in the sensitivity of a pest population that is reflected in the repeated failure of a product to achieve the expected level of control when used according to the label recommendation for that pest species” (IRAC 2009).

Cross-resistance occurs when resistance to one insecticide confers resistance to another insecticide via the same biochemical mechanism. This can happen within insecticide chemical groups or between insecticide chemical groups. Cross-resistance may occur even if the resistant insect has never been exposed to one of the chemical classes of insecticide.

Two of the major mechanisms for diamide resistance include:
(i) Target site resistance, whereby resistance is associated with replacement of one or more amino acids in the insecticide target protein (i.e. the ryanodine receptor); and
(ii) Metabolic resistance, such as enhanced oxidative detoxification of diamides due to overexpression of cytochrome P450 monooxygenases (P450) or conjugation of diamides due to overexpression of UDP-dependent glycosyl transferases (UGT);

Target site resistance has been described in numerous Lepidopteran species incl. Plutella xylostella (Troczka et al. 2012; Steinbach et al. 2015; Guo et al. 2014), Tuta absoluta (Roditakis et al. 2017; Zimmer et al. 2019), Spodoptera frugiperda (Bolzan et al. 2019) Spodoptera exigua (Zuo et al. 2020, 2017) Chilo suppressalis (Yao et al. 2017; Yang et al. 2017). Similar to what has been described for target site resistance against other insecticides e.g. organochlorines affecting the GABA receptor (french-Constant et al. 1998) parallel evolution can also be observed for diamide resistance with two mutations i.e. I4970M and G4946E (P. xylostella numbering) frequently described across species (Richardson et al. 2020). However, that does not exclude that mutations in different positions in the target-site may cause high levels of diamide resistance.

The cytochrome P450 monooxygenases are an important metabolic system involved in the detoxification of xenobiotics in phase I i.e. modification (Dermauw, Van Leeuwen, and Feyereisen 2020; Bard 2000). As such, P450 monooxygenases play an important role in insecticide resistance. P450 monooxygenases have such a phenomenal array of metabolizable substrates because of the presence of numerous P450s (˜26-261) arthropodal species, as well as the broad substrate specificity of some P450s (Dermauw, Van Leeuwen, and Feyereisen 2020). Studies of monooxygenase-mediated resistance have indicated that resistance can be due to increased gene expression of one P450 involved (quantitative changes) in detoxification of the insecticide and might also be due to a mutation in the gene itself altering the amino acid composition (qualitative changes) (Feyereisen, Dermauw, and Van Leeuwen 2015). As such, metabolic cross-resistance mechanisms affect not only insecticides from the given class (e.g. neonicotinoids) but also seemingly unrelated insecticides. For example, cross-resistance relationships between the neonicotinoids and pymetrozine in Bemisia tabaci have been reported (Gorman et al. 2010; Nauen et al. 2013).

Apart from cytochrome P450s other enzyme and transport protein families may lead to insecticide resistance e.g. oxidases, hydrolases, transferases and ABC-transporters (Dermauw and Van Leeuwen 2014; Feyereisen, Dermauw, and Van Leeuwen 2015; Bass et al. 2014). P450s as well as other oxidases, transferases and ABC-transporters have been implicated in diamide resistance (Li et al. 2017; Mallott et al. 2019; Li et al. 2018; Shan et al. 2021).

Therefore, it is highly desirable to find classes of compounds offering a better control of the resistant insects.

It has now been surprisingly found that certain diamide derivatives are able to still control diamide-resistant inects.

The present invention accordingly relates, in a first aspect, to a method for combating and controlling diamide-resistant insects to
(i) reduce damage on a plant, which comprises applying to the insect, to a locus of the insect, or to a plant susceptible to attack by the insect, an effective amount of a compound of formula I; or
(ii) protect plant propagation material, which comprises treating the propagation material or the site, where the propagation material is planted, with an effective amount of a compound of formula I;
   wherein the compound of formula I is

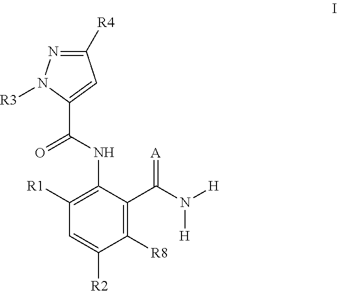

wherein
A is O or S;
R₁ is hydrogen, halogen, C₁-C₆alkyl, C₂-C₆alkenyl, C₂-C₆alkynyl, C₁-C₆haloalkyl, or C₃-C₆cycloalkyl;
R₂ is halogen or cyano;
R₃ is phenyl, or a 6-membered heteroaromatic ring, each of which is unsubstituted or substituted with one to three substituents independently selected from Re;
R₄ is hydrogen, halogen, C₁-C₆alkyl, C₂-C₆alkenyl, C₂-C₆alkynyl, C₁-C₆haloalkyl, C₃-C₆cycloalkyl, C₃-C₆halocycloalkyl, C₃-C₆cyanocycloalkyl, C₁-C₆alkoxy, C₁-C₆haloalkoxy, or X2-Y, where X2 is C₁-C₆alkanediyl or C₁-C₆haloalkanediyl, and Y is cyano, C₂-C₆alkenyl, C₂-C₆alkynyl, C₁-C₆alkoxy, C₁-C₆haloalkoxy, C₃-C₆cycloalkyl, C₃-C₆halocycloalkyl, C₁-C₄alkylsulfanyl, C₁-C₄alkylsulfinyl, C₁-C₄alkylsulfonyl, C₁-C₄haloalkylsulfanyl, C₁-C₄haloalkylsulfinyl, C₁-C₄haloalkylsulfonyl, benzyloxy, halobenzyloxy, 5- or 6-membered heteroaromatic ring, which is unsubstituted or substituted with one to three independently selected R₇, or a 9- or 10-membered heteroaromatic bicyclic system, which is unsubstituted or substituted with one to three independently selected R₇; R₆ is independently selected from halogen, cyano, C₁-C₃alkyl, C₁-C₃haloalkyl, C₁-C₃haloalkylthio, C₁-C₃alkoxy, C₁-C₃haloalkoxy, (C₁-C₃alkyl)NHC(═O), (C₁-C₃alkyl)₂NC(═O), (C₃-C₆cycloalkyl)NHC(═O), and (C₃-C₆cycloalkyl)(C₁-C₃alkyl)NC(═O); R₇ is independently selected from halogen, cyano, C₁-C₃alkyl, C₁-C₃haloalkyl, C₃-C₆cycloalkyl, (C₁-C₃alkyl)NHC(═O), (C₁-C₃alkyl)₂NC(═O), (C₃-C₆cycloalkyl)NHC(═O), and (C₃-C₆cycloalkyl)(C₁-C₃alkyl)NC(═O), phenyl (which may be substituted with one to three substituents independently selected from halogen, cyano, C₁-C₄alkyl, C₁-C₃alkoxy and C₁-C₃haloalkyl), and 6-membered heteroaromatic ring (which may be substituted with one to three substituents independently selected from halogen, cyano, C₁-C₄alkyl, C₁-C₃alkoxy and C₁-C₃haloalkyl); and R₃ is hydrogen, halogen, cyano, C₁-C₆alkyl, C₂-C₆alkenyl, C₂-C₆alkynyl, C₁-C₆haloalkyl, C₃-C₆cycloalkyl, C₁-C₆alkoxy, or C₁-C₆haloalkoxy; or an agronomically acceptable salt, isomer, enantiomer, tautomer and/or N-oxide of the compound of formula I.

It has further now been found that certain novel bicyclic bisamide derivatives provide improved control over these diamide-resistant insects. Accordingly, a second aspect of the present invention relates to a compound of formula I
(i) represented by formula Ia

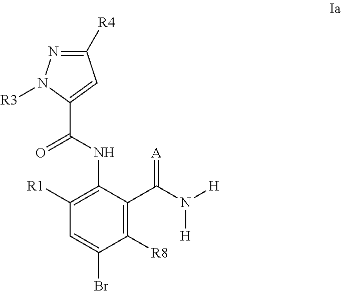

wherein bromine is R₂ from formula I; A is oxygen or sulfur; R₁ is hydrogen, halogen, C₁-C₆alkyl, C₂-C₆alkenyl, C₂-C₆alkynyl, C₁-C₆haloalkyl, or C₃-C₆cycloalkyl; R₄ is as defined in the first aspect; R₃ is 6-membered heteroaromatic ring, which is unsubstituted or substituted with one to three substituents independently selected from R₆; R₆ is independently selected from: halogen, C₁-C₃alkyl, and C₁-C₃haloalkyl; R₈ is hydrogen, halogen, cyano, C₁-C₆alkyl, C₂-C₆alkenyl, C₂-C₆alkynyl, C₁-C₆haloalkyl, C₃-C₆cycloalkyl, C₁-C₆alkoxy, or C₁-C₆haloalkoxy;
in the instance R₁ is chlorine, R₄ is hydrogen, fluorine, iodine, C₂-C₆alkyl, C₂-C₆alkenyl, C₂-C₆alkynyl, C₃-C₆cycloalkyl, C₃-C₆halocycloalkyl, C₃-C₆cyanocycloalkyl, C₁-C₆alkoxy, or X2-Y1;
in the instance R₁ is fluorine or bromine, R₄ is hydrogen, fluorine, iodine, C₁-C₆alkyl, C₂-C₆alkenyl, C₂-C₆alkynyl, C₁-C₆haloalkyl, C₃-C₆cycloalkyl, C₃-C₆halocycloalkyl, C₃-C₆cyanocycloalkyl, C₁-C₆alkoxy, C₁-C₆haloalkoxy, or X2-Y2; or
in the instance R₁ is methyl, R₄ is hydrogen, iodine, C₂-C₆alkyl, C₂-C₆alkenyl, C₂-C₆alkynyl, C₃-C₆cycloalkyl, C₃-C₆halocycloalkyl, C₃-C₆cyanocycloalkyl, C₁-C₆alkoxy, or X2-Y3;
or in the instance R₁ is hydrogen, iodine, C₂-C₆alkyl, C₂-C₆alkenyl, C₂-C₆alkynyl, C₄-C₆haloalkyl, or C₃-C₆cycloalkyl, R₄ is hydrogen, halogen, C₁-C₆alkyl, C₂-C₆alkenyl, C₂-C₆alkynyl, C₁-C₆haloalkyl, C₃-C₆cycloalkyl, C₃-C₆halocycloalkyl, C₃-C₆cyanocycloalkyl, C₁-C₆alkoxy, C₁-C₆haloalkoxy, or X2-Y4; or
(ii) represented by formula Ib

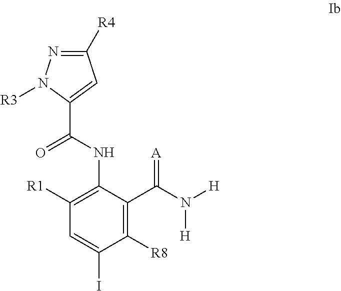

wherein iodine is R₂ from formula I; A is oxygen or sulfur; R₁ is hydrogen, halogen, C₁-C₆alkyl, C₂-C₆alkenyl, C₂-C₆alkynyl, C₁-C₆haloalkyl, or C₃-C₆cycloalkyl; R₄ is as defined in the first aspect; R₃ is 6-membered heteroaromatic ring, which is unsubstituted or substituted with one to three substituents independently selected from R₆; R₆ is independently selected from: halogen, C₄-C₃alkyl, and C₁-C₃haloalkyl; R₈ is hydrogen, halogen, cyano, C₁-C₆alkyl, C₂-C₆alkenyl, C₂-C₆alkynyl, C₁-C₆haloalkyl, C₃-C₆cycloalkyl, C₁-C₆alkoxy, or C₁-C₆haloalkoxy;
in the instance R₁ is hydrogen, halogen, C₂-C₆alkyl, C₂-C₆alkenyl, C₂-C₆alkynyl, C₁-C₆haloalkyl, or C₃-C₆cycloalkyl, R₄ is hydrogen, halogen, C₁-C₆alkyl, C₂-C₆alkenyl, C₂-C₆alkynyl, C₁-C₆haloalkyl, C₃-C₆cycloalkyl, C₃-C₆halocycloalkyl, C₃-C₆cyanocycloalkyl, C₁-C₆alkoxy, C₁-C₆haloalkoxy, or X2-Y5;
or in the instance R₁ is methyl, R₄ is hydrogen, halogen, C₁-C₆alkyl, C₂-C₆alkenyl, C₂-C₆alkynyl, CHF₂, C₂-C₆haloalkyl, C₃-C₆cycloalkyl, C₃-C₆halocycloalkyl, C₃-C₆cyanocycloalkyl, C₁-C₆alkoxy, C₁-C₆haloalkoxy, or X2-Y6; or
(iii) represented by formula Ic

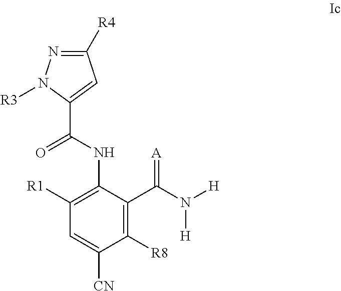

wherein cyano is R₂ from formula I; A is oxygen or sulfur; R₁ is hydrogen, halogen, C₁-C₆alkyl, C₂-C₆alkenyl, C₂-C₆alkynyl, C₁-C₆haloalkyl, or C₃-C₆cycloalkyl; R₄ is as defined in the first aspect; R₃ is 6-membered heteroaromatic ring, which is unsubstituted or substituted with one to three substituents independently selected from R₆; R₆ is independently selected from halogen, C₇-C₃alkyl, and C₁-C₃haloalkyl; R₈ is hydrogen, halogen, cyano, C₁-C₆alkyl, C₂-C₆alkenyl, C₂-C₆alkynyl, C₁-C₆haloalkyl, C₃-C₆cycloalkyl, C₁-C₆alkoxy, or C₁-C₆haloalkoxy;
in the instance R₁ is hydrogen, bromine, iodine, C₂-C₆alkyl, C₂-C₆alkenyl, C₂-C₆alkynyl, C₁-C₆haloalkyl, or C₃-C₆cycloalkyl, R₄ is hydrogen, halogen, C₁-C₆alkyl, C₂-C₆alkenyl, C₂-C₆alkynyl, C₁-C₆haloalkyl, C₃-C₆cycloalkyl, C₃-C₆halocycloalkyl, C₃-C₆cyanocycloalkyl, C₁-C₆alkoxy, C₁-C₆haloalkoxy, X2-Y7;
in the instance R₁ is methyl, R₄ is hydrogen, fluorine, iodine, C₂-C₆alkyl, C₂-C₆alkenyl, C₃-C₆cycloalkyl, C₃-C₆halocycloalkyl, C₃-C₆cyanocycloalkyl, C₁-C₆alkoxy, or X2-Y8;
in the instance R₁ is chlorine or fluorine, R₄ is hydrogen, fluorine, iodine, C₂-C₆alkyl, C₂-C₆alkenyl, C₂-C₆alkynyl, C₃-C₆cycloalkyl, C₃-C₆halocycloalkyl, C₃-C₆cyanocycloalkyl, or X2-Y9; or
(iii) represented by formula Id

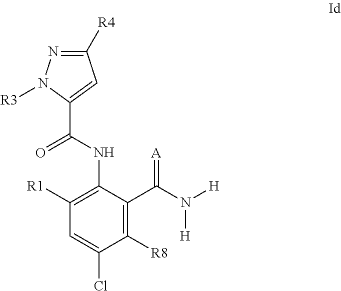

wherein chlorine is R₂ from formula I; A is oxygen or sulfur; R₁ is hydrogen, halogen, C₁-C₆alkyl, C₂-C₆alkenyl, C₂-C₆alkynyl, C₁-C₆haloalkyl, or C₃-C₆cycloalkyl; R₄ is as defined in the first aspect; R₃ is 6-membered heteroaromatic ring, which is unsubstituted or substituted with one to three substituents independently selected from R₆; R₆ is independently selected from halogen, C₁-C₃alkyl, and C₁-C₃haloalkyl; R₈ is hydrogen, halogen, cyano, C₁-C₆alkyl, C₂-C₆alkenyl, C₂-C₆alkynyl, C₁-C₆haloalkyl, C₃-C₆cycloalkyl, C₁-C₆alkoxy, or C₁-C₆haloalkoxy;
in the instance R₁ is chlorine, R₄ is hydrogen, fluorine, iodine, C₂-C₆alkyl, C₃-C₆cycloalkyl, C₃-C₆halocycloalkyl, C₃-C₆cyanocycloalkyl, C₁-C₆alkoxy or X2-Y10;
in the instance R₁ is bromine, R₄ is hydrogen, fluorine, iodine, C₂-C₆alkyl, C₂-C₆alkenyl, C₂-C₆alkynyl, C₆cycloalkyl, C₃-C₆halocycloalkyl, C₃-C₆cyanocycloalkyl, C₁-C₆alkoxy, OCF₃, or X2-Y11;
in the instance R₁ is methyl, R₄ is hydrogen, CH₂Cl, C₂-C₆alkyl, C₂-C₆alkenyl, C₃-C₆cycloalkyl, C₃-C₆cyanocycloalkyl, C₁-C₆alkoxy, OCH₂CHF₂, C₃-C₆haloalkoxy, or X2-Y12;
in the instance R₁ is hydrogen, C₂-C₆alkyl, C₂-C₆alkenyl, C₂-C₆alkynyl, C₁-C₆haloalkyl, or C₃-C₆cycloalkyl, R₄ is hydrogen, halogen, C₁-C₆alkyl, C₂-C₆alkenyl, C₂-C₆alkynyl, C₁-C₆haloalkyl, C₃-C₆cycloalkyl, C₃-C₆halocycloalkyl, C₃-C₆cyanocycloalkyl, C₁-C₆alkoxy, C₁-C₆haloalkoxy, or X2-Y13; where X2 is C₁-C₆alkanediyl or C₁-C₆haloalkanediyl;
Y1 is cyano, C₁-C₆haloalkoxy, C₃-C₆halocycloalkyl, C₁-C₄alkylsulfanyl, C₁-C₄alkylsulfinyl, C₁-C₄alkylsulfonyl, C₁-C₄haloalkylsulfanyl, C₁-C₄haloalkylsulfinyl, C₁-C₄haloalkylsulfonyl, benzyloxy, halobenzyloxy, or Y1.1-Y1.8;

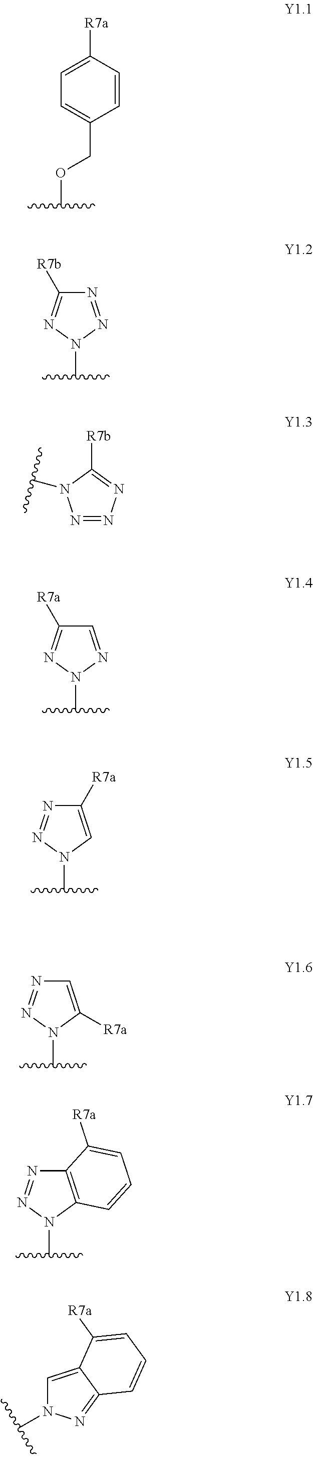

Y2 is cyano, C₁-C₆alkoxy, C₁-C₆haloalkoxy, C₃-C₆halocycloalkyl, C₁-C₄alkylsulfanyl, C₁-C₄alkylsulfinyl, C₁-C₄alkylsulfonyl, C₁-C₄haloalkylsulfanyl, C₁-C₄haloalkylsulfinyl, C₁-C₄haloalkylsulfonyl, benzyloxy, or Y2.1-Y2.8;

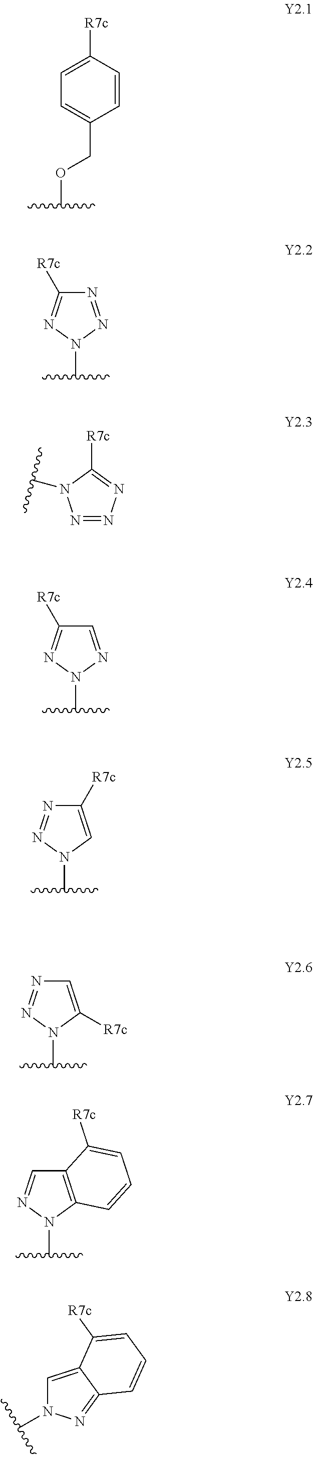

Y3 is cyano, C₉-C₆haloalkoxyC₃-C₆halocycloalkyl, C₁-C₄alkylsulfanyl, C₁-C₄alkylsulfinyl, C₁-C₄alkylsulfonyl, C₁-C₄haloalkylsulfanyl, C₁-C₄haloalkylsulfinyl, C₁-C₄haloalkylsulfonyl, benzyloxy, halobenzyloxy, or Y3.1-Y3.8;

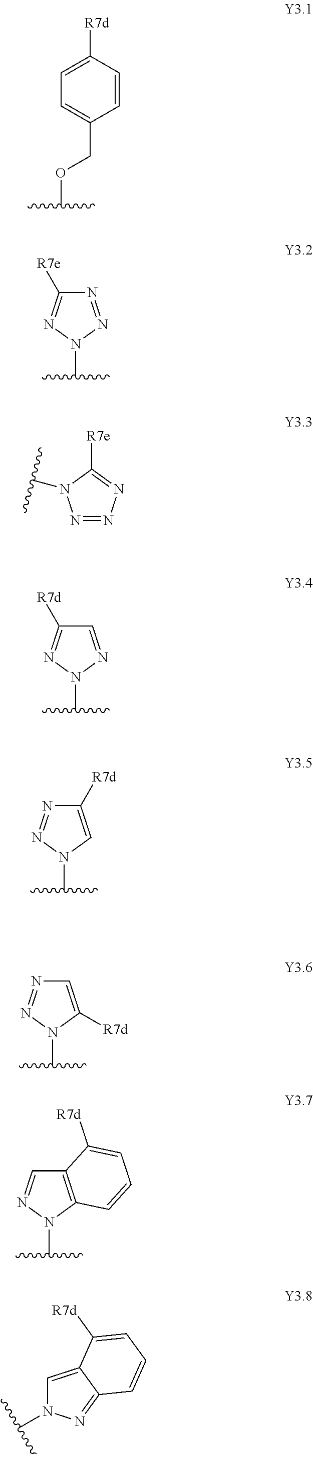

Y4 is cyano, C₁-C₆alkoxy, C₁-C₆haloalkoxy, C₃-C₆halocycloalkyl, C₁-C₄alkylsulfanyl, C₁-C₄alkylsulfinyl, C₁-C₄alkylsulfonyl, C₉-C₄haloalkylsulfanyl, C₁-C₄haloalkylsulfinyl, C₁-C₄haloalkylsulfonyl, benzyloxy, halobenzyloxy, or Y4.1-Y4.8,

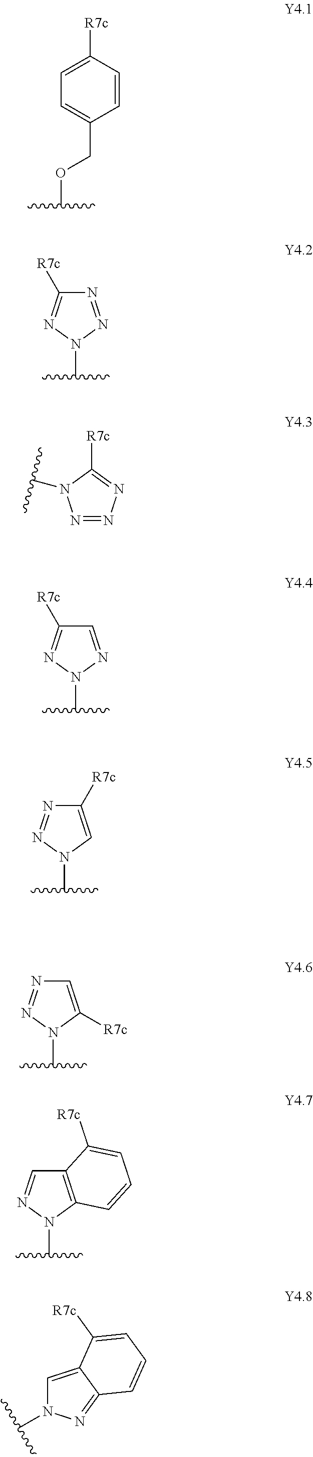

Y5 is cyano, C₁-C₆alkoxy, C₁-C₆haloalkoxy, C₃-C₆halocycloalkyl, C₁-C₄alkylsulfanyl, C₁-C₄alkylsulfinyl, C₁-C₄alkylsulfonyl, C₁-C₄haloalkylsulfanyl, C₁-C₄haloalkylsulfinyl, C₁-C₄haloalkylsulfonyl, benzyloxy, halobenzyloxy, or Y5.1-Y5.8;

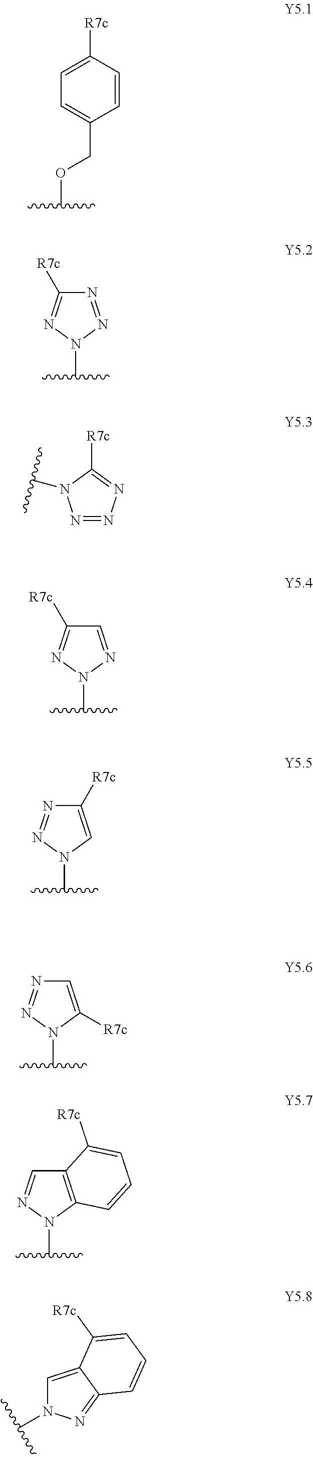

Y6 is cyano, C₁-C₆haloalkoxy, C₃-C₆halocycloalkyl, C₁-C₄alkylsulfanyl, C₁-C₄alkylsulfinyl, C₁-C₄alkylsulfonyl, C₁-C₄haloalkylsulfanyl, C₁-C₄haloalkylsulfinyl, C₁-C₄haloalkylsulfonyl, benzyloxy, halobenzyloxy, or Y6.1-Y6.8;

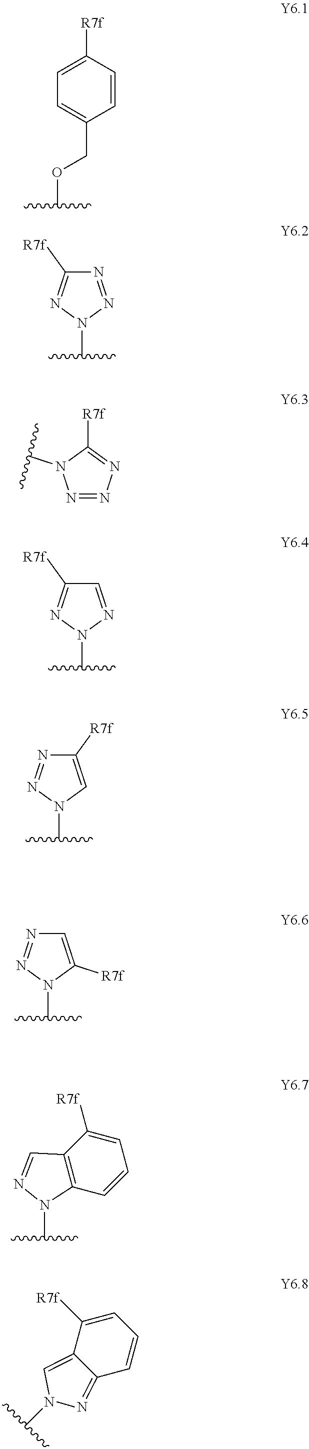

Y7 is cyano, C₁-C₆alkoxy, C₁-C₆haloalkoxy, C₃-C₆halocycloalkyl, C₁-C₄alkylsulfanyl, C₁-C₄alkylsulfinyl, C₁-C₄alkylsulfonyl, C₁-C₄haloalkylsulfanyl, C₁-C₄haloalkylsulfinyl, C₁-C₄haloalkylsulfonyl, benzyloxy, halobenzyloxy, or Y7.1-Y7.8;

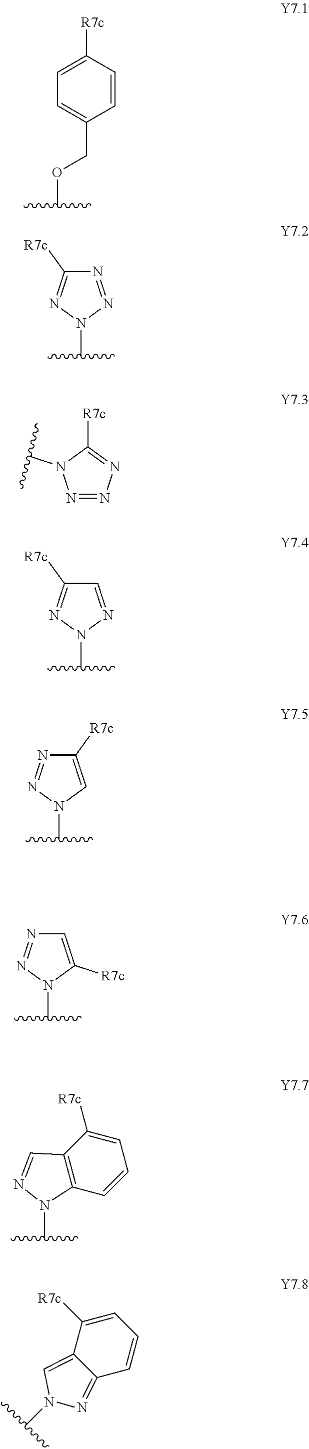

Y8 is cyano, C₁-C₆haloalkoxy, C₃-C₆halocycloalkyl, C₁-C₄alkylsulfanyl, C₁-C₄alkylsulfinyl, C₁-C₄alkylsulfonyl, C₁-C₄haloalkylsulfanyl, C₁-C₄haloalkylsulfinyl, C₁-C₄haloalkylsulfonyl, benzyloxy, halobenzyloxy, or Y8.1-Y8.8:

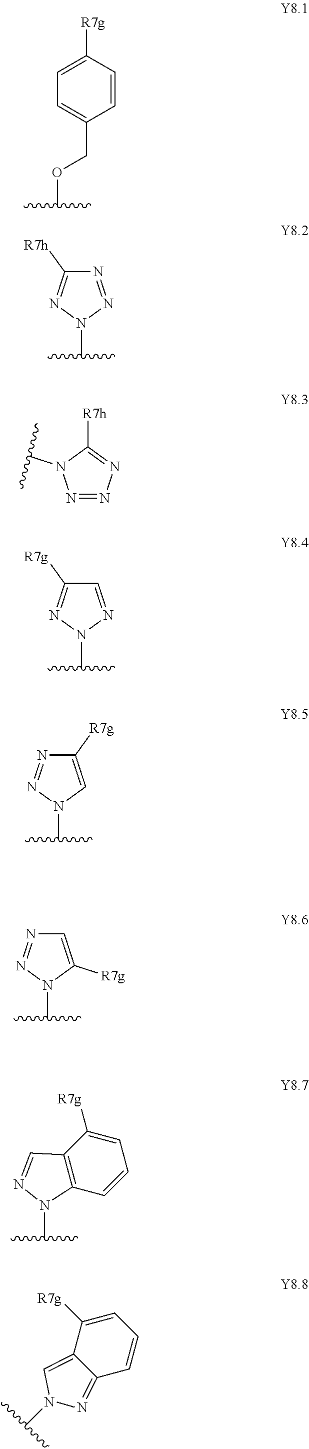

Y9 is cyano, C₁-C₆alkoxy, C₁-C₆haloalkoxy, C₃-C₆halocycloalkyl, C₁-C₄alkylsulfanyl, C₁-C₄alkylsulfinyl, C₁-C₄alkylsulfonyl, C₁-C₄haloalkylsulfanyl, C₁-C₄haloalkylsulfinyl, C₁-C₄haloalkylsulfonyl, benzyloxy, halobenzyloxy, or Y9.1-Y9.8;

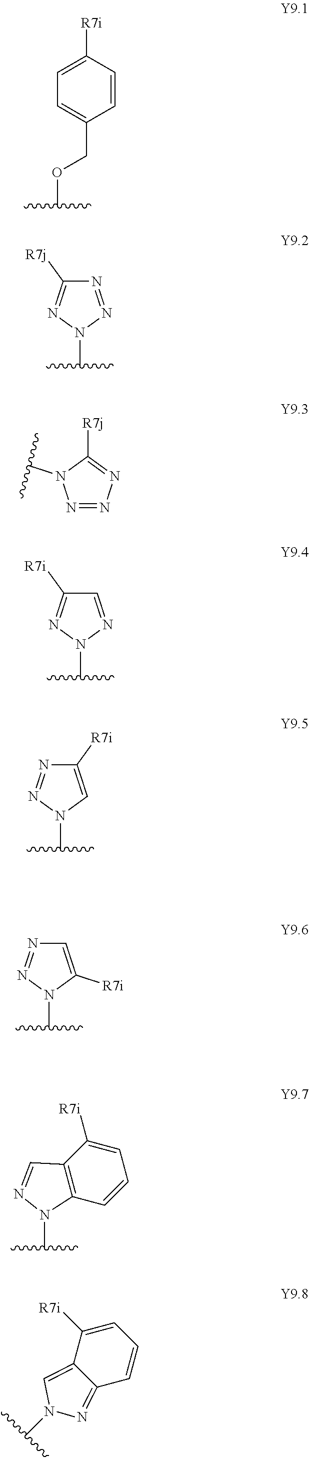

Y10 is cyano, C₃-C₆halocycloalkyl, C₁-C₄alkylsulfanyl, C₁-C₄alkylsulfinyl, C₁-C₄alkylsulfonyl, benzyloxy, halobenzyloxy, or Y10.1-Y10.8,

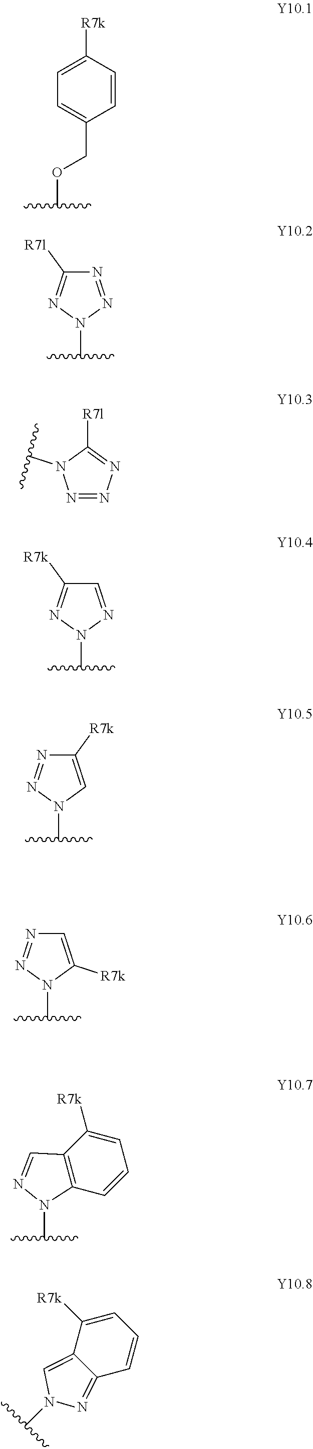

Y11 is cyano, C₁-C₆haloalkoxy, C₃-C₆halocycloalkyl, C₁-C₄alkylsulfanyl, C₁-C₄alkylsulfinyl, C₁-C₄alkylsulfonyl, C₁-C₄haloalkylsulfanyl, C₁-C₄haloalkylsulfinyl, C₁-C₄haloalkylsulfonyl, benzyloxy, halobenzyloxy, or Y11.1-Y11.8

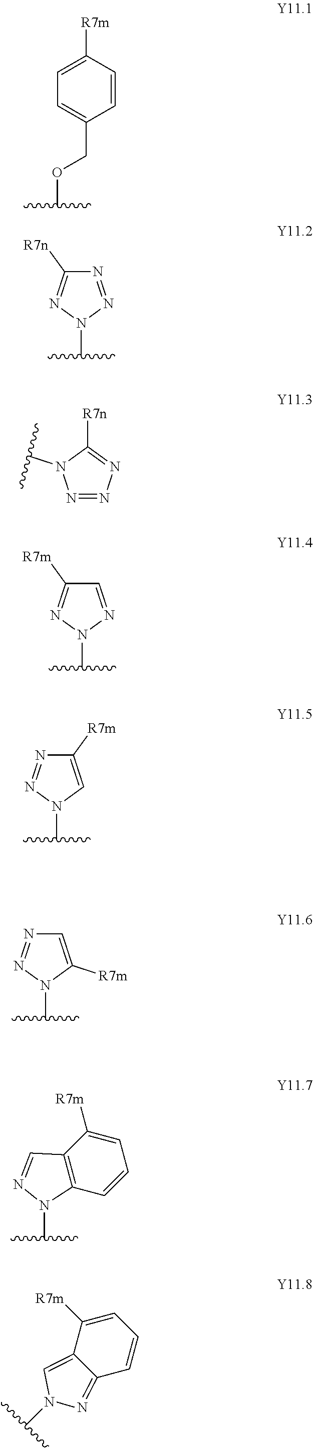

Y12 is cyano, C₂-C₆haloalkoxy, benzyloxy, halobenzyloxy, or Y12.1-Y12.8:

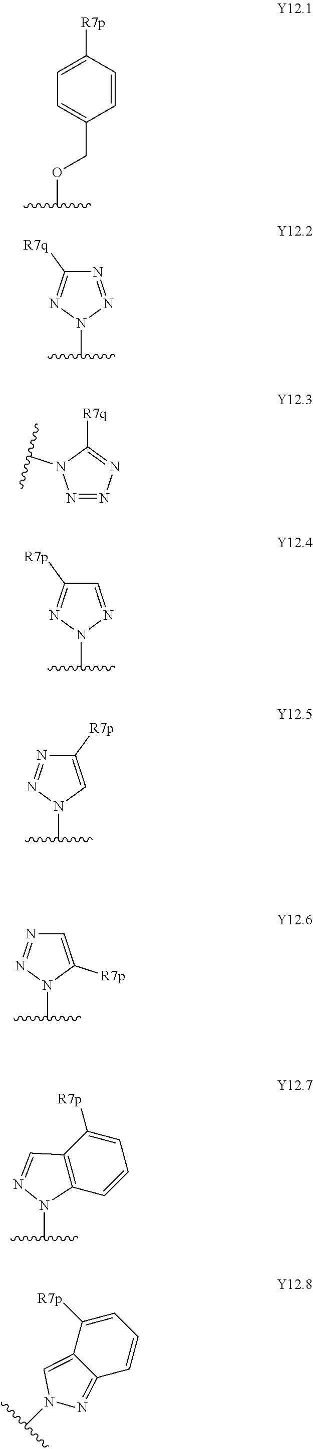

Y13 is cyano, C₁-C₆alkoxy, C₁-C₆haloalkoxy, C₃-C₆halocycloalkyl, C₁-C₄alkylsulfanyl, C₁-C₄alkylsulfinyl, C₁-C₄alkylsulfonyl, C₁-C₄haloalkylsulfanyl, C₁-C₄haloalkylsulfinyl, C₁-C₄haloalkylsulfonyl, benzyloxy, halobenzyloxy, or Y13.1-Y13.8;

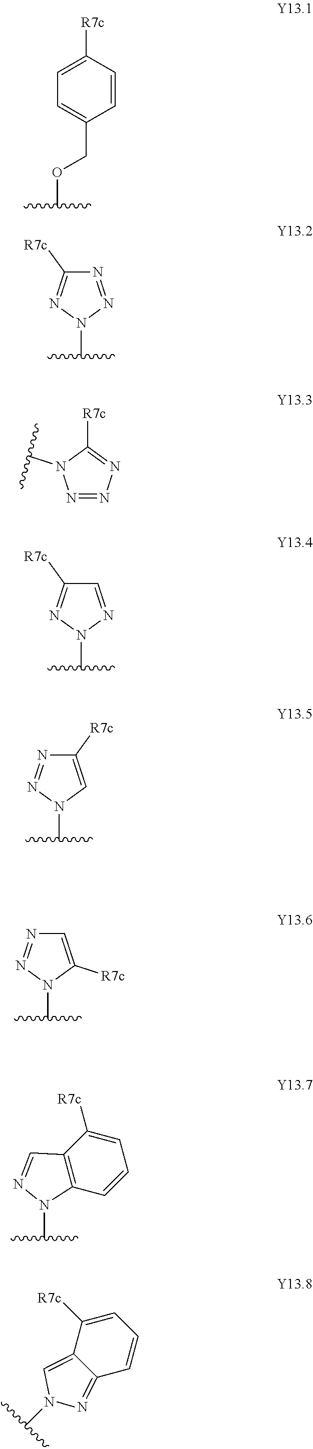

R₇ₐ is independently selected from hydrogen, halogen, cyano, C₁-C₃alkyl, C₁-C₃haloalkyl, C₃-C₆cycloalkyl, (C₁-C₃alkyl)NHC(O), (C₁-C₃alkyl)₂NC(O), (C₁-C₃cycloalkyl)NHC(O), and (C₁-C₃cycloalkyl)(C₁-C₃alkyl)NC(O), phenyl, and 6-membered heteroaromatic ring wherein the phenyl or the 6-membered ring is optionally substituted with one to three substituents independently selected from halogen, cyano, C₁-C₄alkyl, C₁-C₃alkoxy and C₁-C₃haloalkyl;
R_{7b} is independently selected from halogen, cyano, C₁-C₃alkyl, C₂-C₃haloalkyl, CHF₂, C₃-C₆cycloalkyl, (C₁-C₃alkyl)NHC(O), (C₁-C₃alkyl)₂NC(O), (C₁-C₃cycloalkyl)NHC(O), (C₁-C₃cycloalkyl)(C₁-C₃alkyl)NC(O), phenyl, and 6-membered heteroaromatic ring wherein the phenyl or the 6-membered ring is optionally substituted with one to three substituents independently selected from halogen, cyano, C₁-C₄alkyl, C₁-C₃alkoxy and C₁-C₃haloalkyl:
R_{7c} is independently selected from halogen, cyano, C₁-C₃alkyl, C₁-C₃haloalkyl, C₃-C₆cycloalkyl, (C₁-C₃alkyl)NHC(O), (C₁-C₃alkyl)₂NC(O), (C₁-C₃cycloalkyl)NHC(O), (C₁-C₃cycloalkyl)(C₁-C₃alkyl)NC(O), phenyl, and 6-membered heteroaromatic ring wherein the phenyl or the 6-membered ring is optionally substituted with one to three substituents independently selected from halogen, cyano, C₁-C₄alkyl, C₁-C₃alkoxy and C₁-C₃haloalkyl;
R_{7d} is independently selected from hydrogen, halogen, cyano, C₁-C₃alkyl, C₁-C₃haloalkyl, C₃-C₆cycloalkyl, (C₁-C₃alkyl)NHC(O), (C₁-C₃alkyl)₂NC(O), (C₁-C₃cycloalkyl)NHC(O), (C₁-C₃cycloalkyl)(C₁-C₃alkyl)NC(O), and phenyl, wherein the phenyl is unsubstituted or substituted by one to three substituents selected from halogen, cyano, C₁-C₄alkyl, C₁-C₃alkoxy and C₁-C₃haloalkyl;
R₇ₑ is independently selected from: halogen, cyano, C₁-C₃alkyl, C₂-C₃haloalkyl, CHF₂, C₃-C₆cycloalkyl, (C₁-C₃alkyl)NHC(O), (C₁-C₃alkyl)₂NC(O), (C₁-C₃cycloalkyl)NHC(O), (C₁-C₃cycloalkyl)(C₁-C₃alkyl)NC(O), phenyl, and 6-membered heteroaromatic ring wherein the phenyl or the 6-membered ring is optionally substituted with one to three substituents independently selected from halogen, cyano, C₁-C₄alkyl, C₁-C₃alkoxy and C₁-C₃haloalkyl;
R_{7f} is independently selected from hydrogen, halogen, cyano, C₁-C₃alkyl, C₃-C₆cycloalkyl, (C₁-C₃alkyl)NHC(O), (C₁-C₃alkyl)₂NC(O), (C₁-C₃cycloalkyl)NHC(O), (C₁-C₃cycloalkyl)(C₁-C₃alkyl)NC(O), phenyl, and 6-membered heteroaromatic ring wherein the phenyl or the 6-membered ring is optionally substituted with one to three substituents independently selected from halogen, cyano, C₁-C₄alkyl, C₁-C₃alkoxy and C₁-C₃haloalkyl;
R_{7g} is independently selected from: hydrogen, halogen, cyano, C₁-C₃alkyl, C₃-C₆cycloalkyl, (C₁-C₃alkyl)NHC(O), (C₁-C₃alkyl)₂NC(O), (C₁-C₃cycloalkyl)NHC(O), (C₁-C₃cycloalkyl)(C₁-C₃alkyl)NC(O), phenyl, and 6-membered heteroaromatic ring wherein the phenyl or the 6-membered ring is optionally substituted with one to three substituents independently selected from halogen, cyano, C₁-C₄alkyl, C₁-C₃alkoxy and C₁-C₃haloalkyl;
R₇ₕ is independently selected from: halogen, cyano, C₁-C₃alkyl, C₃-C₆cycloalkyl, (C₁-C₃alkyl)NHC(O), (C₁-C₃alkyl)₂NC(O), (C₁-C₃cycloalkyl)NHC(O), (C₁-C₃cycloalkyl)(C₁-C₃alkyl)NC(O), phenyl, and 6-membered heteroaromatic ring wherein the phenyl or the 6-membered ring is optionally substituted with one to three substituents independently selected from halogen, cyano, C₁-C₄alkyl, C₁-C₃alkoxy and C₁-C₃haloalkyl;
R₇ᵢ is independently selected from hydrogen, halogen, cyano, C₁-C₃alkyl, C₁-C₃haloalkyl, C₃-C₆cycloalkyl, (C₁-C₃alkyl)NHC(O), (C₁-C₃alkyl)₂NC(O), (C₁-C₃cycloalkyl)NHC(O), (C₁-C₃cycloalkyl)(C₁-C₃alkyl)NC(O), phenyl, and 6-membered heteroaromatic ring wherein the phenyl or the 6-membered ring is optionally substituted with one to three substituents independently selected from halogen, cyano, C₁-C₄alkyl, C₁-C₃alkoxy and C₁-C₃haloalkyl;
R₇ⱼ is independently selected from halogen, cyano, C₁-C₃alkyl, C₂-C₃haloalkyl, CHF₂, C₃-C₆cycloalkyl, (C₁-C₃alkyl)NHC(O), (C₁-C₃alkyl)₂NC(O), (C₁-C₃cycloalkyl)NHC(O), (C₁-C₃cycloalkyl)(C₁-C₃alkyl)NC(O), phenyl, and 6-membered heteroaromatic ring wherein the phenyl or the 6-membered ring is optionally substituted with one to three substituents independently selected from halogen, cyano, C₁-C₄alkyl, C₁-C₃alkoxy and C₁-C₃haloalkyl;
R₇ₖ is independently selected from hydrogen, halogen, cyano, C₁-C₃alkyl, (C₁-C₃alkyl)NHC(O), (C₁-C₃alkyl)₂NC(O), (C₁-C₃cycloalkyl)NHC(O), (C₁-C₃cycloalkyl)(C₁-C₃alkyl)NC(O), phenyl, and 6-membered heteroaromatic ring wherein the phenyl or the 6-membered ring is optionally substituted with one to three substituents independently selected from halogen, cyano, C₁-C₄alkyl, C₁-C₃alkoxy and C₁-C₃haloalkyl;
R₇ₗ is independently selected from: halogen, cyano, C₁-C₃alkyl, C₁-C₃alkyl)NHC(O), (C₁-C₃alkyl)₂NC(O), (C₁-C₃cycloalkyl)NHC(O), (C₁-C₃cycloalkyl)(C₁-C₃alkyl)NC(O), phenyl, and 6-membered heteroaromatic ring wherein the phenyl or the 6-membered ring is optionally substituted with one to three substituents independently selected from halogen, cyano, C₁-C₄alkyl, C₁-C₃alkoxy and C₁-C₃haloalkyl;
R₇ₘ is independently selected from: hydrogen, halogen, cyano, C₁-C₃alkyl, C₁-C₃haloalkyl, C₃-C₆cycloalkyl, (C₁-C₃alkyl)NHC(O), (C₁-C₃alkyl)₂NC(O), (C₁-C₃cycloalkyl)NHC(O), (C₁-C₃cycloalkyl)(C₁-C₃alkyl)NC(O), phenyl, and 6-membered heteroaromatic ring wherein the phenyl or the 6-membered ring is optionally substituted with one to three substituents independently selected from halogen, cyano, C₁-C₄alkyl, C₁-C₃alkoxy and C₁-C₃haloalkyl;
R₇ₙ is independently selected from halogen, cyano, C₁-C₃alkyl, C₂-C₃haloalkyl, CHF₂, C₃-C₆cycloalkyl, (C₁-C₃alkyl)NHC(O), (C₁-C₃alkyl)₂NC(O), (C₁-C₃cycloalkyl)NHC(O), (C₁-C₃cycloalkyl)(C₁-C₃alkyl)NC(O), phenyl, and 6-membered heteroaromatic ring wherein the phenyl or the 6-membered ring is optionally substituted with one to three substituents independently selected from halogen, cyano, C₁-C₄alkyl, C₁-C₃alkoxy and C₁-C₃haloalkyl;
R₇ₚ is independently selected from hydrogen, halogen, cyano, C₁-C₃alkyl, (C₁-C₃alkyl)NHC(O), (C₁-C₃alkyl)₂NC(O), (C₁-C₃cycloalkyl)NHC(O), (C₁-C₃cycloalkyl)(C₁-C₃alkyl)NC(O), phenyl, and 6-membered heteroaromatic ring wherein the phenyl or the 6-membered ring is optionally substituted with one to three substituents independently selected from halogen, cyano, C₁-C₄alkyl, C₁-C₃alkoxy and C₁-C₃haloalkyl;
R_{7q} is independently selected from halogen, cyano, C₁-C₃alkyl, C₃-C₆cycloalkyl, (C₁-C₃alkyl)NHC(O), (C₁-C₃alkyl)₂NC(O), (C₁-C₃cycloalkyl)NHC(O), (C₁-C₃cycloalkyl)(C₁-C₃alkyl)NC(O), phenyl, and 6-membered heteroaromatic ring wherein the phenyl or the 6-membered ring is optionally substituted with one to three substituents independently selected from halogen, cyano, C₁-C₄alkyl, C₁-C₃alkoxy and C₁-C₃haloalkyl;
or an agronomically acceptable salt, isomer, enantiomer, tautomer and/or N-oxide of the compound of formula Ia, Ib, Ic or Id.

Through the use of a compound of formula I according to each aspect of the present invention the damage caused on plants, especially on useful plants and ornamentals in agriculture, in horticulture and in forests, or on organs, such as fruits, flowers, foliage, stalks, tubers or roots, of such plants, and in some cases even plant organs which are formed at a later point in time, by a diamide-resistant insect is controlled, reduced.

Compounds I which have at least one basic centre can form, for example, acid addition salts, for example with strong inorganic acids such as mineral acids, for example perchloric acid, sulfuric acid, nitric acid, nitrous acid, a phosphorus acid or a hydrohalic acid, with strong organic carboxylic acids, such as C₁-C₄alkanecarboxylic acids which are unsubstituted or substituted, for example by halogen, for example acetic acid, such as saturated or unsaturated dicarboxylic acids, for example oxalic acid, malonic acid, succinic acid, maleic acid, fumaric acid or phthalic acid, such as hydroxycarboxylic acids, for example ascorbic acid, lactic acid, malic acid, tartaric acid or citric acid, or such as benzoic acid, or with organic sulfonic acids, such as C₁-C₄alkane- or arylsulfonic acids which are unsubstituted or substituted, for example by halogen, for example methane- or p-toluenesulfonic acid. Compounds I which have at least one acidic group can form, for example, salts with bases, for example mineral salts such as alkali metal or alkaline earth metal salts, for example sodium, potassium or magnesium salts, or salts with ammonia or an organic amine, such as morpholine, piperidine, pyrrolidine, a mono-, di- or tri-lower-alkylamine, for example ethyl-, diethyl-, triethyl- or dimethylpropylamine, or a mono-, di- or trihydroxy-lower-alkylamine, for example mono-, di- or triethanolamine. Where appropriate, the corresponding internal salts can furthermore be formed. Preferred within the scope of the invention are agrochemically advantageous salts; however, the invention also encompasses salts which have disadvantage for agrochemical use, for example salts which are toxic to bees or fish, and which are employed, for example, for the isolation or purification of free compounds I or agrochemically utilizable salts thereof. Owing to the close relationship between the compounds I in free form and in the form of their salts, for the purposes of the invention the free compounds I or their salts hereinabove and hereinbelow are respectively to be understood as including, where appropriate, the corresponding salts or the free compounds I. The same applies analogously to tautomers of compounds I and salts thereof. In general, the free form is preferred in each case.

N-oxides are oxidized forms of tertiary amines or oxidized forms of nitrogen containing heteroaromatic compounds. They are described for instance in the book “Heterocyclic N-oxides” by A. Albini and S. Pietra, CRC Press, Boca Raton 1991.

The compounds of formula I according to the invention also include hydrates which may be formed during the salt formation.

The term “C₁-Cₙalkyl” as used herein refers to a saturated straight-chain or branched hydrocarbon radical attached via any of the carbon atoms having 1 to n carbon atoms, for example, any one of the radicals methyl, ethyl, n-propyl, 1-methylbutyl, 2-methylbutyl, 3-methylbutyl, 2,2-dimethylpropyl, 1-ethylpropyl, n-hexyl, n-pentyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, or 1-ethyl-2-methylpropyl.

The term “C₁-Cₙhaloalkyl” as used herein refers to a straight-chain or branched saturated alkyl radical attached via any of the carbon atoms having 1 to n carbon atoms (as mentioned above), where some or all of the hydrogen atoms in these radicals may be replaced by a halogen independently selected from fluorine, chlorine, bromine and iodine, i.e., for example, any one of chloromethyl, dichloromethyl, trichloromethyl, fluoromethyl, difluoromethyl, trifluoromethyl, chlorofluoromethyl, dichlorofluoromethyl, chlorodifluoromethyl, 2-fluoroethyl, 2-chloroethyl, 2-bromoethyl, 2-iodoethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl, 2-fluoropropyl, 3-fluoropropyl, 2,2-difluoropropyl, 2,3-difluoropropyl, 2-chloropropyl, 3-chloropropyl, 2,3-dichloropropyl, 2-bromopropyl, 3-bromopropyl, 3,3,3-trifluoropropyl, 3,3,3-trichloropropyl, 2,2,3,3,3-pentafluoropropyl, heptafluoropropyl, 1-(fluoromethyl)-2-fluoroethyl, 1-(chloromethyl)-2-chloroethyl, 1-(bromomethyl)-2-bromoethyl, 4-fluorobutyl, 4-chlorobutyl, 4-bromobutyl or nonafluorobutyl. According a term “C₁-C₂fluoroalkyl” would refer to a C₁-C₂alkyl radical which carries 1, 2, 3, 4, or 5 fluorine atoms, for example, any one of difluoromethyl, trifluoromethyl, 1-fluoroethyl, 2-fluoroethyl, 2,2-difluoroethyl, 2,2,2-trifluoroethyl, 1,1,2,2-tetrafluoroethyl or pentafluoroethyl.

The term “C₁-Cₙalkoxy” as used herein refers to a straight-chain or branched saturated alkyl radical having 1 to n carbon atoms (as mentioned above) which is attached via an oxygen atom, i.e., for example, any one of the radicals methoxy, ethoxy, n-propoxy, 1-methylethoxy, n-butoxy, 1-methylpropoxy, 2-methylpropoxy or 1,1-dimethylethoxy. The term “haloC₁-Cₙalkoxy” as used herein refers to a C₁-Cₙalkoxy radical where one or more hydrogen atoms on the alkyl radical is replaced by the same or different halo atom(s)—examples include trifluoromethoxy, 2-fluoroethoxy, 3-fluoropropoxy, 3,3,3-trifluoropropoxy, 4-chlorobutoxy.

The term “C₃-Cₙcycloalkyl” as used herein refers to 3-n membered cycloalkyl groups such as cyclopropane, cyclobutane, cyclopentane and cyclohexane.

The term “C₂-Cₙhalocycloalkyl” as used herein refers to a C₃-Cₙcycloalkyl moiety substituted with one or more halo atoms which may be the same or different.

The term “C₁-Cₙalkanediyl” as used herein refers to a saturated straight-chain or branched hydrocarbon radical connected via two single bonds from one or more of its carbon atom(s) to two other groups, for example, acting like a spacer between two groups. Examples are methylene (or —CH₂—) and the ethylene (—CH₂CH₂—).

The term “C₁-Cₙhaloalkanediyl” as used herein refers to a C₁-Cₙalkanediyl moiety substituted with one or more halo atoms which may be the same or different.

The term “C₃-Cₙcycloalkylcarbonyloxy” as used herein refers to a 3-n membered cycloalkyl group attached to a carbonyloxy (C(O)₂) group, and oxygen atom of which carbonyloxy group is connected to the rest of the molecule. Similarly the term “C₁-Cₙalkylcarbonyoxy” as used herein refers to an alkyl group attached to a carbonyloxy (C(O)₂) group, and oxygen atom of which carbonyloxy group is connected to the rest of the molecule.

The term “C₁-Cₙalkylsulfanyl” as used herein refers to a C₁-Cₙalkyl moiety linked through a sulfur atom. Similarly, the term “C₁-Cₙhaloalkylthio” or “C₁-Cₙhaloalkylsulfanyl” as used herein refers to a C₁-Cₙhaloalkyl moiety linked through a sulfur atom.

The term “C₁-Cₙalkylsulfinyl” as used herein refers to a C₁-Cₙalkyl moiety linked through the sulfur atom of the S(═O) group. Similarly, the term “C₁-Cₙhaloalkylsulfinyl” or “C₁-Cₙhaloalkylsulfinyl” as used herein refers to a C₁-Cₙhaloalkyl moiety linked through the sulfur atom of the S(═O) group.

The term “C₁-Cₙalkylsulfonyl” as used herein refers to a C₁-Cₙalkyl moiety linked through the sulfur atom of the S(═O)₂ group. Similarly, the term “C₁-Cₙhaloalkylsulfonyl” or “C₁-Cₙhaloalkylsulfonyl” as used herein refers to a C₁-Cₙhaloalkyl moiety linked through the sulfur atom of the S(═O)₂ group.

The term “C₂-Cₙalkenyl” as used herein refers to a straight or branched alkenyl chain having from two to n carbon atoms and one or two double bonds, for example, ethenyl, prop-1-enyl, but-2-enyl.

The term “C₂-Cₙalkynyl” as used herein refers to a straight or branched alkynyl chain having from two to n carbon atoms and one triple bond, for example, ethynyl, prop-2-ynyl, but-3-ynyl.

Halogen or “halo” is generally fluorine, chlorine, bromine or iodine. This also applies, correspondingly, to halogen in combination with other meanings, such as haloalkyl

The term “6-membered heteroaromatic” refers to a 6 membered aromatic ring having 1 to 3 carbon atoms replaced independently by nitrogen, sulfur, or oxygen. Examples are pyridyl (or pyridinyl), pyridazinyl, pyrimidinyl, and pyrazinyl.

Examples of “5- or 6-membered heteroaromatic” refers to a 5 or 6 membered aromatic ring having 1 to 3 carbon atoms replaced independently by nitrogen, sulfur, or oxygen. Examples are pyridyl (or pyridinyl), pyridazinyl, pyrimidinyl, pyrazinyl, pyrrolyl, pyrazolyl, imidazolyl, triazolyl (e.g. 1.2.4 triazoyl), furanyl, thiophenyl, oxazolyl, isoxazolyl, oxadiazolyl, thiazolyl, isothiazolyl and thiadiazolyl.

Examples of “9- or 10-membered heteroaromatic” refers to a 9 or 10 membered aromatic ring made up of two rings, having 1 to 4 carbon atoms replaced independently by nitrogen, sulfur, or oxygen (the heteroatoms can be in one ring or distributed amongst the two). Examples are purinyl, quinolinyl, cinnolinyl, quinoxalinyl, indolyl, indazolyl, benzimidazolyl, benzothiophenyl, benzoxazolyl, benzothiazolyl, imidazo[1,2-a]pyridinyl, and imidazo[4,5-b]pyridinyl.

As used herein, the term “controlling” refers to reducing the number of pests (or insects), eliminating pests and/or preventing further pest damage such that damage to a plant or to a plant derived product is reduced. The insect encompasses all stages in the life cycle of the insect.

As used herein, the term “effective amount” refers to the amount of the compound, or a salt thereof, which, upon single or multiple applications provides the desired effect.

The staggered line as used herein, for example, in Ya1 to Ya17, represent the point of connection/attachment to the rest of the compound.

An effective amount is readily determined by the skilled person in the art, by the use of known techniques and by observing results obtained under analogous circumstances. In determining the effective amount a number of factors are considered including, but not limited to: the type of plant or derived product to be applied; the pest to be controlled & its lifecycle; the particular compound applied; the type of application; and other relevant circumstances.

In a preferred embodiment of the first aspect of the present invention, compound of formula I is represented by formula I-I

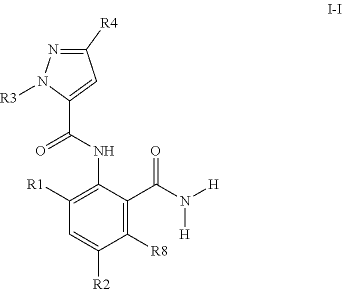

where R₁, R₂, R₃, R₄, and R₈ are as defined in the first aspect.

In a further preferred embodiment of the first aspect of the present invention, compound of formula I is represented by formulae Ia, Ib, Ic, and Id.

Embodiments according to the present invention are provided as set out below.

In an embodiment of each aspect of the invention, A is
A. sulfur; or
B. oxygen.

In an embodiment of each aspect of the invention, R₈ is
A. H, halogen, cyano, or C₁-C₆alkyl; or
B. H, halogen, cyano, or C₁-C₃alkyl; or
C. H, F, Cl, Br, I, cyano, or C₁-C₃alkyl; or
D. H, F, Cl, Br, I, or Me; or
E. H, F, or Cl; or
F. H, or F; or
G. H.

In an embodiment of each aspect of the invention, X2 is
A. C₁-C₃alkanediyl, or C₁-C₃haloalkanediyl; or
B. CH₂ or CF₂; or
C. CH₂.

In an embodiment of the first aspect of the invention, R₁ is
A. hydrogen, halogen, cyano, C₁-C₃alkyl, C₂-C₄alkenyl, C₂-C₄alkynyl, C₁-C₃haloalkyl, C₃-C₄cycloalkyl, C₁-C₃alkoxy, or C₁-C₃haloalkoxy; or
B. halogen, or C₁-C₃alkyl; or
C. methyl, F, Cl, Br, or I; or
D. methyl, Cl, or Br; or
E. methyl or chlorine.

In an embodiment of the first aspect of the invention, R₂ is
A. cyano, F, Cl, Br, or I; or
B. cyano, chlorine, bromine, or iodine; or
C. bromine or chlorine.

In an embodiment of the first aspect of the invention, R₃ is
A. phenyl, or a 6-membered heteroaromatic ring, each of which is substituted with one to three substituents independently selected from R₆; or
B. a 6-membered heteroaromatic ring, each of which is substituted with one to three substituents independently selected from R₆; or
C. pyridyl ring, which is unsubstituted or is substituted with one to three substituents independently selected from R₆; or
D. pyridyl ring, which is substituted with one or two substituents independently selected from halogen, cyano, and C₁-C₃alkyl; or
E. pyridyl ring, which is substituted with one or two substituents independently selected from chlorine, bromine, iodine, cyano, and methyl; or
F. pyridyl ring, which is substituted with one or two substituents independently selected from chlorine, bromine, and iodine; or
G. pyridyl ring, which is substituted with one or two substituents chlorine; or
H. pyrid-2-yl ring, which is substituted with one or two substituents chlorine; or
I. 3-chloro-pyrid-2-yl ring or 3,5-dichloro-pyrid-2-yl ring; or
J. 3-chloro-pyrid-2-yl ring.

In an embodiment of the first aspect of the invention, Re is
A. independently selected from halogen, cyano, C₁-C₃alkyl, C₁-C₃haloalkyl, C₁-C₃haloalkylthio, C₁-C₃alkoxy, and C₁-C₃haloalkoxy; or
B. independently selected from halogen, cyano, and C₁-C₃alkyl; or
C. independently selected from halogen; or
D. chlorine.

In an embodiment of the first aspect of the invention, R₄ is
A. hydrogen, halogen, C₁-C₃alkyl, C₂-C₄alkenyl, C₂-C₄alkynyl, C₁-C₃haloalkyl, C₃-C₄cycloalkyl, C₃-C₄cyanocycloalkyl, C₃-C₄halocycloalkyl, C₁-C₃alkoxy, C₁-C₃haloalkoxy, or X2-Y; or
B. halogen, C₁-C₃haloalkyl, C₁-C₃alkoxy, C₁-C₃haloalkoxy, or X2-Y; or
C. trifluoromethyl, bromine, chlorine, methoxy or X2-Y; or
D. trifluoromethyl; or
E. bromine; or
F. chlorine; or
G. methoxy; or
H. X2-Y.

In an embodiment of the first aspect of the invention, Y is
A. cyano, C₂-C₄alkenyl, C₂-C₄alkynyl, C₁-C₃alkoxy, C₁-C₃haloalkoxy, C₃-C₄halocycloalkyl, C₁-C₃alkylsulfanyl, C₁-C₃alkylsulfinyl, C₁-C₃alkylsulfonyl, C₁-C₃haloalkylsulfanyl, C₁-C₃haloalkylsulfinyl, C₁-C₃haloalkylsulfonyl, benzyloxy, halobenzyloxy, 5- or 6-membered heteroaromatic ring, which is unsubstituted or substituted with one to three groups independently selected from R₇, or a 9- or 10-membered heteroaromatic bicyclic system, which is unsubstituted or substituted with one to three groups independently selected from R₇; or
B. cyano, C₁-C₃alkoxy, C₁-C₃haloalkoxy, C₃-C₄halocycloalkyl, C₁-C₃alkylsulfanyl, C₁-C₃alkylsulfinyl, C₁-C₃alkylsulfonyl, C₁-C₃haloalkylsulfanyl, C₁-C₃haloalkylsulfinyl, C₁-C₃haloalkylsulfonyl, benzyloxy, halobenzyloxy, 5- or 6-membered heteroaromatic ring, which is unsubstituted or substituted with one to three groups independently selected from R₇, or a 9- or 10-membered heteroaromatic bicyclic system, which is unsubstituted or substituted with one to three groups independently selected from R₇; or
C. C₁-C₃alkoxy, C₁-C₃haloalkoxy, halobenzyloxy, 5- or 6-membered heteroaromatic ring, which is unsubstituted or substituted with one to three groups independently selected from R₇, or a 9- or 10-membered heteroaromatic bicyclic system, which is unsubstituted or substituted with one to three groups independently selected from R₇; or
D. C₁-C₃alkoxy, C₁-C₃haloalkoxy, or any one of Ya to Yj; or

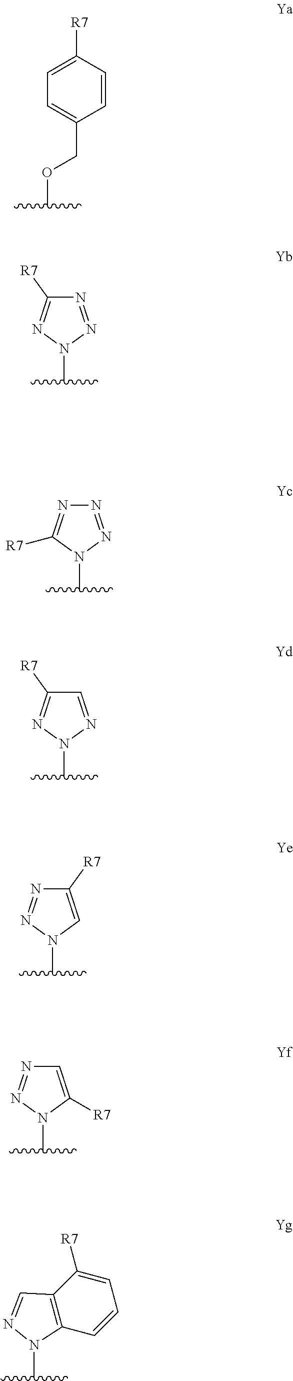

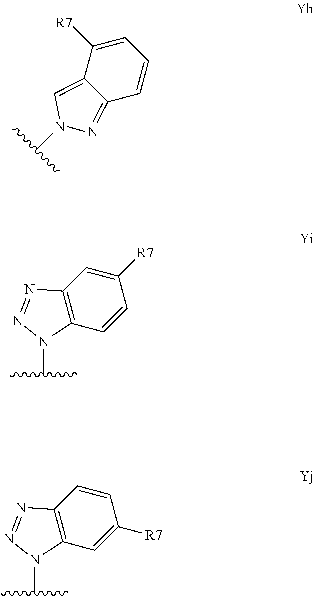

E. selected from Ya to Yj;
F. selected from Ya to Yh; or
G. Yb, Yc, Yd, Ye, Yf, Yg, or Yh; or
H. Yb, Yc, Yd, Ye or Yg, or Yh, where, independently of Y, R₇ is selected from chlorine, bromine, fluorine, difluoromethyl, trifluoromethyl, cyclopropyl or phenyl substituted by trifluoromethyl; or
I. selected from Ya1 to Ya17; or
J. selected from Ya1 to Ya6.

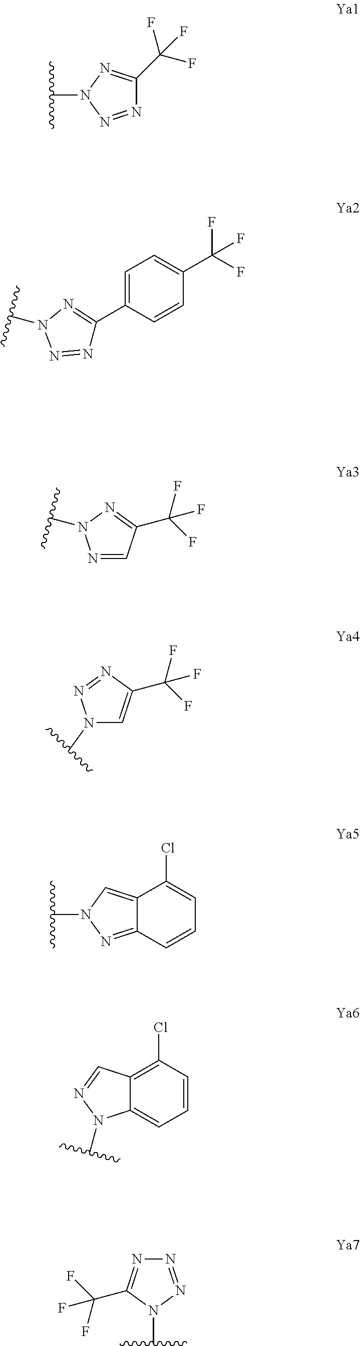

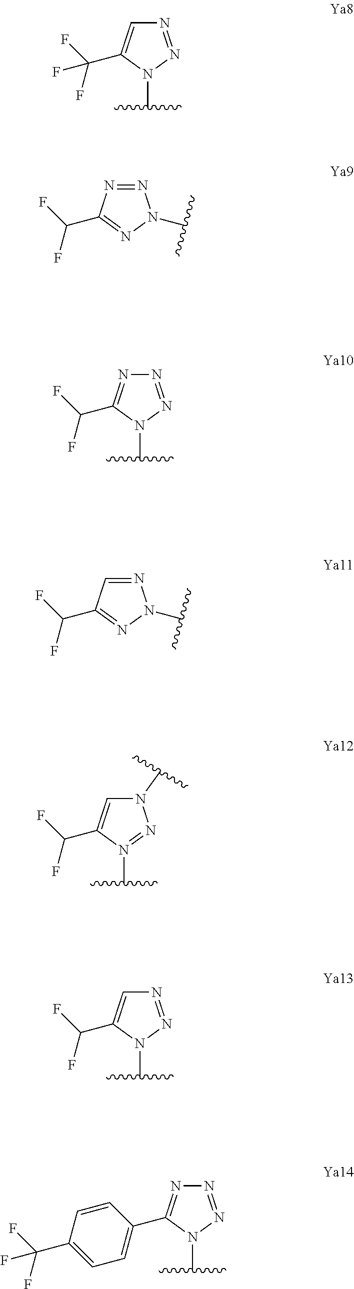

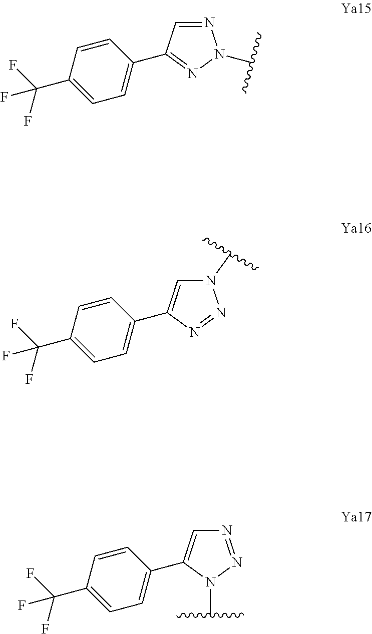 [SMILES: *n1ncc(-c2ccc(C(F)(F)F)cc2)n1 ; *n1cc(-c2ccc(C(F)(F)F)cc2)nn1 ; *n1nncc1-c1ccc(C(F)(F)F)cc1]

In an embodiment of the first aspect of the invention, R₇ is
A. independently selected from halogen, cyano, C₁-C₃alkyl, C₁-C₃haloalkyl, C₃-C₆cycloalkyl, phenyl substituted with one more selected from halogen, cyano, C₁-C₃alkyl, C₁-C₃alkoxy, and C₁-C₃haloalkyl, and pyridinyl which may be substituted with one more selected from halogen, cyano, C₁-C₃alkvl, C₁-C₃alkoxy, and C₁-C₃haloalkyl; or
B. halogen, C₁-C₃alkyl, C₁-C₃haloalkyl, C₃-C₆cycloalkyl and phenyl substituted by chlorine, bromine, trifluoromethyl or difluoromethyl; or
C. halogen, C₁-C₃haloalkyl, C₃-C₆cycloalkyl and phenyl substituted by chlorine, bromine, trifluoromethyl or difluoromethyl or
D. chlorine, bromine, fluorine, difluoromethyl, trifluoromethyl, cyclopropyl or phenyl substituted by trifluoromethyl.

In an embodiment of each aspect of the invention, in respect of formula Ia (where R₂ from formula I is bromine),
R₁ is
   A. halogen, C₁-C₃alkyl, C₂-C₄alkenyl, C₂-C₄alkynyl, C₁-C₃haloalkyl, or C₃-C₄cycloalkyl; or
   B. halogen or C₁-C₆alkyl; or
   C. methyl, F, Cl, Br, or I; or
   D. methyl, C₁, or Br; or
   E. methyl or chlorine.
R₄ is provided above in respect of the first aspect (where Y1 to Y4 corresponds to the equivalent Y embodiment, and Yn.1 to Yn.8 (where n can be 1, 2, 3 or 4) corresponds to Ya to Yh, and its preferred embodiments G, H and I of Y above), wherein,
   Y1, in an embodiment is
      a) C₁-C₃haloalkoxy or Y1.1 to Y1.8; or
      b) trifluoromethoxy, difluoromethoxy or Y1.1 to Y1.8; or
      c) Yb, Yc, Yd, Ye, Yf, Yg, or Yh; or
      d) Yb, Yc, Yd, Ye or Yg, or Yh, where, independently of Y, R₇ is selected from chlorine, bromine, fluorine, difluoromethyl, trifluoromethyl, cyclopropyl or phenyl substituted by trifluoromethyl; or
      e) selected from Ya1 to Ya6.
   Y2, in an embodiment is
      a) C₁-C₃alkoxy, C₁-C₃haloalkoxy or Y2.1 to Y2.8; or
      b) methoxy, trifluoromethoxy, difluoromethoxy or Y2.1 to Y2.8; or
      c) Yb, Yc, Yd, Ye, Yf, Yg, or Yh; or
      d) Yb, Yc, Yd, Ye or Yg, or Yh, where, independently of Y, R₇ is selected from chlorine, bromine, fluorine, difluoromethyl, trifluoromethyl, cyclopropyl or phenyl substituted by trifluoromethyl; or
      e) selected from Ya1 to Ya6.
   Y3, in an embodiment is
      a) C₁-C₃haloalkoxy or Y3.1 to Y3.8; or
      b) trifluoromethoxy, difluoromethoxy or Y3.1 to Y3.8; or
      c) Yb, Yc, Yd, Ye, Yf, Yg, or Yh; or
      d) Yb, Yc, Yd, Ye or Yg, or Yh, where, independently of Y, R₇ is selected from chlorine, bromine, fluorine, difluoromethyl, trifluoromethyl, cyclopropyl or phenyl substituted by trifluoromethyl; or
      e) selected from Ya1 to Ya6.
   Y4, in an embodiment is
      a) C₁-C₃alkoxy, C₁-C₃haloalkoxy or Y4.1 to Y4.8; or
      b) methoxy, trifluoromethoxy, difluoromethoxy or Y4.1 to Y4.8; or
      c) Yb, Yc, Yd, Ye, Yf, Yg, or Yh; or
      d) Yb, Yc, Yd, Ye or Yg, or Yh, where, independently of Y, R₇ is selected from chlorine, bromine, fluorine, difluoromethyl, trifluoromethyl, cyclopropyl or phenyl substituted by trifluoromethyl; or
      e) selected from Ya1 to Ya6.
R₃ is
   A. pyridyl ring, which is unsubstituted or is substituted with one to three substituents independently selected from R₆; or
B. pyridyl ring, which is substituted with one or two substituents independently selected from halogen, cyano, and C₁-C₃alkyl; or
C. pyridyl ring, which is substituted with one or two substituents independently selected from chlorine, bromine, iodine, cyano, and methyl; or
D. pyridyl ring, which is substituted with one or two substituents independently selected from chlorine, bromine, and iodine; or
E. pyridyl ring, which is substituted with one or two substituents chlorine; or
F. pyrid-2-yl ring, which is substituted with one or two substituents chlorine; or
G. 3-chloro-pyrid-2-yl ring or 3,5-dichloro-pyrid-2-yl ring; or
H. 3-chloro-pyrid-2-yl ring.
R₆ is
   A. independently selected from halogen, C₁-C₃alkyl, and C₁-C₃haloalkyl; or
   B. independently selected from chlorine, bromine, and iodine; or
   C. chlorine.
R₈ is provided above in respect of the first aspect;
(I) wherein in the instance R₁ is chlorine,
   a. R₄ is
      i. fluorine, iodine, C₁-C₃alkoxy, or X2-Y1; or
      ii. fluorine, iodine, methoxy, or X2-Ya1 to X2-Ya6;
(II) wherein in the instance R₁ is fluorine or bromine,
   a. R₄ is
      i. fluorine, iodine, C₁-C₃haloalkyl, C₁-C₃alkoxy, C₁-C₃haloalkoxy, or X2-Y2; or
      ii. fluorine, iodine, trifluoromethyl, methoxy or X2-Y2; or
      iii. fluorine, iodine, trifluoromethyl, methoxy or X2-Ya1 to X2-Ya6;
(III) wherein in the instance R₁ is methyl,
   a. R₄ is
      i. iodine, C₁-C₃alkoxy, or X2-Y3; or
      ii. iodine, methoxy, or X2-Y3; or
      iii. iodine, methoxy or Ya1 to Ya6;
(IV) wherein in the instance R₁ is hydrogen, iodine, C₂-C₆alkyl, C₂-C₆alkenyl, C₂-C₆alkynyl, C₁-C₆haloalkyl, or C₃-C₆cycloalkyl,
   a. R₄ is
      i. halogen, C₁-C₃haloalkyl, C₁-C₃alkoxy, C₁-C₃haloalkoxy, or X2-Y4; or
      ii. trifluoromethyl, bromine, chlorine, methoxy, or X2-Y4; or
      iii. trifluoromethyl, bromine, chlorine, methoxy, or X2-Ya1 to X2-Ya6.

In an embodiment of each aspect of the invention, in respect of formula Ib (where R₂ from formula I is iodine),
R₁ is
   A. halogen, C₁-C₃alkyl, C₂-C₄alkenyl, C₂-C₄alkynyl, C₁-C₃haloalkyl, or C₃-C₄cycloalkyl; or
   B. halogen or C₁-C₆alkyl; or
   C. methyl, F, Cl, Br, or I; or
   D. methyl, C₁, or Br; or
   E. methyl or chlorine.
R₄ is provided above in respect of the first aspect (where Y5 and Y6 corresponds to the equivalent Y embodiment and Yn.1 to Yn.8 (where n can be 5 or 6) corresponds to Ya to Yh, and its preferred embodiments G, H and I of Y above), wherein,
   Y5, in an embodiment is
      a) C₁-C₃alkoxy, C₁-C₃haloalkoxy or Y5.1 to Y5.8; or
      b) methoxy, trifluoromethoxy, difluoromethoxy or Y5.1 to Y5.8; or
      c) Yb, Yc, Yd, Ye, Yf, Yg, or Yh; or
      d) Yb, Yc, Yd, Ye or Yg, or Yh, where, independently of Y, R₇ is selected from chlorine, bromine, fluorine, difluoromethyl, trifluoromethyl, cyclopropyl or phenyl substituted by trifluoromethyl; or
      e) selected from Ya1 to Ya6.
   Y6, in an embodiment is
      a) C₁-C₃haloalkoxy or Y6.1 to Y6.8; or
      b) trifluoromethoxy, difluoromethoxy or Y6.1 to Y6.8; or
      c) Yb, Yc, Yd, Ye, Yf, Yg, or Yh; or
      d) Yb, Yc, Yd, Ye or Yg, or Yh, where, independently of Y, R₇ is selected from chlorine, bromine, fluorine, difluoromethyl, trifluoromethyl, cyclopropyl or phenyl substituted by trifluoromethyl; or
      e) selected from Ya1 to Ya6.
R₃ is
   A. pyridyl ring, which is unsubstituted or is substituted with one to three substituents independently selected from R₆; or
   B. pyridyl ring, which is substituted with one or two substituents independently selected from halogen, cyano, and C₁-C₃alkyl; or
   C. pyridyl ring, which is substituted with one or two substituents independently selected from chlorine, bromine, iodine, cyano, and methyl; or
   D. pyridyl ring, which is substituted with one or two substituents independently selected from chlorine, bromine, and iodine; or
   E. pyridyl ring, which is substituted with one or two substituents chlorine; or
   F. pyrid-2-yl ring, which is substituted with one or two substituents chlorine; or
   G. 3-chloro-pyrid-2-yl ring or 3,5-dichloro-pyrid-2-yl ring; or
   H. 3-chloro-pyrid-2-yl ring.
R₆ is
   A. independently selected from halogen, C₁-C₃alkyl, and C₁-C₃haloalkyl; or
   B. independently selected from chlorine, bromine, and iodine; or
   C. chlorine.
R₈ is provided above in respect of the first aspect;
   (I) wherein in the instance R₁ is hydrogen, halogen, C₂-C₆alkyl, C₂-C₆alkenyl, C₂-C₆alkynyl, C₁-C₆haloalkyl, or C₃-C₆cycloalkyl,
      a. R₄ is
         i. hydrogen, halogen, C₁-C₃alkyl, C₂-C₄alkenyl, C₂-C₄alkynyl, C₁-C₃haloalkyl, C₃-C₄cycloalkyl, C₃-C₄halocycloalkyl, C₃-C₄cyanocycloalkyl, C₁-C₃alkoxy, C₁-C₃haloalkoxy, or X2-Y5; or
         ii. halogen, C₁-C₃haloalkyl, C₁-C₃alkoxy, C₁-C₃haloalkoxy, or X2-Y5; or
         iii. trifluoromethyl, bromine, chlorine, methoxy, or X2-Y5; or
         iv. trifluoromethyl, bromine, chlorine, methoxy, or X2-Ya1 to X2-Ya6;
   (II) wherein in the instance R₁ is methyl,
      a. R₄ is
         i. hydrogen, halogen, C₁-C₃alkyl, C₂-C₄alkenyl, C₂-C₄alkynyl, CHF₂, C₂-C₃haloalkyl, C₃-C₄cycloalkyl, C₃-C₄halocycloalkyl, C₃-C₄cyanocycloalkyl, C₁-C₃alkoxy, C₁-C₃haloalkoxy, or X2-Y6; or
         ii. halogen, CHF₂, C₂haloalkyl, C₁-C₃alkoxy, C₁-C₃haloalkoxy, or X2-Y6; or
         iii. bromine, chlorine, methoxy, or X2-Y6; or
         iv. bromine, chlorine, methoxy, or X2-Ya1 to X2-Ya6,

In an embodiment of each aspect of the invention, in respect of formula Ic (where R₂ from formula I is cyano),
R₁ is
   A. halogen, C₁-C₃alkyl, C₂-C₄alkenyl, C₂-C₄alkynyl, C₁-C₃haloalkyl, or C₃-C₄cycloalkyl; or
   B. halogen or C₁-C₆alkyl; or
   C. methyl, F, Cl, Br, or I; or
   D. methyl, C₁, or Br; or
   E. methyl or chlorine.
R₄ is provided above in respect of the first aspect (where Y7 to Y9 corresponds to the equivalent Y embodiment and Yn.1 to Yn.8 (where n can be 7, 8 or 9) corresponds to Ya to Yh, and its preferred embodiments G, H and I of Y above), wherein,
   Y7, in an embodiment is
      a) C₁-C₃alkoxy, C₁-C₃haloalkoxy or Y7.1 to Y7.8; or
      b) methoxy, trifluoromethoxy, difluoromethoxy or Y7.1 to Y7.8; or
      c) Yb, Yc, Yd, Ye, Yf, Yg, or Yh; or
      d) Yb, Yc, Yd, Ye or Yg, or Yh, where, independently of Y, R₇ is selected from chlorine, bromine, fluorine, difluoromethyl, trifluoromethyl, cyclopropyl or phenyl substituted by trifluoromethyl; or
      e) selected from Ya1 to Ya6.
   Y8, in an embodiment is
      a) C₁-C₃haloalkoxy or Y8.1 to Y8.8; or
      b) trifluoromethoxy, difluoromethoxy or Y8.1 to Y8.8; or
      c) Yb, Yc, Yd, Ye, Yf, Yg, or Yh; or
      d) Yb, Yc, Yd, Ye or Yg, or Yh, where, independently of Y, R₇ is selected from chlorine, bromine, fluorine, difluoromethyl, trifluoromethyl, cyclopropyl or phenyl substituted by trifluoromethyl; or
      e) selected from Ya1 to Ya6.
   Y9, in an embodiment is
      a) C₁-C₃alkoxy, C₁-C₃haloalkoxy or Y9.1 to Y9.8; or
      b) methoxy, trifluoromethoxy, difluoromethoxy or Y9.1 to Y9.8; or
      c) Yb, Yc, Yd, Ye, Yf, Yg, or Yh; or
      d) Yb, Yc, Yd, Ye or Yg, or Yh, where, independently of Y, R₇ is selected from chlorine, bromine, fluorine, difluoromethyl, trifluoromethyl, cyclopropyl or phenyl substituted by trifluoromethyl; or
      e) selected from Ya1 to Ya6.
R₃ is
   A. pyridyl ring, which is unsubstituted or is substituted with one to three substituents independently selected from R₆; or
   B. pyridyl ring, which is substituted with one or two substituents independently selected from halogen, cyano, and C₁-C₃alkyl; or
   C. pyridyl ring, which is substituted with one or two substituents independently selected from chlorine, bromine, iodine, cyano, and methyl; or
   D. pyridyl ring, which is substituted with one or two substituents independently selected from chlorine, bromine, and iodine; or
   E. pyridyl ring, which is substituted with one or two substituents chlorine; or
   F. pyrid-2-yl ring, which is substituted with one or two substituents chlorine; or
   G. 3-chloro-pyrid-2-yl ring or 3,5-dichloro-pyrid-2-yl ring; or
   H. 3-chloro-pyrid-2-yl ring.
R₆ is
   A. independently selected from halogen, C₁-C₃alkyl, and C₁-C₃haloalkyl; or
   B. independently selected from chlorine, bromine, and iodine; or
   C. chlorine.
R₈ is provided above in respect of the first aspect;
   (I) wherein in the instance R₁ is hydrogen, bromine, iodine, C₂-C₆alkyl, C₂-C₆alkenyl, C₂-C₆alkynyl, C₁-C₆haloalkyl, or C₃-C₆cycloalkyl,
      a. R₄ is
         i. hydrogen, halogen, C₁-C₃alkyl, C₂-C₄alkenyl, C₂-C₄alkynyl, C₁-C₃haloalkyl, C₃-C₄cycloalkyl, C₃-C₄halocycloalkyl, C₃-C₄cyanocycloalkyl, C₁-C₃alkoxy, C₁-C₃haloalkoxy, X2-Y7; or
         ii. halogen, C₁-C₃haloalkyl, C₁-C₃alkoxy, C₁-C₃haloalkoxy, or X2-Y7; or
         iii. trifluoromethyl, bromine, chlorine, methoxy, or X2-Y7; or
         iv. trifluoromethyl, bromine, chlorine, methoxy, or X2-Ya1 to X2-Ya6;
   (II) wherein in the instance R₁ is methyl,
      a. R₄ is
         i. hydrogen, fluorine, iodine, C₂-C₃alkyl, C₂-C₄alkenyl, C₃-C₄cycloalkyl, C₃-C₄halocycloalkyl, C₃-C₄cyanocycloalkyl, C₁-C₃alkoxy, or X2-Y8; or
         ii. fluorine, iodine, C₁-C₃alkoxy, or X2-Y8; or
         iii. methoxy, or X2-Y8; or
         iv. methoxy, or X2-Ya1 to X2-Ya6;
   (III) wherein in the instance R₁ is chlorine or fluorine,
      a. R₄ is
         i. hydrogen, fluorine, iodine, C₂-C₃alkyl, C₂-C₄alkenyl, C₂-C₄alkynyl, C₃ C₃-C₄cycloalkyl, C₃-C₄halocycloalkyl, C₃-C₄cyanocycloalkyl, or X2-Y9; or
         ii. fluorine, iodine, or X2-Y9; or
         iii. methoxy, or X2-Y9; or
         iv. X2-Ya1 to X2-Ya6.

In an embodiment of each aspect of the invention, in respect of formula Id (where R₂ from formula I is chlorine),
R₁ is
   A. halogen, C₁-C₃alkyl, C₂-C₄alkenyl, C₂-C₄alkynyl, C₁-C₃haloalkyl, or C₃-C₄cycloalkyl; or
   B. halogen or C₁-C₆alkyl; or
   C. methyl, F, Cl, Br, or I; or
   D. methyl, C₁, or Br; or
   E. methyl or chlorine.
R₄ is provided above in respect of the first aspect (where Y10 to Y13 corresponds to the equivalent Y embodiment and Yn.1 to Yn.8 (where n can be 10, 11, 12 or 13) corresponds to Ya to Yh, and its preferred embodiments G, H and I of Y above), wherein,
   Y10, in an embodiment is
      a) Yb, Yc, Yd, Ye, Yf, Yg, or Yh; or
      b) Yb, Yc, Yd, Ye or Yg, or Yh, where, independently of Y, R₇ is selected from chlorine, bromine, fluorine, difluoromethyl, trifluoromethyl, cyclopropyl or phenyl substituted by trifluoromethyl; or
      c) selected from Ya1 to Ya6.
   Y11, in an embodiment is
      a) C₁-C₃haloalkoxy or Y11.1 to Y11.8; or
      b) trifluoromethoxy, difluoromethoxy or Y11.1 to Y11.8; or
      c) Yb, Yc, Yd, Ye, Yf, Yg, or Yh; or
      d) Yb, Yc, Yd, Ye or Yg, or Yh, where, independently of Y, R₇ is selected from chlorine, bromine, fluorine, difluoromethyl, trifluoromethyl, cyclopropyl or phenyl substituted by trifluoromethyl; or
      e) selected from Ya1 to Ya6.
   Y12, in an embodiment is
      a) C₁-C₃haloalkoxy or Y12.1 to Y12.8; or
      b) trifluoromethoxy, difluoromethoxy or Y12.1 to Y12.8; or
      c) Yb, Yc, Yd, Ye, Yf, Yg, or Yh, where, independently of Y, R₇ is selected from chlorine, bromine, fluorine, difluoromethyl, trifluoromethyl, cyclopropyl or phenyl substituted by trifluoromethyl, with the proviso R₇ is not phenyl substituted by trifluoromethyl for Yb, or R₇ is not trifluoromethyl for Yb, Yd and Ye; or
      d) Yb, Yc, Yd, Ye or Yg, or Yh, where, independently of Y, R₇ is selected from chlorine, bromine, fluorine, difluoromethyl, trifluoromethyl, cyclopropyl or phenyl substituted by trifluoromethyl, with the proviso R₇ is not phenyl substituted by trifluoromethyl for Yb, or R₇ is not trifluoromethyl for Yb, Yd and Ye; or
      e) selected from Ya2, Ya5 and Ya6.
   Y13, in an embodiment is
      a) C₁-C₃alkoxy, C₁-C₃haloalkoxy or Y13.1 to Y13.8; or
      b) methoxy, trifluoromethoxy, difluoromethoxy or Y4.1 to Y4.8; or
      c) Yb, Yc, Yd, Ye, Yf, Yg, or Yh; or
      d) Yb, Yc, Yd, Ye or Yg, or Yh, where, independently of Y, R₇ is selected from chlorine, bromine, fluorine, difluoromethyl, trifluoromethyl, cyclopropyl or phenyl substituted by trifluoromethyl; or
      e) selected from Ya1 to Ya6.
R₃ is
   A. pyridyl ring, which is unsubstituted or is substituted with one to three substituents independently selected from R₆; or
   B. pyridyl ring, which is substituted with one or two substituents independently selected from halogen, cyano, and C₁-C₃alkyl; or
   C. pyridyl ring, which is substituted with one or two substituents independently selected from chlorine, bromine, iodine, cyano, and methyl; or
   D. pyridyl ring, which is substituted with one or two substituents independently selected from chlorine, bromine, and iodine; or
   E. pyridyl ring, which is substituted with one or two substituents chlorine; or
   F. pyrid-2-yl ring, which is substituted with one or two substituents chlorine; or
   G. 3-chloro-pyrid-2-yl ring or 3,5-dichloro-pyrid-2-yl ring; or
   H. 3-chloro-pyrid-2-yl ring.
R₆ is
   A. independently selected from halogen, C₁-C₃alkyl, and C₁-C₃haloalkyl; or
   B. independently selected from chlorine, bromine, and iodine; or
   C. chlorine.
R₈ is provided above in respect of the first aspect;
   (I) wherein in the instance R₁ is chlorine,
      a. R₄ is
         i. hydrogen, fluorine, iodine, C₂-C₃alkyl, C₃-C₄cycloalkyl, C₃-C₄halocycloalkyl, C₃-C₄cyanocycloalkyl, C₁-C₃alkoxy or X2-Y10; or
         ii. halogen, C₁-C₃alkoxy, or X2-Y10; or
         iii. bromine, chlorine, methoxy, or X2-Y10; or
         iv. bromine, chlorine, methoxy, or X2-Ya1 to X2-Ya6;
   (II) wherein in the instance R₁ is bromine,
      a. R₄ is
         i. hydrogen, fluorine, iodine, C₂-C₃alkyl, C₂-C₄alkenyl, C₂-C₄alkynyl, C₃-C₆cycloalkyl, C₃-C₆halocycloalkyl, C₃-C₄cyanocycloalkyl, C₁-C₆alkoxy, OCF₃, or X2-Y11; or
         ii. fluorine, iodine, C₁-C₃alkoxy, or X2-Y11; or
         iii. methoxy, or X2-Y11; or
         iv. methoxy, or X2-Ya1 to X2-Ya6;
   (III) wherein in the instance R₁ is methyl,
      a. R₄ is
         i. hydrogen, CH₂Cl, C₂-C₃alkyl, C₂-C₄alkenyl, C₃-C₄cycloalkyl, C₃-C₄cyanocycloalkyl, C₁-C₃alkoxy, OCH₂CHF₂, C₃haloalkoxy, or X2-Y12; or
         ii. methoxy, CH₂Cl, OCH₂CHF₂, C₃haloalkoxy, or X2-Y12; or
         iii. methoxy, or X2-Y12; or
         iv. methoxy, or X2-Ya1 to X2-Ya6;
   (IV) wherein in the instance R₁ is C₂-C₆alkyl, C₂-C₆alkenyl, C₂-C₆alkynyl, C₁-C₆haloalkyl, or C₃-C₆cycloalkyl,
      a. R₄ is
         i. hydrogen, halogen, C₁-C₃alkyl, C₂-C₄alkenyl, C₂-C₄alkynyl, C₁-C₃haloalkyl, C₃-C₄cycloalkyl, C₃-C₄halocycloalkyl, C₃-C₄cyanocycloalkyl, C₁-C₃alkoxy, C₁-C₃haloalkoxy, or X2-Y13;
         ii. halogen, C₁-C₃alkoxy, C₁-C₃haloalkyl, C₁-C₃haloalkoxy, or X2-Y13; or
         iii. trifluoromethyl, bromine, chlorine, methoxy or X2-Y13; or
         iv. trifluoromethyl, bromine, chlorine, methoxy or X2-Ya1 to X2-Ya6.

The present invention, accordingly, makes available a compound of formula I having the substituents A, R₁, R₂, R₃, R₄, and R₈ as defined above in all combinations/each permutation. Accordingly, made available, for example, is a compound of formula I with A being the first aspect (i.e. A is oxygen or sulfur); R₁ being embodiment D (i.e. methyl, chlorine, or bromine); R₂ being an embodiment B (i.e. cyano, chlorine, bromine, or iodine); R₃ being an embodiment F (i.e. pyridyl ring, which is substituted with one or two substituents independently selected from chlorine, bromine, and iodine); R₄ being embodiment C (i.e. trifluoromethyl, bromine, chlorine, methoxy or X2-Y); and R₈ being an embodiment B (i.e. hydrogen, halogen, cyano, or C₁-C₃alkyl).

Similarly, present invention, accordingly, makes available a compound of formula Ia, Ib, Ic or Id having the substituents A, R₁, R₂, R₃, R₄, and R₈ as defined above in all combinations/each permutation. For example, made available, is a compound of formula Id with A being embodiment B aspect (i.e. A is oxygen); R₂ being chlorine; R₁ being embodiment D (i.e. methyl, chlorine, or bromine); R₃ being an embodiment H (i.e. pyrid-2-yl ring, which is substituted with one or two substituents chlorine); R₄ being embodiment C (i.e. trifluoromethyl, bromine, chlorine, methoxy or X2-Y); and R₈ being an embodiment D (i.e. hydrogen, fluorine, chlorine, bromine, iodine, or methyl), wherein in the instance R₁ is chlorine, R₄ is embodiment iv. (i.e. bromine, chlorine, methoxy, or X2-Ya1 to X2-Ya6); in the instance R₁ is bromine, R₄ is embodiment iii. (i.e. methoxy, or X2-Y11); or in the instance R₁ is methyl, R₄ is embodiment i. (i.e. hydrogen, CH₂Cl, C₂-C₃alkyl, C₂-C₄alkenyl, C₃-C₄cycloalkyl, C₃-C₄cyanocycloalkyl, C₁-C₃alkoxy, OCH₂CHF₂, C₃haloalkoxy, or X2-Y12).

In an embodiment of each aspect of the invention, the insects are those resistant to insecticides of the IRAC class 28 (https://irac-online.org>moa-classification), which act on the ryanodine receptors of the insects—such insecticides are generally referred to as diamides or phthalimide insecticides.

In an embodiment, the compounds of formula I (including formula Ia, Ib, Ic and Id) control insects that demonstrate resistance to at least one compound selected from chlorantraniliprole, cyantraniliprole, cyclantraniliprole, fluchordiniliprole, tetrachlorantraniliprole, tetraniliprole, flubendamide and cyhalodiamide, in particular insects that have developed target site resistance.

The insects have developed target site resistance and have, for example, at least one of the mutations i.e. I4970M and G4946E (P. xylostella numbering). A skilled person would however not exclude that mutations in different positions in the target-site may also cause high levels of diamide resistance.

The diamide-resistant insects are preferably from the order Lepidoptera.

Preferred species are Plutella xylostella (Troczka et al. 2012; Steinbach et al. 2015; Guo et al. 2014), Tuta absoluta (Roditakis et al. 2017; Zimmer et al. 2019), Spodoptera frugiperda (Bolzan et al. 2019) Spodoptera exigua (Zuo et al. 2020, 2017) Chilo suppressalis (Yao et al. 2017; Yang et al. 2017).

In an embodiment of the first aspect, the method for combating and controlling diamide-resistant insects is in a defined area/field of plants where the ratio of diamide-resistant insects to their corresponding susceptible strains is greater than 1:20 (based on number of insects), preferably greater than 1:10, especially greater than 1:5.

In an embodiment of the first aspect, a compound of formula I controls the diamide-resistant insect better compared to the secondary amide analog of the compound of formula I. The improvement in control can be more than 20, preferably 30, more preferably 40, and most preferably 50, percent. The improvement in the control is assessed at the same level, for example at 5 ppm.

In an embodiment of the first aspect, the method for combating and controlling diamide-resistant insects is by applying to a plant susceptible to attack by the insect an effective amount of a compound of formula I; or by treating the propagation material with an effective amount of a compound of formula I.

In an embodiment of the first aspect, compounds of formula I are represented by the formula I-I.

In an embodiment, the compounds of formula I are represented by the formula Ia, Ib, Ic, or Id.

In an embodiment of the first aspect of the invention, the compound of formula I-I has as R₁ halogen, or C₁-C₃alkyl; R₂ as cyano, chlorine, bromine, or iodine; R₃ as 3-chloro-2-pyridyl or 3,5-dichloro-2-pyridyl; R₄ as halogen, C₁-C₃haloalkyl, C₁-C₃alkoxy, C₁-C₃haloalkoxy, or X2-Y, where X2 is CH₂ or CF₂, and Y is selected from Ya to Yj; R₇ is chlorine, bromine, fluorine, difluoromethyl, trifluoromethyl, cyclopropyl or phenyl substituted by trifluoromethyl; and R₈ as hydrogen, fluorine, or chlorine.

In an embodiment of the first aspect of the invention, the compound of formula I-I has as R₁ methyl, fluorine, iodine, chlorine or bromine; R₂ as cyano, chlorine, bromine, or iodine; R₃ as 3-chloro-2-pyridyl or 3,5-dichloro-2-pyridyl; R₄ as halogen, C₁-C₃haloalkyl, C₁-C₃alkoxy, C₁-C₃haloalkoxy, or X2-Y, where X2 is CH₂ or CF₂, and Y is selected from Ya to Yj; R₇ is chlorine, bromine, fluorine, difluoromethyl, trifluoromethyl, cyclopropyl or phenyl substituted by trifluoromethyl; and R₈ as hydrogen, fluorine, or chlorine.

In an embodiment of the first aspect of the invention, the compound of formula I-I has as R₁ halogen, or C₁-C₃alkyl; R₂ as cyano, chlorine, bromine, or iodine; R₃ as 3-chloro-2-pyridyl or 3,5-dichloro-2-pyridyl; R₄ as trifluoromethyl, difluoromethyl, bromine, iodine, methoxy, difluoromethoxy, 2,2,2-trifluoroethoxy, 2,2.difluoroethoxy, or X2-Y, where X2 is CH₂ or CF₂, and Y is selected from Ya to Yj; R₇ is chlorine, bromine, fluorine, difluoromethyl, trifluoromethyl, cyclopropyl or phenyl substituted by trifluoromethyl; and R₃ as hydrogen, fluorine or chlorine.

In an embodiment of the first aspect of the invention, the compound of formula I-I has as R₁ methyl, chlorine or bromine; R₂ as cyano, chlorine, bromine, or iodine; R₃ as 3-chloro-2-pyridyl or 3,5-dichloro-2-pyridyl; R₄ as trifluoromethyl, difluoromethyl, bromine, iodine, methoxy, difluoromethoxy, 2,2,2-trifluoroethoxy, 2,2.difluoroethoxy, or X2-Y, where X2 is CH₂ or CF₂, and Y is selected from Ya to Yh; R₇ is chlorine, bromine, fluorine, difluoromethyl, trifluoromethyl, cyclopropyl or phenyl substituted by trifluoromethyl; and R₈ as hydrogen, fluorine, or chlorine

In an embodiment of the first aspect of the invention, the compound of formula I-I has as R₁ methyl, chlorine or bromine; R₂ as cyano, chlorine, bromine, or iodine; R₃ as 3-chloro-2-pyridyl or 3,5-dichloro-2-pyridyl; R₄ as trifluoromethyl, bromine, chlorine, methoxy or X2-Y, where X2 is CH₂ or CF₂, and Y is selected from Ya1 to Ya17; and R₈ as hydrogen, fluorine or chlorine.

In an embodiment of the first aspect of the invention, the compound of formula I-I has as R₁ methyl, chlorine or bromine; R₂ as cyano, chlorine, bromine, or iodine; R₃ as 3-chloro-2-pyridyl or 3,5-dichloro-2-pyridyl; R₄ as trifluoromethyl, bromine, chlorine, methoxy or X2-Y, where X2 is CH₂ or CF₂, and Y is selected from Ya1 to Ya6; and R₈ as hydrogen, fluorine or chlorine.

Preferred compound of formula I for the first aspect are those in tables A-1 to A-4 and P and having the following CAS numbers: 736994-64-2; 736994-59-5; 1032669-86-5; 736994-62-0; 1032670-41-9; 1032672-17-5; 1032672-00-6; 1032671-78-5; 736994-80-2; 736994-71-1; 1229654-71-0; 1622383-17-8; 1229656-45-4; 2447300-35-6; 2447299-98-9; 2447299-81-0; 2447299-63-8; 2447299-40-1; 2447298-29-3; 2447298-02-2; 2447297-75-6; 2447297-49-4; 2447257-99-8; 2447257-90-9; 2447257-81-8; 2447257-72-7; 2447257-51-2; 2447257-42-1; 2447257-25-0; 1353022-95-3; 1339950-02-5; 1339949-41-5; 1339948-14-9; 1229657-60-6; 1070642-80-6; 1070642-47-5; 1070623-54-9; 1070622-10-4; 1070620-76-6; 1070620-59-5; 2446856-10-4; 500007-74-9; 1006621-50-6; 500011-32-5; 500009-11-0; 2446856-47-7; 2446856-38-6; 1622383-16-7; 1622383-14-5; 1070619-56-5; 2446856-95-5; 1070618-71-1; 1229657-58-2; 1229654-70-9; 1339949-52-8; 1070608-13-7; 1070614-31-1; 1622383-19-0; 1070617-78-5; 1070609-67-4; 1070616-93-1; 2446857-04-9; 2446856-77-3; 1070622-23-9; 1070625-39-6; 1070608-19-3; 1355050-95-1; 1070609-61-8; 1070614-24-2; 1339950-11-6; 2446856-86-4; 2446856-68-2; 1070609-55-0; 1070621-75-8; 1070624-09-7; 1070616-17-9; 1070607-42-9; 1070607-22-5; 1070607-28-1; 2447269-89-6; 458543-39-0; 500007-75-0; 1006623-99-9; 2447270-16-6; 2447270-07-5; 2447269-98-7; 1006623-68-2; 2447270-65-5; 1229657-59-3; 2129147-10-8; 2129147-23-3; 1229655-09-7; 2129147-02-8; 1339949-63-1; 1070614-67-3; 1006624-89-0; 1070608-33-1; 2447270-74-6; 2447270-47-3; 1070621-61-2; 1070623-95-8; 1070608-26-2; 1070614-60-6; 2447270-56-4; 1339950-29-6; 1006624-12-9; 1070621-98-5; 1070623-42-5; 2447316-38-1; 500011-46-1; 2414912-28-8; 2447316-89-2; 2447316-71-2; 1622383-18-9; 2414912-31-3; 2447318-02-5; 2447318-29-6; 2447317-40-8; 2447318-11-6; 2447317-76-0; 1070621-12-3; 1070620-07-3; 2447317-93-1; 1070620-42-6; 2447317-58-8; 1070619-17-8; 2447214-39-1; 500007-48-7; 2447214-66-4; 500007-82-9; 458543-44-7; 2447214-57-3; 2447282-99-5; 2447214-48-2; 1622383-15-6; 1006624-64-1; 1006624-37-8; 500007-83-0; 2447283-52-3; 500007-38-5; 2447283-34-1; 2447283-16-9; 2447215-18-9; 500007-57-8; 1229654-98-1; 2447284-36-6; 1006622-25-8; 2447215-27-0; 2447215-00-9; 2447284-63-9; 2447283-96-5; 2447284-45-7; 2447284-18-4; 1070621-18-9; 2447215-09-8; 2447214-91-5; 1070620-13-1; 2447284-27-5; 1070620-48-2; 1006624-81-2; 1070618-35-7; 500007-78-3; 1229657-26-4; 1229655-21-3; 1339949-74-4; 1070623-77-6; 1070621-48-5; 1339950-41-2; 1070621-00-9; and 1070619-95-2 (List Q).

Certain compounds of formula I are disclosed in WO2020117493; WO2004067528; WO2008082502; WO2009111553; WO2009061991; WO2009006061; WO2008070158; WO2009085816; WO2010069502; WO2011128329; WO2004046129; WO2011157778; DE102006042437; Bioorganic & Medicinal Chemistry Letters (2013), 23(23), 6341-6345; WO2014128188; Bioorganic & Medicinal Chemistry Letters (2013), 23(1), 42-46; US20110130427; WO2010010958; WO2006023783; CN102285963; WO2003015518; WO2003024222; CN106928183; WO2009121288; WO2008010897; Chemical Research in Toxicology (2012), 25(8), 1571-1573; CN102391248; Chemical Engineering Journal (Amsterdam, Netherlands) (2015), 264, 720-727; WO2013044791; CN110028489; CN102047914; WO2014053407; CN106977494; CN106588870; WO2002070483; and CN110330455.

In an embodiment of the second or later aspects of the invention, the compound of formula Ia has as R₁ halogen or C₁-C₆alkyl; R₃ as 3-chloro-2-pyridyl or 3,5-dichloro-2-pyridyl; R₄ as halogen, C₁-C₃haloalkyl, C₁-C₃alkoxy, C₁-C₃haloalkoxy, or X2-Y (where X2 is CH₂ or CF₂, and Y is selected from Ya to Yj; R₇ is chlorine, bromine, fluorine, difluoromethyl, trifluoromethyl, cyclopropyl or phenyl substituted by trifluoromethyl); and R₈ is hydrogen, fluorine or chlorine; in the instance R₁ is chlorine, R₄ is fluorine, iodine, methoxy, or X2-Ya1 to X2-Ya6: in the instance R₁ is fluorine or bromine, R₄ is fluorine, iodine, trifluoromethyl, methoxy or X2-Ya1 to X2-Ya6; and in the instance R₁ is methyl, R₄ is iodine, methoxy or Ya1 to Ya6.

In an embodiment of the second or later aspects of the invention, the compound of formula Ia has as R₁ methyl, fluorine, iodine, chlorine or bromine; R₃ as 3-chloro-2-pyridyl or 3,5-dichloro-2-pyridyl; R₄ as halogen, C₁-C₃haloalkyl, C₁-C₃alkoxy, C₁-C₃haloalkoxy, or X2-Y (where X2 is CH₂ or CF₂, and Y is selected from Ya to Yj; R₇ is chlorine, bromine, fluorine, difluoromethyl, trifluoromethyl, cyclopropyl or phenyl substituted by trifluoromethyl); and R₃ is hydrogen, fluorine or chlorine; in the instance R₁ is chlorine, R₄ is fluorine, iodine, methoxy, or X2-Ya1 to X2-Ya6: in the instance R₁ is fluorine or bromine, R₄ is fluorine, iodine, trifluoromethyl, methoxy or X2-Ya1 to X2-Ya6; and in the instance R₁ is methyl, R₄ is iodine, methoxy or Ya1 to Ya6.

In an embodiment of the second or later aspects of the invention, the compound of formula Ia has as R₁ methyl, fluorine, iodine, chlorine or bromine; R₃ as 3-chloro-2-pyridyl or 3,5-dichloro-2-pyridyl; R₄ as trifluoromethyl, difluoromethyl, bromine, iodine, methoxy, difluoromethoxy, 2,2,2-trifluoroethoxy, 2,2.difluoroethoxy, or X2-Y (where X2 is CH₂ or CF₂, and Y is selected from Ya to Yj; R₇ is chlorine, bromine, fluorine, difluoromethyl, trifluoromethyl, cyclopropyl or phenyl substituted by trifluoromethyl); and R₈ is hydrogen, fluorine or chlorine; in the instance R₁ is chlorine, R₄ is fluorine, iodine, methoxy, or X2-Ya1 to X2-Ya6: in the instance R₁ is fluorine or bromine, R₄ is fluorine, iodine, trifluoromethyl, methoxy or X2-Ya1 to X2-Ya6; and in the instance R₁ is methyl, R₄ is iodine, methoxy or Ya1 to Ya6.

In an embodiment of the second or later aspects of the invention, the compound of formula Ib has as R₁ halogen or C₁-C₆alkyl; R₃ as 3-chloro-2-pyridyl or 3,5-dichloro-2-pyridyl; R₄ as halogen, C₁-C₃haloalkyl, C₁-C₃alkoxy, C₁-C₃haloalkoxy, or X2-Y (where X2 is CH₂ or CF₂, and Y is selected from Ya to Yj; R₇ is chlorine, bromine, fluorine, difluoromethyl, trifluoromethyl, cyclopropyl or phenyl substituted by trifluoromethyl); and R₃ is hydrogen, fluorine or chlorine; in the instance R₁ is methyl, R₄ is bromine, chlorine, methoxy, or X2-Ya1 to X2-Ya6.

In an embodiment of the second or later aspects of the invention, the compound of formula Ib has as R₁ methyl, fluorine, iodine, chlorine or bromine; R₃ as 3-chloro-2-pyridyl or 3,5-dichloro-2-pyridyl; R₄ as halogen, C₁-C₃haloalkyl, C₁-C₃alkoxy, C₁-C₃haloalkoxy, or X2-Y (where X2 is CH₂ or CF₂, and Y is selected from Ya to Yj; R₇ is chlorine, bromine, fluorine, difluoromethyl, trifluoromethyl, cyclopropyl or phenyl substituted by trifluoromethyl); and R₃ is hydrogen, fluorine or chlorine; in the instance R₁ is methyl, R₄ is bromine, chlorine, methoxy, or X2-Ya1 to X2-Ya6.

In an embodiment of the second or later aspects of the invention, the compound of formula Ib has as R₁ methyl, fluorine, iodine, chlorine or bromine; R₃ as 3-chloro-2-pyridyl or 3,5-dichloro-2-pyridyl; R₄ as trifluoromethyl, difluoromethyl, bromine, iodine, methoxy, difluoromethoxy, 2,2,2-trifluoroethoxy, 2,2.difluoroethoxy, or X2-Y (where X2 is CH₂ or CF₂, and Y is selected from Ya to Yj; R₇ is chlorine, bromine, fluorine, difluoromethyl, trifluoromethyl, cyclopropyl or phenyl substituted by trifluoromethyl); and R₈ is hydrogen, fluorine or chlorine; in the instance R₁ is methyl, R₄ is bromine, chlorine, methoxy, or X2-Ya1 to X2-Ya6.

In an embodiment of the second or later aspects of the invention, the compound of formula Ic has as R₁ halogen or C₁-C₆alkyl; R₃ as 3-chloro-2-pyridyl or 3,5-dichloro-2-pyridyl; R₄ as halogen, C₁-C₃haloalkyl, C₁-C₃alkoxy, C₁-C₃haloalkoxy, or X2-Y (where X2 is CH₂ or CF₂, and Y is selected from Ya to Yj; R₇ is chlorine, bromine, fluorine, difluoromethyl, trifluoromethyl, cyclopropyl or phenyl substituted by trifluoromethyl); and R₃ is hydrogen, fluorine or chlorine; in the instance R₁ is methyl, R₄ is methoxy, or X2-Ya1 to X2-Ya6; in the instance R₁ is chlorine or fluorine, R₄ is X2-Ya1 to X2-Ya6.

In an embodiment of the second or later aspects of the invention, the compound of formula Ic has as R₁ methyl, fluorine, iodine, chlorine or bromine; R₃ as 3-chloro-2-pyridyl or 3,5-dichloro-2-pyridyl; R₄ as halogen, C₁-C₃haloalkyl, C₁-C₃alkoxy, C₁-C₃haloalkoxy, or X2-Y (where X2 is CH₂ or CF₂, and Y is selected from Ya to Yj; R₇ is chlorine, bromine, fluorine, difluoromethyl, trifluoromethyl, cyclopropyl or phenyl substituted by trifluoromethyl); and R₃ is hydrogen, fluorine or chlorine; in the instance R₁ is methyl, R₄ is methoxy, or X2-Ya1 to X2-Ya6; in the instance R₁ is chlorine or fluorine, R₄ is X2-Ya1 to X2-Ya6.

In an embodiment of the second or later aspects of the invention, the compound of formula Ic has as R₁ methyl, fluorine, iodine, chlorine or bromine; R₃ as 3-chloro-2-pyridyl or 3,5-dichloro-2-pyridyl; R₄ as trifluoromethyl, difluoromethyl, bromine, iodine, methoxy, difluoromethoxy, 2,2,2-trifluoroethoxy, 2,2.difluoroethoxy, or X2-Y (where X2 is CH₂ or CF₂, and Y is selected from Ya to Yj; R₇ is chlorine, bromine, fluorine, difluoromethyl, trifluoromethyl, cyclopropyl or phenyl substituted by trifluoromethyl); and R₈ is hydrogen, fluorine or chlorine; in the instance R₁ is methyl, R₄ is methoxy, or X2-Ya1 to X2-Ya6; in the instance R₁ is chlorine or fluorine, R₄ is X2-Ya1 to X2-Ya6.

In an embodiment of the second or later aspects of the invention, the compound of formula Id has as R₁ halogen or C₁-C₆alkyl; R₃ as 3-chloro-2-pyridyl or 3,5-dichloro-2-pyridyl; R₄ as halogen, C₁-C₃haloalkyl, C₁-C₃alkoxy, C₁-C₃haloalkoxy, or X2-Y (where X2 is CH₂ or CF₂, and Y is selected from Ya to Yj; R₇ is chlorine, bromine, fluorine, difluoromethyl, trifluoromethyl, cyclopropyl or phenyl substituted by trifluoromethyl); and R₈ is hydrogen, fluorine or chlorine; in the instance R₁ is chlorine, R₄ is bromine, chlorine, methoxy, or X2-Ya1 to X2-Ya6; in the instance R₁ is bromine, R₄ is methoxy, or X2-Ya1 to X2-Ya6; in the instance R₁ is methyl, R₄ is methoxy, X2-Ya5 or X2-Ya6 when X2 CH₂; or X2-Ya1 to X2-Ya6 when X2 is CF₂.

In an embodiment of the second or later aspects of the invention, the compound of formula Id has as R₁ methyl, fluorine, iodine, chlorine or bromine; R₃ as 3-chloro-2-pyridyl or 3,5-dichloro-2-pyridyl; R₄ as halogen, C₁-C₃haloalkyl, C₁-C₃alkoxy, C₁-C₃haloalkoxy, or X2-Y (where X2 is CH₂ or CF₂, and Y is selected from Ya to Yj; R₇ is chlorine, bromine, fluorine, difluoromethyl, trifluoromethyl, cyclopropyl or phenyl substituted by trifluoromethyl); and R₃ is hydrogen, fluorine or chlorine; in the instance R₁ is chlorine, R₄ is bromine, chlorine, methoxy, or X2-Ya1 to X2-Ya6; in the instance R₁ is bromine, R₄ is methoxy, or X2-Ya1 to X2-Ya6; in the instance R₁ is methyl, R₄ is methoxy, X2-Ya5 or X2-Ya6 when X2 CH₂; or X2-Ya1 to X2-Ya6 when X2 is CF₂.

In an embodiment of the second or later aspects of the invention, the compound of formula Id has as R₁ methyl, fluorine, iodine, chlorine or bromine; R₃ as 3-chloro-2-pyridyl or 3,5-dichloro-2-pyridyl; R₄ as trifluoromethyl, difluoromethyl, bromine, iodine, methoxy, difluoromethoxy, 2,2,2-trifluoroethoxy, 2,2.difluoroethoxy, or X2-Y (where X2 is CH₂ or CF₂, and Y is selected from Ya to Yj; R₇ is chlorine, bromine, fluorine, difluoromethyl, trifluoromethyl, cyclopropyl or phenyl substituted by trifluoromethyl); and R₈ is hydrogen, fluorine or chlorine; in the instance R₁ is chlorine, R₄ is bromine, chlorine, methoxy, or X2-Ya1 to X2-Ya6; in the instance R₁ is bromine, R₄ is methoxy, or X2-Ya1 to X2-Ya6; in the instance R₁ is methyl, R₄ is methoxy, X2-Ya5 or X2-Ya6 when X2 CH₂; or X2-Ya1 to X2-Ya6 when X2 is CF₂.

In a third aspect, the present invention makes available a composition comprising a compound of formula I as defined in the second aspect, one or more auxiliaries and diluent, and optionally one or more other active ingredient.

In a fourth aspect, the present invention makes available a method of combating and controlling insects, acarines, nematodes or molluscs which comprises applying to a pest, to a locus of a pest, or to a plant susceptible to attack by a pest an insecticidally, acaricidally, nematicidally or molluscicidally effective amount of a compound as defined in the second aspect or a composition as defined in the third aspect.

In a fifth aspect, the present invention makes available a method for the protection of plant propagation material from the attack by insects, acarines, nematodes or molluscs, which comprises treating the propagation material or the site, where the propagation material is planted, with an effective amount of a compound of formula I as defined in the second aspect or a composition as defined in the third aspect.

In a sixth aspect, the present invention makes available a plant propagation material, such as a seed, comprising, or treated with or adhered thereto, a compound of formula I as defined in the second aspect or a composition as defined in the third aspect.

Compounds of formula I can be prepared by those skilled in the art following known methods. More specifically compounds of formula I, and intermediates therefor can be prepared as described below in the schemes and examples. Certain stereogenic centers have been left unspecified for the clarity and are not intended to limit the teaching of the schemes in any way.

The process according to the invention for preparing compounds of formula I is carried out by methods known to those skilled in the art (see above). In particular, the process for preparing compounds of formulae I, and Ia to Id or, where appropriate, a tautomer and/or salt thereof, is carried out analogously to known processes, for example those described in WO 01/70671, WO 03/016284, WO 03/015518 and WO 04/033468. In the following section the substituents A, R₁, R₂, R₃, R₄, R₆, R₇ (including R₇ₐ-R_{7q}), Ra, X2, and Y (including Y1-Y13) are defined as in formulae Ia to Id unless otherwise stated.

Processes for making compounds of formulae Ia₂ to Id₂,

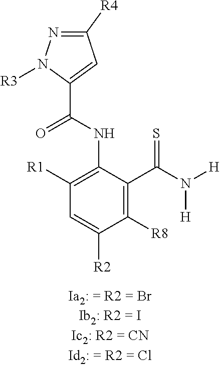

in which R₁, R₂, R₃, R₄, and R₈ have the meanings given for formulae Ia to Id, or, where appropriate, a tautomer and/or salt thereof, can be made from compounds of formulae Ia₁ to Id₁

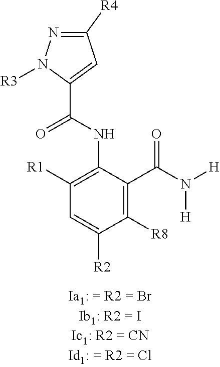

by treatment with a thiation reagent such as Lawesson's reagent or phosphorus pentasulfide. The thiation of amides is well known and many examples are found in the literature. The compounds of formulae Ia₂-Id₂ may have to be separated from regioisomers in case the regioselectivity of the thiation is not sufficient.

A compound of formulae Ia₁ to Id₁, or, where appropriate, a tautomer and/or salt thereof, can be obtained by reacting a compound of formula II

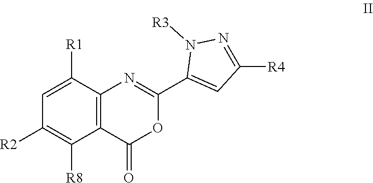

in which, R₁, R₂, R₃, R₄, and R₈ have the meanings given for formulae Ia to Id, with ammonia in either gaseous form, or with a solution of ammonia in a solvent such as ethanol, tetrahydrofuran, or dioxan, or with an alternative form of ammonia, such as ammonium acetate in a suitable solvent, such as ethyl acetate, tetrahydrofuran, or dioxan. The compounds of formula II may be prepared as described in WO04/111030 or WO04/067528. For example, compounds of formula II can be obtained by reaction of amino acids of formula III

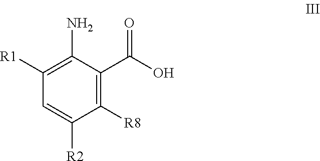

and pyrazole carboxylic acids of formula IV

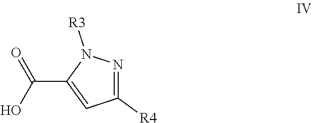

In the presence of a dehydrating reagent such as methanesulfonyl chloride, and a base like a tertiary amine. This transformation is in many cases known in the literature or can be performed according to methods known to a person skilled in the art. Compounds of formula II can be isolated or used directly in the next step as a crude mixture.

The chemistry is described in more detail in Scheme 1.

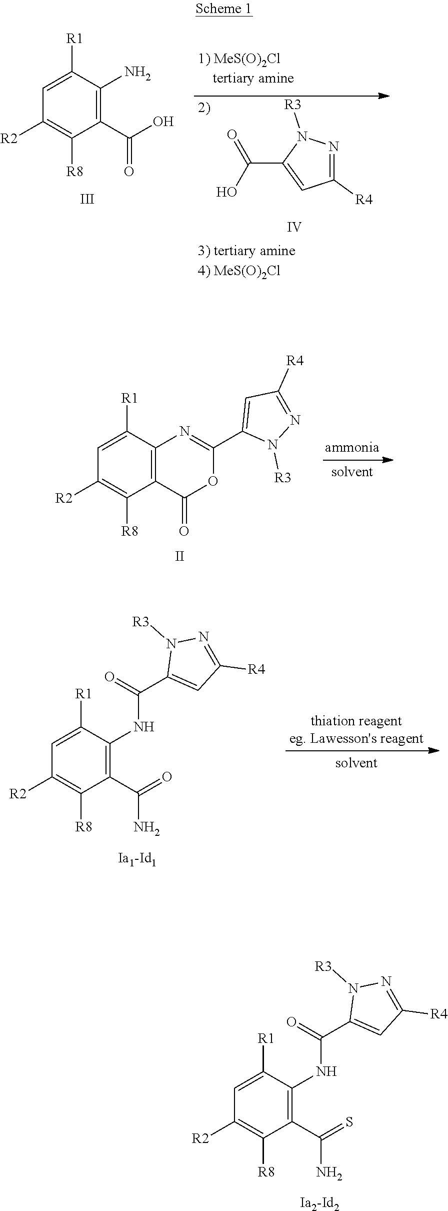

Processes for making compounds of formula III, are generally known or can be easily prepared by those skilled in the art.

Many pyrazole carboxylic acids of formula IV are described in the literature and can be prepared as already described for example in WO04/067528, WO10069502, WO 2011/157664, WO04046129 or in a similar way by a person skilled in the art.

Compounds of formula IVb, in which R₃ has the meaning given for formulae Ia to Id and Y0 is selected from Yn.1-Yn.8 (n=1 to 13) wherein Yn.1-Yn.8 have the meanings given for formulae Ia to Id are generally known or can be prepared by those skilled in the art. A typical example of such a synthesis is shown in Scheme 2.

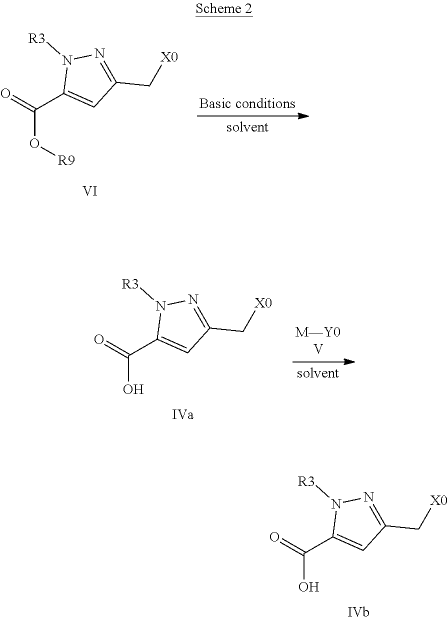

For example, compounds of formula IVb may be prepared by reaction between compounds of formula V, wherein Y0 is defined as above and M is an alkali metal such as lithium, sodium, potassium, and compounds of formula IVa, wherein R₃ has the meaning given for formulae Ia to Id and X0 is chlorine, bromine or iodine, in suitable solvents that may include, for example, acetone or tetrahydrofuran, optionally in the presence of potassium iodide as catalyst.

Compounds of formula IVa, wherein R₃ has the meaning given for formulae Ia to Id and X0 is chlorine, bromine or iodine, can be prepared from compounds of formula VI, wherein R₃ has the meaning given for formulae Ia to Id, R⁹ is a C₁-C₃alkyl group and X0 is chlorine, bromine or iodine, under basic conditions that may include, for example lithium hydroxide or sodium hydroxide, in suitable solvents such as, tetrahydrofuram, methanol or dioxane. Such processes have been described previously, for example, in WO2011073101. Compounds of formula VI are generally known or can be easily prepared by those skilled in the art. A typical example of such a synthesis is described in WO2011073101.

What has been said above for tautomers and/or salts of compounds of formulae Ia to Id applies analogously to starting materials mentioned hereinabove and hereinbelow with regard to the tautomers and/or salts thereof.

The reactions described hereinabove and hereinbelow are carried out in a manner known per se, for example in the absence or, normally, in the presence of a suitable solvent or diluent or of a mixture of these, the process being carried out, as required, with cooling, at room temperature or with heating, for example in a temperature range of from approximately −80° C. to the boiling point of the reaction mixture, preferably from approximately −20° C. to approximately +150° C., and, if required, in a sealed vessel, under reduced, normal or elevated pressure, in an inert gas atmosphere and/or under anhydrous conditions. Especially advantageous reaction conditions can be seen from the examples.

Unless otherwise specified, the starting materials mentioned hereinabove and hereinbelow, which are used for the preparation of the compounds of formulae Ia-Id or, where appropriate, the tautomers thereof, in each case in free form or in salt form, are known or can be prepared by methods known per se, for example in accordance with the information given below.

Depending on the procedure or the reaction conditions, the reactants can be reacted in the presence of a base. Examples of suitable bases are alkali metal or alkaline earth metal hydroxides, alkali metal or alkaline earth metal hydrides, alkali metal or alkaline earth metal amides, alkali metal or alkaline earth metal alkoxides, alkali metal or alkaline earth metal acetates, alkali metal or alkaline earth metal carbonates, alkali metal or alkaline earth metal dialkylamides or alkali metal or alkaline earth metal alkylsilylamides, alkylamines, alkylenediamines, free or N-alkylated saturated or unsaturated cycloalkylamines, basic heterocycles, ammonium hydroxides and carbocyclic amines. Examples which may be mentioned are sodium hydroxide, sodium hydride, sodium amide, sodium methoxide, sodium acetate, sodium carbonate, potassium tert-butoxide, potassium hydroxide, potassium carbonate, potassium hydride, lithium diisopropylamide, potassium bis(trimethylsilyl)amide, calcium hydride, triethylamine, diisopropylethylamine, triethylenediamine, cyclohexylamine, N-cyclohexyl-N,N-dimethylamine, N,N-diethylaniline, pyridine, 4-(N,N-dimethylamino)pyridine, quinuclidine, N-methylmorpholine, benzyltrimethylammonium hydroxide and 1,8-diazabicyclo[5.4.0]undec-7-ene (DBU).

The reactants can be reacted with each other as such, i.e. without adding a solvent or diluent. In most cases, however, it is advantageous to add an inert solvent or diluent or a mixture of these. If the reaction is carried out in the presence of a base, bases which are employed in excess, such as triethylamine, pyridine, N-methylmorpholine or N,N-diethylaniline, may also act as solvents or diluents.

The reaction is advantageously carried out in a temperature range from approximately −80° C. to approximately +140° C., preferably from approximately −30° C. to approximately +100° C., in many cases in the range between room temperature and approximately +80° C.

A compound of formulae Ia to Id can be converted in a manner known per se into another compound of formulae Ia to Id respectively by replacing one or more substituents of the starting compound of formulae Ia to Id in the customary manner by (an)other substituent(s) according to the invention.

For example, compounds of formulae Ia₃ to Id₃ in which, A, R₁, R₂, R₃, R₄ and R₈ have the meanings given for formulae Ia to Id and X0 is iodine, bromine or chlorine, can be converted into compounds of formulae Ia₄ to Id₄ respectively, in which A, R₁, R₂, R₃, R₄ and R₈ have the meanings given for formulae Ia to Id and Y0 is selected from Yn.1-Yn.8 (n=1 to 13) wherein Yn.1-Yn.8 have the meanings given for formulae Ia to Id. A typical example of such a synthesis is shown in Scheme 3.

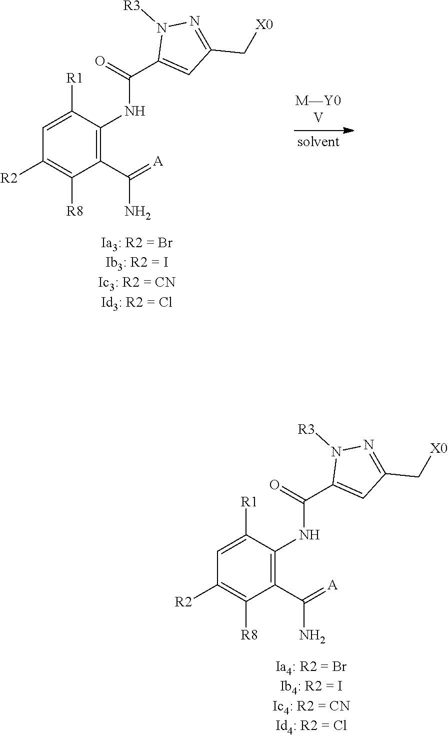

For example, compounds of formulae Ia₄ to Id₄ may be prepared by reaction between compounds of formula V, wherein Y0 is defined as above and M is an alkali metal such as lithium, sodium, potassium, and compounds of formulae Ia₃ to Id₃, wherein A, R₁, R₂, R₃, R₄ and R₈ are defined in formulae Ia to Id and X0 is iodine, bromine or chlorine, in suitable solvents that may include, for example, acetone or tetrahydrofuran, optionally in the presence of potassium iodide as catalyst. Processes for making compounds of formula V, are generally known or can be easily prepared by those skilled in the art.

Compounds of formulae Ia₃ to Id₃ can be made by a similar method than described in Scheme 1 with R₄ is specifically —CH₂Cl, —CH₂Br or —CH₂₁ in this case.

Depending on the choice of the reaction conditions and starting materials which are suitable in each case, it is possible, for example, in one reaction step only to replace one substituent by another substituent according to the invention, or a plurality of substituents can be replaced by other substituents according to the invention in the same reaction step.

Salts of compounds of formulae Ia-Id can be prepared in a manner known per se. Thus, for example, acid addition salts of compounds of formulae Ia-Id are obtained by treatment with a suitable acid or a suitable ion exchanger reagent and salts with bases are obtained by treatment with a suitable base or with a suitable ion exchanger reagent.

Salts of compounds of formulae Ia to Id can be converted in the customary manner into the free compounds of formulae Ia to Id, acid addition salts, for example, by treatment with a suitable basic compound or with a suitable ion exchanger reagent and salts with bases, for example, by treatment with a suitable acid or with a suitable ion exchanger reagent.

Salts of compounds of formulae Ia to Id can be converted in a manner known per se into other salts of compounds of formulae Ia to Id, acid addition salts, for example, into other acid addition salts, for example by treatment of a salt of inorganic acid such as hydrochloride with a suitable metal salt such as a sodium, barium or silver salt, of an acid, for example with silver acetate, in a suitable solvent in which an inorganic salt which forms, for example silver chloride, is insoluble and thus precipitates from the reaction mixture.

Depending on the procedure or the reaction conditions, the compounds of formulae Ia to Id, which have salt-forming properties can be obtained in free form or in the form of salts.

The compounds of formulae Ia to Id and, where appropriate, the tautomers thereof, in each case in free form or in salt form, can be present in the form of one of the isomers which are possible or as a mixture of these, for example in the form of pure isomers, such as antipodes and/or diastereomers, or as isomer mixtures, such as enantiomer mixtures, for example racemates, diastereomer mixtures or racemate mixtures, depending on the number, absolute and relative configuration of asymmetric carbon atoms which occur in the molecule and/or depending on the configuration of non-aromatic double bonds which occur in the molecule; the invention relates to the pure isomers and also to all isomer mixtures which are possible and is to be understood in each case in this sense hereinabove and hereinbelow, even when stereochemical details are not mentioned specifically in each case.

Diastereomer mixtures or racemate mixtures of compounds formulae Ia to Id, in free form or in salt form, which can be obtained depending on which starting materials and procedures have been chosen can be separated in a known manner into the pure diasteromers or racemates on the basis of the physicochemical differences of the components, for example by fractional crystallization, distillation and/or chromatography.

Enantiomer mixtures, such as racemates, which can be obtained in a similar manner can be resolved into the optical antipodes by known methods, for example by recrystallization from an optically active solvent, by chromatography on chiral adsorbents, for example high-performance liquid chromatography (HPLC) on acetyl cellulose, with the aid of suitable microorganisms, by cleavage with specific, immobilized enzymes, via the formation of inclusion compounds, for example using chiral crown ethers, where only one enantiomer is complexed, or by conversion into diastereomeric salts, for example by reacting a basic end-product racemate with an optically active acid, such as a carboxylic acid, for example camphor, tartaric or malic acid, or sulfonic acid, for example camphorsulfonic acid, and separating the diastereomer mixture which can be obtained in this manner, for example by fractional crystallization based on their differing solubilities, to give the diastereomers, from which the desired enantiomer can be set free by the action of suitable agents, for example basic agents.

Pure diastereomers or enantiomers can be obtained according to the invention not only by separating suitable isomer mixtures, but also by generally known methods of diastereoselective or enantioselective synthesis, for example by carrying out the process according to the invention with starting materials of a suitable stereochemistry.

It is advantageous to isolate or synthesize in each case the biologically more effective isomer, for example enantiomer or diastereomer, or isomer mixture, for example enantiomer mixture or diastereomer mixture, if the individual components have a different biological activity.

The compounds according to the invention and, where appropriate, the tautomers thereof, in each case in free form or in salt form, can, if appropriate, also be obtained in the form of hydrates and/or include other solvents, for example those which may have been used for the crystallization of compounds which are present in solid form.

N-oxides can be prepared by reacting a compound of the formula I with a suitable oxidizing agent, for example the H₂O₂/urea adduct in the presence of an acid anhydride, e.g. trifluoroacetic anhydride. Such oxidations are known from the literature, for example from J. Med. Chem., 32 (12), 2561-73, 1989 or WO 2000/15615.

The compounds according to the following Tables A-1 to A-4 below can be prepared according to the methods described above. The examples which follow are intended to illustrate the invention and show preferred compounds of formulae Ia to id, in the form of a compound of formula IAA.

The tables below illustrate specific compounds of the invention.

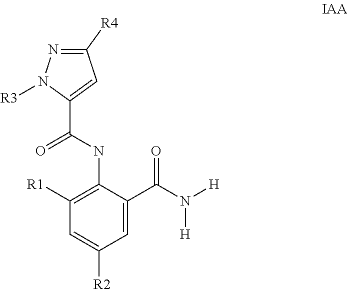

TABLE X
Substituent definitions of R4, R1 and R3:

| Index | R1 | R3 | R4 |
|---|---|---|---|
| 1 | F | 3-chloro- | Br |
| | | 2-pyridyl | |
| 2 | Cl | 3-chloro- | Br |
| | | 2-pyridyl | |
| 3 | Br | 3-chloro- | Br |
| | | 2-pyridyl | |
| 4 | I | 3-chloro- | Br |
| | | 2-pyridyl | |
| 5 | Me | 3-chloro- | Br |
| | | 2-pyridyl | |
| 6 | F | 3-chloro- | I |
| | | 2-pyridyl | |
| 7 | Cl | 3-chloro- | I |
| | | 2-pyridyl | |
| 8 | Br | 3-chloro- | I |
| | | 2-pyridyl | |
| 9 | I | 3-chloro- | I |
| | | 2-pyridyl | |
| 10 | Me | 3-chloro- | I |
| | | 2-pyridyl | |
| 11 | F | 3-chloro- | CF₃ |
| | | 2-pyridyl | |
| 12 | Cl | 3-chloro- | CF₃ |
| | | 2-pyridyl | |
| 13 | Br | 3-chloro- | CF₃ |
| | | 2-pyridyl | |
| 14 | I | 3-chloro- | CF₃ |
| | | 2-pyridyl | |
| 15 | Me | 3-chloro- | CF₃ |
| | | 2-pyridyl | |
| 16 | F | 3-chloro- | CHF₂ |
| | | 2-pyridyl | |
| 17 | Cl | 3-chloro- | CHF₂ |
| | | 2-pyridyl | |
| 18 | Br | 3-chloro- | CHF₂ |
| | | 2-pyridyl | |
| 19 | I | 3-chloro- | CHF₂ |
| | | 2-pyridyl | |
| 20 | Me | 3-chloro- | CHF₂ |
| | | 2-pyridyl | |
| 21 | F | 3-chloro-l | OMe |
| | | 2-pyridy | |
| 22 | Cl | 3-chloro- | OMe |
| | | 2-pyridyl | |
| 23 | Br | 3-chloro- | OMe |
| | | 2-pyridyl | |
| 24 | I | 3-chloro- | OMe |
| | | 2-pyridyl | |
| 25 | Me | 3-chloro- | OMe |
| | | 2-pyridyl | |
| 26 | F | 3-chloro- | OCH₂CF₃ |
| | | 2-pyridyl | |
| 27 | Cl | 3-chloro- | OCH₂CF₃ |
| | | 2-pyridyl | |
| 28 | Br | 3-chloro- | OCH₂CF₃ |
| | | 2-pyridyl | |
| 29 | I | 3-chloro- | OCH₂CF₃ |
| | | 2-pyridyl | |
| 30 | Me | 3-chloro- | OCH₂CF₃ |
| | | 2-pyridyl | |
| 31 | F | 3-chloro- | OCHF₂ |
| | | 2-pyridyl | |
| 32 | Cl | 3-chloro- | OCHF₂ |
| | | 2-pyridyl | |
| 33 | Br | 3-chloro- | OCHF₂ |
| | | 2-pyridyl | |
| 34 | I | 3-chloro- | OCHF₂ |
| | | 2-pyridyl | |
| 35 | Me | 3-chloro- | OCHF₂ |
| | | 2-pyridyl | |
| 36 | F | 3-chloro- | OCH₂CHF₂ |
| | | 2-pyridyl | |
| 37 | Cl | 3-chloro- | OCH₂CHF₂ |
| | | 2-pyridyl | |
| 38 | Br | 3-chloro- | OCH₂CHF₂ |
| | | 2-pyridyl | |
| 39 | I | 3-chloro- | OCH₂CHF₂ |
| | | 2-pyridyl | |
| 40 | Me | 3-chloro- | OCH₂CHF₂ |
| | | 2-pyridyl | |
| 41 | F | 3-chloro- 2-pyridyl | 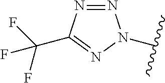 [SMILES: *n1nnc(C(F)(F)F)n1] |
| 42 | Cl | 3-chloro- 2-pyridyl | 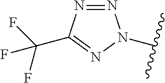 [SMILES: *n1nnc(C(F)(F)F)n1] |
| 43 | Br | 3-chloro- 2-pyridyl | 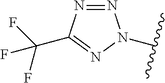 [SMILES: *n1nnc(C(F)(F)F)n1] |
| 44 | I | 3-chloro- 2-pyridyl | 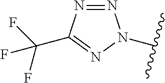 [SMILES: *n1nnc(C(F)(F)F)n1] |
| 45 | Me | 3-chloro- 2-pyridyl | 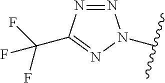 [SMILES: *n1nnc(C(F)(F)F)n1] |
| 46 | F | 3-chloro- 2-pyridyl | 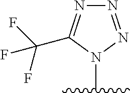 [SMILES: *n1nnnc1C(F)(F)F] |
| 47 | Cl | 3-chloro- 2-pyridyl | 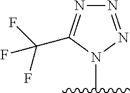 [SMILES: *n1nnnc1C(F)(F)F] |
| 48 | Br | 3-chloro- 2-pyridyl | 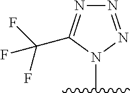 [SMILES: *n1nnnc1C(F)(F)F] |
| 49 | I | 3-chloro- 2-pyridyl | 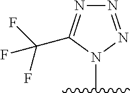 [SMILES: *n1nnnc1C(F)(F)F] |
| 50 | Me | 3-chloro- 2-pyridyl | 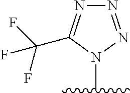 [SMILES: *n1nnnc1C(F)(F)F] |
| 51 | F | 3-chloro- 2-pyridyl | 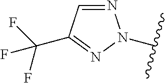 [SMILES: *n1ncc(C(F)(F)F)n1] |
| 52 | Cl | 3-chloro- 2-pyridyl | 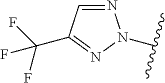 [SMILES: *n1ncc(C(F)(F)F)n1] |
| 53 | Br | 3-chloro- 2-pyridyl | 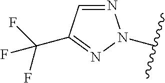 [SMILES: *n1ncc(C(F)(F)F)n1] |
| 54 | I | 3-chloro- 2-pyridyl | 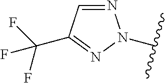 [SMILES: *n1ncc(C(F)(F)F)n1] |
| 55 | Me | 3-chloro- 2-pyridyl | 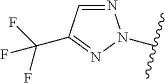 [SMILES: *n1ncc(C(F)(F)F)n1] |
| 56 | F | 3-chloro- 2-pyridyl | 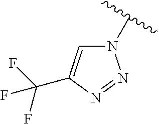 [SMILES: *n1cc(C(F)(F)F)nn1] |
| 57 | Cl | 3-chloro- 2-pyridyl | 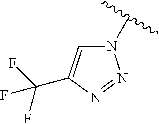 [SMILES: *n1cc(C(F)(F)F)nn1] |
| 58 | Br | 3-chloro- 2-pyridyl | 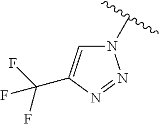 [SMILES: *n1cc(C(F)(F)F)nn1] |
| 59 | I | 3-chloro- 2-pyridyl | 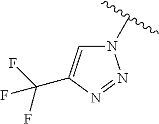 [SMILES: *n1cc(C(F)(F)F)nn1] |
| 60 | Me | 3-chloro- 2-pyridyl | 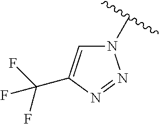 [SMILES: *n1cc(C(F)(F)F)nn1] |
| 61 | F | 3-chloro- 2-pyridyl | 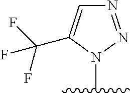 [SMILES: *n1nncc1C(F)(F)F] |
| 62 | Cl | 3-chloro- 2-pyridyl | 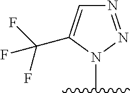 [SMILES: *n1nncc1C(F)(F)F] |
| 63 | Br | 3-chloro- 2-pyridyl | 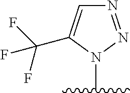 [SMILES: *n1nncc1C(F)(F)F] |
| 64 | I | 3-chloro- 2-pyridyl | 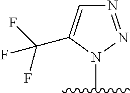 [SMILES: *n1nncc1C(F)(F)F] |
| 65 | Me | 3-chloro- 2-pyridyl | 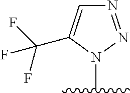 [SMILES: *n1nncc1C(F)(F)F] |
| 66 | F | 3-chloro- 2-pyridyl | 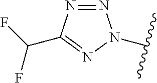 [SMILES: *n1nnc(C(F)F)n1] |
| 67 | Cl | 3-chloro- 2-pyridyl | 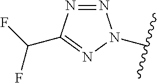 [SMILES: *n1nnc(C(F)F)n1] |
| 68 | Br | 3-chloro- 2-pyridyl | 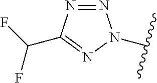 [SMILES: *n1nnc(C(F)F)n1] |
| 69 | I | 3-chloro- 2-pyridyl | 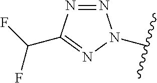 [SMILES: *n1nnc(C(F)F)n1] |
| 70 | Me | 3-chloro- 2-pyridyl | 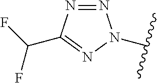 [SMILES: *n1nnc(C(F)F)n1] |
| 71 | F | 3-chloro- 2-pyridyl | 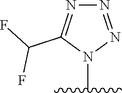 [SMILES: *n1nnnc1C(F)F] |
| 72 | Cl | 3-chloro- 2-pyridyl | 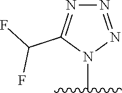 [SMILES: *n1nnnc1C(F)F] |
| 73 | Br | 3-chloro- 2-pyridyl | 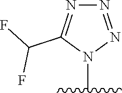 [SMILES: *n1nnnc1C(F)F] |
| 74 | I | 3-chloro- 2-pyridyl | 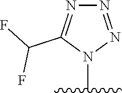 [SMILES: *n1nnnc1C(F)F] |
| 75 | Me | 3-chloro- 2-pyridyl | 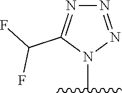 [SMILES: *n1nnnc1C(F)F] |
| 76 | F | 3-chloro- 2-pyridyl | 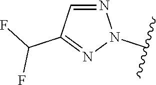 [SMILES: *n1ncc(C(F)F)n1] |
| 77 | Cl | 3-chloro- 2-pyridyl | 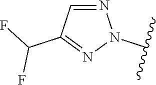 [SMILES: *n1ncc(C(F)F)n1] |
| 78 | Br | 3-chloro- 2-pyridyl | 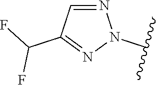 [SMILES: *n1ncc(C(F)F)n1] |
| 79 | I | 3-chloro- 2-pyridyl | 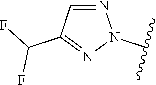 [SMILES: *n1ncc(C(F)F)n1] |
| 80 | Me | 3-chloro- 2-pyridyl | 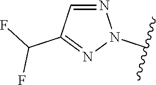 [SMILES: *n1ncc(C(F)F)n1] |
| 81 | F | 3-chloro- 2-pyridyl | 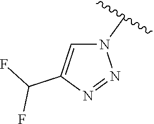 [SMILES: *n1cc(C(F)F)nn1] |
| 82 | Cl | 3-chloro- 2-pyridyl | 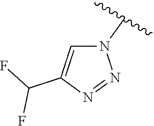 [SMILES: *n1cc(C(F)F)nn1] |
| 83 | Br | 3-chloro- 2-pyridyl | 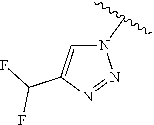 [SMILES: *n1cc(C(F)F)nn1] |
| 84 | I | 3-chloro- 2-pyridyl | 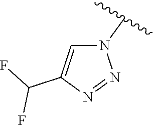 [SMILES: *n1cc(C(F)F)nn1] |
| 85 | Me | 3-chloro- 2-pyridyl | 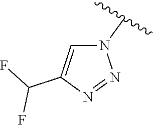 [SMILES: *n1cc(C(F)F)nn1] |
| 86 | F | 3-chloro- 2-pyridyl | 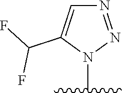 [SMILES: *n1nncc1C(F)F] |
| 87 | Cl | 3-chloro- 2-pyridyl | 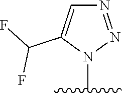 [SMILES: *n1nncc1C(F)F] |
| 88 | Br | 3-chloro- 2-pyridyl | 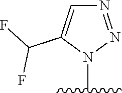 [SMILES: *n1nncc1C(F)F] |
| 89 | I | 3-chloro- 2-pyridyl | 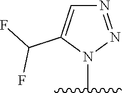 [SMILES: *n1nncc1C(F)F] |
| 90 | Me | 3-chloro- 2-pyridyl | 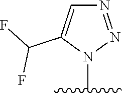 [SMILES: *n1nncc1C(F)F] |
| 91 | F | 3-chloro- 2-pyridyl | 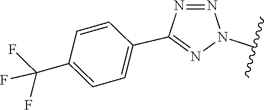 [SMILES: *n1nnc(-c2ccc(C(F)(F)F)cc2)n1] |
| 92 | Cl | 3-chloro- 2-pyridyl | 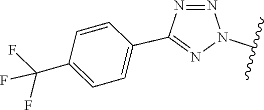 [SMILES: *n1nnc(-c2ccc(C(F)(F)F)cc2)n1] |
| 93 | Br | 3-chloro- 2-pyridyl | 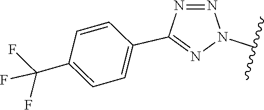 [SMILES: *n1nnc(-c2ccc(C(F)(F)F)cc2)n1] |
| 94 | I | 3-chloro- 2-pyridyl | 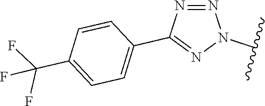 [SMILES: *n1nnc(-c2ccc(C(F)(F)F)cc2)n1] |
| 95 | Me | 3-chloro- 2-pyridyl | 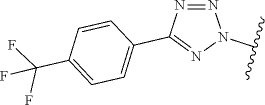 [SMILES: *n1nnc(-c2ccc(C(F)(F)F)cc2)n1] |
| 96 | F | 3-chloro- 2-pyridyl | 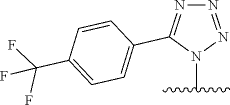 [SMILES: *n1nnnc1-c1ccc(C(F)(F)F)cc1] |
| 97 | Cl | 3-chloro- 2-pyridyl | 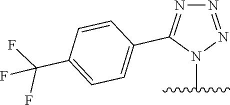 [SMILES: *n1nnnc1-c1ccc(C(F)(F)F)cc1] |
| 98 | Br | 3-chloro- 2-pyridyl | 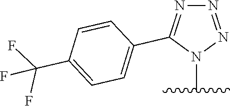 [SMILES: *n1nnnc1-c1ccc(C(F)(F)F)cc1] |
| 99 | I | 3-chloro- 2-pyridyl | 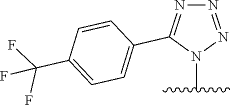 [SMILES: *n1nnnc1-c1ccc(C(F)(F)F)cc1] |
| 100 | Me | 3-chloro- 2-pyridyl | 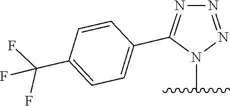 [SMILES: *n1nnnc1-c1ccc(C(F)(F)F)cc1] |
| 101 | F | 3-chloro- 2-pyridyl | 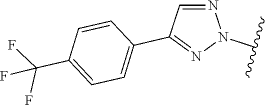 [SMILES: *n1ncc(-c2ccc(C(F)(F)F)cc2)n1] |
| 102 | Cl | 3-chloro- 2-pyridyl | 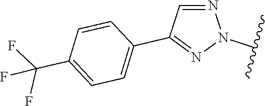 [SMILES: *n1ncc(-c2ccc(C(F)(F)F)cc2)n1] |
| 103 | Br | 3-chloro- 2-pyridyl | 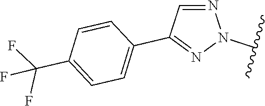 [SMILES: *n1ncc(-c2ccc(C(F)(F)F)cc2)n1] |
| 104 | I | 3-chloro- 2-pyridyl | 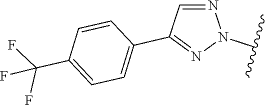 [SMILES: *n1ncc(-c2ccc(C(F)(F)F)cc2)n1] |
| 105 | Me | 3-chloro- 2-pyridyl | 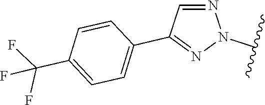 [SMILES: *n1ncc(-c2ccc(C(F)(F)F)cc2)n1] |
| 106 | F | 3-chloro- 2-pyridyl | 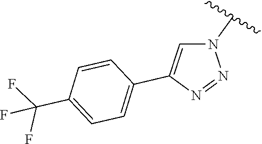 [SMILES: *n1cc(-c2ccc(C(F)(F)F)cc2)nn1] |
| 107 | Cl | 3-chloro- 2-pyridyl | 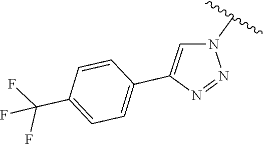 [SMILES: *n1cc(-c2ccc(C(F)(F)F)cc2)nn1] |
| 108 | Br | 3-chloro- 2-pyridyl | 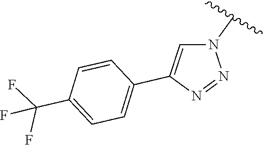 [SMILES: *n1cc(-c2ccc(C(F)(F)F)cc2)nn1] |
| 109 | I | 3-chloro- 2-pyridyl | 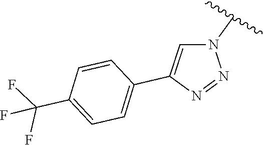 [SMILES: *n1cc(-c2ccc(C(F)(F)F)cc2)nn1] |
| 110 | Me | 3-chloro- 2-pyridyl | 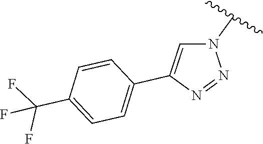 [SMILES: *n1cc(-c2ccc(C(F)(F)F)cc2)nn1] |
| 111 | F | 3-chloro- 2-pyridyl | 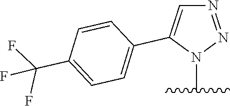 [SMILES: *n1nncc1-c1ccc(C(F)(F)F)cc1] |
| 112 | Cl | 3-chloro- 2-pyridyl | 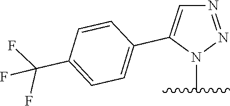 [SMILES: *n1nncc1-c1ccc(C(F)(F)F)cc1] |
| 113 | Br | 3-chloro- 2-pyridyl | 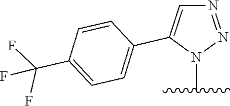 [SMILES: *n1nncc1-c1ccc(C(F)(F)F)cc1] |
| 114 | I | 3-chloro- 2-pyridyl | 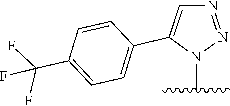 [SMILES: *n1nncc1-c1ccc(C(F)(F)F)cc1] |
| 115 | Me | 3-chloro- 2-pyridyl | 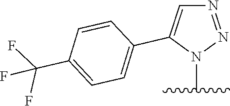 [SMILES: *n1nncc1-c1ccc(C(F)(F)F)cc1] |

wherein the staggered line indicates the point of attachment of R₄ to the rest of the molecule.

Table A-1 provides 115 compounds A-1.001 to A-1.115 of formula IAA wherein R₂ is Br and R₄, R₁, R₃ are as defined in table X (wherein the staggered line indicates the point of attachment of R₄ to the rest of the molecule). For example, compound A1.041 has the following structure:

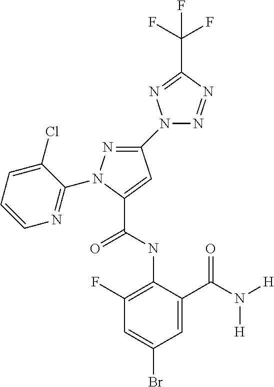 [SMILES: NC(=O)c1cc(Br)cc(F)c1NC(=O)c1cc(-n2nnc(C(F)(F)F)n2)nn1-c1ncccc1Cl]

Table A-2 provides 115 compounds A-2.001 to A-2.115 of formula IAA wherein R₂ is I and R₄, R₁, R₃ are as defined in table X.

Table A-3 provides 115 compounds A-3.001 to A-3.115 of formula IAA wherein R₂ is CN and R₄, R₁, R₃ are as defined in table X.

Table A-4 provides 115 compounds A-4.001 to A-4.115 of formula IAA wherein R₂ is C₁ and R₄, R₁, R₃ are as defined in table X.

The compounds of formula I (including Ia to Id) according to the invention are preventively and/or curatively valuable active ingredients in the field of pest control, even at low rates of application, which have a very favorable biocidal spectrum and are well tolerated by warm-blooded species, fish and plants. The active ingredients according to the invention act against all or individual developmental stages of normally sensitive, but also resistant, animal pests, such as insects or representatives of the order Acarina. The insecticidal or acaricidal activity of the active ingredients according to the invention can manifest itself directly, i. e. in destruction of the pests, which takes place either immediately or only after some time has elapsed, for example during ecdysis, or indirectly, for example in a reduced oviposition and/or hatching rate.

Examples of the above mentioned animal pests are:
from the order Acarina, for example,
Acalitus spp, Aculus spp, Acaricalus spp, Aceria spp, Acarus siro, Amblyomma spp., Argas spp., Boophilus spp., Brevipalpus spp., Bryobia spp, Calipitrimerus spp., Chorioptes spp., Dermanyssus gallinae, Dermatophagoides spp, Eotetranychus spp, Eriophyes spp., Hemitarsonemus spp, Hyalomma spp., Ixodes spp., Olygonychus spp, Ornithodoros spp., Polyphagotarsone latus, Panonychus spp., Phyllocoptruta oleivora, Phytonemus spp, Polyphagotarsonemus spp, Psoroptes spp., Rhipicephalus spp., Rhizoglyphus spp., Sarcoptes spp., Steneotarsonemus spp, Tarsonemus spp. and Tetranychus spp.;
from the order Anoplura, for example,
Haematopinus spp., Linognathus spp., Pediculus spp., Pemphigus spp. and Phylloxera spp.;
from the order Coleoptera, for example,
Agriotes spp., Amphimallon majale, Anomala orientalis, Anthonomus spp., Aphodius spp, Astylus atromaculatus, Ataenius spp, Atomaria linearis, Chaetocnema tibialis, Cerotoma spp, Conoderus spp, Cosmopolites spp., Cotinis nitida, Curculio spp., Cyclocephala spp, Dermestes spp., Diabrotica spp., Diloboderus abderus, Epilachna spp., Eremnus spp., Heteronychus arator, Hypothenemus hampei, Lagria vilosa, Leptinotarsa decemlineata, Lissorhoptrus spp., Liogenys spp, Maecolaspis spp, Maladera castanea, Megascelis spp, Melighetes aeneus, Melolontha spp., Myochrous armatus, Orycaephilus spp., Otiorhynchus spp., Phyllophaga spp, Phlyctinus spp., Popillia spp., Psylliodes spp., Rhyssomatus aubtilis, Rhizopertha spp., Scarabeidae, Sitophilus spp., Sitotroga spp., Somaticus spp, Sphenophorus spp, Sternechus subsignatus, Tenebrio spp., Tribolium spp. and Trogoderma spp.;
from the order Diptera, for example,
Aedes spp., Anopheles spp, Antherigona soccata, Bactrocea oleae, Bibio hortulanus, Bradysia spp, Calliphora erythrocephala, Ceratitis spp., Chrysomyia spp., Culex spp., Cuterebra spp., Dacus spp., Delia spp, Drosophila melanogaster, Fannia spp., Gastrophilus spp., Geomyza tripunctata, Glossina spp., Hypoderma spp., Hyppobosca spp., Liriomyza spp., Lucilia spp., Melanagromyza spp., Musca spp., Oestrus spp., Orseolia spp., Oscinella frit, Pegomyia hyoscyami, Phorbia spp., Rhagoletis spp, Rivelia quadrifasciata, Scatella spp, Sciara spp., Stomoxys spp., Tabanus spp., Tannia spp. and Tipula spp.;
from the order Hemiptera, for example,
Acanthocoris scabrator, Acrosternum spp, Adelphocoris lineolatus, Aleurodes spp., Amblypelta nitida, Bathycoelia thalassina, Blissus spp, Cimex spp., Clavigralla tomentosicollis, Creontiades spp, Distantiella theobroma, Dichelops furcatus, Dysdercus spp., Edessa spp, Euchistus spp., Eurydema pulchrum, Eurygaster spp., Halyomorpha halys, Horcias nobilellus, Leptocorisa spp., Lygus spp, Margarodes spp, Murgantia histrionic, Neomegalotomus spp, Nesidiocoris tenuis, Nezara spp., Nysius simulans, Oebalus insularis, Piesma spp., Piezodorus spp, Rhodnius spp., Sahlbergella singularis, Scaptocoris castanea, Scotinophara spp., Thyanta spp, Triatoma spp., Vatiga illudens; Acyrthosium pisum, Adalges spp, Agalliana ensigera, Agonoscena targionii, Aleurodicus spp, Aleurocanthus spp, Aleurolobus barodensis, Aleurothrixus floccosus, Aleyrodes brassicae, Amarasca biguttula, Amritodus atkinsoni, Aonidiella spp., Aphididae, Aphis spp., Aspidiotus spp., Aulacorthum solani, Bactericera cockerelli, Bemisia spp, Brachycaudus spp, Brevicoryne brassicae, Cacopsylla spp, Cavariella aegopodii Scop., Ceroplaster spp., Chrysomphalus aonidium, Chrysomphalus dictyospermi, Cicadella spp, Cofana spectra, Cryptomyzus spp, Cicadulina spp, Coccus hesperidum, Dalbulus maidis, Dialeurodes spp, Diaphorina citri, Diuraphis noxia, Dysaphis spp, Empoasca spp., Eriosoma larigerum, Erythroneura spp., Gascardia spp., Glycaspis brimblecombei, Hyadaphis pseudobrassicae, Hyalopterus spp, Hyperomyzus pallidus, Idioscopus clypealis, Jacobiasca lybica, Laodelphax spp., Lecanium corni, Lepidosaphes spp., Lopaphis erysimi, Lyogenys maidis, Macrosiphum spp., Mahanarva spp, Metcalfa pruinosa, Metopolophium dirhodum, Myndus crudus, Myzus spp., Neotoxoptera sp, Nephotettix spp., Nilaparvata spp., Nippolachnus piri Mats, Odonaspis ruthae, Oregma lanigera Zehnter, Parabemisia myricae, Paratrioza cockerelli, Parlatoria spp., Pemphigus spp., Peregrinus maidis, Perkinsiella spp, Phorodon humuli, Phylloxera spp, Planococcus spp., Pseudaulacaspis spp., Pseudococcus spp., Pseudatomoscelis seriatus, Psylla spp., Pulvinaria aethiopica, Quadraspidiotus spp., Quesada gigas, Recilia dorsalis, Rhopalosiphum spp., Saissetia spp., Scaphoideus spp., Schizaphis spp., Sitobion spp., Sogatella furcifera, Spissistilus festinus, Tarophagus proserpina, Toxoptera spp, Trialeurodes spp, Tridiscus sporoboli, Trionymus spp, Trioza erytreae, Unaspis citri, Zygina flammigera, Zyginidia scutellaris;
from the order Hymenoptera, for example,
Acromyrmex, Arge spp, Atta spp., Cephus spp., Diprion spp., Diprionidae, Gilpinia polytoma, Hoplocampa spp., Lasius spp., Monomorium pharaonis, Neodiprion spp., Pogonomyrmex spp, Slenopsis invicta, Solenopsis spp. and Vespa spp.;
from the order Isoptera, for example,
Coptotermes spp, Corniternes cumulans, Incisitermes spp, Macrotermes spp, Mastotermes spp, Microtermes spp, Reticulitermes spp.; Solenopsis geminate
from the order Lepidoptera, for example,
Acleris spp., Adoxophyes spp., Aegeria spp., Agrotis spp., Alabama argillaceae, Amylois spp., Anticarsia gemmatalis, Archips spp., Argyresthia spp, Argyrotaenia spp., Autographa spp., Bucculatrix thurberiella, Busseola fusca, Cadra cautella, Carposina nipponensis, Chilo spp., Choristoneura spp., Chrysoteuchia topiaria, Clysia ambiguella, Cnaphalocrocis spp., Cnephasia spp., Cochylis spp., Coleophora spp., Colias lesbia, Cosmophila flava, Crambus spp, Crocidolomia binotalis, Cryptophlebia leucotreta, Cydalima perspectalis, Cydia spp., Diaphania perspectalis, Diatraea spp., Diparopsis castanea, Earias spp., Elasmopalpus lignosellus, Eldana saccharina, Ephestia spp., Epinotia spp, Estigmene acrea, Etiella zinckinella, Eucosma spp., Eupoecilia ambiguella, Euproctis spp., Euxoa spp., Feltia jaculiferia, Grapholita spp., Hedya nubiferana, Heliothis spp., Hellula undalis, Herpetogramma spp, Hyphantria cunea, Keiferia lycopersicella, Lasmopalpus lignosellus, Leucoptera scitella, Lithocollethis spp., Lobesia botrana, Loxostege bifidalis, Lymantria spp., Lyonetia spp., Malacosoma spp., Mamestra brassicae, Manduca sexta, Mythimna spp, Noctua spp, Operophtera spp., Orniodes indica, Ostrinia nubilalis, Pammene spp., Pandemis spp., Panolis flammea, Papaipema nebris, Pectinophora gossypiela, Perileucoptera coffeella, Pseudaletia unipuncta, Phthorimaea operculella, Pieris rapae, Pieris spp., Plutella xylostella, Prays spp., Pseudoplusia spp, Rachiplusia nu, Richia albicosta, Scirpophaga spp., Sesamia spp., Sparganothis spp., Spodoptera spp., Sylepta derogate, Synanthedon spp., Thaumetopoea spp., Tortrix spp., Trichoplusia ni, Tuta absoluta, and Yponomeuta spp.;
from the order Mallophaga, for example,
Damalinea spp. and Trichodectes spp.;
from the order Orthoptera, for example,
Blatta spp., Blattella spp., Gryllotalpa spp., Leucophaea maderae, Locusta spp., Neocurtilla hexadactyla, Periplaneta spp., Scapteriscus spp, and Schistocerca spp.;
from the order Psocoptera, for example,
Liposcelis spp.;
from the order Siphonaptera, for example,
Ceratophyllus spp., Ctenocephalides spp. and Xenopsylla cheopis;
from the order Thysanoptera, for example,
Calliothrips phaseoli, Frankliniella spp., Heliothrips spp, Hercinothrips spp., Parthenothrips spp, Scirtothrips aurantii, Sericothrips variabilis, Taeniothrips spp., Thrips spp;
from the order Thysanura, for example, Lepisma saccharina.

In a further aspect, the invention may also relate to a method of controlling damage to plant and parts thereof by plant parasitic nematodes (Endoparasitic-, Semiendoparasitic- and Ectoparasitic nematodes), especially plant parasitic nematodes such as root knot nematodes, Meloidogyne hapla, Meloidogyne incognita, Meloidogyne javanica, Meloidogyne arenaria and other Meloidogyne species; cyst-forming nematodes, Globodera rostochiensis and other Globodera species; Heterodera avenae, Heterodera glycines, Heterodera schachtii, Heterodera trifolii, and other Heterodera species; Seed gall nematodes, Anguina species; Stem and foliar nematodes, Aphelenchoides species; Sting nematodes, Belonolaimus longicaudatus and other Belonolaimus species; Pine nematodes, Bursaphelenchus xylophilus and other Bursaphelenchus species; Ring nematodes, Criconema species, Criconemella species, Criconemoides species, Mesocriconema species; Stem and bulb nematodes, Ditylenchus destructor, Ditylenchus dipsaci and other Ditylenchus species; Awl nematodes, Dolichodorus species; Spiral nematodes, Heliocotylenchus multicinctus and other Helicotylenchus species; Sheath and sheathoid nematodes, Hemicycliophora species and Hemicriconemoides species; Hirshmanniella species; Lance nematodes, Hoploaimus species; false rootknot nematodes, Nacobbus species; Needle nematodes, Longidorus elongatus and other Longidorus species; Pin nematodes, Pratylenchus species; Lesion nematodes, Pratylenchus neglectus, Pratylenchus penetrans, Pratylenchus curvitatus, Pratylenchus goodeyi and other Pratylenchus species; Burrowing nematodes, Radopholus similis and other Radopholus species; Reniform nematodes, Rotylenchus robustus, Rotylenchus reniformis and other Rotylenchus species; Scutellonema species; Stubby root nematodes, Trichodorus primitivus and other Trichodorus species, Paratrichodorus species; Stunt nematodes, Tylenchorhynchus claytoni, Tylenchorhynchus dubius and other Tylenchorhynchus species; Citrus nematodes, Tylenchulus species; Dagger nematodes, Xiphinema species; and other plant parasitic nematode species, such as Subanguina spp., Hypsoperine spp., Macroposthonia spp., Melinius spp., Punctodera spp., and Quinisulcius spp.

The compounds of the invention may also have activity against the molluscs. Examples of which include, for example, Ampullariidae; Arion (A. ater, A. circumscriptus, A. hortensis, A. rufus); Bradybaenidae (Bradybaena fruticum); Cepaea (C. hortensis, C. nemoralis); ochlodina; Deroceras (D. agrestis, D. empiricorum, D. laeve, D. reticulatum); Discus (D. rotundatus); Euomphalia; Galba (G. trunculata); Helicelia (H. itala, H. obvia); Helicidae Helicigona arbustorum); Helicodiscus; Helix (H. aperta); Limax (L. cinereoniger, L. flavus, L. marginatus, L. maximus, L. tenellus); Lymnaea; Milax (M. gagates, M. marginatus, M. sowerbyi); Opeas; Pomacea (P. canaticulata); Vallonia and Zanitoides.

The active ingredients according to the invention can be used for controlling, i. e. containing or destroying, pests of the abovementioned type which occur in particular on plants, especially on useful plants and ornamentals in agriculture, in horticulture and in forests, or on organs, such as fruits, flowers, foliage, stalks, tubers or roots, of such plants, and in some cases even plant organs which are formed at a later point in time remain protected against these pests.

Suitable target plant or crops are, in particular, cereals, such as wheat, barley, rye, oats, rice, maize or sorghum; beet, such as sugar or fodder beet; fruit, for example pomaceous fruit, stone fruit or soft fruit, such as apples, pears, plums, peaches, almonds, cherries or berries, for example strawberries, raspberries or blackberries; leguminous crops, such as beans, lentils, peas or soya; oil crops, such as oilseed rape, mustard, poppies, olives, sunflowers, coconut, castor, cocoa or ground nuts; cucurbits, such as pumpkins, cucumbers or melons; fibre plants, such as cotton, flax, hemp or jute; citrus fruit, such as oranges, lemons, grapefruit or tangerines; vegetables, such as spinach, lettuce, asparagus, cabbages, carrots, onions, tomatoes, potatoes or bell peppers; Lauraceae, such as avocado, Cinnamonium or camphor; and also tobacco, nuts, coffee, eggplants, sugarcane, tea, pepper, grapevines, hops, the plantain family and latex plants.

The compositions and/or methods of the present invention may be also used on any ornamental and/or vegetable crops, including flowers, shrubs, broad-leaved trees and evergreens.

For example the invention may be used on any of the following ornamental species: Ageratum spp., Alonsoa spp., Anemone spp., Anisodontea capsenisis, Anthemis spp., Antirrhinum spp., Aster spp., Begonia spp. (e.g. B. elatior, B. semperflorens, B. tubereux), Bougainvillea spp., Brachycome spp., Brassica spp. (ornamental), Calceolaria spp., Capsicum annuum, Catharanthus roseus, Canna spp., Centaurea spp., Chrysanthemum spp., Cineraria spp. (C. maritime), Coreopsis spp., Crassula coccinea, Cuphea ignea, Dahlia spp., Delphinium spp., Dicentra spectabilis, Dorotheantus spp., Eustoma grandiflorum, Forsythia spp., Fuchsia spp., Geranium gnaphalium, Gerbera spp., Gomphrena globosa, Heliotropium spp., Helianthus spp., Hibiscus spp., Hortensia spp., Hydrangea spp., Hypoestes phyllostachya, Impatiens spp. (I. Walleriana), Iresines spp., Kalanchoe spp., Lantana camara, Lavatera trimestris, Leonotis leonurus, Lilium spp., Mesembryanthemum spp., Mimulus spp., Monarda spp., Nemesia spp., Tagetes spp., Dianthus spp. (carnation), Canna spp., Oxalis spp., Bellis spp., Pelargonium spp. (P. peltatum, P. Zonale), Viola spp. (pansy), Petunia spp., Phlox spp., Plecthranthus spp., Poinsettia spp., Parthenocissus spp. (P. quinquefolia, P. tricuspidata), Primula spp., Ranunculus spp., Rhododendron spp., Rosa spp. (rose), Rudbeckia spp., Saintpaulia spp., Salvia spp., Scaevola aemola, Schizanthus wisetonensis, Sedum spp., Solanum spp., Surfinia spp., Tagetes spp., Nicotinia spp., Verbena spp., Zinnia spp. and other bedding plants.

For example the invention may be used on any of the following vegetable species: Allium spp. (A. sativum, A. cepa, A. oschaninii, A. Porrum, A. ascalonicum, A. fistulosum), Anthriscus cerefolium, Apium graveolus, Asparagus officinalis, Beta vulgarus, Brassica spp. (B. Oleracea, B. Pekinensis, B. rapa), Capsicum annuum, Cicer arietinum, Cichorium endivia, Cichorum spp. (C. intybus, C. endivia), Citrillus lanatus, Cucumis spp. (C. sativus, C. melo), Cucurbita spp. (C. pepo, C. maxima), Cyanara spp. (C. scolymus, C. cardunculus), Daucus carota, Foeniculum vulgare, Hypericum spp., Lactuca sativa, Lycopersicon spp. (L. esculentum, L. lycopersicum), Mentha spp., Ocimum basilicum, Petroselinum crispum, Phaseolus spp. (P. vulgaris, P. coccineus), Pisum sativum, Raphanus sativus, Rheum rhaponticum, Rosemarinus spp., Salvia spp., Scorzonera hispanica, Solanum melongena, Spinacea oleracea, Valerianella spp. (V. locusta, V. eriocarpa) and Vicia faba.

Preferred ornamental species include African violet, Begonia, Dahlia, Gerbera, Hydrangea, Verbena, Rosa, Kalanchoe, Poinsettia, Aster, Centaurea, Coreopsis, Delphinium, Monarda, Phlox, Rudbeckia, Sedum, Petunia, Viola, Impatiens, Geranium, Chrysanthemum, Ranunculus, Fuchsia, Salvia, Hortensia, rosemary, sage, St. Johnswort, mint, sweet pepper, tomato and cucumber.

The active ingredients according to the invention are especially suitable for controlling Aphis craccivora, Diabrotica balteata, Heliothis virescens, Myzus persicae, Plutella xylostella and Spodoptera littoralis in cotton, vegetable, maize, rice and soya crops. The active ingredients according to the invention are further especially suitable for controlling Mamestra (preferably in vegetables), Cydia pomonella (preferably in apples), Empoasca (preferably in vegetables, vineyards), Leptinotarsa (preferably in potatoes) and Chilo supressalis (preferably in rice).

The compounds of formula I (including formulae Ia to Id) are particularly suitable for control of
a pest of the order Lepidoptera, for example, one or more of the species Spodoptera littoralis, Spodoptera frugiperda, Plutella xylostella, Cnaphalocrocis medinalis, Cydia pomonella, Chrysodeixis includens, Chilo suppressalis, Elasmopalpus lignosellus, Pseudoplusia includens, and Tuta absoluta (preferably in vegetables and corn).

The term “crops” is to be understood as including also crop plants which have been so transformed by the use of recombinant DNA techniques that they are capable of synthesising one or more selectively acting toxins, such as are known, for example, from toxin-producing bacteria, especially those of the genus Bacillus.

Toxins that can be expressed by such transgenic plants include, for example, insecticidal proteins, for example insecticidal proteins from Bacillus cereus or Bacillus popilliae; or insecticidal proteins from Bacillus thuringiensis, such as 6-endotoxins, e.g. Cry1Ab, Cry1Ac, Cry1F, Cry1Fa2, Cry2Ab, Cry3A, Cry3Bb1 or Cry9C, or vegetative insecticidal proteins (Vip), e.g. Vip1, Vip2, Vip3 or Vip3A; or insecticidal proteins of bacteria colonising nematodes, for example Photorhabdus spp. or Xenorhabdus spp., such as Photorhabdus luminescens, Xenorhabdus nematophilus; toxins produced by animals, such as scorpion toxins, arachnid toxins, wasp toxins and other insect-specific neurotoxins; toxins produced by fungi, such as Streptomycetes toxins, plant lectins, such as pea lectins, barley lectins or snowdrop lectins; agglutinins; proteinase inhibitors, such as trypsin inhibitors, serine protease inhibitors, patatin, cystatin, papain inhibitors; ribosome-inactivating proteins (RIP), such as ricin, maize-RIP, abrin, luffin, saporin or bryodin; steroid metabolism enzymes, such as 3-hydroxysteroidoxidase, ecdysteroid-UDP-glycosyl-transferase, cholesterol oxidases, ecdysone inhibitors, HMG-COA-reductase, ion channel blockers, such as blockers of sodium or calcium channels, juvenile hormone esterase, diuretic hormone receptors, stilbene synthase, bibenzyl synthase, chitinases and glucanases.

In the context of the present invention there are to be understood by 6-endotoxins, for example Cry1Ab, Cry1Ac, Cry1F, Cry1Fa2, Cry2Ab, Cry3A, Cry3Bb1 or Cry9C, or vegetative insecticidal proteins (Vip), for example Vip1, Vip2, Vip3 or Vip3A, expressly also hybrid toxins, truncated toxins and modified toxins. Hybrid toxins are produced recombinantly by a new combination of different domains of those proteins (see, for example, WO 02/15701). Truncated toxins, for example a truncated Cry1Ab, are known. In the case of modified toxins, one or more amino acids of the naturally occurring toxin are replaced. In such amino acid replacements, preferably non-naturally present protease recognition sequences are inserted into the toxin, such as, for example, in the case of Cry3A055, a cathepsin-G-recognition sequence is inserted into a Cry3A toxin (see WO 03/018810).

Examples of such toxins or transgenic plants capable of synthesising such toxins are disclosed, for example, in EP-A-0 374 753, WO 93/07278, WO 95/34656, EP-A-0 427 529, EP-A-451 878 and WO 03/052073.

The processes for the preparation of such transgenic plants are generally known to the person skilled in the art and are described, for example, in the publications mentioned above. Cryl-type deoxyribonucleic acids and their preparation are known, for example, from WO 95/34656, EP-A-0 367 474, EP-A-0 401 979 and WO 90/13651.

The toxin contained in the transgenic plants imparts to the plants tolerance to harmful insects. Such insects can occur in any taxonomic group of insects, but are especially commonly found in the beetles (Coleoptera), two-winged insects (Diptera) and moths (Lepidoptera).

Transgenic plants containing one or more genes that code for an insecticidal resistance and express one or more toxins are known and some of them are commercially available. Examples of such plants are: YieldGard® (maize variety that expresses a Cry1Ab toxin); YieldGard Rootworm® (maize variety that expresses a Cry3Bb1 toxin); YieldGard Plus® (maize variety that expresses a Cry1Ab and a Cry3Bb1 toxin); Starlink® (maize variety that expresses a Cry9C toxin); Herculex 1® (maize variety that expresses a Cry1Fa2 toxin and the enzyme phosphinothricine N-acetyltransferase (PAT) to achieve tolerance to the herbicide glufosinate ammonium); NuCOTN 33B® (cotton variety that expresses a Cry1Ac toxin); Bollgard 1® (cotton variety that expresses a Cry1Ac toxin); Bollgard 11® (cotton variety that expresses a Cry1Ac and a Cry2Ab toxin); VipCot® (cotton variety that expresses a Vip3A and a Cry1Ab toxin); NewLeaf® (potato variety that expresses a Cry3A toxin); NatureGard®, Agrisure® GT Advantage (GA21 glyphosate-tolerant trait), Agrisure® CB Advantage (Bt11 corn borer (CB) trait) and Protecta®.

Further examples of such transgenic crops are:

1. Bt11 Maize from Syngenta Seeds SAS, Chemin de l'Hobit 27, F-31 790 St. Sauveur, France, registration number C/FR/96/05/10. Genetically modified Zea mays which has been rendered resistant to attack by the European corn borer (Ostrinia nubilalis and Sesamia nonagrioides) by transgenic expression of a truncated Cry1Ab toxin. Bt11 maize also transgenically expresses the enzyme PAT to achieve tolerance to the herbicide glufosinate ammonium.

2. Bt176 Maize from Syngenta Seeds SAS, Chemin de l'Hobit 27, F-31 790 St. Sauveur, France, registration number C/FR/96/05/10. Genetically modified Zea mays which has been rendered resistant to attack by the European corn borer (Ostrinia nubilalis and Sesamia nonagrioides) by transgenic expression of a Cry1Ab toxin. Bt176 maize also transgenically expresses the enzyme PAT to achieve tolerance to the herbicide glufosinate ammonium.

3. MIR604 Maize from Syngenta Seeds SAS, Chemin de l'Hobit 27, F-31 790 St. Sauveur, France, registration number C/FR/96/05/10. Maize which has been rendered insect-resistant by transgenic expression of a modified Cry3A toxin. This toxin is Cry3A055 modified by insertion of a cathepsin-G-protease recognition sequence. The preparation of such transgenic maize plants is described in WO 03/018810.

4. MON 863 Maize from Monsanto Europe S.A. 270-272 Avenue de Tervuren, B-1150 Brussels, Belgium, registration number C/DE/02/9. MON 863 expresses a Cry3Bb1 toxin and has resistance to certain Coleoptera insects.

5. IPC 531 Cotton from Monsanto Europe S.A. 270-272 Avenue de Tervuren, B-1150 Brussels, Belgium, registration number C/ES/96/02.

6. 1507 Maize from Pioneer Overseas Corporation, Avenue Tedesco, 7 B-1160 Brussels, Belgium, registration number C/NL/00/10. Genetically modified maize for the expression of the protein Cry1F for achieving resistance to certain Lepidoptera insects and of the PAT protein for achieving tolerance to the herbicide glufosinate ammonium.

7. NK603×MON 810 Maize from Monsanto Europe S.A. 270-272 Avenue de Tervuren, B-1150 Brussels, Belgium, registration number C/GB/02/M3/03. Consists of conventionally bred hybrid maize varieties by crossing the genetically modified varieties NK603 and MON 810. NK603×MON 810 Maize transgenically expresses the protein CP4 EPSPS, obtained from Agrobacterium sp. strain CP4, which imparts tolerance to the herbicide Roundup® (contains glyphosate), and also a Cry1Ab toxin obtained from Bacillus thuringiensis subsp. kurstaki which brings about tolerance to certain Lepidoptera, include the European corn borer.

Transgenic crops of insect-resistant plants are also described in BATS (Zentrum fur Biosicherheit und Nachhaltigkeit, Zentrum BATS, Clarastrasse 13, 4058 Basel, Switzerland) Report 2003, (http://bats.ch).

The term “crops” is to be understood as including also crop plants which have been so transformed by the use of recombinant DNA techniques that they are capable of synthesising antipathogenic substances having a selective action, such as, for example, the so-called “pathogenesis-related proteins” (PRPs, see e.g. EP-A-0 392 225). Examples of such antipathogenic substances and transgenic plants capable of synthesising such antipathogenic substances are known, for example, from EP-A-0 392 225, WO 95/33818 and EP-A-0 353 191. The methods of producing such transgenic plants are generally known to the person skilled in the art and are described, for example, in the publications mentioned above.

Crops may also be modified for enhanced resistance to fungal (for example Fusarium, Anthracnose, or Phytophthora), bacterial (for example Pseudomonas) or viral (for example potato leafroll virus, tomato spotted wilt virus, cucumber mosaic virus) pathogens.

Crops also include those that have enhanced resistance to nematodes, such as the soybean cyst nematode.

Crops that are tolerance to abiotic stress include those that have enhanced tolerance to drought, high salt, high temperature, chill, frost, or light radiation, for example through expression of NF-YB or other proteins known in the art.

Antipathogenic substances which can be expressed by such transgenic plants include, for example, ion channel blockers, such as blockers for sodium and calcium channels, for example the viral KP1, KP4 or KP6 toxins; stilbene synthases; bibenzyl synthases; chitinases; glucanases; the so-called “pathogenesis-related proteins” (PRPs; see e.g. EP-A-0 392 225); antipathogenic substances produced by microorganisms, for example peptide antibiotics or heterocyclic antibiotics (see e.g. WO 95/33818) or protein or polypeptide factors involved in plant pathogen defence (so-called “plant disease resistance genes”, as described in WO 03/000906).

Further areas of use of the compositions according to the invention are the protection of stored goods and store rooms and the protection of raw materials, such as wood, textiles, floor coverings or buildings, and also in the hygiene sector, especially the protection of humans, domestic animals and productive livestock against pests of the mentioned type.

The present invention provides a compound of the second aspect for use in therapy. The present invention provides a compound of the first aspect, for use in controlling parasites in or on an animal. The present invention further provides a compound of the first aspect, for use in controlling ectoparasites on an animal. The present invention further provides a compound of the first aspect, for use in preventing and/or treating diseases transmitted by ectoparasites.

The present invention provides the use of a compound of the second aspect, for the manufacture of a medicament for controlling parasites in or on an animal. The present invention further provides the use of a compound of the first aspect, for the manufacture of a medicament for controlling ectoparasites on an animal. The present invention further provides the use of a compound of the first aspect, for the manufacture of a medicament for preventing and/or treating diseases transmitted by ectoparasites.

The present invention provides the use of a compound of the first aspect, in controlling parasites in or on an animal. The present invention further provides the use of a compound of the second aspect, in controlling ectoparasites on an animal.

The term “controlling” when used in context of parasites in or on an animal refers to reducing the number of pests or parasites, eliminating pests or parasites and/or preventing further pest or parasite infestation.

The term “treating” when used in context of parasites in or on an animal refers to restraining, slowing, stopping or reversing the progression or severity of an existing symptom or disease.

The term “preventing” when used in context of parasites in or on an animal refers to the avoidance of a symptom or disease developing in the animal.

The term “animal” when used in context of parasites in or on an animal may refer to a mammal and a non-mammal, such as a bird or fish. In the case of a mammal, it may be a human or non-human mammal. Non-human mammals include, but are not limited to, livestock animals and companion animals. Livestock animals include, but are not limited to, cattle, camellids, pigs, sheep, goats and horses. Companion animals include, but are not limited to, dogs, cats and rabbits.

A “parasite” is a pest which lives in or on the host animal and benefits by deriving nutrients at the host animal's expense. An “endoparasite” is a parasite which lives in the host animal. An “ectoparasite” is a parasite which lives on the host animal. Ectoparasites include, but are not limited to, acari, insects and crustaceans (e.g. sea lice). The Acari (or Acarina) sub-class comprises ticks and mites. Ticks include, but are not limited to, members of the following genera: Rhipicaphalus, for example, Rhipicaphalus (Boophilus) microplus and Rhipicephalus sanguineus; Amblyomrna; Dermacentor; Haemaphysalis; Hyalomma; Ixodes; Rhipicentor; Margaropus; Argas; Otobius; and Ornithodoros. Mites include, but are not limited to, members of the following genera: Chorioptes, for example Chorioptes bovis; Psoroptes, for example Psoroptes ovis; Cheyletiella; Dermanyssus; for example Dermanyssus gallinae; Ortnithonyssus; Demodex, for example Demodex canis; Sarcoptes, for example Sarcoptes scabiei; and Psorergates. Insects include, but are not limited to, members of the orders: Siphonaptera, Diptera, Phthiraptera, Lepidoptera, Coleoptera and Homoptera. Members of the Siphonaptera order include, but are not limited to, Ctenocephalides felis and Ctenocephatides canis. Members of the Diptera order include, but are not limited to, Musca spp.; bot fly, for example Gasterophilus intestinalis and Oestrus ovis; biting flies; horse flies, for example Haematopota spp. and Tabunus spp.; haematobia, for example haematobia irritans; Stomoxys; Lucilia; midges; and mosquitoes. Members of the Phthiraptera class include, but are not limited to, blood sucking lice and chewing lice, for example Bovicola ovis and Bovicola bovis.

The term “effective amount” when used in context of parasites in or on an animal refers to the amount or dose of the compound of the invention, or a salt thereof, which, upon single or multiple dose administration to the animal, provides the desired effect in or on the animal. The effective amount can be readily determined by the attending diagnostician, as one skilled in the art, by the use of known techniques and by observing results obtained under analogous circumstances. In determining the effective amount a number of factors are considered by the attending diagnostician, including, but not limited to: the species of mammal; its size, age, and general health; the parasite to be controlled and the degree of infestation; the specific disease or disorder involved; the degree of involvement or the severity of the disease or disorder; the response of the individual; the particular compound administered; the mode of administration; the bioavailability characteristics of the preparation administered; the dose regimen selected; the use of concomitant medication; and other relevant circumstances.

The compounds of the invention may be administered to the animal by any route which has the desired effect including, but not limited to topically, orally, parenterally’ and subcutaneously. Topical administration is preferred. Formulations suitable for topical administration include, for example, solutions, emulsions and suspensions and may take the form of a pour-on, spot-on, spray-on, spray race or dip. In the alternative, the compounds of the invention may be administered by means of an ear tag or collar.

Salt forms of the compounds of the invention include both pharmaceutically acceptable salts and veterinary acceptable salts, which can be different to agrochemically acceptable salts. Pharmaceutically and veterinary acceptable salts and common methodology for preparing them are well known in the art. See, for example, Gould, P. L., “Salt selection for basic drugs”, International Journal of Pharmaceutics, 33: 201-217 (1986); Bastin, R. J., et al. “Salt Selection and Optimization Procedures for Pharmaceutical New Chemical Entities”, Organic Process Research and Development, 4: 427-435 (2000); and Berge, S. M., et al., “Pharmaceutical Salts”, Journal of Pharmaceutical Sciences, 66: 1-19, (1977). One skilled in the art of synthesis will appreciate that the compounds of the invention are readily converted to and may be isolated as a salt, such as a hydrochloride salt, using techniques and conditions well known to one of ordinary skill in the art. In addition, one skilled in the art of synthesis will appreciate that the compounds of the invention are readily converted to and may be isolated as the corresponding free base from the corresponding salt.

The present invention also provides a method for controlling pests (such as mosquitoes and other disease vectors; see also http://www.who.int/malaria/vector_control/irs/en/). In one embodiment, the method for controlling pests comprises applying the compositions of the invention to the target pests, to their locus or to a surface or substrate by brushing, rolling, spraying, spreading or dipping. By way of example, an IRS (indoor residual spraying) application of a surface such as a wall, ceiling or floor surface is contemplated by the method of the invention. In another embodiment, it is contemplated to apply such compositions to a substrate such as non-woven or a fabric material in the form of (or which can be used in the manufacture of) netting, clothing, bedding, curtains and tents.

In one embodiment, the method for controlling such pests comprises applying a pesticidally effective amount of the compositions of the invention to the target pests, to their locus, or to a surface or substrate so as to provide effective residual pesticidal activity on the surface or substrate. Such application may be made by brushing, rolling, spraying, spreading or dipping the pesticidal composition of the invention. By way of example, an IRS application of a surface such as a wall, ceiling or floor surface is contemplated by the method of the invention so as to provide effective residual pesticidal activity on the surface. In another embodiment, it is contemplated to apply such compositions for residual control of pests on a substrate such as a fabric material in the form of (or which can be used in the manufacture of) netting, clothing, bedding, curtains and tents.

Substrates including non-woven, fabrics or netting to be treated may be made of natural fibres such as cotton, raffia, jute, flax, sisal, hessian, or wool, or synthetic fibres such as polyamide, polyester, polypropylene, polyacrylonitrile or the like. The polyesters are particularly suitable. The methods of textile treatment are known, e.g. WO 2008/151984, WO 2003/034823, U.S. Pat. No. 5,631,072, WO 2005/64072, WO2006/128870, EP 1724392, WO 2005113886 or WO 2007/090739.

Further areas of use of the compositions according to the invention are the field of tree injection/trunk treatment for all ornamental trees as well all sort of fruit and nut trees.

In the field of tree injection/trunk treatment, the compounds according to the present invention are especially suitable against wood-boring insects from the order Lepidoptera as mentioned above and from the order Coleoptera, especially against woodborers listed in the following tables A and B:

TABLE A
Examples of exotic woodborers of economic importance.

| Family | Species | Host or Crop Infested |
|---|---|---|
| Buprestidae | Agrilus planipennis | Ash |
| Cerambycidae | Anoplura glabripennis | Hardwoods |
| Scolytidae | Xylosandrus crassiusculus | Hardwoods |
| | X. mutilatus | Hardwoods |
| | Tomicus piniperda | Conifers |

TABLE B
Examples of native woodborers of economic importance.

| Family | Species | Host or Crop Infested |
|---|---|---|
| Buprestidae | Agrilus anxius | Birch |
| | Agrilus politus | Willow, Maple |
| | Agrilus sayi | Bayberry, Sweetfern |
| | Agrilus vittaticolllis | Apple, Pear, Cranberry, |
| | | Serviceberry, Hawthorn |
| | Chrysobothris femorata | Apple, Apricot, Beech, Boxelder, |
| | | Cherry, Chestnut, Currant, Elm, |
| | | Hawthorn, Hackberry, Hickory, |
| | | Horsechestnut, Linden, Maple, |
| | | Mountain-ash, Oak, Pecan, Pear, |
| | | Peach, Persimmon, Plum, Poplar, |
| | | Quince, Redbud, Serviceberry, |
| | | Sycamore, Walnut, Willow |
| | Texania campestris | Basswood, Beech, Maple, Oak, |
| | | Sycamore, Willow, Yellow-poplar |
| Cerambycidae | Goes pulverulentus | Beech, Elm, Nuttall, Willow, Black |
| | | oak, Cherrybark oak, Water oak, |
| | | Sycamore |
| | Goes tigrinus | Oak |
| | Neoclytus acuminatus | Ash, Hickory, Oak, Walnut, Birch, |
| | | Beech, Maple, Eastern |
| | | hophornbeam, Dogwood, |
| | | Persimmon, Redbud, Holly, |
| | | Hackberry, Black locust, |
| | | Honeylocust, Yellow-poplar, |
| | | Chestnut, Osage-orange, Sassafras, |
| | | Lilac, Mountain-mahogany, Pear, |
| | | Cherry, Plum, Peach, Apple, Elm, |
| | | Basswood, Sweetgum |
| | Neoptychodes trilineatus | Fig, Alder, Mulberry, Willow, Netleaf |
| | | hackberry |
| | Oberea ocellata | Sumac, Apple, Peach, Plum, Pear, |
| | | Currant, Blackberry |
| | Oberea tripunctata | Dogwood, Viburnum, Elm, |
| | | Sourwood, Blueberry, |
| | | Rhododendron, Azalea, Laurel, |
| | | Poplar, Willow, Mulberry |
| | Oncideres cingulata | Hickory, Pecan, Persimmon, Elm, |
| | | Sourwood, Basswood, Honeylocust, |
| | | Dogwood, Eucalyptus, Oak, |
| | | Hackberry, Maple, Fruit trees |
| | Saperda calcarata | Poplar |
| | Strophiona nitens | Chestnut, Oak, Hickory, Walnut, |
| | | Beech, Maple |
| Scolytidae | Corthylus columbianus | Maple, Oak, Yellow-poplar, Beech, |
| | | Boxelder, Sycamore, Birch, |
| | | Basswood, Chestnut, Elm |
| | Dendroctonus frontalis | Pine |
| | Dryocoetes betulae | Birch, Sweetgum, Wild cherry, |
| | | Beech, Pear |
| | Monarthrum fasciatum | Oak, Maple, Birch, Chestnut, |
| | | Sweetgum, Blackgum, Poplar, |
| | | Hickory, Mimosa, Apple, Peach, Pine |
| | Phloeotribus liminaris | Peach, Cherry, Plum, Black cherry, |
| | | Elm, Mulberry, Mountain-ash |
| | Pseudopityophthorus pruinosus | Oak, American beech, Black cherry, |
| | | Chickasaw plum, Chestnut, Maple, |
| | | Hickory, Hornbeam, Hophornbeam |
| Sesiidae | Paranthrene simulans | Oak, American chestnut |
| | Sannina uroceriformis | Persimmon |
| | Synanthedon exitiosa | Peach, Plum, Nectarine, Cherry, |
| | | Apricot, Almond, Black cherry |
| | Synanthedon pictipes | Peach, Plum, Cherry, Beach, Black |
| | | Cherry |
| | Synanthedon rubrofascia | Tupelo |
| | Synanthedon scitula | Dogwood, Pecan, Hickory, Oak, |
| | | Chestnut, Beech, Birch, Black cherry, |
| | | Elm, Mountain-ash, Viburnum, |
| | | Willow, Apple, Loquat, Ninebark, |
| | | Bayberry |
| | Vitacea polistiformis | Grape |

The present invention may be also used to control any insect pests that may be present in turfgrass, including for example beetles, caterpillars, fire ants, ground pearls, millipedes, sow bugs, mites, mole crickets, scales, mealybugs, ticks, spittlebugs, southern chinch bugs and white grubs. The present invention may be used to control insect pests at various stages of their life cycle, including eggs, larvae, nymphs and adults.

In particular, the present invention may be used to control insect pests that feed on the roots of turfgrass including white grubs (such as Cyclocephala spp. (e.g. masked chafer, C. lurida), Rhizotrogus spp. (e.g. European chafer, R. majalis), Cotinus spp. (e.g. Green June beetle, C. nitida), Popillia spp. (e.g. Japanese beetle, P. japonica), Phyllophaga spp. (e.g. May/June beetle), Ataenius spp. (e.g. Black turfgrass ataenius, A. spretulus), Maladera spp. (e.g. Asiatic garden beetle, M. castanea) and Tomarus spp.), ground pearls (Margarodes spp.), mole crickets (tawny, southern, and short-winged; Scapteriscus spp., Gryllotalpa africana) and leatherjackets (European crane fly, Tipula spp.).

The present invention may also be used to control insect pests of turfgrass that are thatch dwelling, including armyworms (such as fall armyworm Spodoptera frugiperda, and common armyworm Pseudaletia unipuncta), cutworms, billbugs (Sphenophorus spp., such as S. venatus verstitus and S. parvulus), and sod webworms (such as Crambus spp. and the tropical sod webworm, Herpetogramma phaeopteralis).

The present invention may also be used to control insect pests of turfgrass that live above the ground and feed on the turfgrass leaves, including chinch bugs (such as southern chinch bugs, Blissus insularis), Bermudagrass mite (Eriophyes cynodoniensis), rhodesgrass mealybug (Antonina graminis), two-lined spittlebug (Propsapia bicincta), leafhoppers, cutworms (Noctuidae family), and greenbugs.

The present invention may also be used to control other pests of turfgrass such as red imported fire ants (Solenopsis invicta) that create ant mounds in turf.

In the hygiene sector, the compositions according to the invention are active against ectoparasites such as hard ticks, soft ticks, mange mites, harvest mites, flies (biting and licking), parasitic fly larvae, lice, hair lice, bird lice and fleas.

Examples of such parasites are:

Of the order Anoplurida: Haematopinus spp., Linognathus spp., Pediculus spp. and Phtirus spp., Solenopotes spp.

Of the order Mallophagida: Trimenopon spp., Menopon spp., Trinoton spp., Bovicola spp., Werneckiella spp., Lepikentron spp., Damalina spp., Trichodectes spp. and Felicola spp.

Of the order Diptera and the suborders Nematocerina and Brachycerina, for example Aedes spp., Anopheles spp., Culex spp., Simulium spp., Eusimulium spp., Phlebotomus spp., Lutzomyia spp., Culicoides spp., Chrysops spp., Hybomitra spp., Atylotus spp., Tabanus spp., Haematopota spp., Philipomyia spp., Braula spp., Musca spp., Hydrotaea spp., Stomoxys spp., Haematobia spp., Morellia spp., Fannia spp., Glossina spp., Calliphora spp., Lucilia spp., Chrysomyia spp., Wohlfahrtia spp., Sarcophaga spp., Oestrus spp., Hypoderma spp., Gasterophilus spp., Hippobosca spp., Lipoptena spp. and Melophagus spp.

Of the order Siphonapterida, for example Pulex spp., Ctenocephalides spp., Xenopsylla spp., Ceratophyllus spp.

Of the order Heteropterida, for example Cimex spp., Triatoma spp., Rhodnius spp., Panstrongylus spp.

Of the order Blattarida, for example Blatta orientalis, Periplaneta americana, Blattelagermanica and Supella spp.

Of the subclass Acaria (Acarida) and the orders Meta- and Meso-stigmata, for example Argas spp., Ornithodorus spp., Otobius spp., Ixodes spp., Amblyomma spp., Boophilus spp., Dermacentor spp., Haemophysalis spp., Hyalomma spp., Rhipicephalus spp., Dermanyssus spp., Raillietia spp., Pneumonyssus spp., Sternostoma spp. and Varroa spp.

Of the orders Actinedida (Prostigmata) and Acaridida (Astigmata), for example Acarapis spp., Cheyletiella spp., Ornithocheyletia spp., Myobia spp., Psorergates spp., Demodex spp., Trombicula spp., Listrophorus spp., Acarus spp., Tyrophagus spp., Caloglyphus spp., Hypodectes spp., Pterolichus spp., Psoroptes spp., Chorioptes spp., Otodectes spp., Sarcoptes spp., Notoedres spp., Knemidocoptes spp., Cytodites spp. and Laminosioptes spp.

The compositions according to the invention may be also suitable for protecting against insect infestation in the case of materials such as wood, textiles, plastics, adhesives, glues, paints, paper and card, leather, floor coverings and buildings.

The compositions according to the invention can be used, for example, against the following pests: beetles such as Hylotrupes bajulus, Chlorophorus pilosis, Anobium punctatum, Xestobium rufovillosum, Ptilinuspecticornis, Dendrobium pertinex, Ernobius mollis, Priobium carpini, Lyctus brunneus, Lyctus africanus, Lyctus planicollis, Lyctus linearis, Lyctus pubescens, Trogoxylon aequale, Minthesrugicollis, Xyleborus spec., Tryptodendron spec., Apate monachus, Bostrychus capucins, Heterobostrychus brunneus, Sinoxylon spec. and Dinoderus minutus, and also hymenopterans such as Sirex juvencus, Urocerus gigas, Urocerus gigas taignus and Urocerus augur, and termites such as Kalotermes flavicollis, Cryptotermes brevis, Heterotermes indicola, Reticulitermes flavipes, Reticulitermes santonensis, Reticulitermes lucifugus, Mastotermes darwiniensis, Zootermopsis nevadensis and Coptotermes formosanus, and bristletails such as Lepisma saccharina.

The compounds of formula I (including formulae Ia to Id), or salts thereof, are especially suitable for controlling one or more pests selected from order Lepidoptera, especially one or more of the species Spodoptera littoralis, Spodoptera frugiperda, Plutella xylostella, Cnaphalocrocis medinalis, Cydia pomonella, Chrysodeixis includens, Chilo suppressalis, Elasmopalpus lignosellus, Pseudoplusia includens, and Tuta absoluta (preferably in vegetables and corn), which insect demonstrates resistance against IRAC MoA Group 28 insecticides. In a preferred embodiment of each aspect, a compound TQ (where the abbreviation “TQ” means “one compound selected from the compounds defined in List Q, and Tables A-1 to A-4 and P”) controls one or more of pests selected from the species Spodoptera littoralis, Spodoptera frugiperda, Plutella xylostella, Cnaphalocrocis medinalis, Cydia pomonella, Chrysodeixis includens, Chilo suppressalis, Elasmopalpus lignosellus, Pseudoplusia includens, and Tuta absoluta (preferably in vegetables and corn), which insect demonstrates resistance against IRAC MoA Group 28 insecticides.

The compounds of formula I (including formulae Ia to Id), or salts thereof, are especially suitable for controlling one or more of the insects having diamide resistance selected from: Spodoptera littoralis, Spodoptera frugiperda, Plutella xylostella, Cnaphalocrocis medinalis, Cydia pomonella, Chrysodeixis includens, Chilo suppressalis, Elasmopalpus lignosellus, Pseudoplusia includens, and Tuta absoluta. In a preferred embodiment of each aspect, a compound TQ (where the abbreviation “TQ” means “one compound selected from the compounds defined in List Q, and Tables A-1 to A-4 and P”) controls one or more of the insects having diamide resistance selected from: Spodoptera littoralis, Spodoptera frugiperda, Plutella xylostella, Cnaphalocrocis medinalis, Cydia pomonella, Chrysodeixis includens, Chilo suppressalis, Elasmopalpus lignosellus, Pseudoplusia includens, and Tuta absoluta.

The compounds of formula I (including formulae Ia to Id), or salts thereof, are especially suitable for controlling one or more of insects having diamide resistance selected from: Plutella xylostella, Chilo suppressalis, and Tuta absoluta.

In a preferred embodiment of each aspect, a compound TQ (where the abbreviation “TQ” means “one compound selected from the compounds defined in List Q and Tables A-1 to A-4 and P”) controls one or more insect selected from Plutella xylostella, Chilo suppressalis, and Tuta absoluta, which insect demonstrates resistance against IRAC MoA Group 28 insecticides, such as Plutella xylostella+TQ, Chilo suppressalis+TQ, and Tuta absoluta+TQ.

Compounds according to the invention may possess any number of benefits including, inter alia, advantageous levels of biological activity for protecting plants against insects or superior properties for use as agrochemical active ingredients (for example, greater biological activity, an advantageous spectrum of activity, an increased safety profile (against non-target organisms above and below ground (such as fish, birds and bees), improved physico-chemical properties, or increased biodegradability). In particular, it has been surprisingly found that certain compounds of formula I may show an advantageous safety profile with respect to non-target arthropods, in particular pollinators such as honey bees, solitary bees, and bumble bees. Most particularly, Apis mellifera.

The compounds according to the invention can be used as pesticidal agents in unmodified form, but they are generally formulated into compositions in various ways using formulation adjuvants, such as carriers, solvents and surface-active substances. The formulations can be in various physical forms, e.g. in the form of dusting powders, gels, wettable powders, water-dispersible granules, water-dispersible tablets, effervescent pellets, emulsifiable concentrates, microemulsifiable concentrates, oil-in-water emulsions, oil-flowables, aqueous dispersions, oily dispersions, suspo-emulsions, capsule suspensions, emulsifiable granules, soluble liquids, water-soluble concentrates (with water or a water-miscible organic solvent as carrier), impregnated polymer films or in other forms known e.g. from the Manual on Development and Use of FAO and WHO Specifications for Pesticides, United Nations, First Edition, Second Revision (2010). Such formulations can either be used directly or diluted prior to use. The dilutions can be made, for example, with water, liquid fertilisers, micronutrients, biological organisms, oil or solvents.

The formulations can be prepared e.g. by mixing the active ingredient with the formulation adjuvants in order to obtain compositions in the form of finely divided solids, granules, solutions, dispersions or emulsions. The active ingredients can also be formulated with other adjuvants, such as finely divided solids, mineral oils, oils of vegetable or animal origin, modified oils of vegetable or animal origin, organic solvents, water, surface-active substances or combinations thereof.

The active ingredients can also be contained in very fine microcapsules. Microcapsules contain the active ingredients in a porous carrier. This enables the active ingredients to be released into the environment in controlled amounts (e.g. slow-release). Microcapsules usually have a diameter of from 0.1 to 500 microns. They contain active ingredients in an amount of about from 25 to 95% by weight of the capsule weight. The active ingredients can be in the form of a monolithic solid, in the form of fine particles in solid or liquid dispersion or in the form of a suitable solution. The encapsulating membranes can comprise, for example, natural or synthetic rubbers, cellulose, styrene/butadiene copolymers, polyacrylonitrile, polyacrylate, polyesters, polyamides, polyureas, polyurethane or chemically modified polymers and starch xanthates or other polymers that are known to the person skilled in the art. Alternatively, very fine microcapsules can be formed in which the active ingredient is contained in the form of finely divided particles in a solid matrix of base substance, but the microcapsules are not themselves encapsulated.

The formulation adjuvants that are suitable for the preparation of the compositions according to the invention are known per se. As liquid carriers there may be used: water, toluene, xylene, petroleum ether, vegetable oils, acetone, methyl ethyl ketone, cyclohexanone, acid anhydrides, acetonitrile, acetophenone, amyl acetate, 2-butanone, butylene carbonate, chlorobenzene, cyclohexane, cyclohexanol, alkyl esters of acetic acid, diacetone alcohol, 1,2-dichloropropane, diethanolamine, p-diethylbenzene, diethylene glycol, diethylene glycol abietate, diethylene glycol butyl ether, diethylene glycol ethyl ether, diethylene glycol methyl ether, N,N-dimethylformamide, dimethyl sulfoxide, 1,4-dioxane, dipropylene glycol, dipropylene glycol methyl ether, dipropylene glycol dibenzoate, diproxitol, alkylpyrrolidone, ethyl acetate, 2-ethylhexanol, ethylene carbonate, 1,1,1-trichloroethane, 2-heptanone, alpha-pinene, d-limonene, ethyl lactate, ethylene glycol, ethylene glycol butyl ether, ethylene glycol methyl ether, gamma-butyrolactone, glycerol, glycerol acetate, glycerol diacetate, glycerol triacetate, hexadecane, hexylene glycol, isoamyl acetate, isobornyl acetate, isooctane, isophorone, isopropylbenzene, isopropyl myristate, lactic acid, laurylamine, mesityl oxide, methoxy-propanol, methyl isoamyl ketone, methyl isobutyl ketone, methyl laurate, methyl octanoate, methyl oleate, methylene chloride, m-xylene, n-hexane, n-octylamine, octadecanoic acid, octylamine acetate, oleic acid, oleylamine, o-xylene, phenol, polyethylene glycol, propionic acid, propyl lactate, propylene carbonate, propylene glycol, propylene glycol methyl ether, p-xylene, toluene, triethyl phosphate, triethylene glycol, xylenesulfonic acid, paraffin, mineral oil, trichloroethylene, perchloroethylene, ethyl acetate, amyl acetate, butyl acetate, propylene glycol methyl ether, diethylene glycol methyl ether, methanol, ethanol, isopropanol, and alcohols of higher molecular weight, such as amyl alcohol, tetrahydrofurfuryl alcohol, hexanol, octanol, ethylene glycol, propylene glycol, glycerol, N-methyl-2-pyrrolidone and the like.

Suitable solid carriers are, for example, talc, titanium dioxide, pyrophyllite clay, silica, attapulgite clay, kieselguhr, limestone, calcium carbonate, bentonite, calcium montmorillonite, cottonseed husks, wheat flour, soybean flour, pumice, wood flour, ground walnut shells, lignin and similar substances.

A large number of surface-active substances can advantageously be used in both solid and liquid formulations, especially in those formulations which can be diluted with a carrier prior to use. Surface-active substances may be anionic, cationic, non-ionic or polymeric and they can be used as emulsifiers, wetting agents or suspending agents or for other purposes. Typical surface-active substances include, for example, salts of alkyl sulfates, such as diethanolammonium lauryl sulfate; salts of alkylarylsulfonates, such as calcium dodecylbenzenesulfonate; alkylphenol/alkylene oxide addition products, such as nonylphenol ethoxylate; alcohol/alkylene oxide addition products, such as tridecylalcohol ethoxylate; soaps, such as sodium stearate; salts of alkylnaphthalenesulfonates, such as sodium dibutylnaphthalenesulfonate; dialkyl esters of sulfosuccinate salts, such as sodium di(2-ethylhexyl)sulfosuccinate; sorbitol esters, such as sorbitol oleate; quaternary amines, such as lauryltrimethylammonium chloride, polyethylene glycol esters of fatty acids, such as polyethylene glycol stearate; block copolymers of ethylene oxide and propylene oxide; and salts of mono- and di-alkylphosphate esters; and also further substances described e.g. in McCutcheon's Detergents and Emulsifiers Annual, MC Publishing Corp., Ridgewood New Jersey (1981).

Further adjuvants that can be used in pesticidal formulations include crystallisation inhibitors, viscosity modifiers, suspending agents, dyes, anti-oxidants, foaming agents, light absorbers, mixing auxiliaries, antifoams, complexing agents, neutralising or pH-modifying substances and buffers, corrosion inhibitors, fragrances, wetting agents, take-up enhancers, micronutrients, plasticisers, glidants, lubricants, dispersants, thickeners, antifreezes, microbicides, and liquid and solid fertilisers.

The compositions according to the invention can include an additive comprising an oil of vegetable or animal origin, a mineral oil, alkyl esters of such oils or mixtures of such oils and oil derivatives. The amount of oil additive in the composition according to the invention is generally from 0.01 to 10%, based on the mixture to be applied. For example, the oil additive can be added to a spray tank in the desired concentration after a spray mixture has been prepared. Preferred oil additives comprise mineral oils or an oil of vegetable origin, for example rapeseed oil, olive oil or sunflower oil, emulsified vegetable oil, alkyl esters of oils of vegetable origin, for example the methyl derivatives, or an oil of animal origin, such as fish oil or beef tallow. Preferred oil additives comprise alkyl esters of C₈-C₂₂ fatty acids, especially the methyl derivatives of C₁₂-C₁₈ fatty acids, for example the methyl esters of lauric acid, palmitic acid and oleic acid (methyl laurate, methyl palmitate and methyl oleate, respectively). Many oil derivatives are known from the Compendium of Herbicide Adjuvants, 10^{th} Edition, Southern Illinois University, 2010.

The inventive compositions generally comprise from 0.1 to 99% by weight, especially from 0.1 to 95% by weight, of compounds of the present invention and from 1 to 99.9% by weight of a formulation adjuvant which preferably includes from 0 to 25% by weight of a surface-active substance. Whereas commercial products may preferably be formulated as concentrates, the end user will normally employ dilute formulations.

The rates of application vary within wide limits and depend on the nature of the soil, the method of application, the crop plant, the pest to be controlled, the prevailing climatic conditions, and other factors governed by the method of application, the time of application and the target crop. As a general guideline compounds may be applied at a rate of from 1 to 2000 l/ha, especially from 10 to 1000 l/ha.

Preferred formulations can have the following compositions (weight %):

### Emulsifiable Concentrates:

active ingredient: 1 to 95%, preferably 60 to 90%
surface-active agent: 1 to 30%, preferably 5 to 20%
liquid carrier: 1 to 80%, preferably 1 to 35%

### Dusts:

active ingredient: 0.1 to 10%, preferably 0.1 to 5%
solid carrier: 99.9 to 90%, preferably 99.9 to 99%

### Suspension Concentrates:

active ingredient: 5 to 75%, preferably 10 to 50%
water: 94 to 24%, preferably 88 to 30%
surface-active agent: 1 to 40%, preferably 2 to 30%

### Wettable Powders:

active ingredient: 0.5 to 90%, preferably 1 to 80%
surface-active agent: 0.5 to 20%, preferably 1 to 15%
solid carrier: 5 to 95%, preferably 15 to 90%

### Granules:

active ingredient: 0.1 to 30%, preferably 0.1 to 15%
solid carrier: 99.5 to 70%, preferably 97 to 85% The following Examples further illustrate, but do not limit, the invention.

| Wettable powders | a) | b) | c) |
|---|---|---|---|
| active ingredients | 25% | 50% | 75% |
| sodium lignosulfonate | 5% | 5% | — |
| sodium lauryl sulfate | 3% | — | 5% |
| sodium diisobutylnaphthalenesulfonate | — | 6% | 10% |
| phenol polyethylene glycol ether | — | 2% | — |
| (7-8 mol of ethylene oxide) | | | |
| highly dispersed silicic acid | 5% | 10% | 10% |
| Kaolin | 62% | 27% | — |

The combination is thoroughly mixed with the adjuvants and the mixture is thoroughly ground in a suitable mill, affording wettable powders that can be diluted with water to give suspensions of the desired concentration.

| Powders for dry seed treatment | a) | b) | c) |
|---|---|---|---|
| active ingredients | 25% | 50% | 75% |
| light mineral oil | 5% | 5% | 5% |
| highly dispersed silicic acid | 5% | 5% | — |
| Kaolin | 65% | 40% | — |
| Talcum | — | | 20% |

The combination is thoroughly mixed with the adjuvants and the mixture is thoroughly ground in a suitable mill, affording powders that can be used directly for seed treatment.

Emulsifiable concentrate

| | |
|---|---|
| active ingredients | 10% |
| octylphenol polyethylene glycol ether (4-5 mol of ethylene | 3% |
| oxide) | |
| calcium dodecylbenzenesulfonate | 3% |
| castor oil polyglycol ether (35 mol of ethylene oxide) | 4% |
| Cyclohexanone | 30% |
| xylene mixture | 50% |

Emulsions of any required dilution, which can be used in plant protection, can be obtained from this concentrate by dilution with water.

| Dusts | a) | b) | c) |
|---|---|---|---|
| Active ingredients | 5% | 6% | 4% |
| Talcum | 95% | — | — |
| Kaolin | — | 94% | — |
| mineral filler | — | — | 96% |

Ready-for-use dusts are obtained by mixing the combination with the carrier and grinding the mixture in a suitable mill. Such powders can also be used for dry dressings for seed.

| Extruder granules | |
|---|---|
| Active ingredients | 15% |
| sodium lignosulfonate | 2% |
| carboxymethylcellulose | 1% |
| Kaolin | 82% |

The combination is mixed and ground with the adjuvants, and the mixture is moistened with water.

The mixture is extruded and then dried in a stream of air.

| Coated granules | |
|---|---|
| Active ingredients | 8% |
| polyethylene glycol (mol. wt. 200) | 3% |
| Kaolin | 89% |

The finely ground combination is uniformly applied, in a mixer, to the kaolin moistened with polyethylene glycol. Non-dusty coated granules are obtained in this manner.

### Suspension Concentrate

| | |
|---|---|
| active ingredients | 40% |
| propylene glycol | 10% |
| nonylphenol polyethylene glycol ether (15 mol of ethylene oxide) | 6% |
| Sodium lignosulfonate | 10% |
| carboxymethylcellulose | 1% |
| silicone oil (in the form of a 75% emulsion in water) | 1% |
| Water | 32% |

The finely ground combination is intimately mixed with the adjuvants, giving a suspension concentrate from which suspensions of any desired dilution can be obtained by dilution with water. Using such dilutions, living plants as well as plant propagation material can be treated and protected against infestation by microorganisms, by spraying, pouring or immersion.

### Flowable Concentrate for Seed Treatment

| | |
|---|---|
| active ingredients | 40% |
| propylene glycol | 5% |
| copolymer butanol PO/EO | 2% |
| Tristyrenephenole with 10-20 moles EO | 2% |
| 1,2-benzisothiazolin-3-one (in the form | 0.5% |
| of a 20% solution in water) | |
| monoazo-pigment calcium salt | 5% |
| Silicone oil (in the form of a | 0.2% |
| 75% emulsion in water) | |
| Water | 45.3% |

The finely ground combination is intimately mixed with the adjuvants, giving a suspension concentrate from which suspensions of any desired dilution can be obtained by dilution with water. Using such dilutions, living plants as well as plant propagation material can be treated and protected against infestation by microorganisms, by spraying, pouring or immersion.

### Slow Release Capsule Suspension

28 parts of the combination are mixed with 2 parts of an aromatic solvent and 7 parts of toluene diisocyanate/polymethylene-polyphenylisocyanate-mixture (8:1). This mixture is emulsified in a mixture of 1.2 parts of polyvinylalcohol, 0.05 parts of a defoamer and 51.6 parts of water until the desired particle size is achieved. To this emulsion a mixture of 2.8 parts 1,6-diaminohexane in 5.3 parts of water is added. The mixture is agitated until the polymerization reaction is completed. The obtained capsule suspension is stabilized by adding 0.25 parts of a thickener and 3 parts of a dispersing agent. The capsule suspension formulation contains 28% of the active ingredients. The medium capsule diameter is 8-15 microns. The resulting formulation is applied to seeds as an aqueous suspension in an apparatus suitable for that purpose.

Formulation types include an emulsion concentrate (EC), a suspension concentrate (SC), a suspo-emulsion (SE), a capsule suspension (CS), a water dispersible granule (WG), an emulsifiable granule (EG), an emulsion, water in oil (EO), an emulsion, oil in water (EW), a micro-emulsion (ME), an oil dispersion (OD), an oil miscible flowable (OF), an oil miscible liquid (OL), a soluble concentrate (SL), an ultra-low volume suspension (SU), an ultra-low volume liquid (UL), a technical concentrate (TK), a dispersible concentrate (DC), a wettable powder (WP), a soluble granule (SG) or any technically feasible formulation in combination with agriculturally acceptable adjuvants.

### PREPARATORY EXAMPLES

“Mp” means melting point in ° C. Free radicals represent methyl groups. 1H NMR measurements were recorded on a Brucker 400 MHz spectrometer, chemical shifts are given in ppm relevant to a TMS standard. Spectra measured in deuterated solvents as indicated. Either one of the LC-MS methods below was used to characterize the compounds. The characteristic LC-MS values obtained for each compound were the retention time (“Rt”, recorded in minutes) and the measured molecular ion (M+H)+.

### LC-MS and GC-MS Methods:

### LC-MS Method 1:

Spectra were recorded on a Mass Spectrometer from Waters (SQD, SQDII Single quadrupole mass spectrometer) equipped with an electrospray source (Polarity: positive and negative ions, Capillary: 3.00 kV, Cone range: 30 V, Extractor: 2.00 V, Source Temperature: 150° C., Desolvation Temperature: 350° C., Cone Gas Flow: 50 I/h, Desolvation Gas Flow: 650 I/h, Mass range: 100 to 900 Da) and an Acquity UPLC from Waters: Binary pump, heated column compartment, diode-array detector and ELSD detector. Column: Waters UPLC HSS T3, 1.8 μm, 30×2.1 mm, Temp: 60° C., DAD Wavelength range (nm): 210 to 500, Solvent Gradient: A=water+5% MeOH+0.05% HCOOH, B=Acetonitrile+0.05% HCOOH, gradient: 10-100% B in 1.2 min; Flow (ml/min) 0.85

### LC-MS Method 2:

Spectra were recorded on a ACQUITY Mass Spectrometer from Waters Corporations (SQD or SQDII Single quadrupole mass spectrometer) equipped with an electrospray source (Polarity: positive or negative ions, Capillary: 3.0 kV, Cone: 30V, Extractor: 3.00 V, Source Temperature: 150° C., Desolvation Temperature: 400° C., Cone Gas Flow: 60 L/hr, Desolvation Gas Flow: 700 L/hr, Mass range: 140 to 800 Da) and an ACQUITY UPLC from Waters Corporations with solvent degasser, binary pump, heated column compartment and diode-array detector. Column: Waters UPLC HSS T3, 1.8 μm, 30×2.1 mm, Temp: 60° C., DAD Wavelength range (nm): 210 to 400, Solvent Gradient: A=Water/Methanol 9:1+0.1% formic acid, B=Acetonitrile+0.1% formic acid, gradient: 0-100% B in 2.5 min; Flow (ml/min) 0.75.

### ESI Method 3:

Spectra were recorded on a Mass Spectrometer from Waters (SQD, SQDII Single quadrupole mass spectrometer) equipped with an electrospray source (Polarity: positive and negative ions, Capillary: 3.20 kV, Cone range: 30 V, Extractor: 3.00 V, Source Temperature: 150° C., Desolvation Temperature: 400° C., Cone Gas Flow: 50 L/h, Desolvation Gas Flow: 1000 L/h, Mass range: 100 to 1000 Da).

### Example 1: Preparation of N-(2-carbamoyl-4-chloro-6-methyl-phenyl)-5-(chloromethyl)-2-(3-chloro-2-pyridyl)pyrazole-3-carboxamide (P7)

### Step A: Preparation of 1-(3-chloropyridin-2-yl)-3-(chloromethyl)-1H-pyrazole-5-carboxylic acid

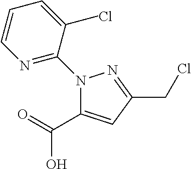 [SMILES: Cc1cccnc1-n1nc(CCl)cc1C(=O)O ; Cl]

To a solution of methyl 5-(chloromethyl)-2-(3-chloro-2-pyridyl)pyrazole-3-carboxylate (1.06 g, 3170 mmol, 1.00 equiv.) in tetrahydrofuran (7.4 mL) and water (3.7 mL) was added lithium hydroxide monohydrate (0.187 g, 4.45 mmol, 1.20 equiv.). The reaction was stirred at room temperature for 1 hour. Then, lithium hydroxide monohydrate (0.045 g, 1.11 mmol, 0.30 equiv.) was added and the solution was stirred for 30 minutes. The reaction mixture was cooled to 0° C., then acidified to pH 2-3 by addition of HCl (1 N). The mixture was extracted twice with ethyl acetate, the combined organic layers were washed with brine, dried over magnesium sulfate, filtered and concentrated under reduced pressure to afford 1-(3-chloropyridin-2-yl)-3-(chloromethyl)-1H-pyrazole-5-carboxylic acid as a white crystal.

LC-MS (Method 1): retention time 0.69 min, m/z 272 [M+H⁺].

¹H NMR (400 MHz, 3-MeOD) δ ppm 8.52 (dd, J=4.72, 1.45 Hz, 1H) 8.13 (dd, J=7.99, 1.45 Hz, 1H) 7.62 (dd, J=8.17, 4.90 Hz, 1H) 7.14 (s, 1H) 4.73 (s, 2H)

Preparation of methyl 5-(chloromethyl)-2-(3-chloro-2-pyridyl)pyrazole-3-carboxylate is known and described in patent WO2011073101.

### Step B: Preparation of N-(2-carbamoyl-4-chloro-6-methyl-phenyl)-5-(chloromethyl)-2-(3-chloro-2-pyridyl)pyrazole-3-carboxamide (P7)

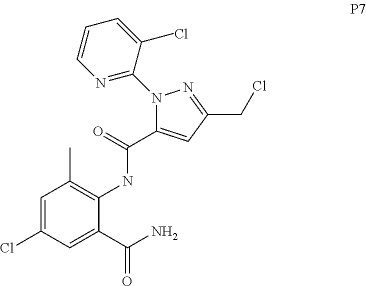 [SMILES: Cc1cccnc1-n1nc(CCl)cc1C(=O)Nc1c(C)cc(Cl)cc1C(N)=O ; Cl]

Under argon, to a solution of methane sulfonyl chloride (0.556 mL, 7.02 mmol, 2.00 equiv.) in acetonitrile (7.0 mL) were added dropwise at 0° C. a solution of 1-(3-chloropyridin-2-yl)-3-(chloromethyl)-1H-pyrazole-5-carboxylic acid (0.955 g, 3.51 mmol, 1.00 equiv.) in acetonitrile (14.0 mL) and pyridine (0.566 mL, 7.02 mmol, 2.00 equiv.). The reaction mixture was stirred at 0° C. for 30 minutes. Then, a suspension of 2-amino-5-chloro-3-methyl-benzoic acid (0.651 g, 3.51 mmol, 1.00 equiv.) in acetonitrile (14.0 mL) was added to the previous solution at room temperature, followed by pyridine (0.283 mL, 3.51 mmol, 1.00 equiv.). The reaction mixture was stirred at room temperature overnight. Ammonia (2 M in ethanol, 23.0 mL, 45.6 mmol, 13.0 equiv.) was added at room temperature and it was stirred for 1 hour at 40° C.

The reaction mixture was diluted with ethyl acetate, washed with water, then brine, dried over magnesium sulfate, filtered, and concentrated under reduced pressure. To the crude was added dichloromethane and the insoluble product was filtered off to afford the desired product N-(2-carbamoyl-4-chloro-6-ethyl-phenyl)-5-(chloroethyl)-2-(3-chloro-2-pyridyl)pyrazole-3-carboxamide. 5 LC-MS (Method 1): retention time 0.87 min, m/z 438 [M+H⁺].

1H NMR (400 MHz, DMSO-d6) δ ppm 10.28 (s, 1H) 8.49 (dd, J=4.54, 1.64 Hz, 1H) 8.14 (dd, J=7.99, 1.45 Hz, 1H) 7.73 (br s, 1H) 7.58 (dd, J=7.99, 4.72 Hz, 1H) 7.50 (br s, 1H) 7.46 (d, J=2.18 Hz, 1H) 7.40 (d, J=2.54 Hz, 1H) 7.31 (s, 1H) 4.87 (s, 2H) 2.50 (dt, J=3.54, 1.68 Hz, 28H) 2.15 (s, 3H)

The preparation of 2-amino-5-chloro-3-methyl-benzoic acid is known and described in patent

### Example 2: Preparation of N-(2-carbamoyl-4-chloro-6-methyl-phenyl)-2-(3-chloro-2-pyridyl)-5-[(5-cyclopropyltetrazol-1-yl)methyl]pyrazole-3-carboxamide (P1); and N-(2-carbamoyl-4-chloro-6-methyl-phenyl)-2-(3-chloro-2-pyridyl)-5-[(5-cyclopropyltetrazol-2-yl)methyl]pyrazole-3-carboxamide (P2)

### Step A: Preparation of (5-cyclopropyltetrazol-2-yl)sodium

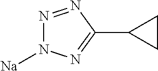

Under argon, to a solution of 5-cyclopropyl-2H-tetrazole (0.103 g, 0.907 mmol, 1 equiv.) in tetrahydrofuran (4.54 mL) was added sodium hydride (60 mass %, 0.036 g, 0.907 mmol, 1 equiv.). The mixture was stirred at room temperature for 3 hours. The reaction mixture was concentrated under reduced pressure to afford (5-cyclopropyltetrazol-2-yl)sodium. The crude material was used as such in step B.

1H NMR (400 MHz, DMSO-d6) δ ppm 1.93 (tt, J=8.40, 5.04 Hz, 1H) 0.72-0.81 (m, 2H) 0.63-0.72 (m, 2H)

The preparation of 5-cyclopropyl-2H-tetrazole is known and described in patent WO2010/100144

### Step B: Preparation of N-(2-carbamoyl-4-chloro-6-methyl-phenyl)-2-(3-chloro-2-pyridyl)-5-[(5-cyclopropyltetrazol-1-yl)methyl]pyrazole-3-carboxamide (P1) and N-(2-carbamoyl-4-chloro-6-methyl-phenyl-2-(3-chloro-2-pyridyl)-5-[(5-cyclopropyltetrazol-2-yl)methyl]pyrazole-3-carboxamide (P2)

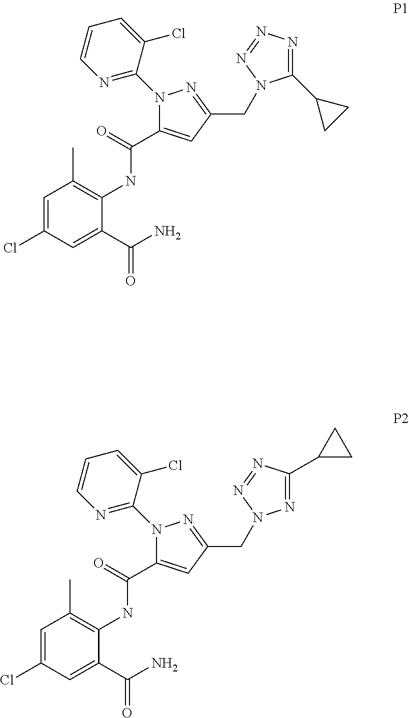 [SMILES: Cc1cc(Cl)cc(C(N)=O)c1NC(=O)c1cc(Cn2nnnc2C2CC2)nn1-c1ncccc1Cl ; Cc1cc(Cl)cc(C(N)=O)c1NC(=O)c1cc(Cn2nnc(C3CC3)n2)nn1-c1ncccc1Cl]

To a solution of N-(2-carbamoyl-4-chloro-6-methyl-phenyl)-5-(chloromethyl)-2-(3-chloro-2-pyridyl)pyrazole-3-carboxamide (P7) (0.080 g, 0.18 mmol, 1.00 equiv.) and (5-cyclopropyltetrazol-2-yl)sodium (0.029 g, 0.19 mmol, 1.03 equiv.) in acetone (0.91 mL) was added potassium iodide (0.003 g, 0.018 mmol, 0.1 equiv.). The mixture was stirred at 60° C. overnight. Then, the reaction was concentrated under reduced pressure and the crude material was purified by flash chromatography (ethyl acetate in cyclohexane) followed by a reverse phase chromatography to afford the desired products N-(2-carbamoyl-4-chloro-6-methyl-phenyl)-2-(3-chloro-2-pyridyl)-5-[(5-cyclopropyltetrazol-1-yl)methyl]pyrazole-3-carboxamide and N-(2-carbamoyl-4-chloro-6-methyl-phenyl)-2-(3-chloro-2-pyridyl)-5-[(5-cyclopropyltetrazol-2-yl)methyl]pyrazole-3-carboxamide

### Compound P1:

LC-MS (Method 1): retention time 0.82 min, 512 m/z [M+H⁺]. 1H NMR (600 MHz, DMSO-d6) δ ppm 10.25 (s, 1H) 8.48 (dd, J=4.5, 1.1 Hz, 1H) 8.15 (d, J=7.3 Hz, 1H) 7.73 (br s, 1H) 7.59 (dd, J=8.0, 4.7 Hz, 1H) 7.47 (br d, J=15.8 Hz, 2H) 7.39 (d, J=1.6 Hz, 1H) 7.19 (s, 1H) 5.91 (s, 2H) 228-2.33 (m, 1H) 2.13 (s, 3H) 1.17 (dd, J=82, 2.7 Hz, 2H) 1.01 (dd, J=4.5, 2.5 Hz, 2H)

### Compound P2:

LC-MS (Method 1): retention time 0.89 min, 512 m/z [M+H⁺]. 1H NMR (600 MHz, DMSO-d6) δ ppm 10.25 (s, 1H) 8.49 (dd, J=4.7, 1.3 Hz, 1H) 8.15 (d, J=8A, 1.2 Hz, 1H) 7.72 (s, 1H) 7.59 (dd, J=82, 4.7 Hz, 1H) 7.43-7.51 (m, 2H) 7.39 (d, J=2.0 Hz, 1H) 722 (s, 1H) 5.99 (s, 2H) 2.19-2.29 (m, 1H) 2.13 (s, 3H) 1.10 (dd, J=8.4, 2.5 Hz, 2H) 0.95 (dd, J=4.8, 2.5 Hz, 2H)

### Example 3: Preparation of N-(2-carbamoyl-4chloro-methyl-phenyl)-5-[(4-chloroindazol-1-yl)methyl]-2-(3-chloro-2-pyridyl)pyrazole-3-carboxamide (P3); and N-(2-carbamoyl-4-chloro-6-methyl-phenyl)-5-[(4-chloroindazol-2-yl)methyl]-2-(3-chloro-2-pyridyl)pyrazole-3-carboxamide (P4)

### Step A: Preparation of (4-chloroindazol-1-yl)sodium

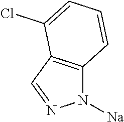

(4-chloroindazol-1-yl)sodium was prepared in using 4-chloro-1H-indazol and the conditions described for (5-cyclopropyltetrazol-2-yl)sodium (Example 2 Step A) 1H NMR (400 MHz, DMSO-d6) δ ppm 7.81 (d, J=0.73 Hz, 1H) 7.32 (d, J=8.31 Hz, 1H) 6.70 (dd, J=8.17, 7.08 Hz, 1H) 6.56 (dd, J=6.90, 0.73 Hz, 1H)

### Step B: Preparation of N-(2-carbamoyl-4-chloro-6-methyl-phenyl)-5-[(4-chloroindazol-1-yl)methyl]-2-(3-chloro-2-pyridyl)pyrazole-3-carboxamide and N-(2-carbamoyl-4-chloro-6-methyl-phenyl)-5-[(4-chloroindazol-2-yl)methyl]-2-(3-chloro-2-pyridyl)pyrazole-3-carboxamide

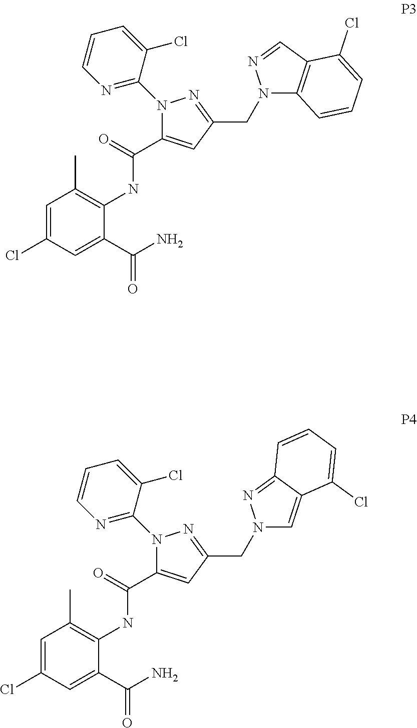 [SMILES: Cc1cc(Cl)cc(C(N)=O)c1NC(=O)c1cc(Cn2ncc3c(Cl)cccc32)nn1-c1ncccc1Cl ; Cc1cc(Cl)cc(C(N)=O)c1NC(=O)c1cc(Cn2cc3c(Cl)cccc3n2)nn1-c1ncccc1Cl]

Compound P3 and Compound P4 were prepared in using (4-chloroindazol-1-yl)sodium and the conditions described for Compound P1 and Compound P2 (Example 2 Step B)

### Compound P3

LC-MS (Method 1): Rt 1.02, m/z=554 (M+H+).

1H NMR (600 MHz, CDCl3) δ ppm 9.72 (s, 1H) 8.49 (dd, J=4.7, 1.5 Hz, 1H) 8.17 (s, 1H) 7.87 (dd, J=8.0, 1.5 Hz, 1H) 7.48 (d, J=8.5 Hz, 1H) 7.39 (dd, J=8.1, 4.7 Hz, 1H) 7.29-7.32 (m, 2H) 7.17 (d, J=7.2 Hz, 1H) 6.84 (s, 1H) 6.04 (br s, 1H) 5.75 (s, 2H) 5.68 (br s, 1H) 2.17 (s, 3H)

### Compound P4:

LC-MS (Method 1): Rt 0.99, m/z=554 (M+H⁺). 1H NMR (400 MHz, CDCl3) δ ppm 9.82 (s, 1H), 8.49 (dd, J=4.7, 1.5 Hz, 1H), 8.15-8.17 (m, 1H), 7.87 (dd, J=8.0, 1.8 Hz, 1H), 7.65 (d, J=8.7 Hz, 1H), 7.39 (dd, J=8.0, 4.7 Hz, 1H), 7.29-7.34 (m, 2H), 7.22 (dd, J=8.5, 7.1 Hz, 1H), 7.08 (d, J=6.9 Hz, 1H), 7.03 (s, 1H), 5.95-6.08 (m, 1H), 5.76 (s, 2H), 5.60-5.72 (m, 1H), 2.19 (s, 3H).

### Example 4: Preparation of N-(2-carbamoyl-4-chloro-6-methyl-phenyl)-5-[(4-chlorophenyl)methoxymethyl]-2-(3-chloro-2-pyridyl)pyrazole-3-carboxamide (P5)

### Step A: Preparation of 5-[(4-chlorophenyl)methoxymethyl]-2-(3-chloro-2-pyridyl)pyrazole-3-carboxylic acid

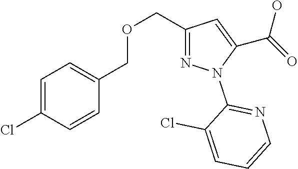 [SMILES: O=C(O)c1cc(COCc2ccc(Cl)cc2)nn1-c1ncccc1Cl]

To a solution of 4-chlorobenzyl alcohol (0.125 g, 0.875 mmol, 3.0 equiv.) in tetrahydrofuran (2.0 mL) was added at 0° C. sodium hydride (60 mass %, 0.035 g, 0.875 mmol, 3.0 equiv.). The mixture was stirred at this temperature for 30 minutes. Then, a solution of 1-(3-chloropyridin-2-yl)-3-(chloromethyl)-1H-pyrazole-5-carboxylic acid (0.0794 g, 0.292 mol, 1.00 equiv.) in tetrahydrofuran (2.0 mL) was added dropwise at 0° C. to the reaction mixture. The solution was warmed to room temperature and stirred for 30 minutes. The yellowish solution was then heated to reflux and stirred at that temperature for 2 hours, then overnight at room temperature. The reaction mixture was quenched by dropwise addition of ammonium chloride aqueous solution (3.0 mL). The aqueous layer was set acidic (pH 2-3) by dropwise addition of HCl solution (2N, aqueous), extracted twice with ethyl acetate. The combined organic layers were washed with water, then with brine, dried over magnesium sulfate, filtered, and evaporated under reduced pressure. The crude material was purified by reverse phase chromatography to afford the desired product, 5-[(4-chlorophenyl)methoxymethyl]-2-(3-chloro-2-pyridyl)pyrazole-3-carboxylic acid, as a yellowish oil.

LC-MS (Method 1): Rt 0.95, m/z=378 (M+H+).

1H NMR (400 MHz, DMSO-d6) δ ppm 8.54 (dd, J=4.72, 1.45 Hz, 1H) 8.21 (dd, J=8.17, 1.27 Hz, 1H) 7.65 (dd, J=7.99, 4.72 Hz, 1H) 7.37-7.44 (m, 4H) 7.03 (s, 1H) 4.58 (s, 2H) 4.56 (s, 2H)

### Step B: Preparation of N-(2-carbamoyl-4-chloro-6-methyl-phenyl)-5-[(4-chlorophenyl)methoxymethyl]-2-(3-chloro-2-pyridyl)pyrazole-3-carboxamide

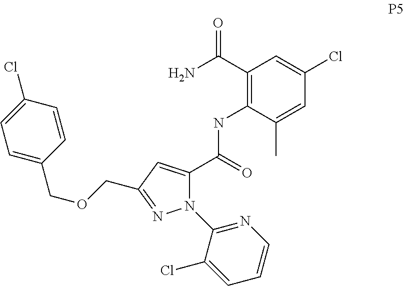 [SMILES: Cc1cc(Cl)cc(C(N)=O)c1NC(=O)c1cc(COCc2ccc(Cl)cc2)nn1-c1ncccc1Cl]

Compound P5 was prepared in using 5-[(4-chlorophenyl)methoxymethyl]-2-(3-chloro-2-pyridyl)pyrazole-3-carboxylic acid and 2-amino-5-chloro-3-methyl-benzoic acid and the conditions described for Compound P7 (Example 1, Step B).

LC-MS (Method 1): Rt 1.04, m/z=544 (M+H+).

1H NMR (400 MHz, acetone-d6) δ ppm 10.35 (br s, 1H), 8.49 (dd, J=4.7, 1.5 Hz, 1H), 8.06 (dd, J=8.0, 1.5 Hz, 1H), 7.51-7.61 (m, 3H), 7.43-7.47 (m, 2H), 7.38-7.42 (m, 3H), 7.20 (s, 1H), 6.96-7.10 (m, 1H), 4.69 (s, 2H), 4.63-4.67 (m, 2H), 2.23 (s, 3H)

### Example 5: Preparation of N-(2-carbamoyl-4-chloro-methyl-phenyl)-2-(3-choro-pyridyl)-5-[[5-[4-(trifluoromethyl)phenyl]tetraol-2-yl]methyl]pyrazole-3-carboxamide (P6); and N-(2-carbamoyl-4-chloro-6-methyl-phenyl)-2-(3-chloro-2-pyridyl)-5-[[5-[4-(trifluoromethyl)phenyl]tetrazol-1-yl]methyl]pyrazole-3-carboxamide (P15)

### Step A: Preparation of [5-[4-(trifluoromethyl)phenyl]tetrazol-2-yl]sodium

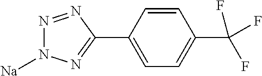

[5-[4-(trifluoromethyl)phenyl]tetrazol-2-yl]sodium was prepared in using 5-[4-(trifluoromethyl)phenyl]-1H-tetrazole and the conditions described for (5-cyclopropyltetrazol-2-yl)sodium (example 2 step A) 1H NMR (400 MHz, DMSO-d6) δ ppm 8.17 (d, J=7.99 Hz, 2H) 7.73 (d, J=7.99 Hz, 2H) ¹⁹F NMR (377 MHz, DMSO-d6) δ ppm −60.71 (s, 3F)

### Step B: Preparation of N-(2-carbamoyl-4-chloro-6-methyl-phenyl)-2-(3-chloro-2-pyridyl)-5-[[5-[4-(trifluoromethyl)phenyl]tetrazol-2-yl]methyl]pyrazole-3-carboxamide (P6); and N-(2-carbamoyl-4-chloro-6-methyl-phenyl)-2-(3-chloro-2-pyridyl)-5-[[5-[4-(trifluoromethyl)phenyl]tetrazol-1-yl]methyl]pyrazole-3-carboxamide (P15)

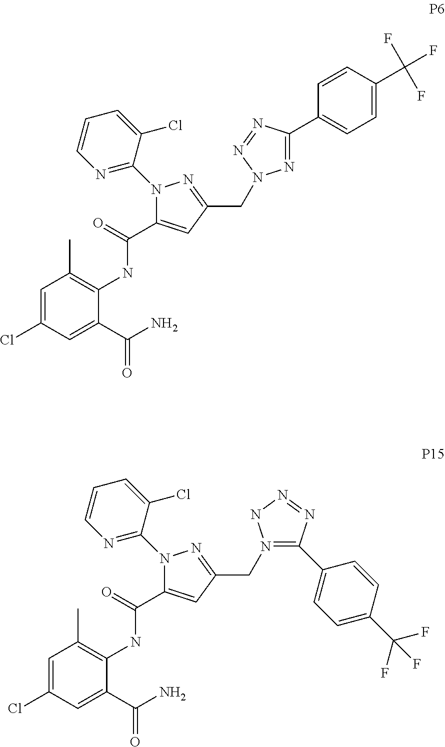

Compound P6 and Compound P15 were prepared in using [5-[4-(trifluoromethyl)phenyl]tetrazol-2-yl]sodium and the conditions described for Compound P1 and Compound P2 (Example 2 Step B)

### Compound P6:

LC-MS (Method 1): Rt 1.08, m/z=616 (M+H+).

1H NMR (600 MHz, DMSO-d6) δ ppm 10.26 (s, 1H) 8.48 (dd, J=4.6, 1.4 Hz, 1H) 8.32 (m, J=8.2 Hz, 2H) 8.15 (dd, J=8.1, 1.4 Hz, 1H) 7.97 (m, J=8.2 Hz, 2H) 7.72 (s, 1H) 7.59 (dd, J=8.0, 4.7 Hz, 1H) 7.46-7.47 (m, 1H) 7.45 (br d, J=2.4 Hz, 1H) 7.38 (d, J=2.2 Hz, 1H) 7.31 (s, 1H) 6.23 (s, 2H) 2.13 (s, 3H).

### Compound P15:

LC-MS (Method 1): Rt 0.99, m/z=616 (M+H+).

1H NMR (400 MHz, CDCl3) δ ppm 9.93-10.06 (m, 1H), 8.45-8.53 (m, 1H), 8.00-8.10 (m, 2H), 7.89 (dd, J=8.0, 1.5 Hz, 1H), 7.82 (d, J=8.4 Hz, 2H), 7.40-7.45 (m, 1H), 7.31-7.37 (m, 2H), 7.11 (s, 1H), 5.97-6.09 (m, 1H), 5.78 (s, 2H), 5.64-5.75 (m, 1H), 2.20 (s, 3H)

### Example 6: Preparation of N-(2-carbamoyl-4-chloro-6-methyl-phenyl)-2-(3-chloro-2-pyridyl)-5-(2,2,3,3,3-pentafluoropropoxy)pyrazole-3-carboxamide (P8)

### Step A: Preparation of methyl 2-(3-chloro-2-pyridyl)-5-(2,2,3,3,3-pentafluoropropoxy)pyrazole-3-carboxylate

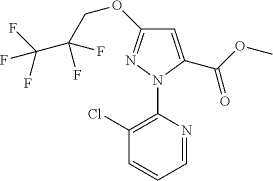 [SMILES: COC(=O)c1cc(OCC(F)(F)C(F)(F)F)nn1-c1ncccc1Cl]

To a suspension of methyl 2-(3-chloro-2-pyridyl)-5-oxo-1H-pyrazole-3-carboxylate (1.00 g, 3.94 mmol, 1 equiv.) in acetonitrile (20 mL) at −5° C. was added potassium carbonate (1.12 g, 8.08 mmol, 2.05 equiv.). The suspension was stirred 15 minutes at 20° C. Then, 2,2,3,3,3-pentafluoropropyl trifluoromethanesulfonate was added dropwise to the suspension at 5° C. The reaction mixture was warmed to room temperature, then heated to reflux overnight. Water was added to the reaction mixture, which was then extracted with ethyl acetate. The organic layer was dried over magnesium sulfate and concentrated under reduced pressure. The crude was purified by flash chromatography (ethyl acetate in cyclohexane) to afford the desired product methyl 2-(3-chloro-2-pyridyl)-5-(2,2,3,3,3-pentafluoropropoxy)pyrazole-3-carboxylate.

LC-MS (Method 1): retention time 1.08 min, m/z 386 [M+H⁺].

1H NMR (400 MHz, CDCl3) δ ppm 8.52 (dd, J=4.72, 1.45 Hz, 1H) 7.92 (dd, J=8.17, 1.63 Hz, 1H) 7.43 (dd, J=8.36, 4.72 Hz, 1H) 6.53 (s, 1H) 4.74 (td, J=12.72, 1.09 Hz, 2H) 3.79 (s, 3H)

19F NMR (377 MHz, CDCl3) δ ppm −83.62 (s, CF₃) −123.94 (s, CF₂)

The preparation of methyl 2-(3-chloro-2-pyridyl)-5-oxo-1H-pyrazole-3-carboxylate is known and described in patent CN110615780

### Step B: Preparation of 2-(3-chloro-2-pyridyl)-5-(2,2,3,3,3-pentafluoropropoxy)pyrazole-3-carboxylic acid

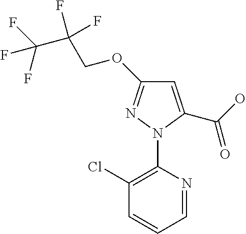 [SMILES: O=C(O)c1cc(OCC(F)(F)C(F)(F)F)nn1-c1ncccc1Cl]

A solution of methyl 2-(3-chloro-2-pyridyl)-5-(2,2,3,3,3-pentafluoropropoxy)pyrazole-3-carboxylate (1.647 g, 4.270 mmol, 1 equiv.) and lithium hydroxide monohydrate (1.075 g, 25.62 mmol, 6 equiv.) in 2-methyltetrahydrofurane (10.68 mL) and water (10.68 mL) was stirred at room temperature for 3 hours. The 2-methyltetrahydrofurane from reaction mixture was evaporated under reduced pressure. Ethyl acetate was added to the resulting aqueous mixture, followed by hydrochloric acid (aqueous, 1 N). The aqueous layer was extracted with ethyl acetate. The combined organic layers were washed with water, then with brine, dried over magnesium sulfate, filtered and evaporated under reduced pressure to afford the desired Compound P2-(3-chloro-2-pyridyl)-5-(2,2,3,3,3-pentafluoropropoxy)pyrazole-3-carboxylic acid as a white powder.

LC-MS (Method 1): retention time 0.93 min, m/z 372 [M+H⁺].

1H NMR (400 MHz, Solvent) 6 ppm 8.51 (dd, J=4.72, 1.45 Hz, 1H) 8.17 (dd, J=7.99, 1.45 Hz, 1H) 7.61 (dd, J=7.99, 4.72 Hz, 1H) 6.59 (br s, 1H) 4.96 (br t, J=13.26 Hz, 2H)

### Step C: Preparation of 6-chloro-2-[2-(3-chloro-2-pyridyl)-5-(2,2,3,3,3-pentafluoropropoxy)pyrazol-3-yl]-8-methyl-3,1-benzoxazin-4-one

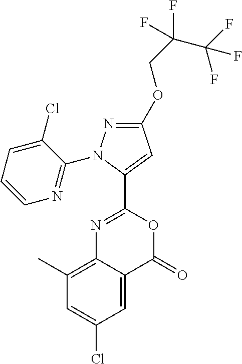 [SMILES: Cc1cc(Cl)cc2c(=O)oc(-c3cc(OCC(F)(F)C(F)(F)F)nn3-c3ncccc3Cl)nc12]

Under argon, to a solution of methane sulfonyl chloride (0.154 mL, 1.94 mmol, 2.00 equiv.) in acetonitrile (1.0 mL) were added dropwise at 0° C. a solution of 2-(3-chloro-2-pyridyl)-5-(2,2,3,3,3-pentafluoropropoxy)pyrazole-3-carboxylic acid (0.360 g, 0.970 mmol, 1.00 equiv.) in acetonitrile (3.0 mL) and pyridine (0.070 mL, 0.867 mmol, 0.90 equiv.). The reaction mixture was stirred at 0° C. for 30 minutes. Then, a suspension of 2-amino-5-chloro-3-methylbenzoic acid (0.180 g, 0.970 mmol, 1.00 equiv.) in acetonitrile (5.7 mL) was added to the previous solution at room temperature, followed by pyridine (0.160 mL, 1.98 mmol, 2.04 equiv.). The reaction mixture was stirred at room temperature for 20 hours. The reaction mixture was diluted with ethyl acetate, washed twice with water, once with brine, dried over sodium sulfate, filtered, and concentrated under reduced pressure. Crude material, 6-chloro-2-[2-(3-chloro-2-pyridyl)-5-(2,2,3,3,3-pentafluoropropoxy)pyrazol-3-yl]-8-methyl-3,1-benzoxazin-4-one was used as such in next step without further purification.

LC-MS (Method 1): retention time 1.29 min, m/z 521 [M+H⁺].

### Step D: Preparation of N-(2-carbamoyl-4-chloro-6-methyl-phenyl)-2-(3-chloro-2-pyridyl)-5-(2,2,3,3,3-pentafluoropropoxy)pyrazole-3-carboxamide

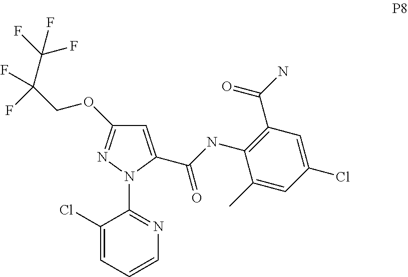 [SMILES: Cc1cc(Cl)cc(C(N)=O)c1NC(=O)c1cc(OCC(F)(F)C(F)(F)F)nn1-c1ncccc1Cl]

To a solution of 6-chloro-2-[2-(3-chloro-2-pyridyl)-5-(2,2,3,3,3-pentafluoropropoxy)pyrazol-3-yl]-8-methyl-3,1-benzoxazin-4-one (0.470 g, 0.902 mmol, 1.00 equiv.) in acetonitrile (18.0 mL) was added ammonia (2M in ethanol, 4.51 mL, 9.02 mmol, 10.0 equiv.). The reaction mixture was stirred at room temperature for 1 hour. The mixture was quenched by addition of water. The aqueous layer was extracted three times with ethyl acetate. The combined organic layers were washed twice with water, then once with brine, dried over magnesium sulfate, filtered, and concentrated under reduced pressure. Purification of the crude material by flash chromatography (ethyl acetate in cyclohexane) afforded the desired product N-(2-carbamoyl-4-chloro-6-methyl-phenyl)-2-(3-chloro-2-pyridyl)-5-(2,2,3,3,3-pentafluoropropoxy)pyrazole-3-carboxamide.

LC-MS (Method 1): Rt 1.04, m/z=538 (M+H+).

1H NMR (400 MHz, DMSO-d6) δ ppm 10.23 (s, 1H) 8.47 (dd, J=4.72, 1.45 Hz, 1H) 8.12 (dd, J=8.17, 1.63 Hz, 1H) 7.74 (br s, 1H) 7.56 (dd, J=7.99, 4.72 Hz, 1H) 7.51 (s, 1H) 7.45-7.48 (m, 1H) 7.41 (d, J=2.54 Hz, 1H) 6.84 (s, 1H) 5.00 (t, J=13.26 Hz, 2H) 2.15 (s, 3H)

The same synthetic route as for Compound P8 (step C and D) was followed to synthesize compounds P42, P46, P60 and P64 starting from 2-(3-chloro-2-pyridyl)-5-(trifluoromethyl)pyrazole-3-carboxylic acid and the corresponding di-substituted anthranilic acids.

The same synthetic route as for Compound P8 (step C and D) was followed to synthesize compounds P65, P61, P63 and P71 starting from 5-bromo-2-(3-chloro-2-pyridyl)pyrazole-3-carboxylic acid and the corresponding di-substituted anthranilic acids.

The same synthetic route as for Compound P8 (step C and D) was followed to synthesize compounds P45, P47, P55 and P59 starting from 2-(3-chloro-2-pyridyl)-5-methoxy-pyrazole-3-carboxylic acid and the corresponding di-substituted anthranilic acids.

The same synthetic route as for Compound P8 (step C and D) was followed to synthesize compound P56 starting from 2-(3-chloro-2-pyridyl)-5-(2,2-difluoroethoxy) pyrazole-3-carboxylic acid and 2-amino-5-chloro-3-methyl-benzoic acid.

The same synthetic route as for Compound P8 (step C and D) was followed to synthesize compound P68 starting from 2-(3-chloro-2-pyridyl)-5-(difluoromethyl)pyrazole-3-carboxylic acid and 2-amino-5-iodo-3-methyl-benzoic acid.

The same synthetic route as for Compound P8 (step C and D) was followed to synthesize compounds P36, P39, P50 and P66 starting from 2-(3-chloro-2-pyridyl)-5-iodo-pyrazole-3-carboxylic acid and the corresponding di-substituted anthranilic acids.

### Example 7: Preparation of N-(2-carbamoyl-4-chloro-6-methyl-phenyl)-2-(3-chloro-2-pyridyl)-5-methoxy-pyrazole-3-carboxamide (P9)

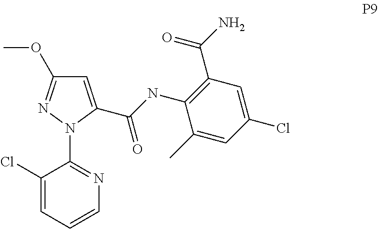 [SMILES: COc1cc(C(=O)Nc2c(C)cc(Cl)cc2C(N)=O)n(-c2ncccc2Cl)n1]

Compound P9 was prepared in using 1-(3-chloropyridin-2-yl)-3-methoxy-1H-pyrazole-5-carboxylic acid and the conditions described for Compound P7 (Example 1 Step B).

LC-MS (Method 1): Rt 0.91, m/z=421 (M+H+).

1H NMR (400 MHz, ACETONITRILE-d3) δ ppm 9.60 (br s, 1H) 8A4 (dd, J=4.7, 1.5 Hz, 1H) 7.95 (dd, J=8.0, 1.5 Hz, 1H) 7.33-7.54 (m, 3H) 6.69 (br s, 1H) 6.49 (s, 1H) 6.12-6.30 (m, 1H) 3.93 (s, 3H) 2.18 (s, 3H)

The preparation of 1-(3-chloropyridin-2-yl)-3-methoxy-1H-pyrazole-5-carboxylic acid is known and described in patent GB2463318

### Example 8: Preparation of N-(2-carbamoyl-4-iodo-6ethyl-phenyl)-2-(3-chloro-2-pyridyl)-5-methoxy-pyrazole-3-carboxamide (P10)

### Step A: Preparation of 2-[2-(3-chloro-2-pyridyl)-5-methoxy-pyrazol-3-yl]-6-iodo-8-methyl-3,1-benzoxazin-4-one

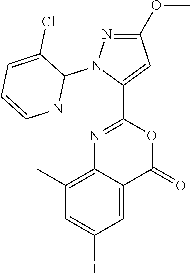

2-[2-(3-chloro-2-pyridyl)-5-methoxy-pyrazol-3-yl]-6-iodo-8-methyl-3,1-benzoxazin-4-one was prepared in using 2-amino-5-iodo-3-methylbenzoic acid and 1-(3-chloropyridin-2-yl)-3-methoxy-1H-pyrazole-5-carboxylic acid and the conditions described for 6-chloro-2-[2-(3-chloro-2-pyridyl)-5-(2,2,3,3,3-pentafluoropropoxy)pyrazol-3-yl]-8-methyl-3,1-benzoxazin-4-one (Example 6 Step C).

LC-MS (Method 1): Rt 1.05, m/z=494 (M+H+).

The preparation of 2-amino-5-iodo-3-methylbenzoic acid is known and described in patent US2015/322037 and in Tetrahedron Lett., 2012, 53, 388-391

The preparation of 1-(3-chloropyridin-2-yl)-3-methoxy-1H-pyrazole-5-carboxylic acid is known and described in patent GB2463318

### Step B: Preparation of N-(2-carbamoyl-4-iodo-6-methyl-phenyl)-2-(3-chloro-2-pyridyl)-5-methoxy-pyrazole-3-carboxamide

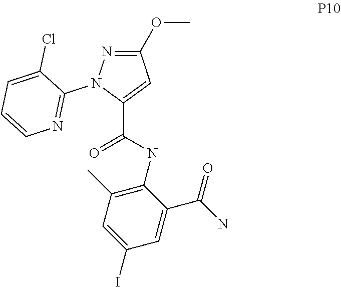

Compound P10 was prepared in using 2-[2-(3-chloro-2-pyridyl)-5-methoxy-pyrazol-3-yl]-6-iodo-8-methyl-3,1-benzoxazin-4-one and the conditions described for Compound P8 (Example 6 Step D).

LC-MS (Method 1): Rt 0.88, m/z=512 (M+H+).

1H NMR (400 MHz, CDCl3) δ ppm 9.83 (s, 1H) 8.42 (dd, J=4.72, 1.82 Hz, 1H) 7.81 (dd, J=7.99, 1.82 Hz, 1H) 7.59 (dd, J=11.81, 1.63 Hz, 2H) 7.28-7.33 (m, 1H) 6.50 (s, 1H) 6.30 (br s, 1H) 5.91 (br s, 1H) 4.00 (s, 3H) 2.12-2.17 (m, 3H).

### Example 9: Preparation of N-(2-carbamoyl-4-iodo-6-methyl-phenyl)-2-(3-chloro-2-pyridyl)-5-(2,2,2-trifluoroethoxy)pyrazole-3-carboxamide (P11)

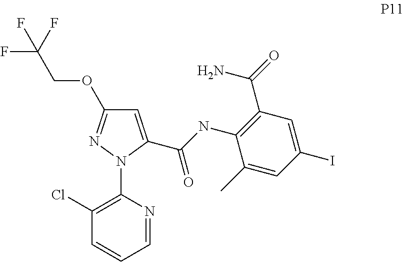 [SMILES: Cc1cc(I)cc(C(N)=O)c1NC(=O)c1cc(OCC(F)(F)F)nn1-c1ncccc1Cl]

Compound P11 was prepared in using 2-amino-5-iodo-3-methylbenzoic acid and 1-(3-chloro-2-pyridinyl)-3-(2,2,2-trifluoroethoxy)-1H-pyrazole-5-carboxylic acid and the conditions described for Compound P7 (Example 1 Step B).

The preparation of 2-amino-5-iodo-3-methylbenzoic acid is known and described in patent US2015/322037 and in Tetrahedron Lett., 2012, 53, 388-391

The preparation of 1-(3-chloro-2-pyridinyl)-3-(2,2,2-trifluoroethoxy)-1H-pyrazole-5-carboxylic acid is known and described in Bioorg. Med. Chem. Lett., 2007, 17, 6274-6279

LC-MS (Method 1): 0.99 Rt, m/z=580 (M+H+).

1H NMR (400 MHz, DMSO-d6) δ ppm 10.23 (s, 1H) 8.47 (dd, J=4.72, 1.45 Hz, 1H) 8.12 (dd, J=7.99, 1.45 Hz, 1H) 7.63-7.77 (m, 3H) 7.55 (dd, J=7.99, 4.72 Hz, 1H) 7.47 (br s, 1H) 6.83 (s, 1H) 4.91 (q, J=9.08 Hz, 2H) 2.11 (s, 3H)

### Example 10: Preparation of N-(2-carbamoyl-4-cyano-6-methyl-phenyl)-5-[(4-chlorphenyl)methoxymethyl]-2-(3-chloro-2-pyridyl)pyrazole-3-carboxamide (P12)

### Step A: Preparation of 2-[5-[(4-chlorophenyl)methoxymethyl]-2-(3-chloro-2-pyridyl)pyrazol-3-yl]-8-methyl-4-oxo-3,1-benzoxazine-6-carbonitrile

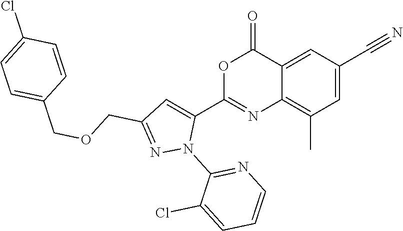 [SMILES: Cc1cc(C#N)cc2c(=O)oc(-c3cc(COCc4ccc(Cl)cc4)nn3-c3ncccc3Cl)nc12]

2-[5-[(4-chlorophenyl)methoxymethyl]-2-(3-chloro-2-pyridyl)pyrazol-3-yl]-8-methyl-4-oxo-3,1-benzoxazine-6-carbonitrile was prepared in using 5-[(4-chlorophenyl)methoxymethyl]-2-(3-chloro-2-pyridyl)pyrazole-3-carboxylic acid and 2-amino-5-cyano-3-methyl-benzoic acid and the conditions described for 6-chloro-2-[2-(3-chloro-2-pyridyl)-5-(2,2,3,3,3-pentafluoropropoxy)pyrazol-3-yl]-8-methyl-3,1-benzoxazin-4-one (Example 6 Step C).

LC-MS (Method 1): Rt 1.20, m/z=518 (M+H+).

The preparation of 5-[(4-chlorophenyl)methoxymethyl]-2-(3-chloro-2-pyridyl)pyrazole-3-carboxylic acid is known and described in step A example 5

The preparation of 2-amino-5-cyano-3-methyl-benzoic acid is known and described in patent WO2021/46315

### Step B: Preparation of N-(2-carbamoyl-4-cyano-6-methyl-phenyl)-5-[(4-chlorophenyl)methoxymethyl]-2-(3-chloro-2-pyridyl)pyrazole-3-carboxamide

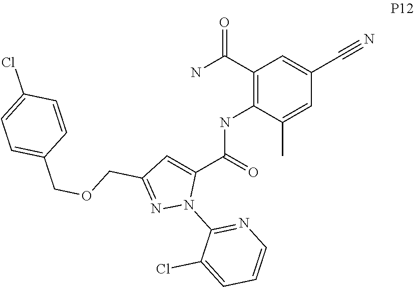 [SMILES: Cc1cc(C#N)cc(C(N)=O)c1NC(=O)c1cc(COCc2ccc(Cl)cc2)nn1-c1ncccc1Cl]

Compound P12 was prepared in using 2-[5-[(4-chlorophenyl)ethoxymethyl]-2-(3-chloro-2-pyridyl)pyrazol-3-yl]-8-methyl-4-oxo-3,1-benzoxazine-6-carbonitrile and the conditions described for Compound P8 (Example 6 Step D).

LC-MS (Method 1): Rt 1.00, m/z=535 (M+H⁺).

1H NMR (400 MHz, acetone-d6) δ ppm 10.85 (s, 1H), 8.50 (dd, J=4.7, 1.5 Hz, 1H), 8.08 (dd, J=8.0, 1.5 Hz, 1H), 7.97 (s, 1H), 7.70-7.82 (m, 2H), 7.57 (dd, J=8.0, 4.7 Hz, 1H), 7.41-7.48 (m, 2H), 7.38-7.41 (m, 2H), 7.22 (s, 2H), 4.69-4.71 (m, 2H), 4.64-4.66 (m, 2H), 2.28 (s, 3H)

### Example 11: Preparation of N-(2-carbamoyl-4-cyano-1-ethyl-phenyl)-2-(3-chloro-2-pyridyl)-5-methoxy-pyrazole-3-carboxamide (P13)

### Step A: Preparation of 2-[2-(3-chloro-2-pyridyl)-5-methoxy-pyrazol-3-yl]-8-methyl-4-oxo-3,1-benzoxazine-6-carbonitrile

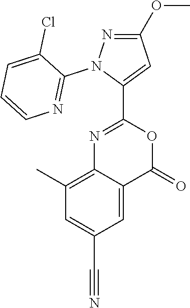 [SMILES: COc1cc(-c2nc3c(C)cc(C#N)cc3c(=O)o2)n(-c2ncccc2Cl)n1]

2-[2-(3-chloro-2-pyridyl)-5-methoxy-pyrazol-3-yl]-8-methyl-4-oxo-3,1-benzoxazine-6-carbonitrile was prepared in using 2-amino-5-cyano-3-methylbenzoic acid and 1-(3-chloropyridin-2-yl)-3-methoxy-1H-pyrazole-5-carboxylic acid and the conditions described for 6-chloro-2-[2-(3-chloro-2-pyridyl)-5-(2,2,3,3,3-pentafluoropropoxy)pyrazol-3-yl]-8-methyl-3,1-benzoxazin-4-one (Example 6 Step C).

LC-MS (Method 1): Rt 0.91, m/z=393 (M+H+).

The preparation of 2-amino-5-cyano-3-methyl-benzoic acid is known and described in patent WO2021/46315

The preparation of 1-(3-chloropyridin-2-yl)-3-methoxy-1H-pyrazole-5-carboxylic acid is known and described in patent GB2463318

### Step B: Preparation of N-(2-carbamoyl-4-cyano-6-ethyl-phenyl)-2-(3-chloro-2-pyridyl)-5-methoxy-pyrazole-3-carboxamide

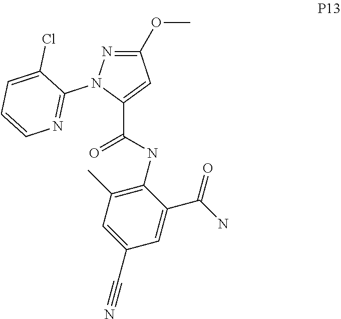

Compound P13 was prepared in using 2-[2-(3-chloro-2-pyridyl)-5-ethoxy-pyrazol-3-yl]-8-methyl-4-oxo-3,1-benzoxazine-6-carbonitrile and the conditions described for Compound P8 (Example 6 Step D).

LC-MS (Method 1): Rt 0.77, m/z=411 (M+H+).

1H NMR (400 MHz, CDCl3) δ ppm 10.32 (s, 1H) 8.45 (dd, J=4.72, 1.45 Hz, 1H) 7.83 (dd, J=7.99, 1.82 Hz, 1H) 7.62 (d, J=1.45 Hz, 1H) 7.56-7.59 (m, 1H) 7.33 (dd, J=7.99, 4.72 Hz, 1H) 6.39-6.50 (m, 2H) 5.90 (br s, 1H) 4.00 (s, 3H) 2.25 (s, 3H)

### Example 12: Preparation of N-(2-carbamoyl-4-chloro-6-methyl-phenyl)-5-[(5-chlorobenzotriazol-1-yl)methyl]-2-(3-chloro-2-pyridyl)pyrazol-3-carboxamide (P14); and N-(2-carbamoyl-4chloro-6-methyl-phenyl)-5-[(6-chlorobenzotriazol-1-yl)methyl]-2-(3chloro-2-pyridyl)pyrazole-3-carboxamide (P16)

### Step A: Preparation of (5-chlorobenzotriazol-1-yl)sodium

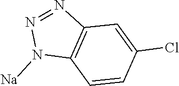

(5-chlorobenzotriazol-1-yl)sodium was prepared in using 5-chlorobenzotriazole and the conditions described for (5-cyclopropyltetrazol-2-yl)sodium (example 1 step A)

1H NMR (400 MHz, DMSO-d6) δ ppm 7.59 (t, J=4.25 Hz, 2H) 6.82-6.85 (m, 1H)

### Step B: Preparation of N-(2-carbamoyl-4-chloro-6-methyl-phenyl)-5-[(5-chlorobenzotriazol-1-yl)methyl]-2-(3-chloro-2-pyridyl)pyrazole-3-carboxamide (P14); and N-(2-carbamoyl-4-chloro-6-methyl-phenyl)-5-[(6-chlorobenzotriazol-1-yl)ethyl]-2-(3-chloro-2-pyridyl)pyrazole-3-carboxamide (P16)

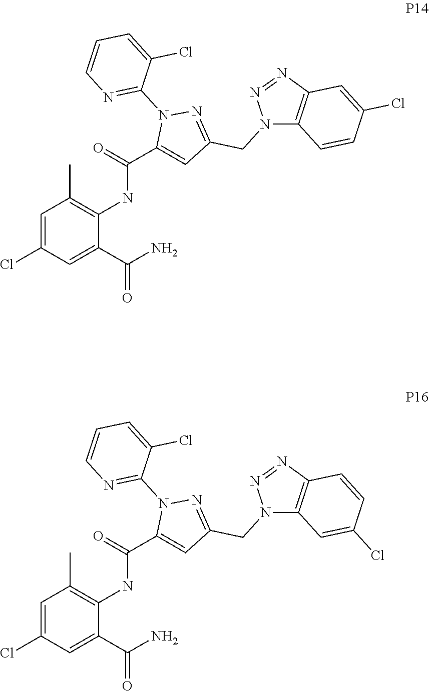

Compound P14 and P16 were prepared in using (5-chlorobenzotriazol-1-yl)sodium and the conditions described for Compound P1 and P2 (Step B)

### Compound P14:

LC-MS (Method 1): retention time 0.95 min, 557 m/z [M+H⁺].

1H NMR (600 MHz, DMSO-d6) δ ppm 10.16-10.24 (m, 1H) 8.47 (ddd, J=4.7, 3.3, 1.5 Hz, 1H) 8.25 (d, J=1.5 Hz, 1H) 8.09-8.19 (m, 1H) T97 (d, J=8.8 Hz, 1H) 7.70 (br s, 1H) 7.65 (dd, J=8.9, 1.8 Hz, 1H) 7.58 (ddd, J=7.9, 4.8, 2.9 Hz, 1H) 7.44 (br s, 2H) 7.37 (d, J=2.3 Hz, 1H) 7.11-7.21 (m, 1H) 6.16 (s, 2H) 2.10 (d, J=2.2 Hz, 3H)

### Compound P16:

LC-MS (method 1): retention time 0.95 min, 557 m/z [M+H⁺].

1H NMR (600 MHz, DMSO-d6) δ ppm 10.16-10.24 (m, 1H) 8.47 (ddd, J=4.7, 3.3, 1.5 Hz, 1H) 8.09-8.19 (m, 3H) 7.70 (br s, 1H) 7.58 (ddd, J=7.9, 4.8, 2.9 Hz, 1H) 7.48 (dd, J=8.8, 1.9 Hz, 1H) 7.44 (br s, 2H) 7.37 (d, J=2.3 Hz, 1H) 7.11-7.21 (m, 1H) 6.14 (s, 2H) 2.10 (d, J=22 Hz, 3H)

### Example 13: Preparation of 5-[[5-(3-bromophenyl)tetrazol-2-yl],ethyl]-N-(2-carbamoyl-4-chloro-6-methyl-phenyl)-2-(3-chloro-2-pyridyl)pyrazole-3-carboxamide (P48)

### Step A: Preparation of [5-(3-bromophenyl)tetrazol-2-yl]sodium

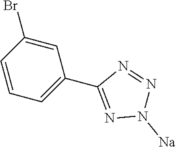

[5-(3-bromophenyl)tetrazol-2-yl]sodium was prepared in using 5-(3-bromophenyl)-1H-tetrazole and the conditions described for (5-cyclopropyltetrazol-2-yl)sodium (example 2 step A).

### Step B: Preparation of 5-[[5-(3-bromophenyl)tetrazol-2-yl]methyl]-N-(2-carbamoyl-4-chloro-6-methyl-phenyl)-2-(3-chloro-2-pyridyl)pyrazole-3-carboxamide

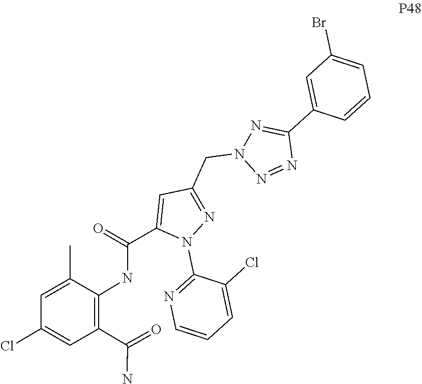

To a solution of N-(2-carbamoyl-4-chloro-6-methyl-phenyl)-5-(chloromethyl)-2-(3-chloro-2-pyridyl)pyrazole-3-carboxamide (P7) (0.500 g, 1.140 mmol, 1 equiv.) in acetone (9 mL) were added [5-(3-bromophenyl)tetrazol-2-yl]sodium (0.281 g, 1.140 mmol, 1 equiv.) and potassium iodide (0.019 g, 0.114 mmol, 0.1 equiv.). The reaction mixture was stirred at 60° C. for 18h.

The reaction mixture was allowed to come back at RT, water was added, and acetone was evaporated.

The residue was purified by reverse phase (water/acetonitrile) to afford the desired compound 5-[[5-(3-bromophenyl)tetrazol-2-yl]methyl]-N-(2-carbamoyl-4-chloro-6-methyl-phenyl)-2-(3-chloro-2-pyridyl)pyrazole-3-carboxamide.

LC-MS (Method 1): Rt 1.08, m/z=628 (M+H+).

¹H NMR (₁-CDCl3) δ: 9.95 (s, 1H), 8.47-8.53 (dd, 1H), 8.38 (d, 1H), 8.15 (d, 1H), 7.86-7.91 (dd, 1H), 7.62 (br d, 1H), 7.33-7.44 (m, 4H), 7.12 (s, 1H), 6.02 (s, 3H), 5.62-5.73 (m, 1H), 2.22 (s, 3H)

### Example 14: Preparation of 5-[[5-(4-bromophenyl)tetrazol-2-ylmethyl]-N-(2-carbamoyl-4-chloro-6-methyl-phenyl)-2-(3-chloro-2-pyridyl)pyrazole-3-carboxamide (P49)

### Step A: Preparation of [5-(4-bromophenyl)tetrazol-2-yl]sodium

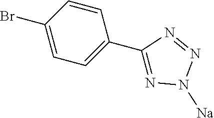

[5-(4-bromophenyl)tetrazol-2-yl]sodium was prepared in using 5-(4-bromophenyl)-1H-tetrazole and the conditions described for (5-cyclopropyltetrazol-2-yl)sodium (example 2 step A).

### Step B: Preparation of 5-[[5-(4-bromophenyl)tetrazol-2-yl]methyl]-N-(2-carbamoyl-4-chloro-6-methyl-phenyl)-2-(3-chloro-2-pyridyl)pyrazole-3-carboxamide

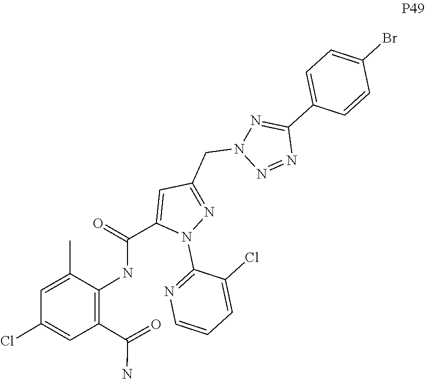

Compound P49 was prepared in using [5-(4-bromophenyl)tetrazol-2-yl]sodium and the conditions described for Compound P48.

LC-MS (Method 1): Rt 1.08, m/z=628 (M+H+).

¹H NMR (₁-CDCl3) δ: 9.94 (s, 1H), 8.46-8.53 (dd, 1H), 8.06-8.12 (m, 2H), 7.86-7.91 (dd, 1H), 7.62-7.67 (m, 2H), 7.38-7.44 (dd, 1H), 7.31-7.37 (m, 2H), 7.11 (s, 1H), 6.03-6.11 (br s, 1H), 5.99-6.02 (m, 2H), 5.67-5.75 (br s, 1H), 2.21 (s, 3H)

### Example 15: Preparation of 5-[[5-(2-bromophenyl)tetrazol-2-yl]methyl]-N-(2-carbamoyl-4-chloro-6-methyl-phenyl)-2-(3-chloro-2-pyridyl)pyrazole-3-carboxamide (P53)

### Step A: Preparation of [5-(2-bromophenyl)tetrazol-2-yl]sodium

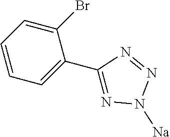

[5-(2-bromophenyl)tetrazol-2-yl]sodium was prepared in using 5-(2-bromophenyl)-1H-tetrazole and the conditions described for (5-cyclopropyltetrazol-2-yl)sodium (example 2 step A).

### Step B: Preparation of 5-[[5-(2-bromophenyl)tetrazol-2-yl]methyl]-N-(2-carbamoyl-4-chloro-6-methyl-phenyl)-2-(3-chloro-2-pyridyl)pyrazole-3-carboxamide (P53)

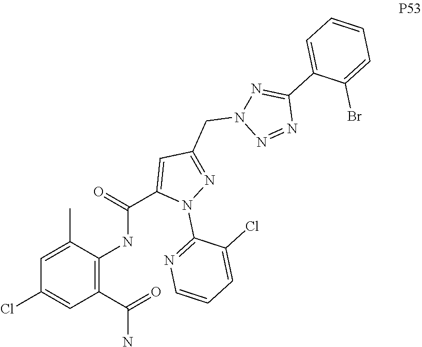 [SMILES: Cc1cc(Cl)cc(C(N)=O)c1NC(=O)c1cc(Cn2nnc(-c3ccccc3Br)n2)nn1-c1ncccc1Cl]

Compound P53 was prepared in using [5-(2-bromophenyl)tetrazol-2-yl]sodium and the conditions described for Compound P48. The residue was purified by flash chromatography (ethyl acetate in cyclohexane).

LC-MS (Method 1): Rt 1.02, m/z=628 (M+H+).

¹H NMR (₁-CDCl3) δ: 9.95 (s, 1H), 8.48-8.52 (dd, 1H), 7.91-7.96 (dd, 1H), 7.86-7.91 (dd, 1H), 7.74-7.79 (dd, 1H), 7.44-7.49 (m, 1H), 7.39-7.43 (m, 1H), 7.32-7.38 (m, 3H), 7.17 (s, 1H), 6.07 (s, 3H), 5.64-5.78 (br s, 1H), 2.22 (s, 3H)

### Example 16: Preparation of N-(2-carbamoyl-4-chloro-1-ethyl-phenyl)-2-(3-chloro-2-pyridyl)-5-[[5-(p-tolyl)tetrazol-2-yl]ethyl]pyrazole-3-carboxamide (P54)

### Step A: Preparation of [5-(p-tolyl)tetrazol-2-yl]sodium

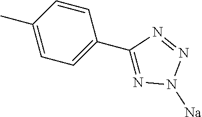

[5-(p-tolyl)tetrazol-2-yl]sodium was prepared in using 5-(p-tolyl)-2H-tetrazole and the conditions described for (5-cyclopropyltetrazol-2-yl)sodium (example 2 step A).

### Step B: Preparation of N-(2-carbamoyl-4-chloro-6-methyl-phenyl)-2-(3-chloro-2-pyridyl)-5-[[5-(p-tolyl)tetrazol-2-yl]methyl]pyrazole-3-carboxamide (P54)

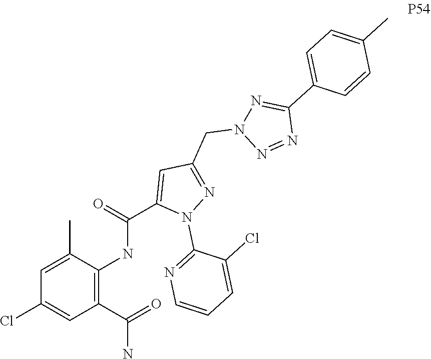 [SMILES: Cc1ccc(-c2nnn(Cc3cc(C(=O)Nc4c(C)cc(Cl)cc4C(N)=O)n(-c4ncccc4Cl)n3)n2)cc1]

Compound P54 was prepared in using [5-(p-tolyl)tetrazol-2-yl]sodium and the conditions described for Compound P48. The residue was purified by flash chromatography (ethyl acetate in cyclohexane).

LC-MS (Method 1): Rt 1.04, m/z=562 (M+H+).

¹H NMR (₁-CDCl3) δ: 9.91 (s, 1H), 8.48-8.51 (dd, 1H), 8.09 (d, 2H), 7.86-7.90 (dd, 1H), 7.38-7.42 (m, 1H), 7.30-7.36 (m, 4H), 7.11 (s, 1H), 6.02-6.14 (br s, 1H), 5.95-6.02 (s, 2H), 5.60-5.76 (br s, 1H), 243 (s, 3H), 2.19-2.25 (s, 3H)

### Example 17: Preparation of N-(2-carbamoyl-4,6-dichloro-phenyl)-2-(3-chloro-2-pyridyl)-5-[[5-[4-(trifluoromethyl) phenyl]tetrazol-2-yl]methyl]pyrazole-3-carboxamide (P58)

### Step A: Preparation of ethyl (E)-4-methoxy-2-oxopent-3-enoate

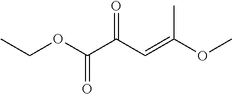

Ethyl 2-chloro-2-oxo-acetate (50.0 g, 366 mmol) in Dichloromethane (120 mL) were charged into a stirred solution of 2-methoxyprop-1-ene (30.6 g, 425 mmol) and pyridine (46.0 mL, 366 mmol) in Dichloromethane (240 mL) at 0° C. for 30 min. Upon the reaction mixture was stirred at 30° C. for 16 h. The progress of the reaction was monitored by TLC (30% EtOAc/hexanes) and it showed no starting material. Reaction mixture was diluted with Dichloromethane (200 mL) washed with 10% hydrochloric acid (100 mL), water (100 mL) and brine solution (100 mL), dried over anhydrous Na2SO4 and solvent was evaporated under reduced pressure to obtain crude mixture. The crude mixture was purified by combi flash column chromatography (combiflash, 0 to 100% Hexane/EtOAC). Pure fractions were combined and evaporated under reduced pressure to afford ethyl (E)-4-methoxy-2-oxo-pent-3-enoate (30.0 g, 174 mmol, yield: 47.6%) as colour less gel.

1H NMR (400 MHz, DMSO-d6): δ 625 (s, 1H), 4.31 (q, J=7.2 Hz, 2H), 3.78 (s, 3H), 2.39 (s, 3H), 1.37 (t, J=7.2 Hz, 3H);

### Step B: Preparation of ethyl (E)-5-bromo-4-methoxy-2-oxopent-3-enoate

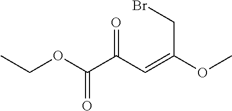

Bromine (7.14 mL, 139 mmol) in Dichloromethane (100 mL) was added drop wise to stirred solution of ethyl (E)-4-methoxy-2-oxo-pent-3-enoate (20.0 g, 116 mmol) in Dichloromethane (50.0 mL), at 0° C. The reaction mixture was stirred at 0° C. for 1 h, followed by addition of a solution of pyridine (8.00 mL, 116 mmol) in Dichloromethane (50.0 mL) at 0° C. Upon the reaction mixture was stirred for 1 h. The progress of the reaction was monitored by TLC (30% EtOAc/hexanes) and it showed no starting material. Reaction mixture was diluted with Dichloromethane (100 mL) and added water (200 mL), 2 N Hydrochloric acid (15.0 mL). The reaction mixture was stirred for 5 min, separated aqueous layer and organic layer. Aqueous layer extract with Dichloromethane (100 mL). The organic layer was washed with water (100 mL), brine solution (100 mL), dried over Na2SO4 and concentrated under reduced pressure to obtain crude mixture. The crude mixture was purified by combi flash column chromatography (combiflash, 0 to 100% Hexane/EtOAC). Pure fractions were combined and evaporated under reduced pressure to afford ethyl (E)-5-bromo-4-methoxy-2-oxo-pent-3-enoate (12.0 g, 47.8 mmol, yield: 41.1%) as a red gel. 1H NMR (400 MHz, DMSO-d6): δ 6.36 (s, 1H), 4.48 (s, 2H), 4.33 (q, J=7.2 Hz, 2H), 3.86 (s, 3H), 1.38 (t, J=7.2 Hz, 3H)

### Step C: Preparation of ethyl 5-(bromomethyl)-2-(3-chloro-2-pyridyl) pyrazole-3-carboxylate

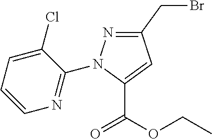 [SMILES: CCOC(=O)c1cc(CBr)nn1-c1ncccc1Cl]

To a stirred solution of ethyl (E)-5-bromo-4-methoxy-2-oxo-pent-3-enoate (10.0 g, 39.8 mmol) in Acetic acid (30.0 mL) was charged with (3-chloro-2-pyridyl) hydrazine (5.72 g, 39.8 mmol) at room temperature. The reaction mixture was stirred for 2 h at 110° C. The progress of the reaction was monitored by TLC (40% EtOAc/hexanes) and it showed no starting material. The reaction mixture was concentrated under reduced pressure to obtain crude mixture. The crude mixture was purified by combi flash column chromatography (combiflash, 0 to 100% Hexane/EtOAC). Pure fractions were combined and evaporated under reduced pressure to afford ethyl 5-(bromomethyl)-2-(3-chloro-2-pyridyl) pyrazole-3-carboxylate (6.00 g, 17.4 mmol, yield: 43.7%) as an off-whit solid. 1H NMR (400 MHz, DMSO-d6): δ 8.57-8.55 (m, 1H), 8.27-8.25 (m, 1H), 7.71-7.68 (m, 1H), 7.21 (s, 1H), 4.72 (s, 2H), 4.15 (q, J=7.2 Hz, 2H), 1.09 (t, J=7.2 Hz, 3H)

### Step D: Preparation of ethyl 2-(3-chloro-2-pyridyl)-5-[[5-[4-(trifluoromethyl) phenyl]tetrazol-2-yl]methyl]pyrazole-3-carboxylate

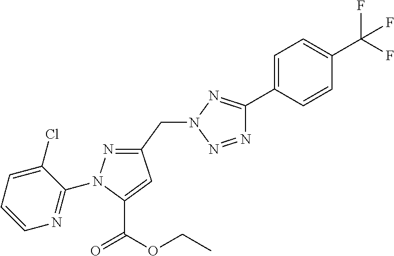 [SMILES: CCOC(=O)c1cc(Cn2nnc(-c3ccc(C(F)(F)F)cc3)n2)nn1-c1ncccc1Cl]

To a stirred solution of ethyl 5-(bromomethyl)-2-(3-chloro-2-pyridyl) pyrazole-3-carboxylate (3.00 g, 8.71 mmol) in Acetonitrile (20.0 mL) were charged with 5-[4-(trifluoromethyl) phenyl]-2H-tetrazole (224 g, 10.4 mmol) and Potassium carbonate (2.41 g, 17.4 mmol) at room temperature. The reaction mixture was stirred for 3 h at 80° C. The progress of the reaction was monitored by TLC ((40% EtOAc/hexanes) and it showed no starting material. The reaction mixture was diluted with water (100 mL), extracted with EtOAc (2×100 mL). The combined organic layers were washed with brine (100 mL), dried over Na₂SO₄ and concentrated under reduced pressure to obtain crude product. The crude product was purified by combi flash column chromatography (combiflash, 0 to 100% Hexane/EtOAC). Pure fractions were combined and evaporated under reduced pressure to afford ethyl 2-(3-chloro-2-pyridyl)-5-[[5-[4-(trifluoromethyl) phenyl]tetrazol-2-yl]methyl]pyrazole-3-carboxylate (1.90 g, 3.98 mmol, yield: 45.7%) as an off-white solid

### Step E: Preparation of 2-(3-chloro-2-pyridyl)-5-[[5-[4-(trifluoromethyl) phenyl]tetrazol-2-yl]methyl]pyrazole-3-carboxylic acid

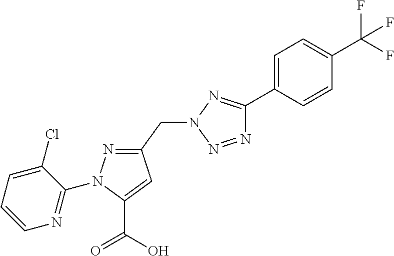 [SMILES: O=C(O)c1cc(Cn2nnc(-c3ccc(C(F)(F)F)cc3)n2)nn1-c1ncccc1Cl]

To a stirred solution of ethyl 2-(3-chloro-2-pyridyl)-5-[[5-[4-(trifluoromethyl) phenyl]tetrazol-2-yl]methyl]pyrazole-3-carboxylate (1.90 g, 3.98 mmol) in Methanol (15.0 mL) were charged into a stirred solution of lithium hydroxide, hydrate (0.834 g, 19.9 mmol) and water (5.00 mL) at 0° C. The reaction mixture was stirred at 30° C. for 3 h. The progress of the reaction was monitored by TLC (10% MeOH/DCM) and it showed no starting material. Most of the solvent was evaporated under reduced pressure and adjust pH 2.0 to 3.0 with 2N HCl at 0° C. and formed white solid and filtered. Filtered cake was washed with n-pentane and dry with rotary evaporator under reduced pressure to afford 2-(3-chloro-2-pyridyl)-5-[[5-[4-(trifluoromethyl) phenyl]tetrazol-2-yl]methyl]pyrazole-3-carboxylic acid (1 50 g, 3.34 mmol, yield: 83.9%) as Off-white solid

1H NMR (400 MHz, DMSO-d6): δ 13.73 (s, 1H), 8.53 (dd, J=1.2 Hz, 4.8 Hz, 1H), 8.29 (d, J=8.4 Hz, 2H), 822 (dd, J=1.6 Hz, 8.0 Hz, 1H), 7.94 (d, J=8.4 Hz, 2H), 7.68-7.64 (m, 1H), 7.16 (s, 1H), 6.17 (s, 2H)

### Step F: Preparation of N-(2-carbamoyl-4,6-dichloro-phenyl)-2-(3-chloro-2-pyridyl)-5-[[5-[4-(trifluoromethyl) phenyl]tetrazol-2-yl]methyl]pyrazole-3-carboxamide (P58)

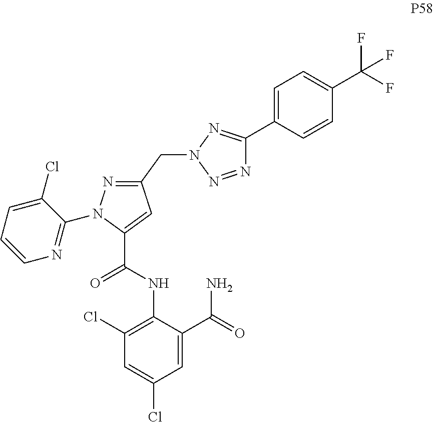 [SMILES: NC(=O)c1cc(Cl)cc(Cl)c1NC(=O)c1cc(Cn2nnc(-c3ccc(C(F)(F)F)cc3)n2)nn1-c1ncccc1Cl]

The same synthetic route as for Compound P8 (step C and D) was followed to synthesize compound P58 starting from 2-(3-chloro-2-pyridyl)-5-[[5-[4-(trifluoromethyl) phenyl]tetrazol-2-yl]methyl]pyrazole-3-carboxylic acid and 2-amino-3,5-dichloro-benzoic acid.

The same synthetic route as for Compound P58 (step F) was followed to synthesize compounds P41, P43, P44, P57, P62 and P67 starting from 2-(3-chloro-2-pyridyl)-5-[[5-[4-(trifluoromethyl) phenyl]tetrazol-2-yl]methyl]pyrazole-3-carboxylic acid and the corresponding di-substituted anthranilic acids

### Example 18: Synthesis of N-(4-bromo-2-carbamoyl-6-chloro-phenyl)-2-(3-chloro-2-pyridyl)-5-[[4-(trifluoromethyl) triazol-1-yl]methyl]pyrazole-3-carboxamide (P40) and N-(4-bromo-2-carbamoyl-6-chloro-phenyl)-2-(3-chloro-2-pyridyl)-5-[[4-(trifluoromethyl)triazol-2-yl]methyl]pyrazole-3-carboxamide (P37)

### Step A: Synthesis of ethyl 2-(3-chloro-2-pyridyl)-5-[[4-(trifluoromethyl) triazol-2-yl]methyl]pyrazole-3-carboxylate and ethyl 2-(3-chloro-2-pyridyl)-5-[[4-(trifluoromethyl) triazol-1-yl]methyl]pyrazole-3-carboxylate

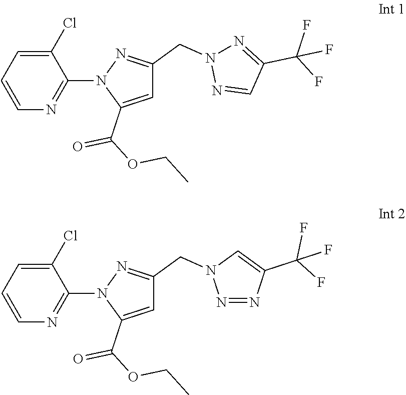 [SMILES: CCOC(=O)c1cc(Cn2ncc(C(F)(F)F)n2)nn1-c1ncccc1Cl ; CCOC(=O)c1cc(Cn2cc(C(F)(F)F)nn2)nn1-c1ncccc1Cl]

To a stirred solution of ethyl 5-(bromomethyl)-2-(3-chloro-2-pyridyl) pyrazole-3-carboxylate (3.00 g, 8.71 mmol) in Acetonitrile (60.0 mL) were added 4-(trifluoromethyl)-1H-triazole (1.43 g, 10.4 mmol) and dipotassium carbonate (3.61 g, 26.1 mmol) at room temperature. The resulting reaction mixture was stirred at 80° C. for 3 h. The progress of the reaction was monitored by TLC (30% Ethyl acetate/Hexane), and it showed no starting material. The reaction mixture was diluted with water (10.0 mL), extracted with EtOAc (2×100 mL). The combined organic layers were washed with brine (20.0 mL), dried over Na2SO4 and concentrated under reduced pressure to obtain crude mixture. The crude mixture was purified by combi flash column chromatography (combiflash, 0 to 100% Hexane/EtOAC). Pure fractions were combined and evaporated under reduced pressure to afford ethyl 2-(3-chloro-2-pyridyl)-5-[[4-(trifluoromethyl) triazol-2-yl]methyl]pyrazole-3-carboxylate (Int 1) (0.700 g, 1.75 mmol, yield: 20.1%) and ethyl 2-(3-chloro-2-pyridyl)-5-[[4-(trifluoromethyl) triazol-1-yl]methyl]pyrazole-3-carboxylate (Int 2) (0.900 g, 2.25 mmol, yield: 25.8%) as an off-white solid.

Int1: 1H NMR (400 MHz, DMSO-d6): δ 8.55 (dd, J=1.6 Hz, 4.8 Hz, 1H), 8.49 (s, 1H), 8.26 (dd, J=1.6 Hz, 8.0 Hz, 1H), 7.71-7.68 (m, 1H), 7.13 (s, 1H), 5.92 (s, 2H), 4.13 (q, J=7.2 Hz, 2H), 1.08 (t, J=7.2 Hz, 3H)

Int 2: 1H NMR (400 MHz, DMSO-d6): δ 9.03 (s, 1H), 8.55 (dd, J=1.6 Hz, 4.8 Hz, 1H), 8.25 (dd, J=1.6 Hz, 7.6 Hz, 1H), 7.71-7.68 (m, 1H), 7.22 (s, 1H), 5.84 (s, 2H), 4.14 (q, J=7.2 Hz, 2H), 1.08 (t, J=7.2 Hz, 3H)

### Step B1: Synthesis of 2-(3-chloro-2-pyridyl)-5-[[4-(trifluoromethyl) triazol-2-yl]methyl]pyrazole-3-carboxylic acid

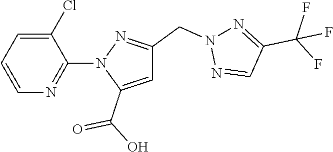 [SMILES: O=C(O)c1cc(Cn2ncc(C(F)(F)F)n2)nn1-c1ncccc1Cl]

To a stirred solution of ethyl 2-(3-chloro-2-pyridyl)-5-[[4-(trifluoromethyl) triazol-2-yl]methyl]pyrazole-3-carboxylate (900 mg, 2.25 mmol) in Methanol (10.0 mL) were added to a stirred solution of lithium hydroxide, hydrate (471 mg, 11.2 mmol) and water (5.0 mL) at 0° C. The reaction mixture was stirred for 2 h at room temperature. The progress of the reaction was monitored by TLC (10% MeOH/DCM) and it showed no starting material. Most of the solvent was evaporated under reduced pressure and adjust pH 2.0 to 3.0 with 2N HCl at 0° C. Reaction mass was extracted with ethyl acetate (3×30.0 mL). The combined organic layers were washed with brine (20.0 mL), dried over Na2SO4 and evaporated under reduced pressure to afford 2-(3-chloro-2-pyridyl)-5-[[4-(trifluoromethyl) triazol-2-yl]methyl]pyrazole-3-carboxylic acid (800 mg, 2.15 mmol, yield: 95.6%) as colour less gel.

### Step B2: Synthesis of 2-(3-chloro-2-pyridyl)-5-[[4-(trifluoromethyl) triazol-1-yl]methyl]pyrazole-3-carboxylic acid

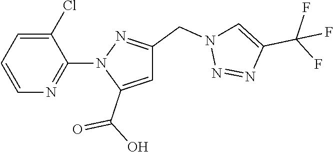 [SMILES: O=C(O)c1cc(Cn2cc(C(F)(F)F)nn2)nn1-c1ncccc1Cl]

To a stirred solution of ethyl 2-(3-chloro-2-pyridyl)-5-[[4-(trifluoromethyl) triazol-1-yl]methyl]pyrazole-3-carboxylate (1.20 g, 2.99 mmol) in methanol (15.0 mL): water (5.00 mL) was added lithium hydroxide hydrate (0.628 g, 15.0 mmol) at 0° C. The resulting reaction mixture was stirred at room temperature for 3 h. Reaction mixture was analysed by TLC (40% Ethyl acetate/Hexane), and it showed no starting material. The reaction mixture was concentrated and diluted with water (100.0 mL), acidify the aqueous layer with 2N HCl then extracted with EtOAc (2×200 mL). The combined organic layers were washed with brine (200 mL), dried over Na2SO4 and concentrated under reduced pressure to afford 2-(3-chloro-2-pyridyl)-5-[[4-(trifluoromethyl) triazol-1-yl]methyl]pyrazole-3-carboxylic acid (1.10 g, 2.95 mmol, yield: 98.6%) as an off-white solid.

### Step C1: Preparation of N-(4-bromo-2-carbamoyl-6-chloro-phenyl)-2-(3-chloro-2-pyridyl)-5-[[4-(trifluoromethyl) triazol-2-yl]methyl]pyrazole-3-carboxamide (P37)

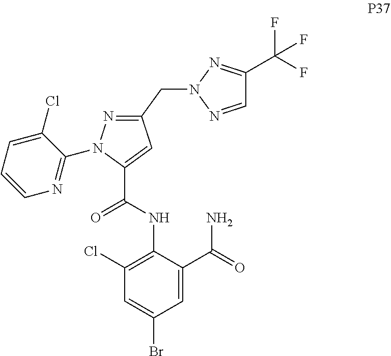 [SMILES: NC(=O)c1cc(Br)cc(Cl)c1NC(=O)c1cc(Cn2ncc(C(F)(F)F)n2)nn1-c1ncccc1Cl]

The same synthetic route as for Compound P8 (step C and D) was followed to synthesize compound P37 starting from 2-(3-chloro-2-pyridyl)-5-[[4-(trifluoromethyl) triazol-2-yl]methyl]pyrazole-3-carboxylic acid and 2-amino-5-bromo-3-chloro-benzoic acid

The same synthetic route as for Compound P37 (step C1) was followed to synthesize compounds P38 and P52 starting from 2-(3-chloro-2-pyridyl)-5-[[4-(trifluoromethyl) triazol-2-yl]methyl]pyrazole-3-carboxylic acid and the corresponding di-substituted anthranilic acids

### Step C2: Preparation of N-(4-bromo-2-carbamoyl-6-chloro-phenyl)-2-(3-chloro-2-pyridyl)-5-[[4-(trifluoromethyl) triazol-1-yl]methyl]pyrazole-3-carboxamide (P40)

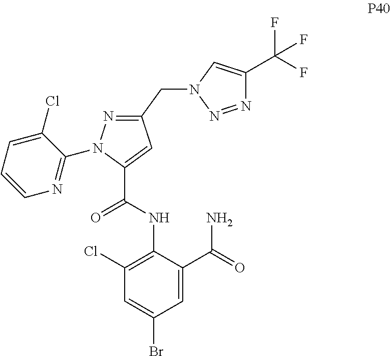 [SMILES: NC(=O)c1cc(Br)cc(Cl)c1NC(=O)c1cc(Cn2cc(C(F)(F)F)nn2)nn1-c1ncccc1Cl]

The same synthetic route as for Compound P8 (step C and D) was followed to synthesize compound P40 starting from 2-(3-chloro-2-pyridyl)-5-[[4-(trifluoromethyl) triazol-1-yl]methyl]pyrazole-3-carboxylic acid and 2-amino-5-bromo-3-chloro-benzoic acid.

The same synthetic route as for Compound P40 (step C₂) was followed to synthesize compounds P51 and P35 starting from 2-(3-chloro-2-pyridyl)-5-[[4-(trifluoromethyl) triazol-1-yl]methyl]pyrazole-3-carboxylic acid and the corresponding di-substituted anthranilic acids

### Example 19: Preparation of N-(2-carbamoyl-chloro-6-methyl-phenyl)-2-(3-chloro-2-pyridyl)-5-(cyanomethyl)pyrazole-3-carboxamide (P70)

### Step A: Preparation of ethyl 2-(3-chloro-2-pyridyl)-5-(cyanomethyl) pyrazole-3-carboxylate

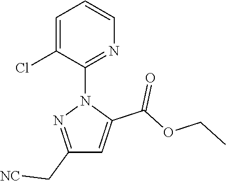 [SMILES: CCOC(=O)c1cc(CC#N)nn1-c1ncccc1Cl]

A 50 mL two-necked round bottom flask was equipped with nitrogen balloon, and it was charged with ethyl 5-(bromomethyl)-2-(3-chloro-2-pyridyl) pyrazole-3-carboxylate (1.50 g, 4.31 mmol) in DMF (15.0 mL) was added sodium cyanide (0.211 g, 4.31 mmol) the resulting reaction mixture was stirred at room temperature for 16 h. Progress of reaction mixture monitor by TLC (40% Ethyl acetate/Hexane), it showed starting material was consumed product formed polar spot reaction was seems to be completed. Upon the reaction mixture was poured in ice water extracted with ethyl acetate (2×20 mL). The combined organic layers were washed with brine (20.0 mL), dried over Na2SO4 and concentrated under reduced pressure to obtain crude product. The crude product was purified by combi flash column chromatography eluted with (20% ethyl acetate/Hexane) pure fraction was collected and concentrated under reduced pressure afford ethyl 2-(3-chloro-2-pyridyl)-5-(cyanomethyl) pyrazole-3-carboxylate (120 g, 3.72 mmol, yield: 86.2%) as a light brown solid.

1H NMR (CDCl3, 400 MHz): δ 8.52 (dd, J=1.6 Hz, 4.8 Hz, 1H), 7.93 (dd, J=1.6 Hz, 8.4 Hz, 1H), 7.47 (dd, J=4.8 Hz, 8.0 Hz, 1H), 7.10 (s, 1H), 4.26 (q, J=7.2 Hz, 2H), 3.88 (s, 2H), 1.25 (t, J=6.8 Hz, 3H);

### Step B: Preparation of 2-(3-chloro-2-pyridyl)-5-(cyanomethyl) pyrazole-3-carboxylic acid

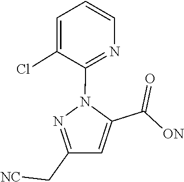

A 50 mL two-necked round bottom flask was equipped with nitrogen balloon and it was charged with ethyl 2-(3-chloro-2-pyridyl)-5-(cyanomethyl) pyrazole-3-carboxylate (400 mg, 1.38 mmol) in ethanol (10.0 mL) was added lithium hydroxide monohydrate (0.289 g, 6.88 mmol) in water (4.0 mL) at 0° C. The resulting reaction mixture was stirred at room temperature for 3 h. Reaction mixture was analysed by TLC (40% Ethyl acetate/Hexane), and it showed no starting material. Upon the reaction mixture was concentrated under reduced pressure, the residue was diluted with water and acidify with 2N HCl to pH 2 then extract the compound in EtOAc (2×100 mL). The combined organic layers were washed with brine (100 mL), dried over Na2SO4 and concentrated under reduced pressure to afford 2-(3-chloro-2-pyridyl)-5-(cyanomethyl) pyrazole-3-carboxylic acid (0.340 g) as an off-white solid. 1H NMR (CDCl3, 400 MHz): δ 13.72 (s, 1H), 8.56 (dd, J=1.6 Hz, 4.4 Hz, 1H), 8.25 (dd, J=1.2 Hz, 8.0 Hz, 1H), 7.69 (dd, J=4.8 Hz, 8.0 Hz, 1H), 7.02 (s, 1H), 4.17 (s, 2H); MS: m/z=261.0 [M−H+].

### Step C: Preparation of 2-[5-(6-chloro-8-methyl-4-oxo-3,1-benzoxazin-2-yl)-1-(3-chloro-2-pyridyl) pyrazol-3-yl]acetonitrile

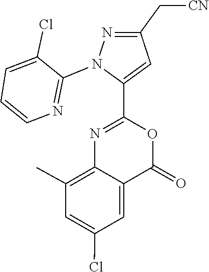 [SMILES: Cc1cc(Cl)cc2c(=O)oc(-c3cc(CC#N)nn3-c3ncccc3Cl)nc12]

A 25 mL two-necked round bottom flask was charged with 2-(3-chloro-2-pyridyl)-5-(cyanomethyl)pyrazole-3-carboxylic acid (350 mg, 1.33 mmol) in acetonitrile (10.0 mL) was added 2-amino-5-chloro-3-methyl-benzoic acid (0.247 g, 1.33 mmol),pyridine (0.316 g, 4.0 mmol) then cooled to 0° C. and added methane sulfonyl chloride (0.227 g, 1.98 mmol). The resulting reaction mixture was stirred at room temperature for 4 h. Reaction mixture was analysed by TLC (30% Ethyl acetate/Hexane), and it showed no starting material. The reaction mixture was diluted with water (50.0 mL), extracted with EtOAc (2×100 mL). The combined organic layers were washed with brine (50.0 mL), dried over Na2SO4 and concentrated under reduced pressure to afford 2-[5-(6-chloro-8-methyl-4-oxo-3,1-benzoxazin-2-yl)-1-(3-chloro-2-pyridyl)pyrazol-3-yl]acetonitrile (0.400 g) as a yellow solid.

Note: Crude product was used for next step.

### Step D: Preparation of N-(2-carbamoyl-4-chloro-6-methyl-phenyl)-2-(3-chloro-2-pyridyl)-5-(cyanomethyl)pyrazole-3-carboxamide

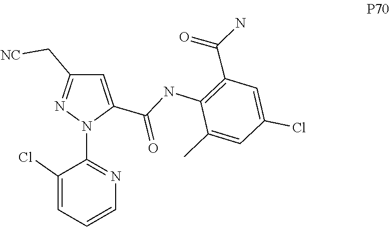

A 25 mL two-necked round bottom flask was equipped with nitrogen balloon and it was charged with 2-[5-(6-chloro-8-methyl-4-oxo-3,1-benzoxazin-2-yl)-1-(3-chloro-2-pyridyl)pyrazol-3-yl]acetonitrile (400 mg, 0.970 mmol) in acetonitrile (10.0 mL) and added ammonia (2000 mmol/L, 4.85 mL, 9.70 mmol) at 0° C. The resulting reaction mixture was stirred at room temperature for 2 h. Reaction mixture was analysed by TLC (40% Ethyl acetate/Hexane), and it showed no starting material. The reaction mixture was diluted with water (100 mL), extracted with EtOAc (2×100 mL). The combined organic layers were washed with brine (50.0 mL), dried over Na2SO4 and concentrated under reduced pressure to obtain crude residue. The crude residue was purified by reverse phase combi flash column chromatography (combiflash, 0 to 100% Water/ACN). Pure fractions were combined and evaporated under reduced pressure to afford N-(2-carbamoyl-4-chloro-6-methyl-phenyl)-2-(3-chloro-2-pyridyl)-5-(cyanomethyl)pyrazole-3-carboxamide (250 mg, 0.582 mmol, yield: 60%) as an off-white solid.

1H NMR (DMSO-d6, 400 MHz): δ 10.28 (s, 1H), 8.49 (dd, J=1.6 Hz, 4.8 Hz, 1H), 8.16 (dd, J=1.6 Hz, 8.0 Hz, 1H), 7.73 (s, 1H), 7.60 (dd, J=4.8 Hz, 8.4 Hz, 1H), 7.49 (s, 1H), 7.46 (d, J=2.0 Hz, 1H), 7.40 (d, J=2.0 Hz, 1H), 7.24 (s, 1H), 4.23 (s, 2H), 2.14 (s, 3H); MS: m/z=429.1 [M+H⁺].

Melting point: 180-185° C.

### Example 20: N-(2-carbamoyl-4-chloro-6-methy-phenyl)-2-(3-chloro-2-pyridyl)-5-(1-cyanocyclopropyl) pyrazole-3carboxamide (P72)

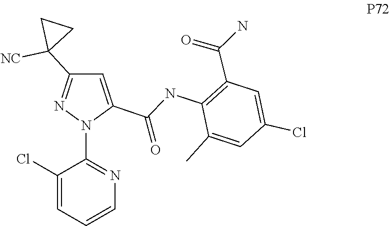

A 10 mL vial was charged with N-(2-carbamoyl-4-chloro-6-methyl-phenyl)-2-(3-chloro-2-pyridyl)-5-(cyanomethyl)pyrazole-3-carboxamide (P70) (200 mg, 0.466 mmol) in 50% NaOH solution (2.0 mL) and added 1,2-dibromoethane (0.175 g, 0.932 mmol), benzyl(triethyl)ammonium chloride (0.212 g, 0.932 mmol) at 0° C. The resulting reaction mixture was stirred at room temperature for 24 h. Reaction mixture was analysed by TLC (80% Ethyl acetate/Hexane), and it showed no starting material. Upon the reaction mixture was diluted with water (50.0 mL), extracted with EtOAc (2×50 mL). The combined organic layers were washed with brine (50.0 mL), dried over Na₂SO₄ and concentrated under reduced pressure to obtain crude mixture. The crude mixture was purified by reverse phase combi flash column chromatography. Pure fractions were combined and evaporated under reduced pressure to afford N-(2-carbamoyl-4-chloro-6-methyl-phenyl)-2-(3-chloro-2-pyridyl)-5-(1-cyanocyclopropyl)pyrazole-3-carboxamide (20.0 mg, 0.0439 mmol, yield: 9.43%) as an off-white solid. 10 ¹H NMR (DMSO-d6 400 MHz): δ 10.24 (s, 1H), 8.48 (dd, J=1.2 Hz, 4.8 Hz, 1H), 8.15 (dd, J=1.6 Hz, 8.0 Hz, 1H), 7.73 (s, 1H), 7.59 (dd, J=4.8 Hz, 8.0 Hz, 1H), 7.50 (s, 1H), 7.46 (d, J=2.0 Hz, 1H), 7.40 (d, J=2.4 Hz, 1H), 7.29 (s, 1H), 2.13 (s, 3H), 1.87 (q, J=4.8 Hz, 2H), 1.58 (q, J=4.4 Hz, 2H); MS: m/z=455.1 [M+H+].

Melting point: 225-230° C.

### Example 21: 5-bromo-2-(3-bromo-2-pyridyl)-N-(2-carbamoyl-chloro-6-methyl-phenyl)pyrazole-3-carboxamide (P73)

### Step A: Preparation of 2-[5-bromo-2-(3-bromo-2-pyridyl)pyrazol-3-yl]-6-chloro-8-methyl-3,1-benzoxazin-4-one

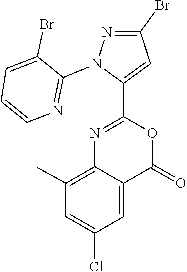 [SMILES: Cc1cc(Cl)cc2c(=O)oc(-c3cc(Br)nn3-c3ncccc3Br)nc12]

To an ice-cooled solution of 2-amino-5-chloro-3-methyl-benzoic acid (0.10 g, 0.54 mmol) and 5-bromo-2-(3-bromo-2-pyridyl)pyrazole-3-carboxylic acid (1 equiv., 0.54 mmol) in acetonitrile (2.7 mL) and pyridine (2.2 mmol, 0.17 mL), MsCl (1.3 mmol, 0.10 mL) was added drop wise.

After 30 minutes the ice-bath was removed and the reactions mixture was stirred at room temperature for 3 h. The reaction mixture was diluted (10 mL) water. After stirring for 15 min, the suspension was filtered off cold, washed with water and vacuum dried. The crude product was used in the next step without further purification.

LC-MS (Method 1): Rt 1.21, m/z=497 (M+H+).

Note: 5-bromo-2-(3-bromo-2-pyridyl)pyrazole-3-carboxylic acid can be easily prepared by following similar procedure than described for 5-bromo-2-(3-chloro-2-pyridyl)pyrazole-3-carboxylic acid in CN111620850

### Step B: Preparation of 5-bromo-2-(3-bromo-2-pyridyl)-N-(2-carbamoyl-4-chloro-6-methyl-phenyl)pyrazole-3-carboxamide

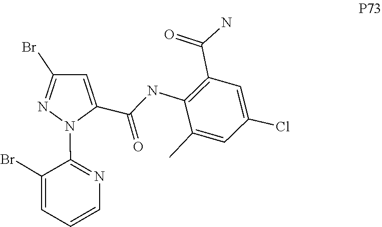

The same synthetic route as for Compound P8 (step D) was followed to synthesize compounds P73 5 LC-MS (Method 1): Rt 0.90, m/z=514 (M+H+).

1H NMR (400 MHz, DMSO-d6) δ ppm 2.15 (s, 3H) 7.35-7.42 (m, 2H) 7.44-7.54 (m, 3H) 7.74 (br s, 1H) 8.29 (dd, J=7.99, 1.09 Hz, 1H) 8.52 (dd, J=4.54, 1.27 Hz, 1H) 10.27 (s, 1H)

### Example 22: N-(2-carbamoyl-chloro-6-ethyl-phenyl)-2-(3-chloro-2-pyridyl)-5-(methylsulfanylmethyl)pyrazole-3-carboxamide (P79)

### Step A: Preparation of ethyl 2-(3-chloro-2-pyridyl)-5-(methylsulfanylmethyl)pyrazole-3-carboxylate

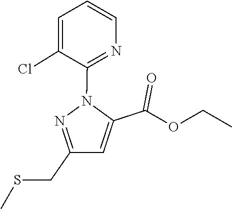 [SMILES: CCOC(=O)c1cc(CSC)nn1-c1ncccc1Cl]

A 100 mL two-necked round bottom flask was equipped with nitrogen balloon and it was charged with ethyl 5-(bromomethyl)-2-(3-chloro-2-pyridyl)pyrazole-3-carboxylate (1.20 g, 3.48 mmol) in tetrahydrofuran (100 mL) was added drop wise sodium methane thiolate Sodium Salt (ca. 15% in Water) (10.0 mL, 3.48 mmol) to stirred solution 60° C. The reaction mass was stirred 3 h. Reaction mixture was analysed by TLC (40% EtOAc/hexanes) and it showed no starting material. Upon the reaction mixture was diluted with water (50.0 mL) and extracted with EtOAc (2×50 mL). The combined organic layers were washed with water (2×50 mL), brine (50.0 mL), dried over Na2SO4 and concentrated under reduced pressure to afford crude ethyl 2-(3-chloro-2-pyridyl)-5-(methylsulfanylmethyl)pyrazole-3-carboxylate (0.800 g, 2.57 mmol, yield: 73.7%) as a colour less gel.

1H NMR (DMSO-d6, 400 MHz): δ 8.56 (dd, J=1.2 Hz, 4.4 Hz, 1H), 8.23 (dd, J=1.2 Hz, 8.0 Hz, 1H), 7.69 (dd, J=4.8 Hz, 8.0 Hz, 1H), 7.05 (s, 1H), 4.16 (q, J=7.2 Hz, 2H), 3.75 (s, 2H), 2.03 (s, 3H), 1.10 (t, J=4.4 Hz, 3H);

### Step B: Preparation of 2-(3-chloro-2-pyridyl)-5-(methylsulfanylmethyl)pyrazole-3-carboxylic acid

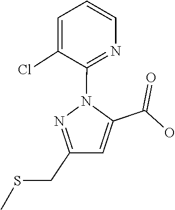

A 25 mL two-necked round bottom flask was equipped with nitrogen balloon and it was charged with ethyl 2-(3-chloro-2-pyridyl)-5-(methylsulfanylmethyl)pyrazole-3-carboxylate (250 mg, 0.802 mmol) in Tetrahydrofuran (10.0 mL) were added to a stirred solution of lithium hydroxide monohydrate (40.4 g, 0.962 mmol) and water (3.0 mL) at 0° C. The reaction mixture was stirred for 4 h at room temperature. Reaction mixture was analysed by TLC (10% MeOH/DCM) and it showed no starting material. Most of the solvent was evaporated under reduced pressure and adjust pH 2.0 to 3.0 with 2N HCl at 0° C. Reaction mass was extracted with 10% MeOH/DCM (3×30 mL). The combined organic layers were washed with brine (20.0 mL), dried over Na2SO4 and evaporated under reduced pressure, to afford 2-(3-chloro-2-pyridyl)-5-(methylsulfanylmethyl)pyrazole-3-carboxylic acid (160 mg, 0.564 mmol, yield: 70.3%) as an off-whit solid.

MS: m/z=282.0 [M−H+].

### Step C: Preparation of 6-chloro-2-[2-(3-chloro-2-pyridyl)-5-(methylsulfanylmethyl)pyrazol-3-yl]-8-methyl-3,1-benzoxazin-4-one

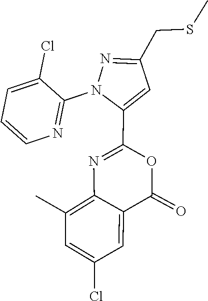 [SMILES: CSCc1cc(-c2nc3c(C)cc(Cl)cc3c(=O)o2)n(-c2ncccc2Cl)n1]

A 25 mL two-necked round bottom flask was equipped with nitrogen balloon and it was charged with 2-(3-chloro-2-pyridyl)-5-(methylsulfanylmethyl)pyrazole-3-carboxylic acid (160 mg, 0.564 mol) in acetonitrile (5.0 mL) were added 2-amino-5-chloro-3-methyl-benzoic acid (126 mg, 0.677 mmol), pyridine (0.182 mL, 2.26 mmol) and methanesulfonyl chloride (0.131 mL, 1.69 mmol) at 0° C. The reaction mixture was stirred for 2 h at room temperature. Reaction mixture was analysed by TLC (30% Ethyl acetate/Hexane) and it showed no starting material. The reaction mixture was diluted with water (30.0 mL), extracted with EtOAc (2×50 mL). The combined organic layers were washed with brine (30.0 mL), dried over Na2SO4 and concentrated under reduced pressure to afford crude 6-chloro-2-[2-(3-chloro-2-pyridyl)-5-(methylsulfanylmethyl)pyrazol-3-yl]-8-methyl-3,1-benzoxazin-4-one (200 mg, 0.369 mol, yield: 65.5%) as an off-white gummy solid.

Note: Crude compound was used for next step.

### Step D: Preparation of N-(2-carbamoyl-4-chloro-6-methyl-phenyl)-2-(3-chloro-2-pyridyl)-5-(methylsulfanylmethyl)pyrazole-3-carboxamide

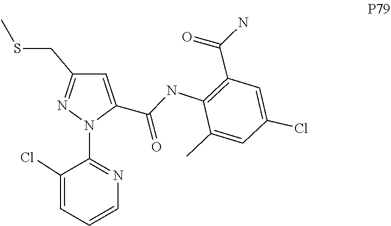

A 25 mL two-necked round bottom flask was equipped with nitrogen balloon and it was charged with 6-chloro-2-[2-(3-chloro-2-pyridyl)-5-(methylsulfanylmethyl)pyrazol-3-yl]-8-methyl-3,1-benzoxazin-4-one (200 mg, 0.462 mmol) in Acetonitrile (5.0 mL) and added 2M ammonia in ethanol (3.0 mL) at room temperature. The resulting reaction mixture was stirred at room temperature for 2 h. Reaction mixture was analysed by TLC (10% MeOH/DCM) and it showed no starting material. The reaction mixture was concentrated under reduced pressure to obtain crude product. The crude was purified by combi flash column chromatography (combiflash, 0 to 100% Hexane/EtOAC). Pure fractions were combined and evaporated under reduced pressure to afford N-(2-carbamoyl-4-chloro-6-methyl-phenyl)-2-(3-chloro-2-pyridyl)-5-(methylsulfanylmethyl)pyrazole-3-carboxamide (45.0 mg, 0.0999 mol, yield: 21.6%) as an off-white solid.

1H NMR (DMSO-d6, 400 MHz): δ 10.24 (s, 1H), 8.48 (dd, J=1.6 Hz, 4.8 Hz, 1H), 8.14 (dd, J=1.6 Hz, 8.0 Hz, 1H), 7.74 (s, 1H), 7.58-7.53 (m, 2H), 7.46 (d, J=2.4 Hz, 1H), 7.39 (d, J=2.4 Hz, 1H), 7.17 (s, 1H), 3.77 (s, 2H), 2.14 (s, 3H), 2.08 (s, 3H); MS: m/z=450.1 [M+H⁺].

Melting point: 175-180° C.

### Example 23: N-(2-carbamoyl-4-chloro-6-methyl-phenyl)-2-(3-chloro-2-pyridyl)-5-(methylsulfonylmethyl)pyrazole-3-carboxamide (P78)

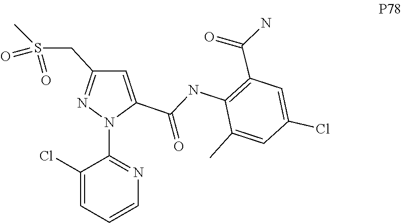

A 25 mL two necked round bottom flask was equipped with nitrogen balloon and it was charged with N-(2-carbamoyl-4-chloro-6-methyl-phenyl)-2-(3-chloro-2-pyridyl)-5-(methylsulfanylmethyl)pyrazole-3-carboxamide (80.0 mg, 0.178 mmol) in Dichloromethane (15.0 mL) and added 3-chlorobenzenecarboperoxoic acid (61.3 mg, 0.355 mol) portion wise at 0° C. for 2 in. The resulting reaction mixture was stirred at room temperature for 6 h. The reaction mixture was analysed by TLC (10 MeOH/DCM), it showed no starting material. The reaction mixture was diluted with dichloromethane and washed with NaHCO₃ (3×20 mL) and organic layer was dried over Na2SO4, concentrated under reduced pressure to obtained crude residue. The crude residue was purified by combi flash column chromatography (combiflash, 0 to 100% Hexane/EtOAC). Pure fractions were combined and evaporated under reduced pressure to obtain N-(2-carbamoyl-4-chloro-6-methyl-phenyl)-2-(3-chloro-2-pyridyl)-5-(methylsulfonylmethyl)pyrazole-3-carboxamide (45.0 mg, 0.0933 mmol, yield: 52.5%) as an off-white solid.

1H NMR (DMSO-d6, 400 MHz): δ 10.35 (s, 1H), 8.50 (t, J=1.6 Hz, 1H), 8.17-8.15 (m, 1H), 7.75 (s, 1H), 7.61-7.57 (m, 1H), 7.52 (s, 1H), 7.46 (s, 1H), 7.39 (s, 1H), 7.32 (s, 1H), 4.69 (s, 2H), 3.03 (s, 3H), 2.14 (s, 3H); MS: m/z=482.1 [M+H+].

Melting point: 140-145° C.

### Example 24: Preparation of N-(2-carbamoyl-4-chloro-6-methyl-phenyl)-2-(3-chloro-2-pyridyl)-5-(rnethyisulfinylmethyl)pyrazole-3-carboxamide (P74)

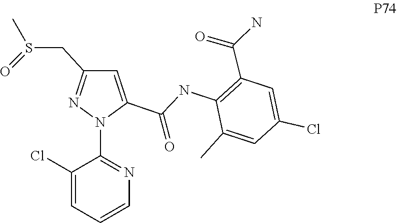

A 25 mL two-necked round bottom flask was equipped with nitrogen balloon and it was charged with N-(2-carbamoyl-4-chloro-6-methyl-phenyl)-2-(3-chloro-2-pyridyl)-5-(methyl methyl)pyrazole-3-carboxamide (70.0 mg, 0.155 mmol) in Ethanol (8.0 mL) were added to a stirred solution of sodium periodate (66.5 mg, 0.311 mmol) and water (2.0 mL) at 0° C. The reaction mixture was stirred for 2 h at room temperature. Reaction mixture was analysed by TLC (10% MeOH/DCM) and it showed no starting material. The solvent was evaporated under reduced pressure to obtain crude mixture. The crude mixture was purified by reversed-phase combi flash column chromatography (combiflash, 0 to 100% Water/Acetonitrile). Pure fractions were combined and evaporated under reduced pressure to afford N-(2-carbamoyl-4-chloro-6-methyl-phenyl)-2-(3-chloro-2-pyridyl)-5-(methylsulfinylmethyl)pyrazole-3-carboxamide (45.0 mg, 0.0965 mmol, yield: 62.1%) as an off-white solid.

1H NMR (DMSO-d6, 400 MHz): δ 10.31 (s, 1H), 8.49 (dd, J=1.6 Hz, 4.8 Hz, 1H), 8.15 (dd, J=1.2 Hz, 8.0 Hz, 1H), 7.75 (s, 1H), 7.59 (dd, J=4.8 Hz, 8.4 Hz, 1H), 7.51 (s, 1H), 7.44 (s, 1H), 7.40 (s, 1H), 7.21 (s, 1H), 4.29 (d, J=13.6 Hz, 1H), 4.12 (d, J=13.6 Hz, 1H), 2.59 (s, 3H), 2.13 (s, 3H); MS: m/z=466.0 [M+H+].

Melting point: 125-130° C.

### Example 25: N-(2-carbamoyl-4-chloro-6-methyl-phenyl)-2-(3-chloro-2-pyridyl)-5-ethyl-pyrazole-carboxamide (P75)

### Step A: Preparation of ethyl (2E)-2-methoxyimino-4-oxo-hexanoate

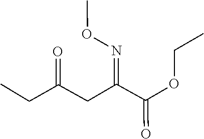

A 50 mL two-necked round bottom flask was equipped with nitrogen balloon and it was charged with ethyl 2,4-dioxohexanoate (2.00 g, 11.6 mmol) in N,N-dimethylformamide (20.0 mL) and added O-methylhydroxylamine;hydrochloride (0.970 g, 11.6 mmol) at 0° C. The resulting reaction mixture was stirred at room temperature for 15 h. Reaction mixture was analysed by TLC (40% Ethyl acetate/Hexane) and it showed no starting material. Upon the reaction mixture was diluted with water (100 mL), extracted with EtOAc (2×200 mL). The combined organic layers were washed with NaHCO₃solution (100 mL), dried over Na2SO4 and concentrated under reduced pressure to obtain crude mixture. The crude reaction mixture was purified by combi flash column chromatography (combiflash, 0 to 100% Hexane/EtOAC). Pure fractions were combined and evaporated under reduced pressure to afford ethyl (2E)-2-methoxyimino-4-oxo-hexanoate (10 g, 5.47 mmol, yield: 47.1%) as brown gum. 1H NMR (CDCl3, 400 MHz): δ 4.29 (q, J=7.2 Hz, 2H), 3.99 (s, 3H), 3.64 (s, 2H), 2.47 (q, J=7.2 Hz, 2H), 1.30 (t, J=7.2 Hz, 3H), 1.03 (t, J=7.6 Hz, 3H); MS: m/z=202.1 [M+H+].

### Step B: Preparation of ethyl 2-(3-chloro-2-pyridyl)-5-ethyl-pyrazole-3-carboxylate

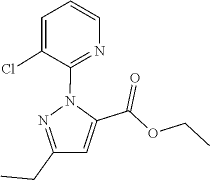 [SMILES: CCOC(=O)c1cc(CC)nn1-c1ncccc1Cl]

A 50 mL two-necked round bottom flask was equipped with nitrogen balloon and it was charged with ethyl (2E)-2-methoxyimino-4-oxo-hexanoate (1.00 g, 4.97 mmol) in acetic acid (15.0 mL) and added (3-chloro-2-pyridyl)hydrazine (0.714 g, 4.97 mmol). The resulting reaction mixture was stirred at 100° C. for 5 h. Reaction mixture was analysed by TLC (40% Ethyl acetate/Hexane) and it showed no starting material. Upon the reaction mixture was diluted with water (100 mL), extracted with EtOAc (2×100 mL). The combined organic layers were washed with brine (50.0 mL), dried over Na2SO4 and concentrated under reduced pressure to obtain crude mixture. The crude mixture was purified by combi flash column chromatography (combiflash, 0 to 100% Hexane/EtOAC). Pure fractions were combined and evaporated under reduced pressure to afford ethyl 2-(3-chloro-2-pyridyl)-5-ethyl-pyrazole-3-carboxylate (0.650 g, 2.32 mmol, yield: 46.8%) as an off white solid.

1H NMR (DMSO-d6, 400 MHz): δ 8.55 (dd, J=1.6 Hz, 4.8 Hz, 1H), 8.23 (dd, J=1.6 Hz, 8.0 Hz, 1H), 7.67 (dd, J=4.4 Hz, 8.0 Hz, 1H), 6.99 (s, 1H), 4.15 (q, J=7.2 Hz, 2H), 2.70 (q, J=7.6 Hz, 2H), 1.25 (t, J=7.6 Hz, 3H), 1.10 (t, J=7.2 Hz, 3H).

### Step C: Preparation of 2-(3-chloro-2-pyridyl)-5-ethyl-pyrazole-3-carboxylic acid

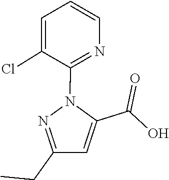 [SMILES: CCc1cc(C(=O)O)n(-c2ncccc2Cl)n1]

A 25 mL two-necked round bottom flask was equipped with nitrogen balloon and it was charged with ethyl 2-(3-chloro-2-pyridyl)-5-ethyl-pyrazole-3-carboxylate (600 mg, 2.14 mmol) in ethanol (10.0 mL) was added lithium hydroxide monohydrate (0.450 g, 10.7 mmol) in water (4.00 mL) at 0° C. The resulting reaction mixture was stirred at room temperature for 3 h. Reaction mixture was analysed by TLC (40% Ethyl acetate/Hexane) and it showed no starting material. Upon the reaction mixture was concentrated under reduced pressure the residue was diluted with water and acidify with 2N HCl to pH 2 then extract the compound in EtOAc (2×100 mL). The combined organic layers were washed with brine (100 mL), dried over Na2SO4 and concentrated under reduced pressure to afford 2-(3-chloro-2-pyridyl)-5-ethyl-pyrazole-3-carboxylic acid (0.500 g) as an off-white solid

1H NMR (DMSO-d6, 400 MHz): δ 13.35 (brs, 1H), 8.52 (dd, J=1.6 Hz, 4.8 Hz, 1H), 8.20 (dd, J=1.6 Hz, 8.0 Hz, 1H), 7.63 (dd, J=4.8 Hz, 8.0 Hz, 1H), 6.89 (s, 1H), 2.68 (q, J=7.6 Hz, 2H), 1.24 (t, J=7.6 Hz, 3H); MS: m/z=251.9 [M+H+].

### Step D: Preparation of 6-chloro-2-[2-(3-chloro-2-pyridyl)-5-ethyl-pyrazol-3-yl]-8-methyl-3,1-benzoxazin-4-one

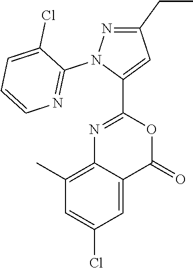 [SMILES: CCc1cc(-c2nc3c(C)cc(Cl)cc3c(=O)o2)n(-c2ncccc2Cl)n1]

A 25 mL two-necked round bottom flask was equipped with nitrogen balloon, and it was charged with 2-(3-chloro-2-pyridyl)-5-ethyl-pyrazole-3-carboxylic acid (400 mg, 1.59 mmol) in acetonitrile (15.0 mL) were added 2-amino-5-chloro-3-methyl-benzoic acid (0.295 g, 1.59 mmol), pyridine (0.377 g, 4.77 mmol) and methane sulfonyl chloride (0.728 g, 6.36 mmol) at 0° C. The reaction mixture was stirred for 3 h at room temperature. Reaction mixture was analysed by TLC (30% Ethyl acetate/Hexane) and it showed no starting material. The reaction mixture was diluted with water (30.0 mL), extracted with EtOAc (2×50 mL). The combined organic layers were washed with brine (30.0 mL), dried over Na2SO4 and concentrated under reduced pressure to afford crude 6-chloro-2-[2-(3-chloro-2-pyridyl)-5-ethyl-pyrazol-3-yl]-8-methyl-3,1-benzoxazin-4-one (0.360 g, 0.897 mmol, yield: 56.4%) as a light-yellow solid.

Note: Crude compound directly used for next step.

1H NMR (DMSO-d6, 400 MHz): δ 8.60 (dd, J=1.6 Hz, 4.8 Hz, 1H), 8.00 (dd, J=1.6 Hz, 8.4 Hz, 1H), 7.88 (dd, J=0.8 Hz, 2.8 Hz, 1H), 7.76-7.69 (m, 2H), 7.18 (s, 1H), 2.76 (q, J=7.6 Hz, 2H), 1.72 (s, 3H), 1.30 (t, J=7.6 Hz, 3H); MS: m/z=400.9 [M+H+].

### Step E: Preparation of N-(2-carbamoyl-4-chloro-6-ethyl-phenyl)-2-(3-chloro-2-pyridyl)-5-ethyl-pyrazole-3-carboxamide (P75)

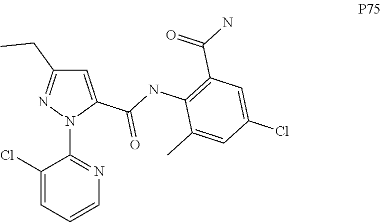 [SMILES: CCc1cc(C(=O)Nc2c(C)cc(Cl)cc2C(N)=O)n(-c2ncccc2Cl)n1]

A 25 mL two-necked round bottom flask was equipped with nitrogen balloon and it was charged with 6-chloro-2-[2-(3-chloro-2-pyridyl-pyrazol-3-yl]-8-methyl-3,1-benzoxazin-4-one (150 mg, 0.374 mmol) in acetonitrile (10.0 mL) and added ammonia (2000 mmol/L, 1.87 mL, 3.74 mmol) at 0° C. The resulting reaction mixture was stirred at room temperature for 2 h. Reaction mixture was analysed by TLC (40% Ethyl acetate/Hexane) and it showed no starting material. The reaction mixture was diluted with water (100.0 mL), extracted with EtOAc (2×100 mL). The combined organic layers were washed with brine (50.0 mL), dried over Na2SO4 and concentrated under reduced pressure to obtain crude mixture. The crude reaction mixture was purified by combi flash column chromatography (combiflash, 0 to 100% Hexane/EtOAC). Pure fractions were combined and evaporated under reduced pressure to afford N-(2-carbamoyl-4-chloro-6-methyl-phenyl)-2-(3-chloro-2-pyridyl)-5-ethyl-pyrazole-3-carboxamide (65 mg, 0.155 mmol, yield: 41.6%) as a light brown solid.

1H NMR (DMSO-d6, 400 MHz): δ 10.17 (s, 1H), 8.47 (dd, J=1.6 Hz, J=4.4 Hz, 1H), 8.12 (dd, J=1.6 Hz, 8.0 Hz, 1H), 7.75 (s, 1H), 7.56-7.52 (m, 2H), 7.46 (s, 1H), 7.46-7.39 (m, 1H), 7.06 (s, 1H), 2.72 (q, J=7.2 Hz, J=7.6 Hz, 2H), 2.14 (s, 3H), 1.28 (t, J=7.6 Hz, 3H); MS: m/z=418.0 [M+H+].

Melting point: 245-250° C.

The same synthetic route as for Compound P75 (step A to E) was followed to synthesize compounds P76 starting from 4-cyclopropyl-2,4-dioxo-butanoate.

### Example 26: N-(2-carbamoyl-4-chloro-6-methyl-phenyl)-2-(3-chloro-2-pyridyl)-5-ethyl-pyrazole-3-carboxamide (P77)

### Step A: Preparation of 2-(3-bromopyrazol-1-yl)pyrimidine

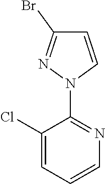 [SMILES: Clc1cccnc1-n1ccc(Br)n1]

A solution of 2-chloropyrimidine (450 mg, 3.9 mmol), 3-bromo-1H-pyrazole (3.7 mmol, 0.95 g) and DBU (3.9291 mmol, 0.6 mL) in N,N-Dimethylformamide (8 mL) was stirred at 120° C. for 12 hours.

The reaction mixture was diluted in water and extracted with ethylacetate. The organic phase was washed with HCl 1N, NaHCO3 (aq) and brine, dried over MgSO4, filtered and concentrated to give 2-(3-bromopyrazol-1-yl)pyrimidine (752 mg).

1H NMR (400 MHz, DMSO) δ ppm 6.72-6.82 (m, 1H) 7.47-7.56 (m, 1H) 8.58-8.70 (m, 1H) 8.82-8.94 (m, 2H)

### Step B: Preparation of 5-bromo-2-pyrimidin-2-yl-pyrazole-3-carboxylic acid

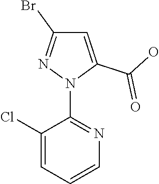 [SMILES: O=C(O)c1cc(Br)nn1-c1ncccc1Cl]

To a solution of 2-(3-bromopyrazol-1-yl)pyrimidine (150 mg, 0.665 mmol) in Tetrahydrofuran (3 mL) was added LDA freshly prepared dropwise at −78° C. and stirred for 10 min, then CO2 was bubbled into the mixture for 20 min at −78° C., then slowly warmed to 15° C. for 1h. The reaction mixture was warm up to RT and CO₂ was bubbled into the mixture for 1h. Water was added to the mixture and extracted with ethyl acetate, the water layer was adjusted to pH ˜3 with 1N HCl and extracted with ethyl acetate. The organic layers were dried over MgSO4, filtered, and concentrated to give preparation of 5-bromo-2-pyrimidin-2-yl-pyrazole-3-carboxylic acid (88 mg).

1H NMR (400 MHz, DMSO) δ ppm 7.09-7.21 (m, 1H) 7.62-7.72 (m, 1H) 8.91-9.02 (m, 2H) 13.52-14.40 (m, 1H)

### Step C: Preparation of N-(2-carbamoyl-4-chloro-6-methyl-phenyl)-2-(3-chloro-2-pyridyl)-5-ethyl-pyrazole-3-carboxamide (P77)

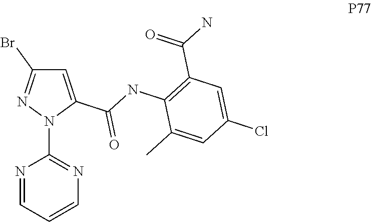 [SMILES: Cc1cc(Cl)cc(C(N)=O)c1NC(=O)c1cc(Br)nn1-c1ncccn1]

Compound P77 was prepared in using 5-bromo-2-pyrimidin-2-yl-pyrazole-3-carboxylic acid and 2-amino-5-chloro-3-methyl-benzoic acid and the conditions described for Compound P8 (Example 6 Step C and D).

LC-MS (Method 1): Rt 0.78, m/z=436 (M+H+).

1H NMR (400 MHz, DMSO) δ ppm 2.25-2.31 (m, 3H) 6.93-7.11 (m, 1H) 7.38-7.48 (m, 1H) 7.49-7.52 (m, 1H) 7.52-7.58 (m, 1H) 7.58-7.63 (m, 1H) 7.69-7.82 (m, 1H) 8.84-8.93 (m, 2H) 10.25-10.41 (m, 1H)

### Example 27: N-(2-carbamoyl-4-chloro-6-methyl-phenyl)-2-(3-chloro-2-pyridyl)-5-(methoxymethyl)pyrazole-3-carboxamide (P80)

### Step A: Preparation of methyl 2-(3-chloro-2-pyridyl)-5-(methoxymethyl)pyrazole-3-carboxylate

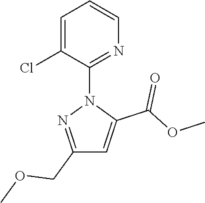

A 25 mL two-necked round bottom flask was charged with ethyl 5-(bromomethyl)-2-(3-chloro-2-pyridyl)pyrazole-3-carboxylate (300 mg, 0.871 mmol) in methanol (5.0 mL) was added sodium;methanolate (33%, 0.300 mL, 1.73 mmol) at 0° C. The reaction mixture was stirred for 2 h at room temperature. Reaction mixture was analysed by TLC (30% EtOAc/hexanes) and it showed no starting material. The reaction mixture was concentrated under reduced pressure to obtain crude product. The crude product was purified by combi flash column chromatography (combiflash, 0 to 100% Hexane/EtOAC). Pure fractions were combined and evaporated under reduced pressure to afford methyl 2-(3-chloro-2-pyridyl)-5-(methoxymethyl)pyrazole-3-carboxylate (200 mg, 0.710 mmol, yield: 81.6%) as an off-white solid.

Note: transesterification was happened due to methanol solution

1H NMR (DMSO-d6, 400 MHz): δ 8.56 (dd, J=1.2 Hz, 4.4 Hz, 1H), 8.26 (dd, J=1.6 Hz, 8.0 Hz, 1H), 7.70 (dd, J=4.8 Hz, 8.4 Hz, 1H), 7.10 (s, 1H), 4.47 (s, 2H), 3.71 (s, 3H), 3.30 (s, 3H); MS: m/z=282.0 [M+H⁺].

### Step B: Preparation of 2-(3-chloro-2-pyridyl)-5-(methoxyethyl)pyrazole-3-carboxylic acid

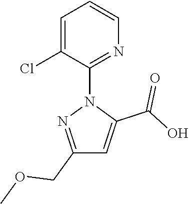 [SMILES: COCc1cc(C(=O)O)n(-c2ncccc2Cl)n1]

A 25 mL two-necked round bottom flask was charged with ethyl 2-(3-chloro-2-pyridyl)-5-ethoxy-pyrazole-3-carboxylate (280 mg, 0.994 mmol) in Methanol (10.0 mL) were added to a stirred solution of lithium hydroxide monohydrate (167 mg, 3.98 mmol) and water (5.0 mL) at 0° C. The reaction mixture was stirred at room temperature for 3 h. Reaction mixture was analysed by TLC (10% MeOH/DCM) and it showed no starting material. Most of the solvent was evaporated under reduced pressure and adjust pH 2.0 to 3.0 with 2N HCl at 0° C. Reaction mass was extracted with 10% MeOH/DCM (4×50 mL). The combined organic layers were washed with brine (10.0 mL), dried over Na2SO4 and evaporated under reduced pressure to afford 2-(3-chloro-2-pyridyl)-5-(methoxymethyl)pyrazole-3-carboxylic acid (210 mg, 0.828 mmol, yield: 83.3%) as an off-white solid.

1H NMR (DMSO-d6, 400 MHz): δ 13.77 (s, 1H), 8.55 (dd, J=2.4 Hz, 4.8 Hz, 1H), 8.23 (dd, J=1.6 Hz, 8.0 Hz, 1H), 7.66 (dd, J=4.8 Hz, 8.0 Hz, 1H), 7.01 (s, 1H), 4.45 (s, 2H), 3.29 (s, 3H);

### Step C: Preparation of 6-chloro-2-[2-(3-chloro-2-pyridyl)-5-(methoxymethyl)pyrazol-3-yl]-8-methyl-3,1-benzoxazin-4-one

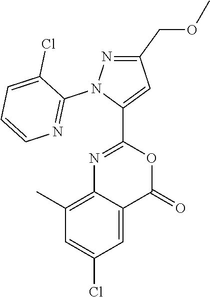 [SMILES: COCc1cc(-c2nc3c(C)cc(Cl)cc3c(=O)o2)n(-c2ncccc2Cl)n1]

A 25 mL two-necked round bottom flask was charged with 2-(3-chloro-2-pyridyl)-5-(methoxymethyl)pyrazole-3-carboxylic acid (210 mg, 0.785 mmol) in acetonitrile (5.0 mL) were added 2-amino-5-chloro-3-methyl-benzoic acid (175 mg, 0.941 mmol), pyridine (0.0633 mL, 0.785 mmol) and methane sulfonyl chloride (0.243 mL, 3.14 mmol) at 0° C. The reaction mixture was stirred for 2 h at room temperature. Reaction mixture was analysed by TLC (30% Ethyl acetate/Hexane) and it showed no starting material. Upon the reaction mixture was diluted with water (30.0 mL), extracted with EtOAc (2×50 mL). The combined organic layers were washed with brine (30.0 mL), dried over Na2SO4 and concentrated under reduced pressure to afford crude 6-chloro-2-[2-(3-chloro-2-pyridyl)-5-(methoxymethyl)pyrazol-3-yl]-8-methyl-3,1-benzoxazin-4-one (70%, 220 mg, 0.369 mmol, yield: 47%) as an off-white solid.

MS: m/z=416.9 [M−H+].

### Step D: Preparation of N-(2-carbamoyl-4-chloro-6-methyl-phenyl)-2-(3-chloro-2-pyridyl)-5-(methoxymethyl)pyrazole-3-carboxamide (P80)

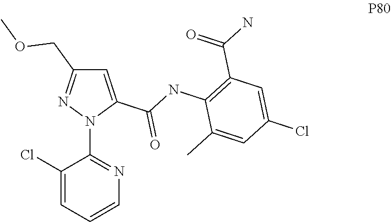 [SMILES: COCc1cc(C(=O)Nc2c(C)cc(Cl)cc2C(N)=O)n(-c2ncccc2Cl)n1]

A 25 mL two-necked round bottom flask was equipped with a nitrogen balloon and it was charged with 6-chloro-2-[2-(3-chloro-2-pyridyl)-5-(methoxymethyl)pyrazol-3-yl]-8-methyl-3,1-benzoxazin-4-one (150 mg, 0.360 mmol) in Acetonitrile (5.0 mL) and added 2M ammonia in ethanol (3.0 mL) at room temperature. The resulting reaction mixture was stirred at room temperature for 2 h. Reaction mixture was analysed by TLC (10% MeOH/DCM) and it showed no starting material. Upon the reaction mixture was concentrated under reduced pressure to obtain crude mixture. The crude mixture was purified by combi flash column chromatography (combiflash, 0 to 100% Hexane/EtOAC). Pure fractions were combined and evaporated under reduced pressure to afford N-(2-carbamoyl-4-chloro-6-methyl-phenyl)-2-(3-chloro-2-pyridyl)-5-(methoxymethyl)pyrazole-3-carboxamide (48.0 mg, 0.111 mmol, yield: 30.7%) as an off-white solid.

1H NMR (DMSO-d6, 400 MHz): δ 10.22 (s, 1H), 8.48 (dd, J=1.6 Hz, 4.8 Hz, 1H), 8.14 (dd, J=1.6 Hz, 8.0 Hz, 1H), 7.72 (s, 1H), 7.58 (dd, J=4.8 Hz, 8.0 Hz, 1H), 7.51 (s, 1H), 7.46 (d, J=2.0 Hz, 1H), 7.40 (d, J=2.4 Hz, 1H), 7.21 (s, 1H), 4.49 (s, 2H), 3.34 (s, 3H), 2.14 (s, 3H); MS: m/z=433.9 [M+H+].

Melting point: 225-230° C.

### Example 28: N-(2-carbamoyl-4-chloro-6-methyl-phenyl)-2-(3-chloro-2-pyridyl)-5-[[5-(3-pyridyl)tetrazol-2-yl]methyl]pyrazole-3-carboxamide (P81)

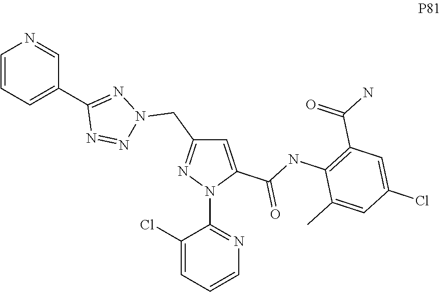 [SMILES: Cc1cc(Cl)cc(C(N)=O)c1NC(=O)c1cc(Cn2nnc(-c3cccnc3)n2)nn1-c1ncccc1Cl]

To a solution of N-(2-carbamoyl-4-chloro-6-methyl-phenyl)-5-(chloromethyl)-2-(3-chloro-2-pyridyl)pyrazole-3-carboxamide (P7) (15.3 mg) and 3-(2H-tetrazol-5-yl)pyridine (7.7 mg) in acetone (0.9 mL) were added DIPEA (12 μL) and potassium iodide (0.12 mg). The reaction mixture was stirred at 60° C. for 4h. The reaction mixture was evaporated and the purification was made on reverse phase preparative HPLC to give N-(2-carbamoyl-4-chloro-6-methyl-phenyl)-2-(3-chloro-2-pyridyl)-5-[[5-(3-pyridyl)tetrazol-2-yl]methyl]pyrazole-3-carboxamide (5.1 mg, yield:27%) LC-MS (Method 2): Rt 1.19, m/z=549 (M+H⁺).

Compounds P83-P105 were prepared via the conditions described for Compound P81 in using N-(2-carbamoyl-4-chloro-6-methyl-phenyl)-5-(chloromethyl)-2-(3-chloro-2-pyridyl)pyrazole-3-carboxamide (P7) and the corresponding substituted tetrazoles.

### Example 29: N-(2-carbamoyl-4-chloro-6-methyl-phenyl)-2-(3-chloro-2-pyridyl)-5-(1-fluorocyclopropyl)pyrazole-3-carboxamide (P69)

### Step A: Preparation of ethyl 4-(1-fluorocyclopropyl)-2,4-dioxo-butanoate

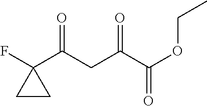

A 25 mL two-necked round bottom flask was equipped with nitrogen balloon was charged with 1-(1-fluorocyclopropyl)ethanone (800 mg, 7.83 mmol) in ethanol (40.00 mL), were added sodium ethanolate (21.0%, 4.00 mL, 15.7 mmol),diethyl oxalate (126 mL, 8.62 mmol) at 0° C. The reaction mixture was stirred at room temperature for 6 h. Reaction mixture was analysed by TLC (20% Ethyl acetate/Hexane), it showed no starting material. The reaction mixture was concentrated under reduced pressure and crude compound was dissolved in ethyl acetate (2×50 mL) and washed with water (10 ml) and the combined organic layers were washed with brine (20 mL), dried over Na2SO4 and concentrated under reduced pressure to afford crude product ethyl 4-(1-fluorocyclopropyl)-2,4-dioxo-butanoate (580 mg) gummy solid.

MS: m/z=201.1 [M−H+].

Note: Crude product was used for next step

### Step B: Preparation of N-(2-carbamoyl-4-chloro-6-methyl-phenyl)-2-(3-chloro-2-pyridyl)-5-(1-fluorocyclopropyl)pyrazole-3-carboxamide (P69)

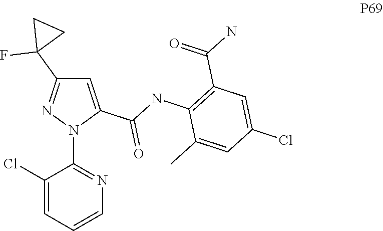 [SMILES: Cc1cc(Cl)cc(C(N)=O)c1NC(=O)c1cc(C2(F)CC2)nn1-c1ncccc1Cl]

The same synthetic route as for Compound P75 (step A to E) was followed to synthesize compounds P69 starting from ethyl 4-(1-fluorocyclopropyl)-2,4-dioxo-butanoate.

TABLE P

| Entry | IUPAC name | STRUCTURE | RT (min) | [M + H] (measured) | Method | MP ° C. | NMR |
|---|---|---|---|---|---|---|---|
| P1 | N-(2-carbamoyl-4-chloro-6- methyl-phenyl)-2-(3-chloro-2- pyridyl)-5-[(5- cyclopropyltetrazol-1- yl)methyl]pyrazole-3- carboxamide | 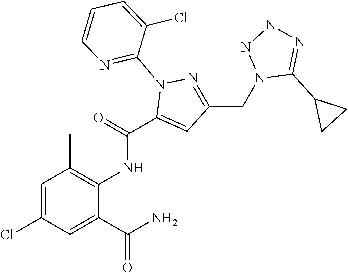 [SMILES: Cc1cc(Cl)cc(C(N)=O)c1NC(=O)c1cc(CN2NNN=C2C2CC2)nn1-c1ncccc1Cl] | 0.82 | 512 | 1 | 167-170 | 1H NMR (600 MHz, DMSO- d6) δ ppm 10.25 (s, 1 H) 8.48 (dd, J = 4.5, 1.1 Hz, 1 H) 8.15 (d, J = 7.3 Hz, 1 H) 7.73 (br s, 1 H) 7.59 (dd, J = 8.0, 4.7 Hz, 1 H) 7.47 (br d, J = 15.8 Hz, 2 H) 7.39 (d, J = 1.6 Hz, 1 H) 7.19 (s, 1 H) 5.91 (s, 2 H) 2.28-2.33 (m, 1 H) 2.13 (s, 3 H) 1.17 (dd, J = 8.2, 2.7 Hz, 2 H) 1.01 (dd, J = 4.5, 2.5 Hz, 2 H) |
| P2 | N-(2-carbamoyl-4-chloro-6- methyl-phenyl)-2-(3-chloro-2- pyridyl)-5-[(5- cyclopropyltetrazol-2- yl)methyl]pyrazole-3- carboxamide | 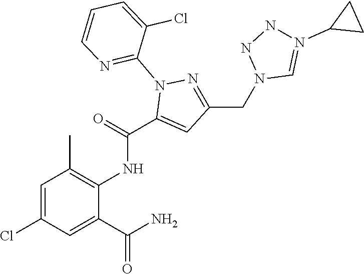 | 0.89 | 512 | 1 | | 1H NMR (600 MHz, DMSO- d6) δ ppm 10.25 (s, 1 H) 8.49 (dd, J = 4.7, 1.3 Hz, 1 H) 8.15 (d, J = 8.1, 1.2 Hz, 1 H) 7.72 (s, 1 H) 7.59 (dd, J = 8.2, 4.7 Hz, 1 H) 7.43-7.51 (m, 2 H) 7.39 (d, J = 2.0 Hz, 1 H) 7.22 (s, 1 H) 5.99 (s, 2 H) 2.19- 2.29 (m, 1 H) 2.13 (s, 3 H) 1.10 (dd, J = 8.4, 2.5 Hz, 2 H) 0.95 (dd, J = 4.8, 2.5 Hz, 2 H) |
| P3 | N-(2-carbamoyl-4-chloro-6- methyl-phenyl)-5-[(4-chloroindazol- 1-yl)methyl]-2-(3-chloro-2-pyridyl) pyrazole-3-carboxamide | 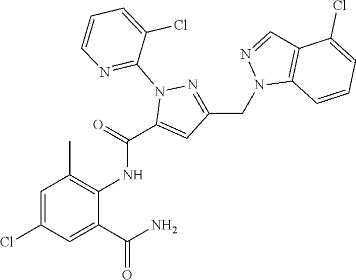 [SMILES: Cc1cc(Cl)cc(C(N)=O)c1NC(=O)c1cc(Cn2ncc3c(Cl)cccc32)nn1-c1ncccc1Cl] | 1.02 | 554 | 1 | | 1H NMR (600 MHz, CHLOROFORM-d) δ ppm 9.72 (s, 1 H) 8.49 (dd, J = 4.7, 1.5 Hz, 1 H) 8.17 (s, 1 H) 7.87 (dd, J = 8.0, 1.5 Hz, 1 H) 7.48 (d, J = 8.5 Hz, 1 H) 7.39 (dd, J = 8.1, 4.7 Hz, 1 H) 7.29- 7.32 (m, 2 H) 7.17 (d, J = 7.2 Hz, 1 H) 6.84 (s, 1 H) 6.04 (br s, 1 H) 5.75 (s, 2 H) 5.68 (br s, 1 H) 2.17 (s, 3 H) |
| P4 | N-(2-carbamoyl-4-chloro-6-methyl- phenyl)-5-[(4-chloroindazol-2-yl) methyl]-2-(3-chloro-2-pyridyl) pyrazole-3-carboxamide | 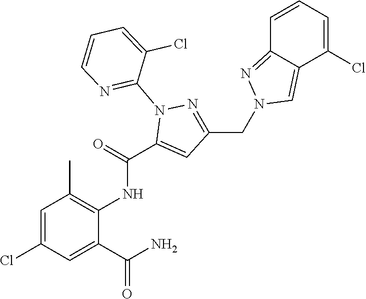 [SMILES: Cc1cc(Cl)cc(C(N)=O)c1NC(=O)c1cc(Cn2cc3c(Cl)cccc3n2)nn1-c1ncccc1Cl] | 0.99 | 554 | 1 | | 1H NMR (400 MHz, CHLOROFORM-d) δ ppm 9.82 (s, 1 H), 8.49 (dd, J = 4.7, 1.5 Hz, 1 H), 8.15-8.17 (m, 1 H), 7.87 (dd, J = 8.0, 1.8 Hz, 1 H), 7.65 (d, J = 8.7 Hz, 1 H), 7.39 (dd, J = 8.0, 4.7 Hz, 1 H), 7.29-7.34 (m, 2 H), 7.22 (dd, J = 8.5, 7.1 Hz, 1 H), 7.08 (d, J = 6.9 Hz, 1 H), 7.03 (s, 1 H), 5.95-6.08 (m, 1 H), 5.76 (s, 2 H), 5.60-5.72 (m, 1 H), 2.19 (s, 3 H) |
| P5 | N-(2-carbamoyl-4-chloro-6-methyl- phenyl)-5-[(4-chlorophenyl) methoxymethyl]-2-(3-chloro-2- pyridyl)pyrazole-3-carboxamide | 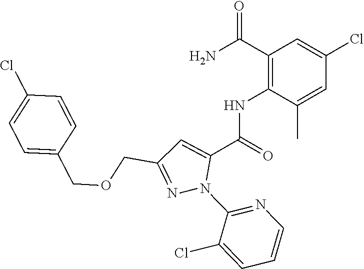 [SMILES: Cc1cc(Cl)cc(C(N)=O)c1NC(=O)c1cc(COCc2ccc(Cl)cc2)nn1-c1ncccc1Cl] | 1.04 | 544 | 1 | 174-176 | 1H NMR (400 MHz, acetone- d6) δ ppm 10.35 (br s, 1 H), 8.49 (dd, J = 4.7, 1.5 Hz, 1 H), 8.06 (dd, J = 8.0, 1.5 Hz, 1 H), 7.51-7.61 (m, 3 H), 7.43- 7.47 (m, 2 H), 7.38-7.42 (m, 3 H), 7.20 (s, 1 H), 6.96-7.10 (m, 1 H), 4.69 (s, 2 H), 4.63- 4.67 (m, 2 H), 2.23 (s, 3 H) |
| P6 | N-(2-carbamoyl-4-chloro-6-methyl- phenyl)-2-(3-chloro-2-pyridyl)-5- [[5-[4-(trifluoromethyl)phenyl] tetrazol-2-yl]methyl]pyrazole-3- carboxamide Disclosed in: US2013/0324560 | 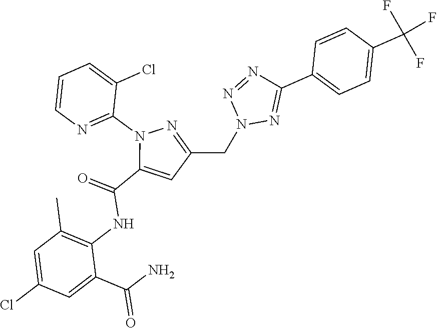 [SMILES: Cc1cc(Cl)cc(C(N)=O)c1NC(=O)c1cc(Cn2nnc(-c3ccc(C(F)(F)F)cc3)n2)nn1-c1ncccc1Cl] | 1.08 | 616 | 1 | | 1H NMR (600 MHz, DMSO- d6) δ ppm 10.26 (s, 1 H) 8.48 (dd, J = 4.6, 1.4 Hz, 1 H) 8.32 (m, J = 8.2 Hz, 2 H) 8.15 (dd, J = 8.1, 1.4 Hz, 1 H) 7.97 (m, J = 8.2 Hz, 2 H) 7.72 (s, 1 H) 7.59 (dd, J = 8.0, 4.7 Hz, 1 H) 7.46-7.47 (m, 1 H) 7.45 (br d, J = 2.4 Hz, 1 H), 7.38 (d, J = 2.2 Hz, 1 H) 7.31 (s, 1 H) 6.23 (s, 2 H) 2.13 (s, 3 H) |
| P7 | N-(2-carbamoyl-4-chloro-6-methyl- phenyl)-5-(chloromethyl)-2-(3- chloro-2-pyridyl)pyrazole-3-carboxamide | 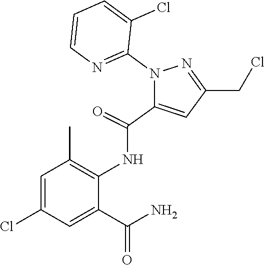 [SMILES: Cc1cc(Cl)cc(C(N)=O)c1NC(=O)c1cc(CCl)nn1-c1ncccc1Cl] | 0.87 | 439 | 1 | | 1H NMR (400 MHz, DMSO- d6) δ ppm 10.27 (s, 1 H) 8.49 (dd, J = 4.72, 1.45 Hz, 1 H) 8.14 (dd, J = 7.99, 1.45 Hz, 1 H) 7.73 (s, 1 H) 7.58 (dd, J = 7.99, 4.72 Hz, 1 H) 7.50 (s, 1 H) 7.45-7.48 (m, 1 H) 7.40 (d, J = 2.18 Hz, 1 H) 7.31 (s, 1 H) 4.87 (s, 2 H) 2.15 (s, 3 H) |
| P8 | N-(2-carbamoyl-4-chloro-6-methyl- phenyl)-2-(3-chloro-2-pyridyl)-5- (2,2,3,3,3-pentafluoropropoxy)pyrazole- 3-carboxamide | 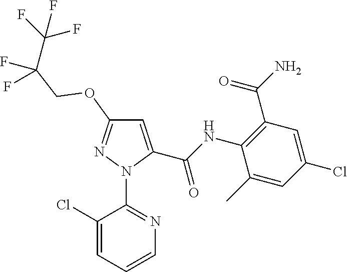 [SMILES: Cc1cc(Cl)cc(C(N)=O)c1NC(=O)c1cc(OCC(F)(F)C(F)(F)F)nn1-c1ncccc1Cl] | 1.04 | 538 | 1 | 203-205 | 1H NMR (400 MHz, DMSO- d6) δ ppm 10.23 (s, 1 H) 8.47 (dd, J = 4.72, 1.45 Hz, 1 H) 8.12 (dd, J = 8.17, 1.63 Hz, 1 H) 7.74 (br s, 1 H) 7.56 (dd, J = 7.99, 4.72 Hz, 1 H) 7.51 (s, 1 H) 7.45-7.48 (m, 1 H) 7.41 (d, J = 2.54 Hz, 1 H) 6.84 (s, 1 H) 5.00 (t, J = 13.26 Hz, 2 H) 2.15 (s, 3 H) |
| P9 | N-(2-carbamoyl-4-chloro-6- methyl-phenyl)-2-(3-chloro-2-pyridyl)- 5-methoxy-pyrazole-3-carboxamide | 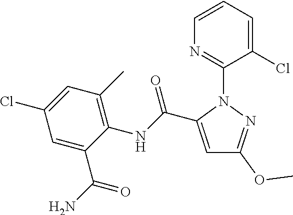 [SMILES: COc1cc(C(=O)Nc2c(C)cc(Cl)cc2C(N)=O)n(-c2ncccc2Cl)n1] | 0.91 | 421 | 1 | 235-237 | 1H NMR (400 MHz, ACETONITRILE-d3) δ ppm 9.60 (br s, 1 H) 8.44 (dd, J = 4.7, 1.5 Hz, 1 H) 7.95 (dd, J = 8.0, 1.5 Hz, 1 H) 7.33- 7.54 (m, 3 H) 6.69 (br s, 1 H) 6.49 (s, 1 H) 6.12-6.30 (m, 1 H) 3.93 (s, 3 H) 2.18 (s, 3 H) |
| P10 | N-(2-carbamoyl-4-iodo-6-methyl- phenyl)-2-(3-chloro-2-pyridyl)-5- methoxy-pyrazole-3-carboxamide | 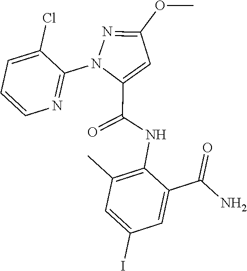 [SMILES: COc1cc(C(=O)Nc2c(C)cc(I)cc2C(N)=O)n(-c2ncccc2Cl)n1] | 0.88 | 512 | 1 | 214-220 | 1H NMR (400 MHz, CHLOROFORM-d) δ ppm 9.83 (s, 1 H) 8.42 (dd, J = 4.72, 1.82 Hz, 1 H) 7.81 (dd, J = 7.99, 1.82 Hz, 1 H) 7.59 (dd, J = 11.81, 1.63 Hz, 2 H) 7.28-7.33 (m, 1 H) 6.50 (s, 1 H) 6.30 (br s, 1 H) 5.91 (br s, 1 H) 4.00 (s, 3 H) 2.12- 2.17 (m, 3 H) |
| P11 | N-(2-carbamoyl-4-iodo-6-methyl- phenyl)-2-(3-chloro-2-pyridyl)-5-(2,2,2- trifluoroethoxy)pyrazole-3-carboxamide | 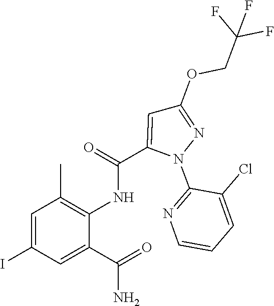 [SMILES: Cc1cc(I)cc(C(N)=O)c1NC(=O)c1cc(OCC(F)(F)F)nn1-c1ncccc1Cl] | | | | 228-230 | |
| P12 | N-(2-carbamoyl-4-cyano-6-methyl- phenyl)-5-[(4-chlorophenyl)methoxy- methyl]-2-(3-chloro-2-pyridyl)pyrazole- 3-carboxamide | 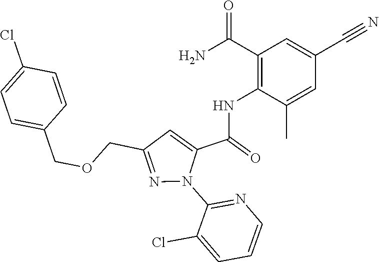 [SMILES: Cc1cc(C#N)cc(C(N)=O)c1NC(=O)c1cc(COCc2ccc(Cl)cc2)nn1-c1ncccc1Cl] | 1.00 | 535 | 1 | 109-112 | 1H NMR (400 MHz, acetone- d6) δ ppm 10.85 (s, 1 H), 8.50 (dd, J = 4.7, 1.5 Hz, 1 H), 8.08 (dd, J = 8.0, 1.5 Hz, 1 H), 7.97 (s, 1 H), 7.70-7.82 (m, 2 H), 7.57 (dd, J = 8.0, 4.7 Hz, 1 H), 7.41-7.48 (m, 2 H), 7.38-7.41 (m, 2 H), 7.22 (s, 2 H), 4.69-4.71 (m, 2 H), 4.64-4.66 (m, 2 H), 2.28 (s, 3 H) |
| P13 | N-(2-carbamoyl-4-cyano-6-methyl- phenyl)-2-(3-chloro-2-pyridyl)-5- methoxy-pyrazole-3-carboxamide | 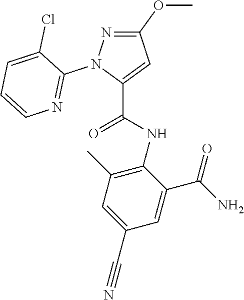 [SMILES: COc1cc(C(=O)Nc2c(C)cc(C#N)cc2C(N)=O)n(-c2ncccc2Cl)n1] | 0.77 | 411 | 1 | 125-140 | 1H NMR (400 MHz, CHLOROFORM-d) δ ppm 10.32 (s, 1 H) 8.45 (dd, J = 4.72, 1.45 Hz, 1 H) 7.83 (dd, J = 7.99, 1.82 Hz, 1 H) 7.62 (d, J = 1.45 Hz, 1 H) 7.56-7.59 (m, 1 H) 7.33 (dd, J = 7.99, 4.72 Hz, 1 H) 6.39- 6.50 (m, 2 H) 5.90 (br s, 1 H) 4.00 (s, 3 H) 2.25 (s, 3 H) |
| P14 | N-(2-carbamoyl-4-chloro-6- methyl-phenyl)-5-[(5-chlorobenzo- triazol-1-yl)methyl]-2-(3-chloro-2- pyridyl)pyrazole-3-carboxamide | 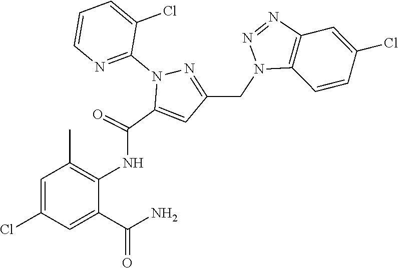 [SMILES: Cc1cc(Cl)cc(C(N)=O)c1NC(=O)c1cc(Cn2nnc3cc(Cl)ccc32)nn1-c1ncccc1Cl] | 0.95 | 557 | 1 | | 1H NMR (600 MHz, DMSO- d6) δ ppm 10.16-10.24 (m, 1 H) 8.47 (ddd, J = 4.7, 3.3, 1.5 Hz, 1 H) 8.25 (d, J = 1.5 Hz, 1 H) 8.09-8.19 (m, 1 H) 7.97 (d, J = 8.8 Hz, 1 H) 7.70 (br s, 1 H) 7.65 (dd, J = 8.9, 1.8 Hz, 1 H) 7.58 (ddd, J = 7.9, 4.8, 2.9 Hz, 1 H) 7.44 (br s, 2 H), 7.37 (d, J = 2.3 Hz, 1 H) 7.11-7.21 (m, 1 H) 6.16 (s, 2 H) 2.10 (d, J = 2.2 Hz, 3 H) |
| P15 | N-(2-carbamoyl-4-chloro-6-methyl- phenyl)-2-(3-chloro-2-pyridyl)-5-[[5-[4- (trifluoromethyl)phenyl]tetrazol-1-yl] methyl]pyrazole-3-carboxamide | 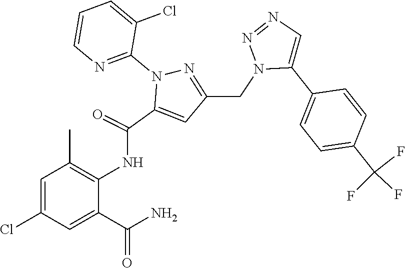 [SMILES: Cc1cc(Cl)cc(C(N)=O)c1NC(=O)c1cc(Cn2nncc2-c2ccc(C(F)(F)F)cc2)nn1-c1ncccc1Cl] | 0.99 | 616 | 1 | | 1H NMR (400 MHz, CHLOROFORM-d) δ ppm 9.93-10.06 (m, 1 H), 8.45- 8.53 (m, 1 H), 8.00-8.10 (m, 2 H), 7.89 (dd, J = 8.0, 1.5 Hz, 1 H), 7.82 (d, J = 8.4 Hz, 2 H), 7.40-7.45 (m, 1 H), 7.31- 7.37 (m, 2 H), 7.11 (s, 1 H), 5.97-6.09 (m, 1 H), 5.78 (s, 2 H), 5.64-5.75 (m, 1 H), 2.20 (s, 3 H) |
| P16 | N-(2-carbamoyl-4-chloro-6-methyl- phenyl)-5-[(6-chlorobenzotriazol-1-yl) methyl]-2-(3-chloro-2-pyridyl)pyrazole- 3-carboxamide | 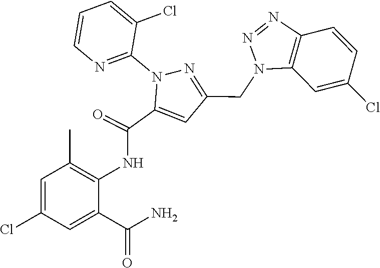 [SMILES: Cc1cc(Cl)cc(C(N)=O)c1NC(=O)c1cc(Cn2nnc3ccc(Cl)cc32)nn1-c1ncccc1Cl] | 0.95 | 557 | 1 | | 1H NMR (600 MHz, DMSO- d6) δ 10.16-10.14 (m, 1 H) 8.47 (ddd, J = 4.7, 3.3, 1.5 Hz, 1 H) 8.09-8.19 (m, 3 H) 7.70 (br s, 1 H) 7.58 (ddd, J = 7.9, 4.8, 2.9 Hz, 1 H) 7.48 (dd, J = 8.8, 1.9 Hz, 1 H) 7.44 (br s, 2 H) 7.37 (d, J = 2.3 Hz, 1 H) 7.11-7.21 (m, 1 H) 6.14 (s, 2 H) 2.10 (d, J = 2.2 Hz, 3 H) |
| P17 | N-(2-carbamoyl-4-chloro-6-methyl- phenyl)-5-[(5-chlorobenzotriazol-2-yl) methyl]-2-(3-chloro-2-pyridyl)pyrazole- 3-carboxamide | 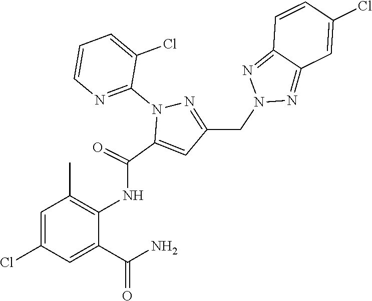 [SMILES: Cc1cc(Cl)cc(C(N)=O)c1NC(=O)c1cc(Cn2nc3ccc(Cl)cc3n2)nn1-c1ncccc1Cl] | 1.00 | 555 | 1 | | 1H NMR (600 MHz, DMSO- d6) δ 10.21 (s, 1 H) 8.48 (dd, J = 4.7, 1.6 Hz, 1 H) 8.16 (dd, J = 1.9, 0.6 Hz, 1 H) 8.15 (dd, J = 8.0, 1.4 Hz, 1 H) 8.05 (dd, J = 9.1, 0.6 Hz, 1 H) 7.70 (s, 1 H) 7.58 (dd, J = 8.1, 4.7 Hz, 1 H) 7.50 (dd, J = 8.9, 1.9 Hz, 1 H) 7.44 (br d, J = 2.3 Hz, 2 H) 7.37 (d, J = 2.2 Hz, 1 H) 7.23 (s, 1 H) 6.14 (s, 2 H) 2.11 (s, 3 H) |
| P18 | N-(2-carbamoyl-4-chloro-6-methyl- phenyl)-2-(3-chloro-2-pyridyl)-5-[[5- (trifluoromethyl)tetrazol-2-yl]methyl] pyrazole-3-carboxamide Disclosed in: US2010/256195 | 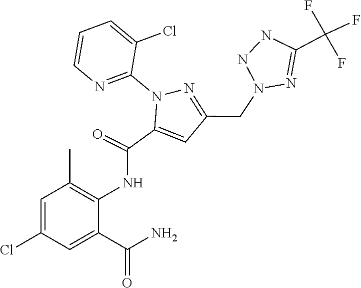 [SMILES: Cc1cc(Cl)cc(C(N)=O)c1NC(=O)c1cc(CN2N=C(C(F)(F)F)NN2)nn1-c1ncccc1Cl] | 0.96 | 540 | 1 | | 1H NMR (400 MHz, CHLOROFORM-d) δ ppm 9.99 (s, 1 H), 8.48 (dd, J = 4.7, 1.5 Hz, 1 H), 7.88 (dd, J = 8.0, 1.5 Hz, 1 H), 7.40 (dd, J = 8.0, 4.7 Hz, 1 H), 7.31-7.33 (m, 2 H), 7.14 (s, 1 H), 6.04 (s, 3 H), 5.66 (br s, 1 H), 2.20 (s, 3 H) |
| P19 | N-(2-carbamoyl-4-chloro-6- methyl-phenyl)-2-(3-chloro-2-pyridyl)- 5-[[4-(trifluoromethyl)triazol-2-yl] methyl]pyrazole-3-carboxamide Disclosed in: US2011/275676 | 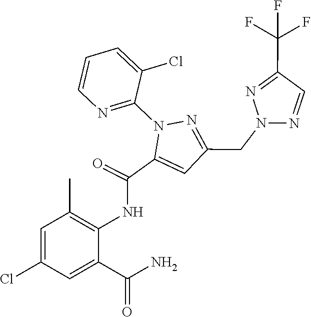 [SMILES: Cc1cc(Cl)cc(C(N)=O)c1NC(=O)c1cc(Cn2ncc(C(F)(F)F)n2)nn1-c1ncccc1Cl] | 0.97 | 539 | 1 | | 1H NMR (400 MHz, CHLOROFORM-d) δ ppm 9.87 (s, 1 H) 8.47 (dd, J = 4.72, 1.45 Hz, 1 H) 7.89- 7.93 (m, 1 H) 7.86 (dd, J = 8.17, 1.63 Hz, 1 H) 7.36- 7.41 (m, 1 H) 7.31-7.35 (m, 2 H) 7.03 (s, 1 H) 5.93-6.14 (m, 1 H) 5.82 (s, 2 H) 5.55- 5.74 (m, 1 H) 2.21 (s, 3 H) |
| P20 | N-(2-carbamoyl-4-chloro-6-methyl- phenyl)-2-(3-chloro-2-pyridyl)-5-[[4- (trifluoromethyl)triazol-1-yl]methyl] pyrazole-3-carboxamide Disclosed in: US2011/275676 | 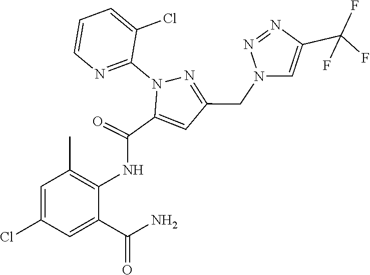 [SMILES: Cc1cc(Cl)cc(C(N)=O)c1NC(=O)c1cc(Cn2cc(C(F)(F)F)nn2)nn1-c1ncccc1Cl] | 0.91 | 539 | 1 | 213-217 | 1H NMR (400 MHz, CHLOROFORM-d) δ ppm 10.03 (s, 1 H) 8.50 (dd, J = 4.72, 1.45 Hz, 1 H) 8.02- 8.06 (m, 1 H) 7.89 (dd, J = 8.17, 1.64 Hz, 1 H), 7.42 (dd, J = 7.99, 4.72 Hz, 1 H) 7.28-7.33 (m, 2 H) 7.09 (s, 1 H), 6.12 (br s, 1 H) 5.68-5.87 (m, 3 H) 2.19 (s, 3 H) |
| P21 | N-(2-carbamoyl-4-chloro-6-methyl- phenyl)-2-(3-chloro-2-pyridyl)-5- (difluoromethoxy)pyrazole-3- carboxamide Disclosed in: WO2014/128188 | 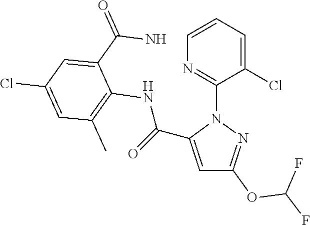 [SMILES: Cc1cc(Cl)cc(C(N)=O)c1NC(=O)c1cc(OC(F)F)nn1-c1ncccc1Cl] | 0.96 | 456 | 1 | 195-200 | 1H NMR (400 MHz, DMSO- d6) δ ppm 10.30 (s, 1 H), 8.49 (dd, J = 4.7, 1.5 Hz, 1 H) 8.15 (dd, J = 8.0, 1.5 Hz, 1 H) 7.74 (s, 1 H) 7.59 (dd, J = 8.2, 4.5 Hz, 1 H) 7.49 (br s, 1 H) 7.47 (d, J = 2.2 Hz, 1 H) 7.36- 7.44 (m, 2 H) 7.03 (s, 1 H) 2.15 (s, 3 H) |
| P22 | N-(2-carbamoyl-4-chloro-6-methyl- phenyl)-2-(3-chloro-2-pyridyl)-5- (difluoromethyl)pyrazole-3-carboxamide Disclosed in: WO2014/128188 | 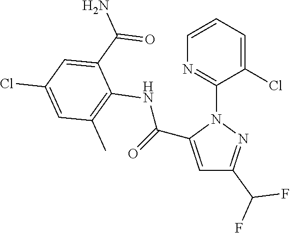 [SMILES: Cc1cc(Cl)cc(C(N)=O)c1NC(=O)c1cc(C(F)F)nn1-c1ncccc1Cl] | 0.94 | 440 | 1 | | 1H NMR (DMSO-d6) δ ppm 10.34 (s, 1 H) 8.51 (dd, J = 4.7, 1.8 Hz, 1 H) 8.18 (dd, J = 8.2, 1.6 Hz, 1 H) 7.74 (s, 1 H) 7.63 (dd, J = 8.0, 4.7 Hz, 1 H) 7.52 (s, 1 H) 7.48-7.50 (m, 1 H) 7.47 (d, J = 1.8 Hz, 1 H) 7.40 (d, J = 2.5 Hz, 1 H) 7.04-7.37 (m, 1 H) 2.15 (s, 3 H) |
| P23 | N-(2-carbamoyl-4-chloro-6-methyl- phenyl)-2-(3-chloro-2-pyridyl)-5-(2,2,2- trifluoroethoxy)pyrazole-3-carboxamide Disclosed in: WO2020/117493 | 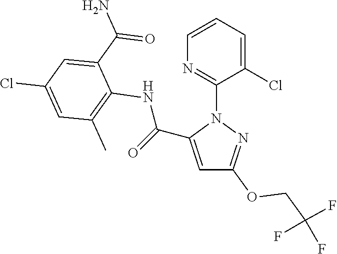 [SMILES: Cc1cc(Cl)cc(C(N)=O)c1NC(=O)c1cc(OCC(F)(F)F)nn1-c1ncccc1Cl] | 1.01 | 488 | 1 | | 1H NMR (400 MHz, DMSO- d6) δ ppm 10.23 (s, 1 H) 8.47 (dd, J = 4.4, 1.5 Hz, 1 H) 8.12 (dd, J = 8.0, 1.5 Hz, 1 H) 7.74 (br s, 1 H) 7.56 (dd, J = 8.0, 4.7 Hz, 1 H) 7.51 (br s, 1 H) 7.47 (d, J = 2.2 Hz, 1 H) 7.41 (d, J = 2.2 Hz, 1 H) 6.84 (s, 1 H) 4.91 (q, J = 9.0 Hz, 2 H) 2.15 (s, 3 H) |
| P24 | 5-bromo-N-(2-carbamoyl-4- chloro-6-methyl-phenyl)-2-(3- chloro-2-pyridyl)pyrazole-3- carboxamide Disclosed in: CN107033135 | 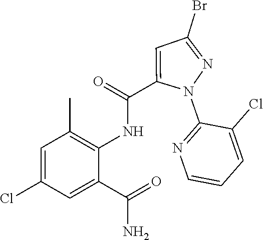 [SMILES: Cc1cc(Cl)cc(C(N)=O)c1NC(=O)c1cc(Br)nn1-c1ncccc1Cl] | | | | 246-248 | |
| P25 | N-(2-carbamoyl-4-chloro-6- methyl-phenyl)-2-(3-chloro-2- pyridyl)-5-(trifluoromethyl)pyrazole-3- carboxamide Disclosed in: WO2014/128188 | 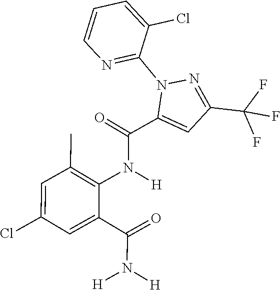 [SMILES: Cc1cc(Cl)cc(C(N)=O)c1NC(=O)c1cc(C(F)(F)F)nn1-c1ncccc1Cl] | 1.00 | 458 | 1 | 230-233 | 1H NMR (2-DMSO) δ: 10.41 (s, 1H), 8.54 (dd, J = 4.7, 1.5 Hz, 1H), 8.22 (dd, J = 8.4, 1.5 Hz, 1H), 7.77 (br s, 1H), 7.72 (s, 1H), 7.66 (dd, J = 8.0, 4.7 Hz, 1H), 7.48 (br d, J = 2.5 Hz, 2H), 7.41 (d, J = 2.2 Hz, 1H), 2.16 (s, 3H) |
| P26 | N-(4-bromo-2-carbamoyl-6-methyl- phenyl)-2-(3-chloro-2-pyridyl)-5- (trifluoromethyl)pyrazole-3-carboxamide Disclosed in: WO2003/015518 | 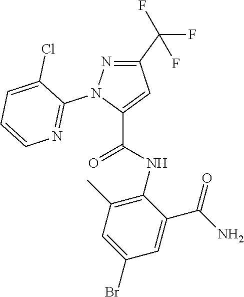 [SMILES: Cc1cc(Br)cc(C(N)=O)c1NC(=O)c1cc(C(F)(F)F)nn1-c1ncccc1Cl] | 0.97 | 503 | 1 | 242-242 | 1H NMR (400 MHz, DMSO- d6) δ ppm 10.40 (s, 1 H) 8.54 (dd, J = 4.72, 1.45 Hz, 1 H) 8.22 (dd, J = 7.99, 1.45 Hz, 1 H) 7.77 (br s, 1 H), 7.72 (s, 1 H) 7.66 (dd, J = 8.36, 4.72 Hz, 1 H) 7.61 (d, J = 1.82 Hz, 1 H) 7.53 (d, J = 2.18 Hz, 1 H) 7.48 (br s, 1 H) 2.15 (s, 3 H) |
| P27 | N-(2-carbamoyl-4-iodo-6-methyl- phenyl)-2-(3-chloro-2-pyridyl)-5- (trifluoromethyl)pyrazole-3-carboxamide Disclosed in: WO2003/015518 | 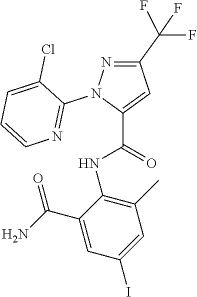 [SMILES: Cc1cc(I)cc(C(N)=O)c1NC(=O)c1cc(C(F)(F)F)nn1-c1ncccc1Cl] | 0.99 | 550 | 1 | | 1H NMR (400 MHz, MeOD) δ ppm 8.53 (dd, J = 4.72, 1.45 Hz, 1 H) 8.13 (dd, J = 7.99, 1.45 Hz, 1 H) 7.81 (s, 2 H) 7.63 (dd, J = 7.99, 4.72 Hz, 1 H) 7.56 (s, 1 H) 2.25 (s, 3 H) |
| P28 | N-(2-carbamoyl-4-cyano-6-methyl- phenyl)-2-(3-chloro-2-pyridyl)-5- (difluoromethyl)pyrazole-3-carboxamide Disclosed in: WO2014/128188 | 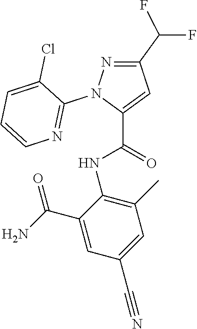 [SMILES: Cc1cc(C#N)cc(C(N)=O)c1NC(=O)c1cc(C(F)F)nn1-c1ncccc1Cl] | 0.82 | 431 | 1 | | 1H NMR (400 MHz, MeOD) δ ppm 8.47-8.55 (m, 1 H) 8.11 (d, J = 7.99 Hz, 1 H) 7.82- 7.86 (m, 1 H) 7.79 (s, 1 H) 7.56-7.64 (m, 1 H) 7.49 (s, 1 H) 6.98 (t, 1 H) 2.34 (s, 3 H) |
| P29 | N-(2-carbamoyl-4-cyano-6-methyl- phenyl)-5-chloro-2-(3-chloro-2-pyridyl) pyrazole-3-carboxamide Disclosed in: WO2004/67528 | 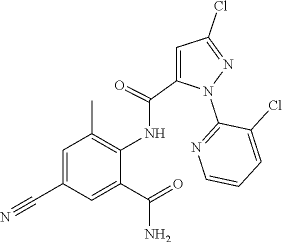 [SMILES: Cc1cc(C#N)cc(C(N)=O)c1NC(=O)c1cc(Cl)nn1-c1ncccc1Cl] | | | | | |
| P30 | 5-bromo-N-(2-carbamoyl-4-cyano-6- methyl-phenyl)-2-(3-chloro-2-pyridyl) pyrazole-3-carboxamide Disclosed in: CN107033135 | 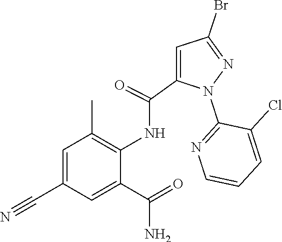 [SMILES: Cc1cc(C#N)cc(C(N)=O)c1NC(=O)c1cc(Br)nn1-c1ncccc1Cl] | | | | | |
| P31 | N-(2-carbamoyl-4-cyano-6- methyl-phenyl)-2-(3-chloro-2- pyridyl)-5- (trifluoromethyl)pyrazole-3- carboxamide Disclosed in: WO2004/67528 | 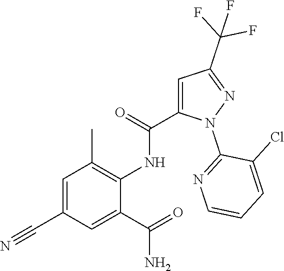 [SMILES: Cc1cc(C#N)cc(C(N)=O)c1NC(=O)c1cc(C(F)(F)F)nn1-c1ncccc1Cl] | | | | | |
| P32 | N-(2-carbamoyl-4-cyano-6-methyl- phenyl)-2-(3-chloro-2-pyridyl)-5-(2,2,2- trifluoroethoxy)pyrazole-3-carboxamide Disclosed in: WO2014/128188 | 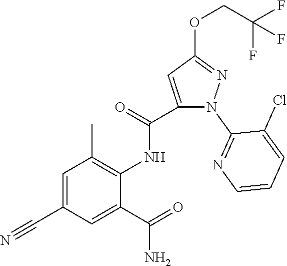 [SMILES: Cc1cc(C#N)cc(C(N)=O)c1NC(=O)c1cc(OCC(F)(F)F)nn1-c1ncccc1Cl] | | | | | |
| P33 | 5-bromo-N-(2-carbamoyl-4,6-dichloro- phenyl)-2-(3,5-dichloro-2-pyridyl) pyrazole-3-carboxamide Disclosed in: WO2013/044791 | 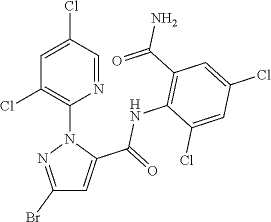 [SMILES: NC(=O)c1cc(Cl)cc(Cl)c1NC(=O)c1cc(Br)nn1-c1ncc(Cl)cc1Cl] | 0.98 | 523 | 1 | | 1H NMR (400 MHz, DMSO- d6) δ ppm 10.52 (s, 1 H) 8.62 (d, J = 2.18 Hz, 1 H) 8.51 (d, J = 2.18v Hz, 1 H) 7.84 (d, J = 2.18 Hz, 1 H) 7.76-7.82 (m, 1H) 7.50-7.61 (m, 2 H) 7.44 (s, 1 H) 1.13-1.21 (m, 1H) |
| P34 | 5-bromo-N-(2-bromo-6-carbamoyl-4- chloro-phenyl)-2-(3-chloro-2-pyridyl) pyrazole-3-carboxamide Disclosed in: WO2020/117493 | 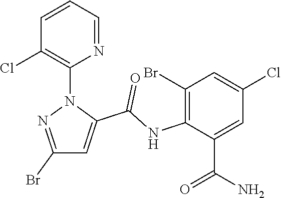 [SMILES: NC(=O)c1cc(Cl)cc(Br)c1NC(=O)c1cc(Br)nn1-c1ncccc1Cl] | 0.90 | 535 | 1 | | 1H NMR (400 MHz, DMSO- d6) δ ppm 10.40-10.51 (m, 1 H) 8.49-8.54 (m, 1 H) 8.12- 8.20 (m, 1 H) 7.92-7.97 (m, 1 H) 7.71-7.77 (m, 1 H) 7.51- 7.66 (m, 3 H) 7.37-7.46 (m, 1 H) |
| P35 | N-(4-bromo-2-carbamoyl-6-methyl- phenyl)-2-(3-chloro-2-pyridyl)-5-[[4- (trifluoromethyl)triazol-1-yl]methyl] pyrazole-3-carboxamide | 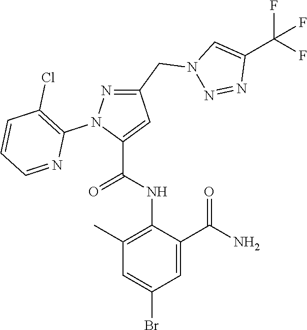 [SMILES: Cc1cc(Br)cc(C(N)=O)c1NC(=O)c1cc(Cn2cc(C(F)(F)F)nn2)nn1-c1ncccc1Cl] | | | | 190-195 | 1H NMR (400 MHz, DMSO- d6): δ 10.25 (s, 1H), 9.06 (s, 1H), 8.48 (dd, J = 1.6 Hz, 4.8 Hz, 1H), 8.15 (dd, J = 1.6 Hz, 8.0 Hz, 1H), 7.76 (s, 1H), 7.59-7.56 (m, 2H), 7.5 (s, 1H), 7.45 (s, 1H), 7.22 (s, 1H), 5.87 (s, 2H), 2.11 (s, 3H) |
| P36 | N-(4-bromo-2-carbamoyl-6-chloro- phenyl)-2-(3-chloro-2-pyridyl)-5-iodo- pyrazole-3-carboxamide | 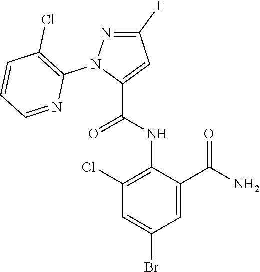 [SMILES: NC(=O)c1cc(Br)cc(Cl)c1NC(=O)c1cc(I)nn1-c1ncccc1Cl] | | | | >250 | 1H NMR (400 MHz, DMSO- d6): δ 10.40 (s, 1H), 8.49 (dd, J = 1.2 Hz, 4.4 Hz, 1H), 8.15 (dd, J = 1.6 Hz, 8.0 Hz, 1H), 7.93 (d, J = 2.0 Hz, 1H), 7.79 (s, 1H), 7.65-7.58 (m, 3H), 7.46 (s, 1H) |
| P37 | N-(4-bromo-2-carbamoyl-6-chloro- phenyl)-2-(3-chloro-2-pyridyl)-5-[[4- (trifluoromethyl)triazol-2-yl]methyl] pyrazole-3-carboxamide | 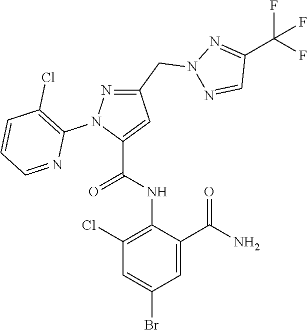 [SMILES: NC(=O)c1cc(Br)cc(Cl)c1NC(=O)c1cc(Cn2ncc(C(F)(F)F)n2)nn1-c1ncccc1Cl] | | | | 185-190 | 1H NMR (400 MHz, DMSO- d6): δ 10.40 (s, 1H), 8.53 (s, 1H), 8.48 (dd, J = 1.6 Hz, 4.8 Hz, 1H), 8.14 (dd, J = 1.2 Hz, 8.0 Hz, 1H), 7.91 (d, J = 2.0 Hz, 1H), 7.74 (s, 1H), 7.63 (d, J = 2.0 Hz, 1H), 7.59- 7.56 (m, 1H), 7.54 (s, 1H), 7.29 (s, 1H), 5.94 (s, 2H) |
| P38 | N-(4-bromo-2-carbamoyl-6- methyl-phenyl)-2-(3-chloro-2-pyridyl)- 5-[[4-(trifluoromethyl)triazol-2- yl]methyl]pyrazole-3- carboxamide | 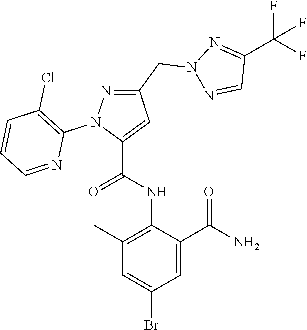 [SMILES: Cc1cc(Br)cc(C(N)=O)c1NC(=O)c1cc(Cn2ncc(C(F)(F)F)n2)nn1-c1ncccc1Cl] | | | | 205-210 | 1H NMR (400 MHz, DMSO- d6): δ 10.25 (s, 1H), 8.53 (s, 1H), 8.48 (dd, J = 1.6 Hz, 4.8 Hz, 1H), 8.15 (dd, J = 1.2 Hz, 8.0 Hz, 1H), 7.71 (s, 1H), 7.59-7.56 (m, 2H), 7.50 (s, 1H), 7.45 (s, 1H), 7.23 (s, 1H), 5.94 (s, 2H), 2.12 (s, 3H) |
| P39 | N-(4-bromo-2-carbamoyl-6-methyl- phenyl)-2-(3-chloro-2-pyridyl)-5-iodo- pyrazole-3-carboxamide | 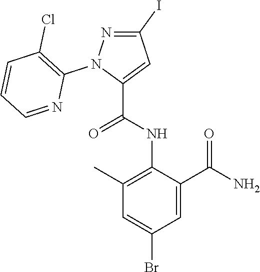 [SMILES: Cc1cc(Br)cc(C(N)=O)c1NC(=O)c1cc(I)nn1-c1ncccc1Cl] | | | | >250 | 1H NMR (400 MHz, DMSO- d6): δ 10.24 (s, 1H), 8.49 (dd, J = 1.6 Hz, 4.8 Hz, 1H), 8.15 (dd, J = 1.6 Hz, 8.0 Hz, 1H), 7.75 (s, 1H), 7.61-7.57 (m, 2H), 7.52-7.50 (m, 2H), 7.41 (s, 1H), 2.13 (s, 3H) |
| P40 | N-(4-bromo-2-carbamoyl-6-chloro- phenyl)-2-(3-chloro-2-pyridyl)-5-[[4- (trifluoromethyl)triazol-1-yl]methyl] pyrazole-3-carboxamide | 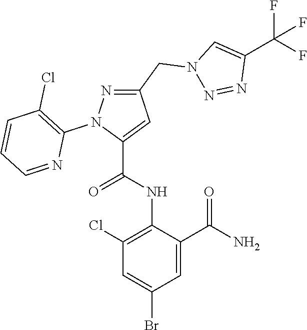 [SMILES: NC(=O)c1cc(Br)cc(Cl)c1NC(=O)c1cc(Cn2cc(C(F)(F)F)nn2)nn1-c1ncccc1Cl] | | | | 135-140 | 1H NMR (400 MHz, DMSO- d6): δ 9.07 (s, 1H), 8.48 (dd, J = 1.6 Hz, 4.8 Hz, 1H), 8.14 (d, J = 1.6 Hz, 1H), 7.91 (d, J = 2.4 Hz, 1H), 7.75 (s, 1H), 7.63 (d, J = 2.0 Hz, 1H), 7.59- 7.56 (m, 1H), 7.53 (s, 1H), 7.26 (s, 1H), 5.83 (s, 2H) |
| P41 | N-(4-bromo-2-carbamoyl-6- fluoro-phenyl)-2-(3-chloro-2- pyridyl)-5-[[5-(trifluoromethyl) tetrazol-2-yl]methyl]pyrazole-3- carboxamide | 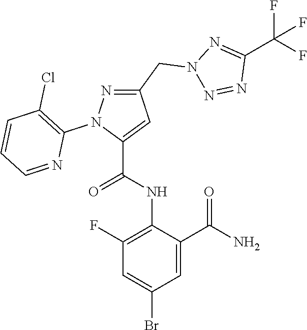 [SMILES: NC(=O)c1cc(Br)cc(F)c1NC(=O)c1cc(Cn2nnc(C(F)(F)F)n2)nn1-c1ncccc1Cl] | | | | 110-115 | 1H NMR (400 MHz, DMSO- d6): δ 10.4 (s, 1H), 8.49 (dd, J = 1.6 Hz, 4.8 Hz, 1H), 8.16 (dd, J = 1.6 Hz, 8.0 Hz, 1H), 7.92 (s, 1H), 7.75 (d, J = 2.0 Hz, 1H), 7.61-7.57 (m, 3H), 7.40 (s, 1H), 6.31 (s, 2H) |
| P42 | N-(4-bromo-2-carbamoyl-6- fluoro-phenyl)-2-(3-chloro-2- pyridyl)-5- (trifluoromethyl)pyrazole-3- carboxamide | 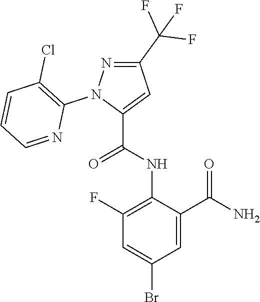 [SMILES: NC(=O)c1cc(Br)cc(F)c1NC(=O)c1cc(C(F)(F)F)nn1-c1ncccc1Cl] | | | | 180-185 | 1H NMR (400 MHz, DMSO- d6): δ 10.55 (s, 1H), 8.54 (dd, J = 1.6 Hz, 4.8 Hz, 1H), 8.23 (dd, J = 1.6 Hz, 8.4 Hz, 1H), 7.96 (s, 1H), 7.79 (s, 1H), 7.76 (d, J = 2.0 Hz, 1H), 7.69- 7.60 (m, 2H), 7.59 (s, 1H). |
| P43 | N-(4-bromo-2-carbamoyl-6-methyl- phenyl)-2-(3-chloro-2-pyridyl)-5-[[5-[4- (trifluoromethyl)phenyl]tetrazol-2-yl] methyl]pyrazole-3-carboxamide | 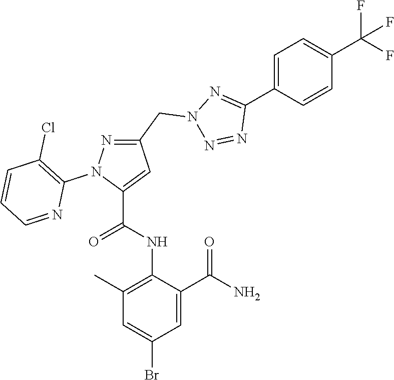 [SMILES: Cc1cc(Br)cc(C(N)=O)c1NC(=O)c1cc(Cn2nnc(-c3ccc(C(F)(F)F)cc3)n2)nn1-c1ncccc1Cl] | | | | 115-120 | 1H NMR (400 MHz, DMSO- d6): δ 10.25 (s, 1H), ), 8.48 (dd, J = 1.6 Hz, 4.8 Hz, 1H), 8.32 (d, J = 8.4 Hz, 2H), 8.16 (dd, J = 1.2 Hz, 8.0 Hz, 1H), 7.97 (d, J = 8.4 Hz, 2H), 7.73 (s, 1H), 7.60-7.57 (m, 2H), 7.50 (d, J = 2.4 Hz, 1H), 7.45 (s, 1H), 7.30 (s, 1H), 6.22 (s, 2H), 2.11 (s, 3H) |
| P44 | N-(4-bromo-2-carbamoyl-6-chloro- phenyl)-2-(3-chloro-2-pyridyl)-5-[[5-[4- (trifluoromethyl)phenyl]tetrazol-2-yl] methyl]pyrazole-3-carboxamide | 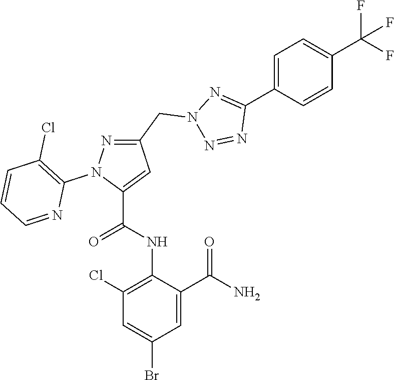 [SMILES: NC(=O)c1cc(Br)cc(Cl)c1NC(=O)c1cc(Cn2nnc(-c3ccc(C(F)(F)F)cc3)n2)nn1-c1ncccc1Cl] | | | | 135-140 | 1H NMR (400 MHz, DMSO- d6): δ 10.41 (s, 1H), 8.48 (dd, J = 1.2 Hz, 4.8 Hz, 1H), 8.32 (d, J = 8.0 Hz, 2H), 8.15 (dd, J = 0.8 Hz, 8.0 Hz, 2H), 7.97 (d, J = 8.4 Hz, 1H), 7.91 (d, J = 2.0 Hz, 1H), 7.71 (s, 1H), 7.63 (d, J = 2.0 Hz, 1H), 7.62- 7.57 (m, 1H), 7.54 (s, 1H), 7.36 (s, 1H), 6.23 (s, 2H) |
| P45 | N-(4-bromo-2-carbamoyl-6-methyl- phenyl)-2-(3-chloro-2-pyridyl)-5- methoxy-pyrazole-3-carboxamide | 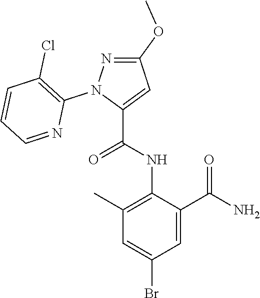 [SMILES: COc1cc(C(=O)Nc2c(C)cc(Br)cc2C(N)=O)n(-c2ncccc2Cl)n1] | | | | 210-215 | 1H NMR (400 MHz, DMSO- d6): δ 10.16 (s, 1H), 8.46 (dd, J = 1.6 Hz, 4.4 Hz, 1H), 8.10 (dd, J = 1.6 Hz, 8.0 Hz, 1H), 7.73 (s, 1H), 7.59 (d, J = 2 Hz, 1H), 7.54-7.51 (m, 3H), 6.70 (s, 1H), 3.87 (s, 3H), 2.14 (s, 3H) |
| P46 | 2-(3-chloro-2-pyridyl)-N-(2,4-dibromo- 6-carbamoyl-phenyl)-5-(trifluoromethyl)- pyrazole-3-carboxamide | 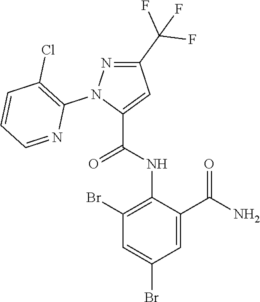 [SMILES: NC(=O)c1cc(Br)cc(Br)c1NC(=O)c1cc(C(F)(F)F)nn1-c1ncccc1Cl] | | | | 205-210 | 1H NMR (DMSO-d6, 400 MHz): 10.60 (s, 1H), δ 8.54 (dd, J = 1.2 Hz, 4.4 Hz, 1H), 8.21 (dd, J = 1.6 Hz, 8.4 Hz, 1H), 8.07 (d, J = 2.0 Hz, 1H,), 7.79-7.77 (m, 2H), 7.69- 7.64 (m, 2H), 7.55 (s, 1H). |
| P47 | N-(4-bromo-2-carbamoyl-6-chloro- phenyl)-2-(3-chloro-2-pyridyl)-5- methoxy-pyrazole-3-carboxamide | 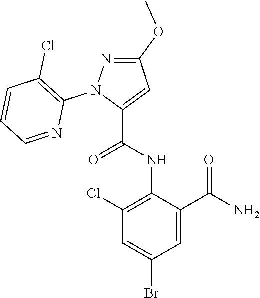 [SMILES: COc1cc(C(=O)Nc2c(Cl)cc(Br)cc2C(N)=O)n(-c2ncccc2Cl)n1] | | | | 160-165 | 1H NMR (400 MHz, DMSO- d6): δ 10.29 (s, 1H), 8.46 (dd, J = 1.6 Hz, 4.8 Hz, 1H), 8.10 (dd, J = 1.2 Hz, 8.0 Hz, 1H), 7.93 (d, J = 2 Hz, 1H), 7.76 (s, 1H), 7.65 (s, 1H), 7.64 (d, J = 2.4 Hz, 1H), 7.54-7.51 (m, 1H), 6.70 (s, 1H), 3.87 (s, 3H) |
| P48 | 5-[[5-(3-bromophenyl)tetrazol-2-yl] methyl]-N-(2-carbamoyl-4-chloro-6- methyl-phenyl)-2-(3-chloro-2-pyridyl) pyrazole-3-carboxamide | 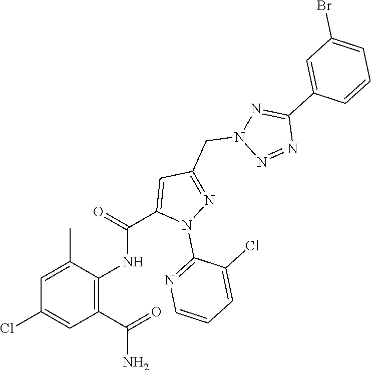 [SMILES: Cc1cc(Cl)cc(C(N)=O)c1NC(=O)c1cc(Cn2nnc(-c3cccc(Br)c3)n2)nn1-c1ncccc1Cl] | 1.08 | 628 | 1 | | ¹H NMR (₁-CDCl₃) δ: 9.95 (s, 1H), 8.47-8.53 (dd, 1H), 8.38 (d, 1H), 8.15 (d, 1H), 7.86- 7.91 (dd, 1H), 7.62 (br d, 1H), 7.33-7.44 (m, 4H), 7.12 (s, 1H), 6.02 (s, 3H), 5.62-5.73 (m, 1H), 2.22 (s, 3H) |
| P49 | 5-[[5-(4-bromophenyl)tetrazol- 2-yl]methyl]-N-(2-carbamoyl-4- chloro-6-methyl-phenyl)-2-(3-chloro-2- pyridyl)pyrazole-3-carboxamide | 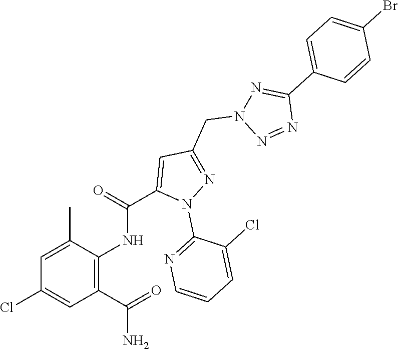 [SMILES: Cc1cc(Cl)cc(C(N)=O)c1NC(=O)c1cc(Cn2nnc(-c3ccc(Br)cc3)n2)nn1-c1ncccc1Cl] | 1.08 | 628 | 1 | | ¹H NMR (₁-CDCl₃) δ: 9.94 (s, 1H), 8.46-8.53 (dd, 1H), 8.06- 8.12 (m, 2H), 7.86-7.91 (dd, 1H), 7.62-7.67 (m, 2H), 7.38- 7.44 (dd, 1H), 7.31-7.37 (m, 2H), 7.11 (s, 1H), 6.03-6.11 (br s, 1H), 5.99-6.02 (m, 2H), 5.67-5.75 (br s, 1H), 2.21 (s, 3H) |
| P50 | N-(2-carbamoyl-4,6-dichloro-phenyl)- 2-(3-chloro-2-pyridyl)-5-iodo-pyrazole- 3-carboxamide | 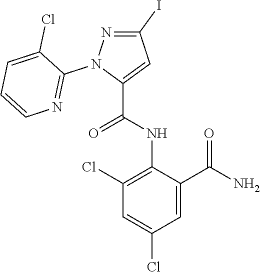 [SMILES: NC(=O)c1cc(Cl)cc(Cl)c1NC(=O)c1cc(I)nn1-c1ncccc1Cl] | | | | >250 | 1H NMR (400 MHz, DMSO- d6): δ 10.41 (s, 1H), 8.49 (dd, J = 1.6 Hz, 4.8 Hz, 1H), 8.15 (dd, J = 1.6 Hz, 8.0 Hz, 1H), 7.83 (d, J = 2.4 Hz, 1H), 7.79 (s, 1H), 7.61-7.57 (m, 2H), 7.54 (d, J = 2.4 Hz, 1H), 7.46 (s, 1H) |
| P51 | N-(2-bromo-6-carbamoyl-4-chloro- phenyl)-2-(3-chloro-2-pyridyl)-5-[[4- (trifluoromethyl)triazol-1-yl]methyl] pyrazole-3-carboxamide | 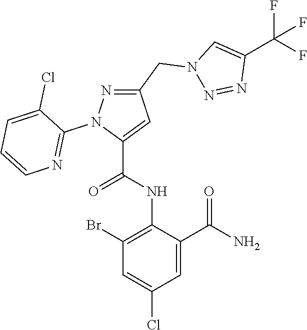 [SMILES: NC(=O)c1cc(Cl)cc(Br)c1NC(=O)c1cc(Cn2cc(C(F)(F)F)nn2)nn1-c1ncccc1Cl] | | | | >250 | 1H NMR (400 MHz, DMSO- d6): δ 10.41 (s, 1H), 9.07 (s, 1H), 8.48 (dd, J = 1.6 Hz, 4.8 Hz, 1H), 8.14 (dd, J = 1.2 Hz, 8.0 Hz, 1H), 7.93 (d, J = 2.0 Hz, 1H), 7.69 (s, 1H), 7.59- 7.52 (m, 3H), 7.28 (s, 1H), 5.88 (s, 2H) |
| P52 | N-(2-bromo-6-carbamoyl-4-chloro- phenyl)-2-(3-chloro-2-pyridyl)-5-[[4- (trifluoromethyl)triazol-2-yl]methyl] pyrazole-3-carboxamide | 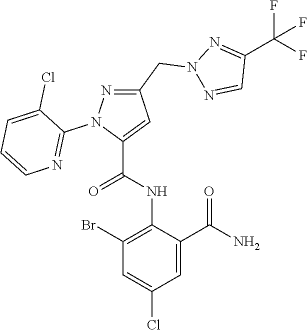 [SMILES: NC(=O)c1cc(Cl)cc(Br)c1NC(=O)c1cc(Cn2ncc(C(F)(F)F)n2)nn1-c1ncccc1Cl] | | | | 203-208 | 1H NMR (400 MHz, DMSO- d6): δ 10.41 (s, 1H), 8.54 (s, 1H), 8.48 (dd, J = 1.6 Hz, 4.4 Hz, 1H), 8.13 (dd, J = 1.6 Hz, 8.0 Hz, 1H), 7.93 (d, J = 2.4 Hz, 1H), 7.68 (s, 1H), 7.59- 7.52 (m, 3H), 7.29 (s, 1H), 5.94 (s, 2H) |
| P53 | 5-[[5-(2-bromophenyl)tetrazol-2-yl] methyl]-N-(2-carbamoyl-4-chloro-6- methyl-phenyl)-2-(3-chloro-2- pyridyl)pyrazole-3-carboxamide | 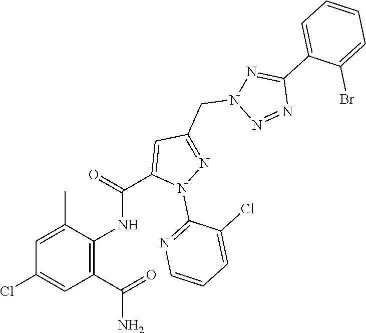 [SMILES: Cc1cc(Cl)cc(C(N)=O)c1NC(=O)c1cc(Cn2nnc(-c3ccccc3Br)n2)nn1-c1ncccc1Cl] | 1.02 | 628 | 1 | | ¹H NMR (₁-CDCl₃) δ: 9.95 (s, 1H), 8.48-8.52 (dd, 1H), 7.91- 7.96 (dd, 1H), 7.86-7.91 (dd, 1H), 7.74-7.79 (dd, 1H), 7.44- 7.49 (m, 1H), 7.39-7.43 (m, 1H), 7.32-7.38 (m, 3H), 7.17 (s, 1H), 6.07 (s, 3H), 5.64- 5.78 (br s, 1H), 2.22 (s, 3H) |
| P54 | N-(2-carbamoyl-4-chloro-6-methyl- phenyl)-2-(3-chloro-2-pyridyl)-5-[[5- (p-tolyl)tetrazol-2-yl]methyl]pyrazole-3- carboxamide | 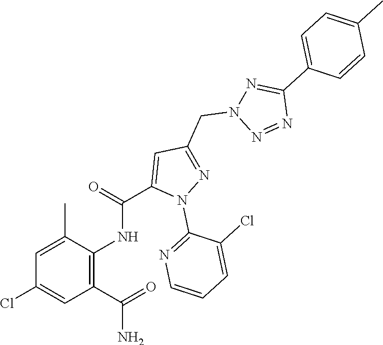 [SMILES: Cc1ccc(-c2nnn(Cc3cc(C(=O)Nc4c(C)cc(Cl)cc4C(N)=O)n(-c4ncccc4Cl)n3)n2)cc1] | 1.04 | 562 | 1 | | ¹H NMR (₁-CDCl₃) δ: 9.91 (s, 1H), 8.48-8.51 (dd, 1H), 8.09 (d, 2H), 7.86-7.90 (dd, 1H), 7.38-7.42 (m, 1H), 7.30-7.36 (m, 4H), 7.11 (s, 1H), 6.02- 6.14 (br s, 1H), 5.95-6.02 (s, 2H), 5.60-5.76 (br s, 1H), 2.43 (s, 3H), 2.19-2.25 (s, 3H) |
| P55 | N-(2-bromo-6-carbamoyl-4-chloro- phenyl)-2-(3-chloro-2-pyridyl)-5- methoxy-pyrazole-3-carboxamide | 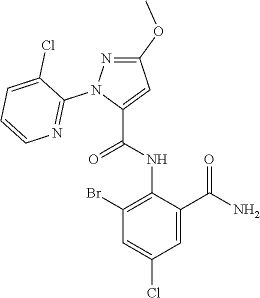 [SMILES: COc1cc(C(=O)Nc2c(Br)cc(Cl)cc2C(N)=O)n(-c2ncccc2Cl)n1] | | | | 205-210 | 1H NMR (400 MHz, DMSO- d6): δ 10.28 (s, 1H), 8.46 (dd, J = 1.6 Hz, J = 4.8 Hz, 1H), 8.09 (d, J = 1.6 Hz, 1H), 7.94 (d, J = 2.4 Hz, 1H), 7.67 (s, 1H), 7.57-7.51 (m, 3H), 6.76 (s, 1H), 3.87 (s, 3H) |
| P56 | N-(2-carbamoyl-4-chloro-6-methyl- phenyl)-2-(3-chloro-2-pyridyl)-5-(2,2- difluoroethoxy)pyrazole-3-carboxamide | 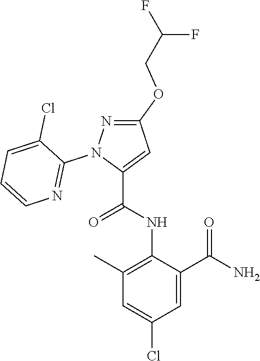 [SMILES: Cc1cc(Cl)cc(C(N)=O)c1NC(=O)c1cc(OCC(F)F)nn1-c1ncccc1Cl] | | | | 113-118 | 1H NMR (400 MHz, DMSO- d6): δ 10.22 (s, 1H), 8.47 (dd, J = 1.6 Hz, 4.8 Hz, 1H), 8.12 (dd, J = 1.6 Hz, 8.4 Hz, 1H), 7.51 (s, 1H), 7.56-7.53 (m, 2H), 7.47 (d, J = 2.4 Hz, 1H), 7.40 (d, J = 2.4 Hz, 1H), 6.79 (s, 1H), 6.54-6.25 (m, 1H), 4.52 (td, J = 2.8 Hz, 14.8 Hz, 2H), 2.14 (s, 3H) |
| P57 | N-(2-bromo-6-carbamoyl-4-chloro- phenyl)-2-(3-chloro-2-pyridyl)-5-[[5-[4- (trifluoromethyl)phenyl]tetrazol-2-yl] methyl]pyrazole-3-carboxamide | 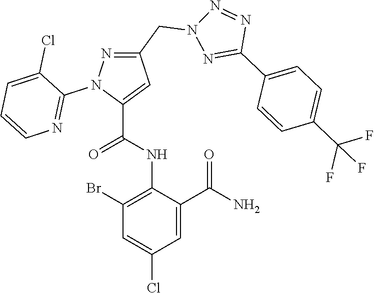 [SMILES: NC(=O)c1cc(Cl)cc(Br)c1NC(=O)c1cc(Cn2nnc(-c3ccc(C(F)(F)F)cc3)n2)nn1-c1ncccc1Cl] | | | | 230-250 | 1H NMR (400 MHz, DMSO- d6): δ 10.4 (s, 1H), 8.48 (d, J = 1.2 Hz, 1H), 8.35 (d, J = 10.8 Hz, 2H), 8.14 (d, J = 7.2 Hz, 1H), 7.99 (d, J = 8.0 Hz, 3H), 7.71 (s, 1H), 7.63-7.56 (m, 3H), 7.37 (s, 1H), 6.23 (s, 2H) |
| P58 | N-(2-carbamoyl-4,6-dichloro-phenyl)- 2-(3-chloro-2-pyridyl)-5-[[5-[4- (trifluoromethyl)phenyl]tetrazol-2-yl] methyl]pyrazole-3-carboxamide | 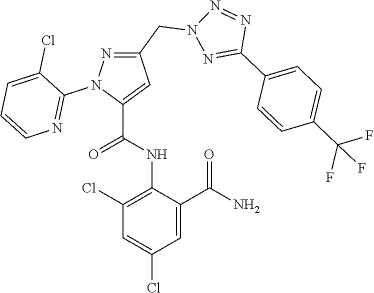 [SMILES: NC(=O)c1cc(Cl)cc(Cl)c1NC(=O)c1cc(Cn2nnc(-c3ccc(C(F)(F)F)cc3)n2)nn1-c1ncccc1Cl] | | | | 195-200 | 1H NMR (400 MHz, DMSO- d6): δ 10.41 (s, 1H), 8.48 (dd, J = 1.6 H, J = 4.8 Hz, 1H), 8.32 (d, J = 8.0 Hz, 2H), 8.15 (d, J = 1.2 Hz, 8.0 Hz, 1H), 7.97 (d, J = 8.4 Hz, 2H), 7.81 (d, J = 2.4 Hz, 2H), 7.60- 7.57 (m, 2H), 7.52 (d, J = 2.0 Hz, 1H), 7.36 (s, 1H), 6.23 (s, 2H) |
| P59 | N-(2-carbamoyl-4,6-dichloro-phenyl)-2- (3-chloro-2-pyridyl)-5-methoxy- pyrazole-3-carboxamide | 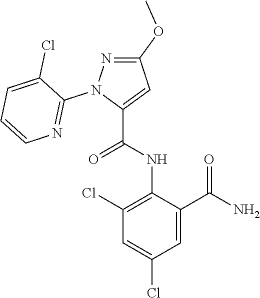 [SMILES: COc1cc(C(=O)Nc2c(Cl)cc(Cl)cc2C(N)=O)n(-c2ncccc2Cl)n1] | | | | 155-160 | 1H NMR (400 MHz, DMSO- d6): δ 10.3 (s, 1H), 8.46 (dd, J = 1.2 Hz, 4.8 Hz, 1H), 8.10 (dd, J = 1.2 Hz, 8.0 Hz, 1H), 7.82 (d, J = 2.4 Hz, 1H), 7.76 (s, 1H), 7.61 (s, 1H), 7.54- 7.51 (m, 2H), 6.77 (s, 1H), 3.87 (s, 3H) |
| P60 | N-(2-carbamoyl-6-chloro-4-iodo- phenyl)-2-(3-chloro-2-pyridyl)-5- (trifluoromethyl)pyrazole-3-carboxamide | 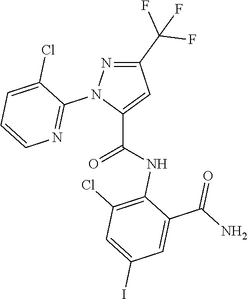 [SMILES: NC(=O)c1cc(I)cc(Cl)c1NC(=O)c1cc(C(F)(F)F)nn1-c1ncccc1Cl] | | | | 200-205 | 1H NMR (400 MHz, DMSO- d6): δ 10.57 (s, 1H), 8.54 (dd, J = 1.2 Hz, 4.4 Hz, 1H), 8.22 (dd, J = 1.6 Hz, 8.0 Hz, 1H), 8.03 (d, 1H, J = 2.0 Hz), 7.80- 7.76 (m, 3H), 7.67-7.64 (m, 1H), 7.53 (s, 1H) |
| P61 | 5-bromo-N-(2-carbamoyl-6-chloro-4- iodo-phenyl)-2-(3-chloro-2-pyridyl) pyrazole-3-carboxamide | 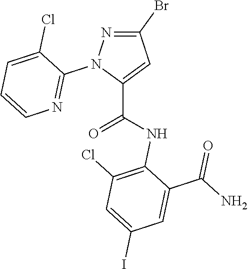 [SMILES: NC(=O)c1cc(I)cc(Cl)c1NC(=O)c1cc(Br)nn1-c1ncccc1Cl] | | | | 210-215 | 1H NMR (400 MHz, DMSO- d6): δ 10.42 (s, 1H), 8.50 (dd, J = 1.6 Hz, J = 4.8 Hz, 1H), 8.16 (dd, J = 1.6 Hz, J = 8 Hz, 1H), 8.01 (d, J = 1.6 Hz, 1H), 7.78 (d, J = 1.6 Hz, 2H), 7.62-7.58 (m, 1H), 7.54 (s, 1H), 7.41 (s, 1H) |
| P62 | N-(2-carbamoyl-4-iodo-6-methyl- phenyl)-2-(3-chloro-2-pyridyl)-5-[[5-[4- (trifluoromethyl)phenyl]tetrazol-2-yl] methyl]pyrazole-3-carboxamide | 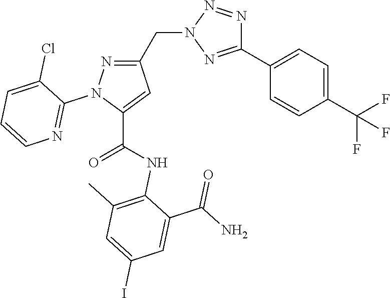 [SMILES: Cc1cc(I)cc(C(N)=O)c1NC(=O)c1cc(Cn2nnc(-c3ccc(C(F)(F)F)cc3)n2)nn1-c1ncccc1Cl] | | | | 125-130 | 1H NMR (400 MHz, DMSO- d6): δ 10.24 (s, 1H), ), 8.48 (dd, J = 1.6 Hz, J = 4.8 Hz, 1H), 8.32 (d, J = 8.4 Hz, 2H), 8.16 (dd, J = 1.6 Hz, J = 8 Hz, 1H), 7.97 (d, J = 8.4 Hz, 2H), 7.72 (d, J = 6.4 Hz, 2H), 7.64 (d, J = 1.6 Hz, 1H), 7.60- 7.57 (m, 1H), 7.43 (s, 1H), 7.29 (s, 1H), 6.22 (s, 1H), 2.08 (s, 3H) |
| P63 | 5-bromo-N-(2-carbamoyl-4-iodo-6- methyl-phenyl)-2-(3-chloro-2-pyridyl) pyrazole-3-carboxamide | 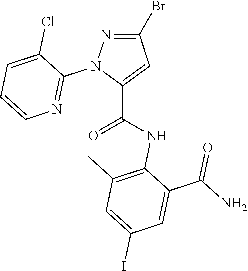 [SMILES: Cc1cc(I)cc(C(N)=O)c1NC(=O)c1cc(Br)nn1-c1ncccc1Cl] | | | | 210-215 | 1H NMR (400 MHz, DMSO- d6): δ 10.42 (s, 1H), 8.50 (dd, J = 1.6 Hz, 4.8 Hz, 1H), 8.16 (dd, J = 1.6 Hz, 8.0 Hz, 1H), 8.01 (d, J = 1.6 Hz, 1H), 7.78 (d, J = 1.6 Hz, 2H), 7.62- 7.58 (m, 1H), 7.54 (s, 1H), 7.41 (s, 1H), 2.11 (s, 3H) |
| P64 | N-(2-carbamoyl-6-fluoro-4-iodo- phenyl)-2-(3-chloro-2-pyridyl)- 5-(trifluoromethyl)pyrazole-3- carboxamide | 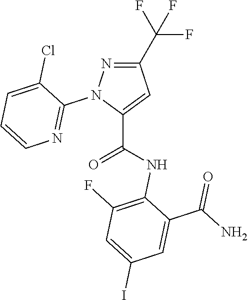 [SMILES: NC(=O)c1cc(I)cc(F)c1NC(=O)c1cc(C(F)(F)F)nn1-c1ncccc1Cl] | | | | 160-165 | 1H NMR (400 MHz, DMSO- d6): δ 10.53 (s, 1H), 8.54 (dd, 1H, J = 1.6 Hz, 4.8 Hz), 8.23 (dd, 1H, J = 1.6 Hz, 8.0 Hz), 7.93 (s, 1H), 7.86 (dd, 1H, J = 1.6 Hz, 9.2 Hz), 7.78 (s, 1H), 7.72 (s, 1H), 7.68-7.65 (m, 1H), 7.59 (s, 1H) |
| P65 | 5-bromo-N-(2-carbamoyl-6-fluoro-4- iodo-phenyl)-2-(3-chloro-2-pyridyl) pyrazole-3-carboxamide | 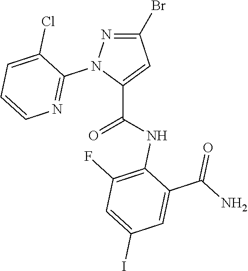 [SMILES: NC(=O)c1cc(I)cc(F)c1NC(=O)c1cc(Br)nn1-c1ncccc1Cl] | | | | 210-215 | 1H NMR (400 MHz, DMSO- d6): δ 10.40 (s, 1H), 8.51 (dd, J = 1.6 Hz, J = 4.8 Hz, 1H), 8.19 (dd, J = 1.6 Hz, J = 8.4 Hz, 1H), 7.93 (s, 1H), 7.85- 7.82 (m, 1H), 7.72 (s, 1H), 7.63-7.60 (m, 2H), 7.43 (s, 1H) |
| P66 | N-(2-carbamoyl-4-cyano-6-methyl- phenyl)-2-(3-chloro-2-pyridyl)-5-iodo- pyrazole-3-carboxamide | 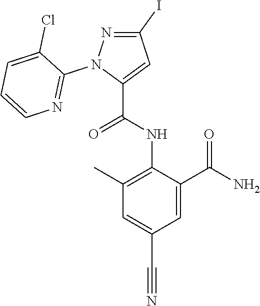 [SMILES: Cc1cc(C#N)cc(C(N)=O)c1NC(=O)c1cc(I)nn1-c1ncccc1Cl] | | | | >250 | 1H NMR (400 MHz, DMSO- d6): δ 10.52 (s, 1H), 8.49 (dd, J = 1.2 Hz, 4.4 Hz, 1H), 8.16 (dd, J = 1.2 Hz, 8.0 Hz, 1H), 7.88 (d, J = 4.1 Hz, 2H), 7.82 (s, 1H), 7.61-7.58 (m, 2H), 7.43 (s, 1H), 2.18 (s, 3H) |
| P67 | N-(2-carbamoyl-4-cyano-6-methyl- phenyl)-2-(3-chloro-2-pyridyl)-5-[[5- [4-(trifluoromethyl)phenyl]tetrazol-2- yl]methyl]pyrazole-3-carboxamide | 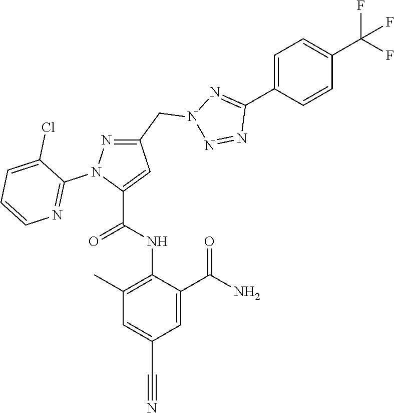 [SMILES: Cc1cc(C#N)cc(C(N)=O)c1NC(=O)c1cc(Cn2nnc(-c3ccc(C(F)(F)F)cc3)n2)nn1-c1ncccc1Cl] | | | | 210-215 | 1H NMR (400 MHz, DMSO- d6): δ 10.54 (s, 1H), 8.49 (dd, J = 1.6 Hz, 4.8 Hz, 1H), 8.32 (d, J = 8.0 Hz, 2H), 8.16 (d, J = 1.2 Hz, 8.0 Hz, 1H), 7.97 (d, J = 8.4 Hz, 2H), 7.85 (s, 3H), 7.60-7.55 (m, 2H), 7.32 (s, 1H), 6.23 (s, 2H), 2.16 (s, 3H) |
| P68 | N-(2-carbamoyl-4-iodo-6-methyl- phenyl)-2-(3-chloro-2-pyridyl)-5- (difluoromethyl)pyrazole-3-carboxamide | 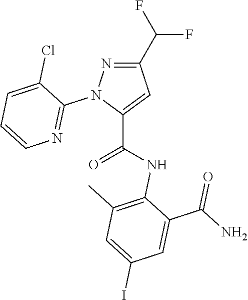 [SMILES: Cc1cc(I)cc(C(N)=O)c1NC(=O)c1cc(C(F)F)nn1-c1ncccc1Cl] | | | | 155-160 | 1H NMR (400 MHz, DMSO- d6): δ 10.35 (s, 1H), 8.65 (dd, J = 1.6 Hz, 4.8 Hz, 1H), 8.38 (dd, J = 1.6 Hz, 8.0 Hz, 1H), 7.93 (s, 1H), 7.80-7.72 (m, 3H), 7.57 (s, 1H), 7.41 (t, J = 52.8 Hz, 1H), 7.36 (s, 1H), 2.18 (s, 3H); |
| P69 | N-(2-carbamoyl-4-chloro-6-methyl- phenyl)-2-(3-chloro-2-pyridyl)-5-(1- fluorocyclopropyl)pyrazole-3- carboxamide | 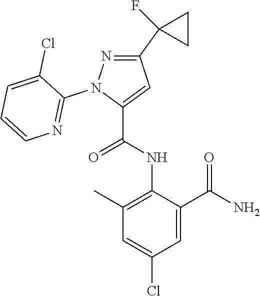 [SMILES: Cc1cc(Cl)cc(C(N)=O)c1NC(=O)c1cc(C2(F)CC2)nn1-c1ncccc1Cl] | | | | 225-230 | 1H NMR (DMSO-d6, 400 MHz): δ 10.24 (s, 1H), 8.66 (dd, J = 1.6 Hz, 4.8 Hz, 1H), 8.35 (dd, J = 1.2 Hz, 8.0 Hz, 1H), 7.91 (s, 1H), 7.77-7.47 (m, 1H), 7.59 (s, 1H), 7.51 (d, J = 2.0 Hz, 1H), 7.45 (d, J = 2.4 Hz, 1H), 7.10 (d, J = 2.4 Hz, 1H), 2.21 (s, 3H), 1.35- 1.25 (m, 2H), 1.23-1.17 (m, 2H); |
| P70 | N-(2-carbamoyl-4-chloro-6-methyl- phenyl)-2-(3-chloro-2-pyridyl)-5- (cyanomethyl)pyrazole-3-carboxamide | 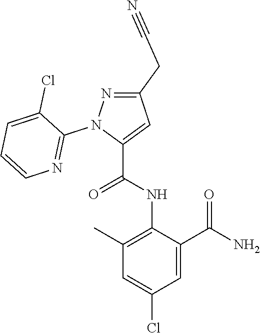 [SMILES: Cc1cc(Cl)cc(C(N)=O)c1NC(=O)c1cc(CC#N)nn1-c1ncccc1Cl] | | | | 180-185 | 1H NMR (DMSO-d6, 400 MHz): δ 10.28 (s, 1H), 8.49 (dd, J = 1.6 Hz, 4.8 Hz, 1H), 8.16 (dd, J = 1.6 Hz, 8.0 Hz, 1H), 7.73 (s, 1H), 7.60 (dd, J = 4.8 Hz, 8.4 Hz, 1H), 7.49 (s, 1H), 7.46 (d, J = 2.0 Hz, 1H), 7.40 (d, J = 2.0 Hz, 1H), 7.24 (s, 1H), 4.23 (s, 2H), 2.14 (s, 3H); |
| P71 | 5-bromo-N-(2-carbamoyl-4,6- dichloro-phenyl)-2-(3-chloro-2- pyridyl)pyrazole-3-carboxamide Disclosed in WO2008/010897 | 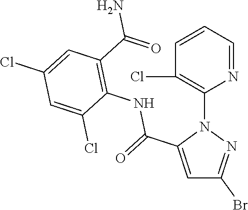 [SMILES: NC(=O)c1cc(Cl)cc(Cl)c1NC(=O)c1cc(Br)nn1-c1ncccc1Cl] | 0.91 | 492 | 1 | | ¹H NMR (400 MHz, DMSO- d6) δ ppm 10.46 (s, 1 H), 8.50 (dd, J = 4.72, 1.45 Hz, 1 H), 8.16 (dd, J = 7.99, 1.45 Hz, 1 H), 7.70-7.90 (m, 2 H), 7.49-7.65 (m, 3 H), 7.41 (s, 1 H) |
| P72 | N-(2-carbamoyl-4-chloro-6-methyl- phenyl)-2-(3-chloro-2-pyridyl)-5-(1- cyanocyclopropyl)pyrazole-3- carboxamide | 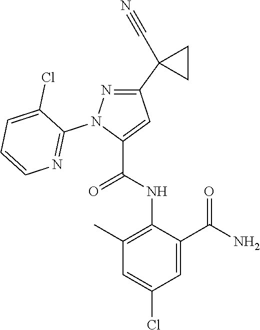 [SMILES: Cc1cc(Cl)cc(C(N)=O)c1NC(=O)c1cc(C2(C#N)CC2)nn1-c1ncccc1Cl] | | | | 225-230 | 1H NMR (DMSO-d6, 400 MHz): δ 10.24 (s, 1H), 8.48 (dd, J = 1.2 Hz, 4.8 Hz, 1H), 8.15 (dd, J = 1.6 Hz, 8.0 Hz, 1H), 7.73 (s, 1H), 7.59 (dd, J = 4.8 Hz, 8.0 Hz, 1H), 7.50 (s, 1H), 7.46 (d, J = 2.0 Hz, 1H), 7.40 (d, J = 2.4 Hz, 1H), 7.29 (s, 1H), 2.13 (s, 3H), 1.87 (q, J = 4.8 Hz, 2H), 1.58 (q, J = 4.4 Hz, 2H); |
| P73 | 5-bromo-2-(3-bromo-2-pyridyl)- N-(2-carbamoyl-4-chloro-6-methyl- phenyl)pyrazole-3-carboxamide Disclosed in WO2008/010897 | 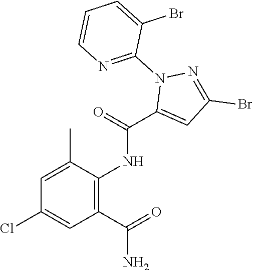 [SMILES: Cc1cc(Cl)cc(C(N)=O)c1NC(=O)c1cc(Br)nn1-c1ncccc1Br] | 0.90 | 514 | 1 | | 1H NMR (400 MHz, DMSO- d6) δ ppm 2.15 (s, 3 H) 7.35- 7.42 (m, 2 H) 7.44-7.54 (m, 3 H) 7.74 (br s, 1 H) 8.29 (dd, J = 7.99, 1.09 Hz, 1 H) 8.52 (dd, J = 4.54, 1.27 Hz, 1 H) 10.27 (s, 1 H) |
| P74 | N-(2-carbamoyl-4-chloro-6-methyl- phenyl)-2-(3-chloro-2-pyridyl)-5- (methylsulfinylmethyl)pyrazole-3- carboxamide Disclosed in WO2004/046129 | 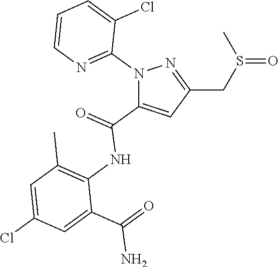 [SMILES: Cc1cc(Cl)cc(C(N)=O)c1NC(=O)c1cc(CS(C)=O)nn1-c1ncccc1Cl] | | | | 125-130 | 1H NMR (DMSO-d6, 400 MHz): δ 10.31 (s, 1H), 8.49 (dd, J = 1.6 Hz, 4.8 Hz, 1H), 8.15 (dd, J = 1.2 Hz, 8.0 Hz, 1H), 7.75 (s, 1H), 7.59 (dd, J = 4.8 Hz, 8.4 Hz, 1H), 7.51 (s, 1H), 7.44 (s, 1H), 7.40 (s, 1H), 7.21 (s, 1H), 4.29 (d, J = 13.6 Hz, 1H), 4.12 (d, J = 13.6 Hz, 1H), 2.59 (s, 3H), 2.13 (s, 3H); |
| P75 | N-(2-carbamoyl-4-chloro-6-methyl- phenyl)-2-(3-chloro-2-pyridyl)-5-ethyl- pyrazole-3-carboxamide | 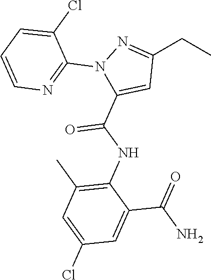 [SMILES: CCc1cc(C(=O)Nc2c(C)cc(Cl)cc2C(N)=O)n(-c2ncccc2Cl)n1] | | | | 245-250 | 1H NMR (DMSO-d6, 400 MHz): δ 10.17 (s, 1H), 8.47 (dd, J = 1.6 Hz, J = 4.4 Hz, 1H), 8.12 (dd, J = 1.6 Hz, 8.0 Hz, 1H), 7.75 (s, 1H), 7.56- 7.52 (m, 2H), 7.46 (s, 1H), 7.46-7.39 (m, 1H), 7.06 (s, 1H), 2.72 (q, J = 7.2 Hz, J = 7.6 Hz, 2H), 2.14 (s, 3H), 1.28 (t, J = 7.6 Hz, 3H); |
| P76 | N-(2-carbamoyl-4-chloro-6-methyl- phenyl)-2-(3-chloro-2-pyridyl)-5- cyclopropyl-pyrazole-3-carboxamide | 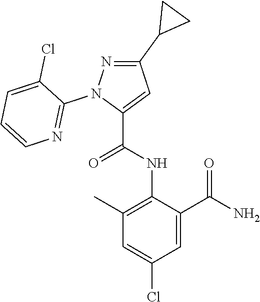 [SMILES: Cc1cc(Cl)cc(C(N)=O)c1NC(=O)c1cc(C2CC2)nn1-c1ncccc1Cl] | | | | 240-245 | 1H NMR (DMSO-d6, 400 MHz): δ 10.13 (s, 1H), 8.46 (dd, J = 1.6 Hz, 4.8 Hz, 1H), 8.11 (dd, J = 1.6 Hz, 8.0 Hz, 1H), 7.74 (s, 1H), 7.55-7.52 (m, 2H), 7.46 (s, 1H), 7.46- 7.39 (m, 1H), 6.93 (s, 1H), 2.13 (s, 3H), 2.05-1.98 (m, 1H), 1.00-0.97 (m, 2H), 0.79-0.77 (m, 2H); |
| P77 | 5-bromo-N-(2-carbamoyl-4- chloro-6-methyl-phenyl)-2- pyrimidin-2-yl-pyrazole-3-carboxamide | 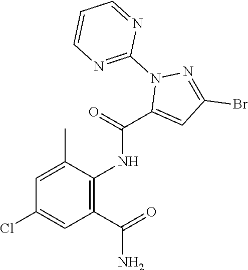 [SMILES: Cc1cc(Cl)cc(C(N)=O)c1NC(=O)c1cc(Br)nn1-c1ncccn1] | 0.78 | 437 | | 187-198 | 1H NMR (400 MHz, DMSO) δ ppm 2.25-2.31 (m, 3 H) 6.93- 7.11 (m, 1 H) 7.38-7.48 (m, 1 H) 7.49-7.52 (m, 1 H) 7.52- 7.58 (m, 1 H) 7.58-7.63 (m, 1 H) 7.69-7.82 (m, 1 H) 8.84- 8.93 (m, 2 H) 10.25-10.41 (m, 1 H) |
| P78 | N-(2-carbamoyl-4-chloro-6-methyl- phenyl)-2-(3-chloro-2-pyridyl)-5- (methylsulfonylmethyl)pyrazole-3- carboxamide Disclosed in WO2004/046129 | 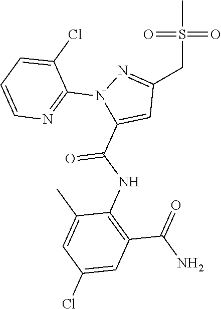 [SMILES: Cc1cc(Cl)cc(C(N)=O)c1NC(=O)c1cc(CS(C)(=O)=O)nn1-c1ncccc1Cl] | | | | 140-145 | 1H NMR (DMSO-d6, 400 MHz): δ 10.35 (s, 1H), 8.50 (t, J = 1.6 Hz, 1H), 8.17-8.15 (m, 1H), 7.75 (s, 1H), 7.61- 7.57 (m, 1H), 7.52 (s, 1H), 7.46 (s, 1H), 7.39 (s, 1H), 7.32 (s, 1H), 4.69 (s, 2H), 3.03 (s, 3H), 2.14 (s, 3H); |
| P79 | N-(2-carbamoyl-4-chloro-6-methyl- phenyl)-2-(3-chloro-2-pyridyl)-5- (methylsulfanylmethyl)pyrazole-3- carboxamide Disclosed in WO2004/046129 | 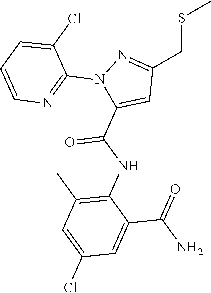 [SMILES: CSCc1cc(C(=O)Nc2c(C)cc(Cl)cc2C(N)=O)n(-c2ncccc2Cl)n1] | | | | 175-180 | 1H NMR (DMSO-d6, 400 MHz): δ 10.24 (s, 1H), 8.48 (dd, J = 1.6 Hz, 4.8 Hz, 1H), 8.14 (dd, J = 1.6 Hz, 8.0 Hz, 1H), 7.74 (s, 1H), 7.58-7.53 (m, 2H), 7.46 (d, J = 2.4 Hz, 1H), 7.39 (d, J = 2.4 Hz, 1H), 7.17 (s, 1H), 3.77 (s, 2H), 2.14 (s, 3H), 2.08 (s, 3H); |
| P80 | N-(2-carbamoyl-4-chloro-6-methyl- phenyl)-2-(3-chloro-2-pyridyl)-5- (methoxymethyl)pyrazole-3-carboxamide Disclosed in WO2004/046129 | 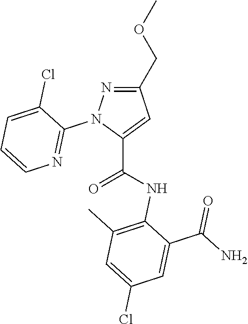 [SMILES: COCc1cc(C(=O)Nc2c(C)cc(Cl)cc2C(N)=O)n(-c2ncccc2Cl)n1] | | | | 225-230 | 1H NMR (DMSO-d6, 400 MHz): δ 10.22 (s, 1H), 8.48 (dd, J = 1.6 Hz, 4.8 Hz, 1H), 8.14 (dd, J = 1.6 Hz, 8.0 Hz, 1H), 7.72 (s, 1H), 7.58 (dd, J = 4.8 Hz, 8.0 Hz, 1H), 7.51 (s, 1H), 7.46 (d, J = 2.0 Hz, 1H), 7.40 (d, J = 2.4 Hz, 1H), 7.21 (s, 1H), 4.49 (s, 2H), 3.34 (s, 3H), 2.14 (s, 3H); |
| P81 | N-(2-carbamoyl-4-chloro-6-methyl- phenyl)-2-(3-chloro-2-pyridyl)-5-[[5- (3-pyridyl)tetrazol-2-yl]methyl] pyrazole-3-carboxamide | 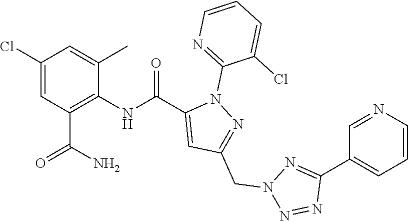 [SMILES: Cc1cc(Cl)cc(C(N)=O)c1NC(=O)c1cc(Cn2nnc(-c3cccnc3)n2)nn1-c1ncccc1Cl] | 1.19 | 549 | 2 | | |
| P82 | N-(2-carbamoyl-4-chloro-6-methyl- phenyl)-2-(3-chloro-2-pyridyl)-5-[[5- (2-pyridyl)tetrazol-2-yl]methyl] pyrazole-3-carboxamide | 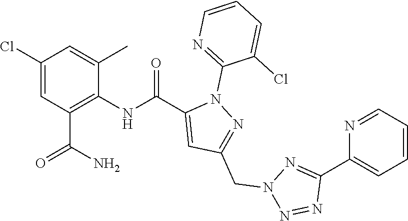 [SMILES: Cc1cc(Cl)cc(C(N)=O)c1NC(=O)c1cc(Cn2nnc(-c3ccccn3)n2)nn1-c1ncccc1Cl] | 1.27 | 549 | 2 | | |
| P83 | N-(2-carbamoyl-4-chloro-6- methyl-phenyl)-2-(3-chloro-2-pyridyl)- 5-[[5-(4-methoxyphenyl)tetrazol-2-yl] methyl]pyrazole-3-carboxamide | 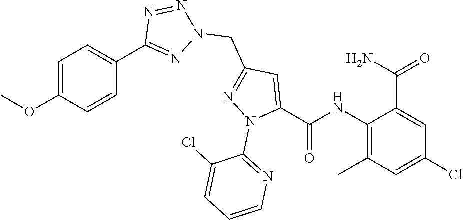 [SMILES: COc1ccc(-c2nnn(Cc3cc(C(=O)Nc4c(C)cc(Cl)cc4C(N)=O)n(-c4ncccc4Cl)n3)n2)cc1] | 1.5 | 578 | 2 | | |
| P84 | N-(2-carbamoyl-4-chloro-6-methyl- phenyl)-2-(3-chloro-2-pyridyl)-5-[[5- (4-fluorophenyl)tetrazol-2-yl]methyl] pyrazole-3-carboxamide | 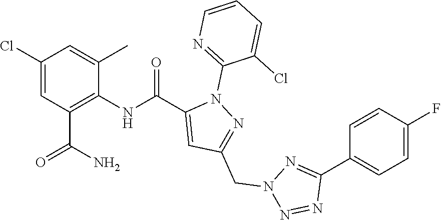 [SMILES: Cc1cc(Cl)cc(C(N)=O)c1NC(=O)c1cc(Cn2nnc(-c3ccc(F)cc3)n2)nn1-c1ncccc1Cl] | 1.54 | 566 | 2 | | |
| P85 | N-(2-carbamoyl-4-chloro-6-methyl- phenyl)-2-(3-chloro-2-pyridyl)-5-[[5- (3-methoxyphenyl)tetrazol-2-yl]methyl] pyrazole-3-carboxamide | 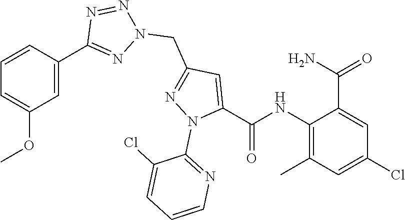 [SMILES: COc1cccc(-c2nnn(Cc3cc(C(=O)Nc4c(C)cc(Cl)cc4C(N)=O)n(-c4ncccc4Cl)n3)n2)c1] | 1.53 | 578 | 2 | | |
| P86 | 5-[[5-(4-bromo-2-fluoro-phenyl) tetrazol-2-yl]methyl]-N-(2-carbamoyl- 4-chloro-6-methyl-phenyl)-2-(3- chloro-2-pyridyl)pyrazole-3- carboxamide | 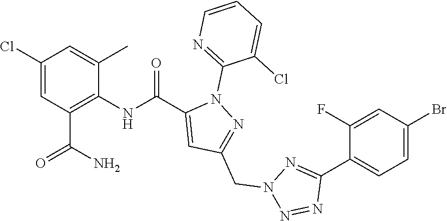 [SMILES: Cc1cc(Cl)cc(C(N)=O)c1NC(=O)c1cc(Cn2nnc(-c3ccc(Br)cc3F)n2)nn1-c1ncccc1Cl] | 1.67 | 644 | 2 | | |
| P87 | N-(2-carbamoyl-4-chloro-6-methyl- phenyl)-2-(3-chloro-2-pyridyl)-5-[[5- (2-chloro-4-pyridyl)tetrazol-2-yl] methyl]pyrazole-3-carboxamide | 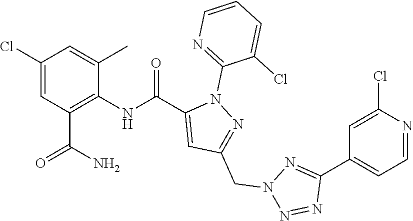 [SMILES: Cc1cc(Cl)cc(C(N)=O)c1NC(=O)c1cc(Cn2nnc(-c3ccnc(Cl)c3)n2)nn1-c1ncccc1Cl] | 1.45 | 583 | 2 | | |
| P88 | N-(2-carbamoyl-4-chloro-6-methyl- phenyl)-2-(3-chloro-2-pyridyl)-5-[[5- (3-cyanophenyl)tetrazol-2-yl]methyl] pyrazole-3-carboxamide | 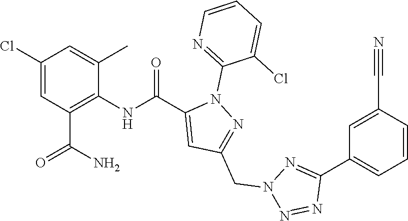 [SMILES: Cc1cc(Cl)cc(C(N)=O)c1NC(=O)c1cc(Cn2nnc(-c3cccc(C#N)c3)n2)nn1-c1ncccc1Cl] | 1.45 | 573 | 2 | | |
| P89 | 5-[[5-(5-bromo-2-fluoro- phenyl)tetrazol-2-yl]methyl]-N- (2-carbamoyl-4-chloro-6-methyl- phenyl)-2-(3-chloro-2-pyridyl) pyrazole-3-carboxamide | 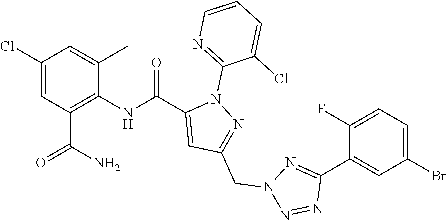 [SMILES: Cc1cc(Cl)cc(C(N)=O)c1NC(=O)c1cc(Cn2nnc(-c3cc(Br)ccc3F)n2)nn1-c1ncccc1Cl] | 1.65 | 644 | 2 | | |
| P90 | N-(2-carbamoyl-4-chloro-6-methyl- phenyl)-2-(3-chloro-2-pyridyl)-5- [[5-[2-(trifluoromethyl)phenyl] tetrazol-2-yl]methyl]pyrazole-3- carboxamide | 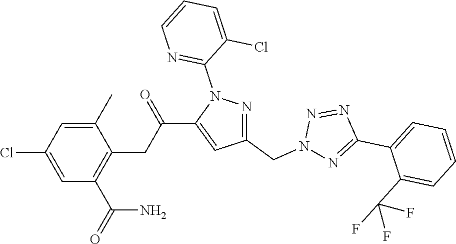 [SMILES: Cc1cc(Cl)cc(C(N)=O)c1CC(=O)c1cc(Cn2nnc(-c3ccccc3C(F)(F)F)n2)nn1-c1ncccc1Cl] | 1.58 | 616 | 2 | | |
| P91 | N-(2-carbamoyl-4-chloro-6-methyl- phenyl)-2-(3-chloro-2-pyridyl)-5- [[5-(2,4-dichlorophenyl)tetrazol-2- yl]methyl]pyrazole-3-carboxamide | 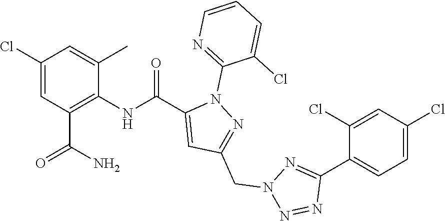 [SMILES: Cc1cc(Cl)cc(C(N)=O)c1NC(=O)c1cc(Cn2nnc(-c3ccc(Cl)cc3Cl)n2)nn1-c1ncccc1Cl] | 1.72 | 616 | 2 | | |
| P92 | N-(2-carbamoyl-4-chloro-6-methyl- phenyl)-2-(3-chloro-2-pyridyl)-5-[[5- (2,3-dichlorophenyl)tetrazol-2-yl] methyl]pyrazole-3-carboxamide | 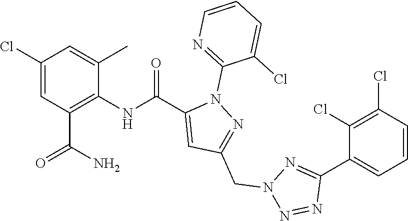 [SMILES: Cc1cc(Cl)cc(C(N)=O)c1NC(=O)c1cc(Cn2nnc(-c3cccc(Cl)c3Cl)n2)nn1-c1ncccc1Cl] | 1.67 | 616 | 2 | | |
| P93 | N-(2-carbamoyl-4-chloro-6-methyl- phenyl)-2-(3-chloro-2-pyridyl)-5-[[5- (m-tolyl)tetrazol-2-yl]methyl]pyrazole- 3-carboxamide | 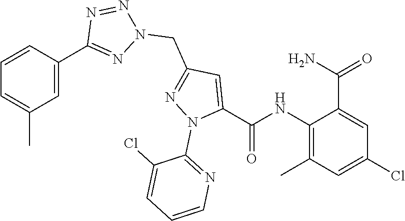 [SMILES: Cc1cccc(-c2nnn(Cc3cc(C(=O)Nc4c(C)cc(Cl)cc4C(N)=O)n(-c4ncccc4Cl)n3)n2)c1] | 1.61 | 562 | 2 | | |
| P94 | 5-[[5-(4-tert-butylphenyl)tetrazol-2- yl)methyl]-N-(2-carbamoyl-4-chloro-6- methyl-phenyl)-2-(3-chloro-2-pyridyl) pyrazole-3-carboxamide | 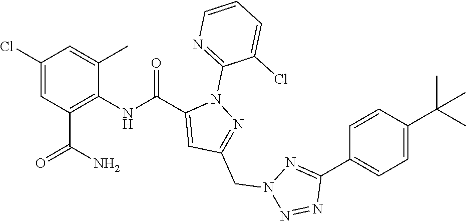 [SMILES: Cc1cc(Cl)cc(C(N)=O)c1NC(=O)c1cc(Cn2nnc(-c3ccc(C(C)(C)C)cc3)n2)nn1-c1ncccc1Cl] | 1.87 | 604 | 2 | | |
| P95 | N-(2-carbamoyl-4-chloro-6-methyl- phenyl)-2-(3-chloro-2-pyridyl)-5-[[5- pyrimidin-4-yltetrazol-2-yl)methyl] pyrazole-3-carboxamide | 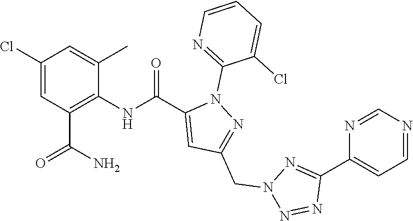 [SMILES: Cc1cc(Cl)cc(C(N)=O)c1NC(=O)c1cc(Cn2nnc(-c3ccncn3)n2)nn1-c1ncccc1Cl] | 1.16 | 550 | 2 | | |
| P96 | 5-[[5-(5-bromo-3-pyridyl)tetrazol-2-yl] methyl]-N-(2-carbamoyl-4-chloro-6- methyl-phenyl)-2-(3-chloro-2-pyridyl) pyrazole-3-carboxamide | 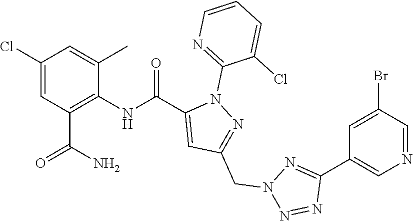 [SMILES: Cc1cc(Cl)cc(C(N)=O)c1NC(=O)c1cc(Cn2nnc(-c3cncc(Br)c3)n2)nn1-c1ncccc1Cl] | 1.48 | 627 | 2 | | |
| P97 | N-(2-carbamoyl-4-chloro-6-methyl- phenyl)-2-(3-chloro-2-pyridyl)-5-[[5-[3- (trifluoromethyl)phenyl]tetrazol-2-yl] methyl]pyrazole-3-carboxamide | 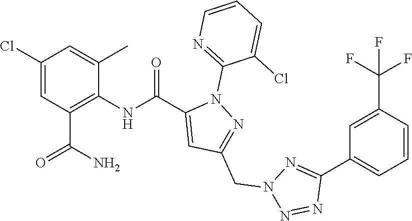 [SMILES: Cc1cc(Cl)cc(C(N)=O)c1NC(=O)c1cc(Cn2nnc(-c3cccc(C(F)(F)F)c3)n2)nn1-c1ncccc1Cl] | 1.71 | 616 | 2 | | |
| P98 | N-(2-carbamoyl-4-chloro-6-methyl- phenyl)-2-(3-chloro-2-pyridyl)-5-[[5- (o-tolyl)tetrazol-2-yl]methyl]pyrazole- 3-carboxamide | 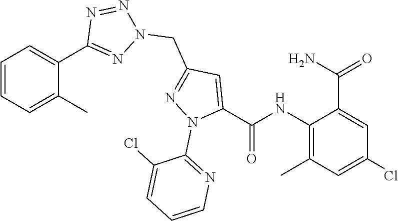 [SMILES: Cc1ccccc1-c1nnn(Cc2cc(C(=O)Nc3c(C)cc(Cl)cc3C(N)=O)n(-c3ncccc3Cl)n2)n1] | 1.59 | 562 | 2 | | |
| P99 | N-(2-carbamoyl-4-chloro-6-methyl- phenyl)-5-[[5-(4-chloro-3-methyl- phenyl)tetrazol-2-yl]methyl]-2-(3- chloro-2-pyridyl)pyrazole-3- carboxamide | 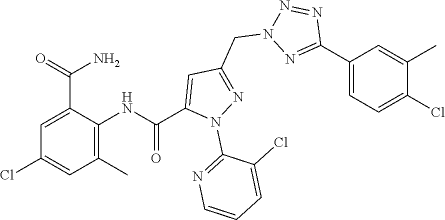 [SMILES: Cc1cc(-c2nnn(Cc3cc(C(=O)Nc4c(C)cc(Cl)cc4C(N)=O)n(-c4ncccc4Cl)n3)n2)ccc1Cl] | 1.77 | 596 | 2 | | |
| P100 | 5-[[5-(4-bromo-2-methyl-phenyl) tetrazol-2-yl]methyl]-N-(2- carbamoyl-4-chloro-6-methyl-phenyl)- 2-(3-chloro-2-pyridyl)pyrazole-3- carboxamide | 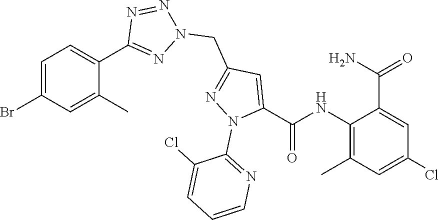 [SMILES: Cc1cc(Br)ccc1-c1nnn(Cc2cc(C(=O)Nc3c(C)cc(Cl)cc3C(N)=O)n(-c3ncccc3Cl)n2)n1] | 1.78 | 640 | 2 | | |
| P101 | N-(2-carbamoyl-4-chloro-6-methyl- phenyl)-2-(3-chloro-2-pyridyl)-5-[[5- (2,6-dichlorophenyl)tetrazol-2-yl] methyl]pyrazole-3-carboxamide | 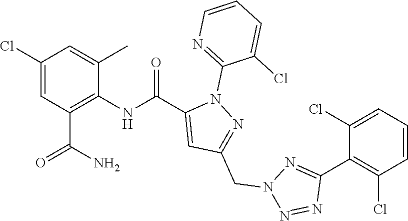 [SMILES: Cc1cc(Cl)cc(C(N)=O)c1NC(=O)c1cc(Cn2nnc(-c3c(Cl)cccc3Cl)n2)nn1-c1ncccc1Cl] | 1.58 | 616 | 2 | | |
| P102 | 5-[[5-(3-bromo-4-fluoro-phenyl) tetrazol-2-yl]methyl]-N-(2-carbamoyl- 4-chloro-6-methyl-phenyl)-2-(3-chloro- 2-pyridyl)pyrazole-3-carboxamide | 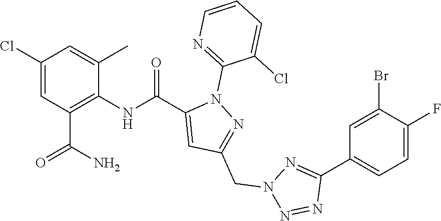 [SMILES: Cc1cc(Cl)cc(C(N)=O)c1NC(=O)c1cc(Cn2nnc(-c3ccc(F)c(Br)c3)n2)nn1-c1ncccc1Cl] | 1.71 | 644 | 2 | | |
| P103 | N-(2-carbamoyl-4-chloro-6-methyl- phenyl)-2-(3-chloro-2-pyridyl)-5-[[5- (4-pyridyl)tetrazol-2-yl]methyl] pyrazole-3-carboxamide | 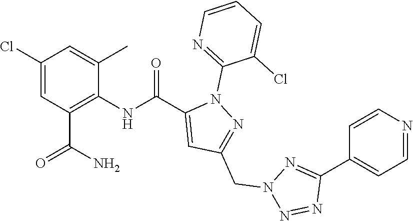 [SMILES: Cc1cc(Cl)cc(C(N)=O)c1NC(=O)c1cc(Cn2nnc(-c3ccncc3)n2)nn1-c1ncccc1Cl] | 1.13 | 549 | 2 | | |
| P104 | N-(2-carbamoyl-4-chloro-6-methyl- phenyl)-5-[[5-(4-chlorophenyl)tetrazol- 2-yl]methyl]-2-(3-chloro-2-pyridyl) pyrazole-3-carboxamide | 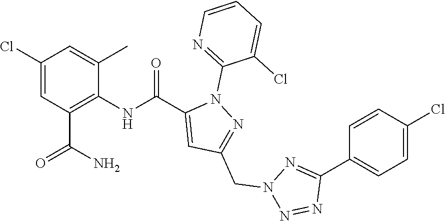 [SMILES: Cc1cc(Cl)cc(C(N)=O)c1NC(=O)c1cc(Cn2nnc(-c3ccc(Cl)cc3)n2)nn1-c1ncccc1Cl] | 1.66 | 582 | 2 | | |
| P105 | N-(2-carbamoyl-4-chloro-6- methyl-phenyl)-2-(3-chloro-2-pyridyl)- 5-[(5-phenyltetrazol-2-yl)methyl] pyrazole-3-carboxamide | 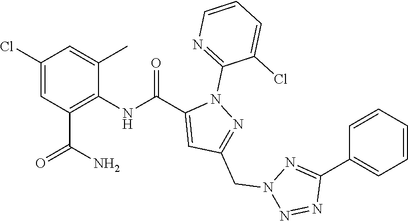 [SMILES: Cc1cc(Cl)cc(C(N)=O)c1NC(=O)c1cc(Cn2nnc(-c3ccccc3)n2)nn1-c1ncccc1Cl] | 1.51 | 548 | 2 | | |

TABLE I
Intermediates

| | | | RT | [M + H] | | |
|---|---|---|---|---|---|---|
| Entry | IUPAC name | STRUCTURE | (min) | (measured) | Method | NMR |
| I1 | 5-[(4- clorophenyl) methoxymethyl]- 2-(3-chloro-2- pyridyl)pyrazole- 3-carboxylic acid | 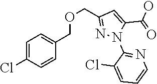 | 0.95 | 378 | 1 | 1H NMR (400 MHz, DMSO-d6) δ ppm 8.54 (dd, J = 4.72, 1.45 Hz, 1 H) 8.21 (dd, J = 8.17, 1.27 Hz, 1 H) 7.65 (dd, J = 7.99, 4.72 Hz, 1 H) 7.37 − 7.44 (m, 4 H) 7.03 (s, 1 H) 4.58 (s, 2 H) 4.56 (s, 2 H) |
| I2 | 6-chloro-2-[5-[(4- chlorophenyl) methoxymethyl]- 2-(3-chloro-2- pyridyl)pyrazol-3- yl]-8-methyl-3,1 -benzoxazin-4- one | 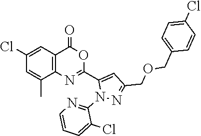 [SMILES: Cc1cc(Cl)cc2c(=O)oc(-c3cc(COCc4ccc(Cl)cc4)nn3-c3ncccc3Cl)nc12] | 1.30 | 527 | 1 | |
| I3 | 5-(chloromethyl)- 2-(3-chloro-2- pyridyl)pyrazole- 3-carboxylic acid | 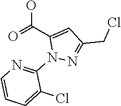 | 0.69 | 272 | 1 | 1H NMR (400 MHz, 3-MeOD) δ ppm 8.52 (dd, J = 4.72, 1.45 Hz, 1 H) 8.13 (dd, J = 7.99, 1.45 Hz, 1 H) 7.62 (dd, J = 8.17, 4.90 Hz, 1 H) 7.14 (s, 1 H) 4.73 (s, 2 H) |
| I4 | 6-chloro-2-[5- (chloromethyl)-2- (3-chloro-2-pyridyl) pyrazol-3- yl]-8-methyl-3,1- benzoxazin-4- one | 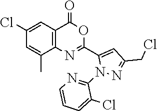 [SMILES: Cc1cc(Cl)cc2c(=O)oc(-c3cc(CCl)nn3-c3ncccc3Cl)nc12] | 1.17 | 422/423 | 1 | |
| I5 | 2-(3-chloro-2-pyridyl)-5- (2,2,3,3,3- pentafluoropropoxy) pyrazole-3- carboxylic acid | 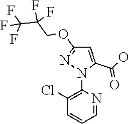 | 0.93 | 372 | 1 | 1H NMR (400 MHz, Solvent) δ ppm 8.51 (dd, J = 4.72, 1.45 Hz, 1 H) 8.17 (dd, J = 7.99, 1.45 Hz, 1 H) 7.61 (dd, J = 7.99, 4.72 Hz, 1 H) 6.59 (br s, 1 H) 4.96 (br t, J = 13.26 Hz, 2 H) |
| I6 | 6-chloro-2-[2- (3-chloro-2- pyridyl)-5-(2,2,3,3,3- pentafluoropropoxy) pyrazol-3- yl]-8-methyl-3,1- benzoxazin-4- one | 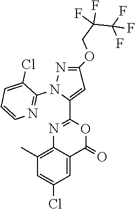 [SMILES: Cc1cc(Cl)cc2c(=O)oc(-c3cc(OCC(F)(F)C(F)(F)F)nn3-c3ncccc3Cl)nc12] | 1.29 | 521 | 1 | |
| I7 | 2-[2-(3-chloro-2-pyridyl)-5- methoxy-pyrazol- 3-yl]-6-iodo-8- methyl-3,1- benzoxazin-4-one | 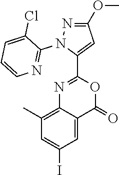 | 1.05 | 494 | 1 | |
| I8 | 2-[5-[(4- chlorophenyl) methoxymethyl]- 2-(3-chloro-2- pyridyl)pyrazol-3- yl]-8-methyl-4-oxo-3,1- benzoxazine-6-carbonitrile | 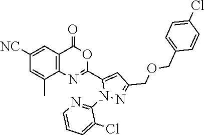 [SMILES: Cc1cc(C#N)cc2c(=O)oc(-c3cc(COCc4ccc(Cl)cc4)nn3-c3ncccc3Cl)nc12] | 1.20 | 518 | 1 | |
| I9 | 6-chloro-2-[2-(3-chloro-2- pyridyl)-5-[[5- (trifluoromethyl)tetrazol-2- yIjmethyl]pyrazol-3-yl]-8- methyl-3,1-benzoxazin-4-one | 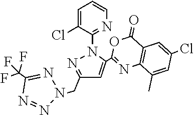 | 1.18 | 523 | 1 | |
| I10 | 6-bromo-2-[2-(3-chloro-2- pyridyl)-5-iodo- pyrazol-3-yl]-8- methyl-3,1-benzoxazin-4-one | 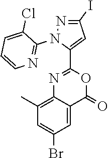 | | 544.7 | 3 | |
| I11 | 6-bromo-8-chloro-2-[2-(3- chloro-2-pyridyl)-5-iodo- pyrazol-3-yl]-3, 1-benzoxazin-4- one | 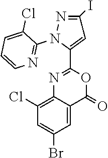 | | 564.6 | 3 | |
| I12 | | 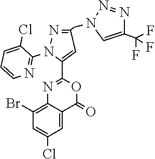 | | | | 1H NMR (400 MHz, DMSO-d6): δ 9.09 (s, 1H), 8.57 (dd, J = 1.2 Hz, 4.8 Hz, 1H), 8.30 (dd, J = 1.6 Hz, 8.0 Hz, 1H), 8.24 (d, J = 2.4 Hz, 1H), 8.07 (d, J = 2.4 Hz, 1H), 7.71 (dd, J = 4.8 Hz, 8.0 Hz, 1H), 7.45 (s, 1H), 5.91 (s, 2H); |
| I13 | 8-bromo-6-chloro-2-[2-(3- chloro-2-pyridyl)-5-[[4- (trifluoromethyl)triazol-1- yijmethyl]pyrazol-3-yl]-3,1- benzoxazin-4-one | 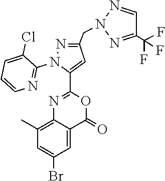 | | | | 1H NMR (400 MHz, DMSO-d6): δ 8.61 (dd, J = 1.2 Hz, 4.8 Hz, 1H), 8.52 (s, 1H), 8.33 (dd, J = 1.2 Hz, 8.0 Hz, 1H), 8.01 (d, J = 2.4 Hz, 1H), 7.89 (s, 1H), 7.76 − 7.72 (m, 1H), 7.30 (s, 1H), 5.98 (s, 2H), 1.71 (s, 3H); |
| I14 | 6-bromo-8-chloro-2-[2-(3- chloro-2-pyridyl)-5-[4- (trifluoromethyl)triazol-2- yl]methyl]pyrazol-3-yl]-3,1- benzoxazin-4-one | 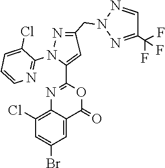 | | 587.8 | 3 | |
| I15 | 2-[2-(3-chloro-2- pyridyl)-5-iodo- pyrazol-3-yl]-8- methyl-4-oxo- 3, 1-benzoxazine-6- carbonitrile | 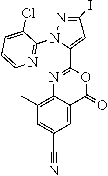 | | | | 1H NMR (400 MHz, DMSO-d6): δ 8.63 (dd, J = 1.6 Hz, 4.8 Hz, 1H), 8.37 − 8.33 (m, 2H), 8.09 (s, 1H), 7.77 − 7.74 (m, 1H), 7.59 (s, 1H), 1.72 (s, 3H); |
| I16 | 6,8-dichloro-2- [2-(3-chloro-2- pyridyl)-5-iodo- pyrazol-3-yl]- 3, 1-benzoxazin-4-one | 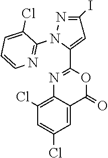 [SMILES: O=c1oc(-c2cc(I)nn2-c2ncccc2Cl)nc2c(Cl)cc(Cl)cc12] | | 520.7 | 3 | |
| I17 | 6-bromo-2-[2-(3-chloro-2- pyridyl)-5-[[5- (trifluoromethyl)tetrazol-2- yl]methyl]pyrazol- 3-yl]-8-fluoro- 3,1-benzoxazin-4-one | 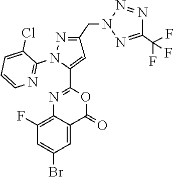 | | | | 1H NMR (400 MHz, DMSO-d6): δ 8.55 (dd, J = 1.2 Hz, 4.4 Hz, 1H), 8.26 (dd, J = 1.2 Hz, 8.0 Hz, 1H), 7.72 − 7.68 (m, 1H), 7.29 (s, 1H), 6.26 (s, 2H), 4.15 (q, J = 7.2 Hz, 2H), 1.08 (t, J = 6.8 Hz, 3H); |
| I18 | 2-[2-(3-chloro- 2-pyridyl)-5-[[5- [4- (trifluoromethyl) phenyl]tetrazol- 2-yljmethyl]pyrazol- 3-yl]-6-iodo- 8-methyl-3,1- benzoxazin-4-one | 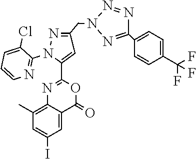 | | | | 1H NMR (400 MHz, DMSO-d6): δ 8.59 (dd, J = 1.6 Hz, 8.8 Hz, 1H), 8.33 (d, J = 1.2 Hz, 1H), 8.3 (d, J = 8.0 Hz, 2H), 8.16 (s, 1H), 8.03 (s, 1H), 7.95 (d, J = 8.4 Hz, 2H), 7.75 − 7.72 (m, 1H), 7.42 (s, 1H), 6.25 (s, 2H); |
| I19 | 2-(3-chloro-2- pyridyl)-5-[[5-[4- (trifluoromethyl) phenyl]tetrazol- 2-yl]methyl]pyrazole-3- carboxylic acid | 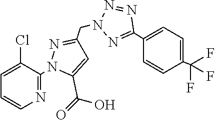 | | | | 1H NMR (400 MHz, DMSO-d6): 8 13.73 (s, 1H), 8.53 (dd, J = 1.2 Hz, 4.8 Hz, 1H), 8.29 (d, J = 8.4 Hz, 2H), 8.22 (dd, J = 1.6 Hz, 8.0 Hz, 1H), 7.94 (d, J = 8.4 Hz, 2H), 7.68 − 7.64 (m, 1H), 7.16 (s, 1H), 6.17 (s, 2H); |
| I20 | 6-bromo-8-chloro-2-[2-(3- chloro-2-pyridyl)-5-[[5-[4- (trifluoromethyl) phenyl]tetrazol- 2-yl]methyl] pyrazol-3-yl]-3,1- benzoxazin-4-one | 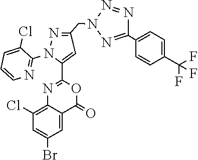 | | | | 1H NMR (400 MHz, DMSO-d6): δ 8.56 (dd, J = 1.6 Hz, 4.8 Hz, 1H), 8.31 − 8.29 (m, 3H), 8.22 (d, J = 2.0 Hz, 1H), 8.13 (d, J = 2.0 Hz, 1H), 7.96 (d, J = 8.4 Hz, 2H), 7.72 − 7.69 (m, 1H), 7.47 (s, 1H), 6.26 (s, 2H); |
| I21 | 8-bromo-6-chloro-2-[2-(3- chloro-2-pyridyl)-5-[[5-[4- (trifluoromethyl) phenyl]tetrazol- 2-yl]methyl] pyrazol-3-yl]-3,1- benzoxazin-4-one | 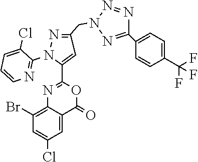 | | | | 1H NMR (400 MHz, DMSO-d6): δ 8.56 (dd, J = 1.2 Hz, 4.4 Hz, 1H), 8.31 − 8.29 (m, 3H), 8.24 (d, J = 2.4 Hz, 1H), 8.06 (d, J = 2.0 Hz, 1H), 7.95 (d, J = 8.4 Hz, 2H), 7.71 − 7.68 (m, 1H), 7.46 (s, 1H), 6.25 (s, 2H); |
| I22 | 6-bromo-2-[2-(3-chloro-2- pyridyl)-5-[[5-[4- (trifluoromethyl) phenyl]tetrazol- 2-yljmethyl]pyrazol-3-yl]-8- methyl-3,1-benzoxazin-4-one | 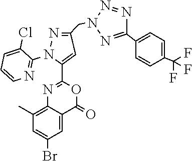 | | 645.0 | 3 | |
| I23 | 6-bromo-8-chloro-2-[2-(3- chloro-2-pyridyl)-5-methoxy- pyrazol-3-yl]-3,1- benzoxazin-4- one | 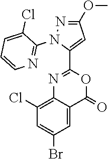 | | | | 1H NMR (400 MHz, DMSO-d6): δ 8.55 (dd, J = 1.6 Hz, 4.8 Hz, 1H), 8.24 (dd, J = 1.6 Hz, 8.0 Hz, 1H), 8.2 (d, J = 2.4 Hz, 1H), 8.12 (d, J = 2.0 Hz, 1H), 7.67 − 7.64 (m, 1H), 6.85 (s, 1H), 3.91 (s, 3H). |
| I24 | 8-bromo-6-chloro-2-[2-(3- chloro-2-pyridyl)- 5-methoxy- pyrazol-3-yl]- 3,1-benzoxazin-4- one | 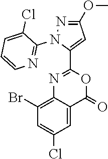 | | 468.8 | 3 | |
| I25 | 2-[2-(3-chloro- 2-pyridyl)-5-iodo- pyrazol-3-yl]-8- methyl-4-oxo- 3,1-benzoxazine- 6-carbonitrile | 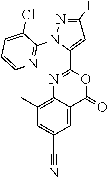 | | | | 1H NMR (400 MHz, DMSO-d6): δ 8.63 (dd, J = 1.6 Hz, 4.8 Hz, 1H), 8.37 − 8.33 (m, 2H), 8.09 (s, 1H), 7.77 − 7.74 (m, 1H), 7.59 (s, 1H), 1.72 (s, 3H); |
| I26 | 2-[2-(3-chloro- 2-pyridyl)-5- (difluoromethyl) pyrazol-3-yl]-6- iodo-8-methyl-3, 1-benzoxazin- 4-one | 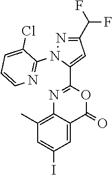 | | 552.8 | 3 | |
| I27 | 6-chloro-2-[2-(3-chloro-2- pyridyl)-5-(1- fluorocyclopropyl) pyrazol-3-yl]- 8-methyl-3,1- benzoxazin-4-one | 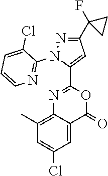 | | 430.9 | 3 | |
| I28 | 2-(3-chloro-2-pyridyl)-5-(1- fluorocyclopropyl) pyrazole-3- carboxylic acid | 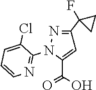 | | | | 1H NMR (DMSO-d6, 400 MHz): δ 13.00 (Brs, 1H), 8.63 (dd, J = 1.2 Hz, 4.4 Hz, 1H), 8.33 (dd, J = 1.6 Hz, 8.4 Hz, 1H), 7.76 − 7.72 (m, 1H), 7.05 (d, J = 2.4 Hz, 1H), 1.31 − 1.15 (m, 4H); |
| I29 | 6-chloro-2-[2-(3-chloro-2- pyridyl)-5- (methoxymethyl) pyrazol-3-yl]-8- methyl-3,1-benzoxazin- 4-one | 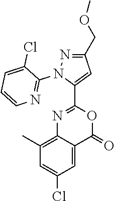 | | 416.9 | 3 | |
| I30 | 2-(3-chloro-2-pyridyl)-5- (methoxymethyl) pyrazole-3- carboxylic acid | 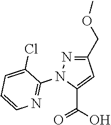 | | | | 1H NMR (DMSO-d6, 400 MHz): δ 13.77 (s, 1H), 8.55 (dd, J = 2.4 Hz, 4.8 Hz, 1H), 8.23 (dd, J = 1.6 Hz, 8.0 Hz, 1H), 7.66 (dd, J = 4.8 Hz, 8.0 Hz, 1H), 7.01 (s, 1H), 4.45 (s, 2H), 3.29 (s, 3H); |
| I31 | 2-(3-chloro-2-pyridyl)-5- (methylsulfanylmethyl) pyrazole- 3-carboxylic acid | 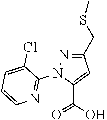 | | 282.0 | 3 | |
| I32 | 6-chloro-2-[2-(3-chloro-2- pyridyl)-5- cyclopropyl-pyrazol- (3-yl]-8-methyl- 3,1-benzoxazin- 4-one | 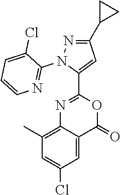 | | 412.9 | 3 | |
| I33 | 2-(3-chloro-2-pyridyl)-5- cyclopropyl-pyrazole-3- carboxylic acid | 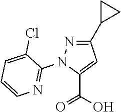 | | | | 1H NMR (DMSO-d6, 400 MHz): δ 12.90 (brs, 1H), 8.52 (dd, J = 1.6 Hz, 4.8 Hz, 1H), 8.19 (dd, J = 1.6 Hz, 8.0) Hz, 1H), 7.63 (dd, J = 4.8 Hz, 8.4 Hz, 1H), 6.77 (s, 1H), 2.01 − 1.94 (m, 1H), 0.96 -0.94 (m, 2H), 0.79 − 0.74 (m, 2H); |
| I34 | 6-chloro-2-[2-(3-chloro-2- pyridyl)-5-ethyl- pyrazol-3-yl]-8- methyl-3,1-benzoxazin- 4-one | 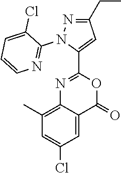 | | | | 1H NMR (DMSO-d6, 400 MHz): δ 8.60 (dd, J = 1.6 Hz, 4.8 Hz, 1H), 8.00 (dd, J = 1.6 Hz, 8.4 Hz, 1H), 7.88 (dd, J = 0.8 Hz, 2.8 Hz, 1H), 7.76 − 7.69 (m, 2H), 7.18 (s, 1H), 2.76 (q, J = 7.6 Hz, 2H), 1.72 (s, 3H), 1.30 (t, J = 7.6 Hz 3H); |
| I35 | 2-(3-chloro-2- pyridyl)-5-ethyl- pyrazole-3-carboxylic acid | 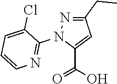 | | | | 1H NMR (DMSO-d6, 400 MHz): δ 13.35 (brs, 1H), 8.52 (dd, J = 1.6 Hz, 4.8 Hz, 1H), 8.20 (dd, J = 1.6 Hz, 8.0 Hz, 1H), 7.63 (dd, J = 4.8 Hz, 8.0 Hz, 1H), 6.89 (s, 1H), 2.68 (q, J = 7.6 Hz, 2H), 1.24 (t, J = 7.6 Hz, 3H); |
| I36 | 2-(3-chloro-2-pyridyl)-5- (cyanomethyl)pyrazole-3- carboxylic acid | 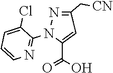 | | | | 1H NMR (CDCl3, 400 MHz): δ 13.72 (s, 1H), 8.56 (dd, J = 1.6 Hz, 4.4 Hz, 1H), 8.25 (dd, J = 1.2 Hz, 8.0 Hz, 1H), 7.69 (dd, J = 4.8 Hz, 8.0 Hz, 1H), 7.02 (s, 1H), 4.17 (s, 2H); |
| I37 | 5-bromo-2-pyrimidin-2-yl- pyrazole-3-carboxylic acid | 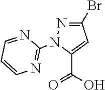 | | | | 1H NMR (400 MHz, DMSO) δ ppm 7.09 − 7.21 (m, 1 H) 7.62 − 7.72 (m, 1 H) 8.91 − 9.02 (m, 2 H) 13.52 − 14.40 (m, 1 H) |
| I38 | 2-(5-bromo-2- pyrimidin-2-yl- pyrazol-3-yl)-6- chloro-8-methyl- 3,1-benzoxazin-4-one | 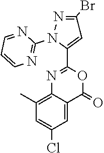 | | | | 1H NMR (400 MHz, DMSO) δ ppm 1.94 − 2.02 (m, 3 H) 7.41 − 7.51 (m, 1 H) 7.72 − 7.79 (m, 1 H) 7.82 − 7.90 (m, 1 H) 7.89 − 7.97 (m, 1 H) 8.96 − 9.07 (m, 2 H) |

The activity of the compositions according to the invention can be broadened considerably, and adapted to prevailing circumstances, by adding other insecticidally, acaricidally and/or fungicidally active ingredients. The mixtures of the compounds of formula I with other insecticidally, acaricidally and/or fungicidally active ingredients may also have further surprising advantages which can also be described, in a wider sense, as synergistic activity. For example, better tolerance by plants, reduced phytotoxicity, insects can be controlled in their different development stages or better behaviour during their production, for example during grinding or mixing, during their storage or during their use.

Suitable additions to active ingredients here are, for example, representatives of the following classes of active ingredients: organophosphorus compounds, nitrophenol derivatives, thioureas, juvenile hormones, formamidines, benzophenone derivatives, ureas, pyrrole derivatives, carbamates, pyrethroids, chlorinated hydrocarbons, acylureas, pyridylmethyleneamino derivatives, macrolides, neonicotinoids and Bacillus thuringiensis preparations.

The following mixtures of a compound of formula I with an active substance are preferred (the abbreviation “TX” means “one compound selected from the compounds defined in Tables A-1 to A-4 & P”):
an adjuvant selected from the group of substances consisting of petroleum oils (alternative name) (628)+TX;
abamectin+TX, acequinocyl+TX, acetamiprid+TX, acetoprole+TX, acrinathrin+TX, acynonapyr+TX, afidopyropen+TX, afoxolaner+TX, alanycarb+TX, allethrin+TX, alpha-cypermethrin+TX, alphamethrin+TX, amidoflumet+TX, aminocarb+TX, azocyclotin+TX, bensultap+TX, benzoximate+TX, benzpyrimoxan+TX, betacyfluthrin+TX, beta-cypermethrin+TX, bifenazate+TX, bifenthrin+TX, binapacryl+TX, bioallethrin+TX, S-bioallethrin+TX, bioresmethrin+TX, bistrifluron+TX, broflanilide+TX, brofluthrinate+TX, bromophos-ethyl+TX, buprofezine+TX, butocarboxim+TX, cadusafos+TX, carbaryl+TX, carbosulfan+TX, cartap+TX, CAS number: 1632218-00-8+TX, CAS number: 1808115-49-2+TX, CAS number: 2032403-97-5+TX, CAS number: 2044701-44-0+TX, CAS number: 2128706-05-6+TX, CAS number: 2095470-94-1+TX, CAS number: 2377084-09-6+TX, CAS number: 1445683-71-5+TX, CAS number: 2408220-94-8+TX, CAS number: 2408220-91-5+TX, CAS number: 1365070-72-9+TX, CAS number: 2171099-09-3+TX, CAS number: 2396747-83-2+TX, CAS number: 2133042-31-4+TX, CAS number: 2133042-44-9+TX, CAS number: 1445684-82-1+TX, CAS number: 1445684-82-1+TX, CAS number: 1922957-45-6+TX, CAS number: 1922957-46-7+TX, CAS number: 1922957-47-8+TX, CAS number: 1922957-48-9+TX, CAS number: 2415706-16-8+TX, CAS number: 1594624-87-9+TX, CAS number: 1594637-65-6+TX, CAS number: 1594626-19-3+TX, CAS number: 1990457-52-7+TX, CAS number: 1990457-55-0+TX, CAS number: 1990457-57-2+TX, CAS number: 1990457-77-6+TX, CAS number: 1990457-66-3+TX, CAS number: 1990457-85-6+TX, CAS number: 2220132-55-6+TX, CAS number: 1255091-74-7+TX, CAS number: RNA (Leptinotarsa decemlineata-specific recombinant double-stranded interfering GS2)+TX, CAS number: 2719848-60-7+TX, CAS number: 1956329-03-5+TX, chlorantraniliprole+TX, chlordane+TX, chlorfenapyr+TX, chloroprallethrin+TX, chromafenozide+TX, clenpirin+TX, cloethocarb+TX, clothianidin+TX, 2-chlorophenyl N-methylcarbamate (CPMC)+TX, cyanofenphos+TX, cyantraniliprole+TX, cyclaniliprole+TX, cyclobutrifluram+TX, cycloprothrin+TX, cycloxaprid+TX, cyenopyrafen+TX, cyetpyrafen (or etpyrafen)+TX, cyflumetofen+TX, cyfluthrin+TX, cyhalodiamide+TX, cyhalothrin+TX, cypermethrin+TX, cyphenothrin+TX, cyproflanilide+TX, cyromazine+TX, deltamethrin+TX, diafenthiuron+TX, dialifos+TX, dibrom+TX, dicloromezotiaz+TX, diflovidazine+TX, diflubenzuron+TX, dimpropyridaz+TX, dinactin+TX, dinocap+TX, dinotefuran+TX, dioxabenzofos+TX, emamectin (or emamectin benzoate)+TX, empenthrin+TX, epsilon-momfluorothrin+TX, epsilon-metofluthrin+TX, esfenvalerate+TX, ethion+TX, ethiprole+TX, etofenprox+TX, etoxazole+TX, famphur+TX, fenazaquin+TX, fenfluthrin+TX, fenmezoditiaz+TX, fenitrothion+TX, fenobucarb+TX, fenothiocarb+TX, fenoxycarb+TX, fenpropathrin+TX, fenpyroximate+TX, fensulfothion+TX, fenthion+TX, fentinacetate+TX, fenvalerate+TX, fipronil+TX, flometoquin+TX, flonicamid+TX, fluacrypyrim+TX, fluazaindolizine+TX, fluazuron+TX, flubendiamide+TX, flubenzimine+TX, fluchlordiniliprole+TX, flucitrinate+TX, flucycloxuron+TX, flucythrinate+TX, fluensulfone+TX, flufenerim+TX, flufenprox+TX, flufiprole+TX, fluhexafon+TX, flumethrin+TX, fluopyram+TX, flupentiofenox+TX, flupyradifurone+TX, flupyrimin+TX, fluralaner+TX, fluvalinate+TX, fluxametamide+TX, fosthiazate+TX, gamma-cyhalothrin+TX, guadipyr+TX, halofenozide+TX, halfenprox+TX, heptafluthrin+TX, hexythiazox+TX, hydramethylnon+TX, imicyafos+TX, imidacloprid+TX, imiprothrin+TX, indazapyroxamet+TX, indoxacarb+TX, iodomethane+TX, iprodione+TX, isocycloseram+TX, isothioate+TX, ivermectin+TX, kappa-bifenthrin+TX, kappa-tefluthrin+TX, lambda-Cyhalothrin+TX, lepimectin+TX, lotilaner+TX, lufenuron+TX, metaflumizone+TX, metaldehyde+TX, metam+TX, methomyl+TX, methoxyfenozide+TX, metofluthrin+TX, metolcarb+TX, mexacarbate+TX, milbemectin+TX, momfluorothrin+TX, niclosamide+TX, nicofluprole+TX; nitenpyram+TX, nithiazine+TX, omethoate+TX, oxamyl+TX, oxazosulfyl+TX, parathion-ethyl+TX, permethrin+TX, phenothrin+TX, phosphocarb+TX, piperonylbutoxide+TX, pirimicarb+TX, pirimiphos-ethyl+TX, pirimiphos-methyl+TX, Polyhedrosis virus+TX, prallethrin+TX, profenofos+TX, profluthrin+TX, propargite+TX, propetamphos+TX, propoxur+TX, prothiophos+TX, protrifenbute+TX, pyflubumide+TX, pymetrozine+TX, pyraclofos+TX, pyrafluprole+TX, pyridaben+TX, pyridalyl+TX, pyrifluquinazon+TX, pyrimidifen+TX, pyriminostrobin+TX, pyriprole+TX, pyriproxyfen+TX, resmethrin+TX, sarolaner+TX, selamectin+TX, silafluofen+TX, spinetoram+TX, spinosad+TX, spirobudifen+TX; spirodiclofen+TX, spiromesifen+TX, spiropidion+TX, spirotetramat+TX, spidoxamat+TX, sulfoxaflor+TX, tebufenozide+TX, tebufenpyrad+TX, tebupirimiphos+TX, tefluthrin+TX, temephos+TX, tetrachlorantraniliprole+TX, tetradiphon+TX, tetramethrin+TX, tetramethylfluthrin+TX, tetranactin+TX, tetraniliprole+TX, theta-cypermethrin+TX, thiacloprid+TX, thiamethoxam+TX, thiocyclam+TX, thiodicarb+TX, thiofanox+TX, thiometon+TX, thiosultap+TX, tigolaner+TX, tiorantraniliprole+TX; tioxazafen+TX, tolfenpyrad+TX, toxaphene+TX, tralomethrin+TX, transfluthrin+TX, triazamate+TX, triazophos+TX, trichlorfon+TX, trichloronate+TX, trichlorphon+TX, trifluenfuronate+TX, triflumezopyrim+TX, tyclopyrazoflor+TX, zeta-cypermethrin+TX, Extract of seaweed and fermentation product derived from melasse+TX, Extract of seaweed and fermentation product derived from melasse comprising urea+TX, amino acids+TX, potassium and molybdenum and EDTA-chelated manganese+TX, Extract of seaweed and fermented plant products+TX, Extract of seaweed and fermented plant products comprising phytohormones+TX, vitamins+TX, EDTA-chelated copper+TX, zinc+TX, and iron+TX, azadirachtin+TX, Bacillus aizawai+TX, Bacillus chitinosporus AQ746 (NRRL Accession No B-21 618)+TX, Bacillus firmus+TX, Bacillus kurstaki+TX, Bacillus mycoides AQ726 (NRRL Accession No. B-21664)+TX, Bacillus pumilus (NRRL Accession No B-30087)+TX, Bacillus pumilus AQ717 (NRRL Accession No. B-21662)+TX, Bacillus sp. AQ178 (ATCC Accession No. 53522)+TX, Bacillus sp. AQ175 (ATCC Accession No. 55608)+TX, Bacillus sp. AQ177 (ATCC Accession No. 55609)+TX, Bacillus subtilis unspecified+TX, Bacillus subtilis AQ153 (ATCC Accession No. 55614)+TX, Bacillus subtilis AQ30002 (NRRL Accession No. B-50421)+TX, Bacillus subtilis AQ30004 (NRRL Accession No. B-50455)+TX, Bacillus subtilis AQ713 (NRRL Accession No. B-21661)+TX, Bacillus subtilis AQ743 (NRRL Accession No. B-21665)+TX, Bacillus thuringiensis AQ52 (NRRL Accession No. B-21619)+TX, Bacillus thuringiensis BD #32 (NRRL Accession No B-21530)+TX, Bacillus thuringiensis subspec. kurstaki BMP 123+TX, Beauveria bassiana+TX, D-limonene+TX, Granulovirus+TX, Harpin+TX, Helicoverpa armigera Nucleopolyhedrovirus+TX, Helicoverpa zea Nucleopolyhedrovirus+TX, Heliothis virescens Nucleopolyhedrovirus+TX, Heliothis punctigera Nucleopolyhedrovirus+TX, Metarhizium spp.+TX, Muscodor albus 620 (NRRL Accession No. 30547)+TX, Muscodor roseus A3-5 (NRRL Accession No. 30548)+TX, Neem tree based products+TX, Paecilomyces fumosoroseus+TX, Paecilomyces lilacinus+TX, Pasteuria nishizawae+TX, Pasteuria penetrans+TX, Pasteuria ramosa+TX, Pasteuria thornei+TX, Pasteuria usgae+TX, P-cymene+TX, Plutella xylostella Granulosis virus+TX, Plutella xylostella Nucleopolyhedrovirus+TX, Polyhedrosis virus+TX, pyrethrum+TX, QRD 420 (a terpenoid blend)+TX, QRD 452 (a terpenoid blend)+TX, QRD 460 (a terpenoid blend)+TX, Quillaja saponaria+TX, Rhodococcus globerulus AQ719 (NRRL Accession No B-21663)+TX, Spodoptera frugiperda Nucleopolyhedrovirus+TX, Streptomyces galbus (NRRL Accession No. 30232)+TX, Streptomyces sp. (NRRL Accession No. B-30145)+TX, Terpenoid blend+TX, and Verticillium spp.+TX;
an algicide selected from the group of substances consisting of bethoxazin [CCN]+TX, copper dioctanoate (IUPAC name) (170)+TX, copper sulfate (172)+TX, cybutryne [CCN]+TX, dichlone (1052)+TX, dichlorophen (232)+TX, endothal (295)+TX, fentin (347)+TX, hydrated lime [CCN]+TX, nabam (566)+TX, quinoclamine (714)+TX, quinonamid (1379)+TX, simazine (730)+TX, triphenyltin acetate (IUPAC name) (347) and triphenyltin hydroxide (IUPAC name) (347)+TX;
an anthelmintic selected from the group of substances consisting of abamectin (1)+TX, crufomate (1011)+TX, cyclobutrifluram+TX, doramectin (alternative name) [CCN]+TX, emamectin (291)+TX, emamectin benzoate (291)+TX, eprinomectin (alternative name) [CCN]+TX, ivermectin (alternative name) [CCN]+TX, milbemycin oxime (alternative name) [CCN]+TX, moxidectin (alternative name) [CCN]+TX, piperazine [CCN]+TX, selamectin (alternative name) [CCN]+TX, spinosad (737) and thiophanate (1435)+TX;
an avicide selected from the group of substances consisting of chloralose (127)+TX, endrin (1122)+TX, fenthion (346)+TX, pyridin-4-amine (IUPAC name) (23) and strychnine (745)+TX; a bactericide selected from the group of substances consisting of 1-hydroxy-1H-pyridine-2-thione (IUPAC name) (1222)+TX, 4-(quinoxalin-2-ylamino)benzenesulfonamide (IUPAC name) (748)+TX, 8-hydroxyquinoline sulfate (446)+TX, bronopol (97)+TX, copper dioctanoate (IUPAC name) (170)+TX, copper hydroxide (IUPAC name) (169)+TX, cresol [CCN]+TX, dichlorophen (232)+TX, dipyrithione (1105)+TX, dodicin (1112)+TX, fenaminosulf (1144)+TX, formaldehyde (404)+TX, hydrargaphen (alternative name) [CCN]+TX, kasugamycin (483)+TX, kasugamycin hydrochloride hydrate (483)+TX, nickel bis(dimethyldithiocarbamate) (IUPAC name) (1308)+TX, nitrapyrin (580)+TX, octhilinone (590)+TX, oxolinic acid (606)+TX, oxytetracycline (611)+TX, potassium hydroxyquinoline sulfate (446)+TX, probenazole (658)+TX, streptomycin (744)+TX, streptomycin sesquisulfate (744)+TX, tecloftalam (766)+TX, and thiomersal (alternative name) [CCN]+TX;
a biological agent selected from the group of substances consisting of Adoxophyes orana GV (alternative name) (12)+TX, Agrobacterium radiobacter (alternative name) (13)+TX, Amblyseius spp. (alternative name) (19)+TX, Anagrapha falcifera NPV (alternative name) (28)+TX, Anagrus atomus (alternative name) (29)+TX, Aphelinus abdominalis (alternative name) (33)+TX, Aphidius colemani (alternative name) (34)+TX, Aphidoletes aphidimyza (alternative name) (35)+TX, Autographa californica NPV (alternative name) (38)+TX, Bacillus firmus (alternative name) (48)+TX, Bacillus sphaericus Neide (scientific name) (49)+TX, Bacillus thuringiensis Berliner (scientific name) (51)+TX, Bacillus thuringiensis subsp. aizawai (scientific name) (51)+TX, Bacillus thuringiensis subsp. israelensis (scientific name) (51)+TX, Bacillus thuringiensis subsp. japonensis (scientific name) (51)+TX, Bacillus thuringiensis subsp. kurstaki (scientific name) (51)+TX, Bacillus thuringiensis subsp. tenebrionis (scientific name) (51)+TX, Beauveria bassiana (alternative name) (53)+TX, Beauveria brongniartii (alternative name) (54)+TX, Chrysoperla carnea (alternative name) (151)+TX, Cryptolaemus montrouzieri (alternative name) (178)+TX, Cydia pomonella GV (alternative name) (191)+TX, Dacnusa sibirica (alternative name) (212)+TX, Diglyphus isaea (alternative name) (254)+TX, Encarsia formosa (scientific name) (293)+TX, Eretmocerus eremicus (alternative name) (300)+TX, Helicoverpa zea NPV (alternative name) (431)+TX, Heterorhabditis bacteriophora and H. megidis (alternative name) (433)+TX, Hippodamia convergens (alternative name) (442)+TX, Leptomastix dactylopii (alternative name) (488)+TX, Macrolophus caliginosus (alternative name) (491)+TX, Mamestra brassicae NPV (alternative name) (494)+TX, Metaphycus helvolus (alternative name) (522)+TX, Metarhizium anisopliae var. acridum (scientific name) (523)+TX, Metarhizium anisopliae var. anisopliae (scientific name) (523)+TX, Neodiprion sertifer NPV and N. lecontei NPV (alternative name) (575)+TX, Orius spp. (alternative name) (596)+TX, Paecilomyces fumosoroseus (alternative name) (613)+TX, Phytoseiulus persimilis (alternative name) (644)+TX, Spodoptera exigua multicapsid nuclear polyhedrosis virus (scientific name) (741)+TX, Steinernema bibionis (alternative name) (742)+TX, Steinernema carpocapsae (alternative name) (742)+TX, Steinernema feltiae (alternative name) (742)+TX, Steinernema glaseri (alternative name) (742)+TX, Steinernema riobrave (alternative name) (742)+TX, Steinernema riobravis (alternative name) (742)+TX, Steinernema scapterisci (alternative name) (742)+TX, Steinernema spp. (alternative name) (742)+TX, Trichogramma spp. (alternative name) (826)+TX, Typhlodromus occidentalis (alternative name) (844) and Verticillium lecanii (alternative name) (848)+TX;
a soil sterilant selected from the group of substances consisting of iodomethane (IUPAC name) (542) and methyl bromide (537)+TX;
a chemosterilant selected from the group of substances consisting of apholate [CCN]+TX, bisazir (alternative name) [CCN]+TX, busulfan (alternative name) [CCN]+TX, diflubenzuron (250)+TX, dimatif (alternative name) [CCN]+TX, hemel [CCN]+TX, hempa [CCN]+TX, metepa [CCN]+TX, methiotepa [CCN]+TX, methyl apholate [CCN]+TX, morzid [CCN]+TX, penfluron (alternative name) [CCN]+TX, tepa [CCN]+TX, thiohempa (alternative name) [CCN]+TX, thiotepa (alternative name) [CCN]+TX, tretamine (alternative name) [CCN] and uredepa (alternative name) [CCN]+TX;
an insect pheromone selected from the group of substances consisting of (E)-dec-5-en-1-yl acetate with (E)-dec-5-en-1-ol (IUPAC name) (222)+TX, (E)-tridec-4-en-1-yl acetate (IUPAC name) (829)+TX, (E)-6-methylhept-2-en-4-ol (IUPAC name) (541)+TX, (E,Z)-tetradeca-4,10-dien-1-yl acetate (IUPAC name) (779)+TX, (Z)-dodec-7-en-1-yl acetate (IUPAC name) (285)+TX, (Z)-hexadec-11-enal (IUPAC name) (436)+TX, (Z)-hexadec-11-en-1-yl acetate (IUPAC name) (437)+TX, (Z)-hexadec-13-en-11-yn-1-yl acetate (IUPAC name) (438)+TX, (Z)-icos-13-en-10-one (IUPAC name) (448)+TX, (Z)-tetradec-7-en-1-al (IUPAC name) (782)+TX, (Z)-tetradec-9-en-1-ol (IUPAC name) (783)+TX, (Z)-tetradec-9-en-1-yl acetate (IUPAC name) (784)+TX, (7E,9Z)-dodeca-7,9-dien-1-yl acetate (IUPAC name) (283)+TX, (9Z,11E)-tetradeca-9,11-dien-1-yl acetate (IUPAC name) (780)+TX, (9Z,12E)-tetradeca-9,12-dien-1-yl acetate (IUPAC name) (781)+TX, 14-methyloctadec-1-ene (IUPAC name) (545)+TX, 4-methylnonan-5-ol with 4-methylnonan-5-one (IUPAC name) (544)+TX, alpha-multistriatin (alternative name) [CCN]+TX, brevicomin (alternative name) [CCN]+TX, codlelure (alternative name) [CCN]+TX, codlemone (alternative name) (167)+TX, cuelure (alternative name) (179)+TX, disparlure (277)+TX, dodec-8-en-1-yl acetate (IUPAC name) (286)+TX, dodec-9-en-1-yl acetate (IUPAC name) (287)+TX, dodeca-8+TX, 10-dien-1-yl acetate (IUPAC name) (284)+TX, dominicalure (alternative name) [CCN]+TX, ethyl 4-methyloctanoate (IUPAC name) (317)+TX, eugenol (alternative name) [CCN]+TX, frontalin (alternative name) [CCN]+TX, Gossyplure® (alternative name; 1:1 mixture of the (Z,E) and (Z,Z) isomers of hexadeca-7,11-dien-1-yl-acetate) (420)+TX, grandlure (421)+TX, grandlure I (alternative name) (421)+TX, grandlure II (alternative name) (421)+TX, grandlure III (alternative name) (421)+TX, grandlure IV (alternative name) (421)+TX, hexalure [CCN]+TX, ipsdienol (alternative name) [CCN]+TX, ipsenol (alternative name) [CCN]+TX, japonilure (alternative name) (481)+TX, lineatin (alternative name) [CCN]+TX, litlure (alternative name) [CCN]+TX, looplure (alternative name) [CCN]+TX, medlure [CCN]+TX, megatomoic acid (alternative name) [CCN]+TX, methyl eugenol (alternative name) (540)+TX, muscalure (563)+TX, octadeca-2,13-dien-1-yl acetate (IUPAC name) (588)+TX, octadeca-3,13-dien-1-yl acetate (IUPAC name) (589)+TX, orfralure (alternative name) [CCN]+TX, oryctalure (alternative name) (317)+TX, ostramone (alternative name) [CCN]+TX, siglure [CCN]+TX, sordidin (alternative name) (736)+TX, sulcatol (alternative name) [CCN]+TX, tetradec-11-en-1-yl acetate (IUPAC name) (785)+TX, trimedlure (839)+TX, trimedlure A (alternative name) (839)+TX, trimedlure B₁ (alternative name) (839)+TX, trimedlure B2 (alternative name) (839)+TX, trimedlure C (alternative name) (839) and trunc-call (alternative name) [CCN]+TX;
an insect repellent selected from the group of substances consisting of 2-(octylthio)ethanol (IUPAC name) (591)+TX, butopyronoxyl (933)+TX, butoxy(polypropylene glycol) (936)+TX, dibutyl adipate (IUPAC name) (1046)+TX, dibutyl phthalate (1047)+TX, dibutyl succinate (IUPAC name) (1048)+TX, diethyltoluamide [CCN]+TX, dimethyl carbate [CCN]+TX, dimethyl phthalate [CCN]+TX, ethyl hexanediol (1137)+TX, hexamide [CCN]+TX, methoquin-butyl (1276)+TX, methylneodecanamide [CCN]+TX, oxamate [CCN] and picaridin [CCN]+TX;
a molluscicide selected from the group of substances consisting of bis(tributyltin) oxide (IUPAC name) (913)+TX, bromoacetamide [CCN]+TX, calcium arsenate [CCN]+TX, cloethocarb (999)+TX, copper acetoarsenite [CCN]+TX, copper sulfate (172)+TX, fentin (347)+TX, ferric phosphate (IUPAC name) (352)+TX, metaldehyde (518)+TX, methiocarb (530)+TX, niclosamide (576)+TX, niclosamide-olamine (576)+TX, pentachlorophenol (623)+TX, sodium pentachlorophenoxide (623)+TX, tazimcarb (1412)+TX, thiodicarb (799)+TX, tributyltin oxide (913)+TX, trifenmorph (1454)+TX, trimethacarb (840)+TX, triphenyltin acetate (IUPAC name) (347) and triphenyltin hydroxide (IUPAC name) (347)+TX, pyriprole [394730-71-3]+TX;
a nematicide selected from the group of substances consisting of AKD-3088 (compound code)+TX, 1,2-dibromo-3-chloropropane (IUPAC/Chemical Abstracts name) (1045)+TX, 1,2-dichloropropane (IUPAC/Chemical Abstracts name) (1062)+TX, 1,2-dichloropropane with 1,3-dichloropropene (IUPAC name) (1063)+TX, 1,3-dichloropropene (233)+TX, 3,4-dichlorotetrahydrothiophene 1,1-dioxide (IUPAC/Chemical Abstracts name) (1065)+TX, 3-(4-chlorophenyl)-5-methylrhodanine (IUPAC name) (980)+TX, 5-methyl-6-thioxo-1,3,5-thiadiazinan-3-ylacetic acid (IUPAC name) (1286)+TX, 6-isopentenylaminopurine (alternative name) (210)+TX, abamectin (1)+TX, acetoprole [CCN]+TX, alanycarb (15)+TX, aldicarb (16)+TX, aldoxycarb (863)+TX, AZ 60541 (compound code)+TX, benclothiaz [CCN]+TX, benomyl (62)+TX, butylpyridaben (alternative name)+TX, cadusafos (109)+TX, carbofuran (118)+TX, carbon disulfide (945)+TX, carbosulfan (119)+TX, chloropicrin (141)+TX, chlorpyrifos (145)+TX, cloethocarb (999)+TX, cyclobutrifluram+TX, cytokinins (alternative name) (210)+TX, dazomet (216)+TX, DBCP (1045)+TX, DCIP (218)+TX, diamidafos (1044)+TX, dichlofenthion (1051)+TX, dicliphos (alternative name)+TX, dimethoate (262)+TX, doramectin (alternative name) [CCN]+TX, emamectin (291)+TX, emamectin benzoate (291)+TX, eprinomectin (alternative name) [CCN]+TX, ethoprophos (312)+TX, ethylene dibromide (316)+TX, fenamiphos (326)+TX, fenpyrad (alternative name)+TX, fensulfothion (1158)+TX, fosthiazate (408)+TX, fosthietan (1196)+TX, furfural (alternative name) [CCN]+TX, GY-81 (development code) (423)+TX, heterophos [CCN]+TX, iodomethane (IUPAC name) (542)+TX, isamidofos (1230)+TX, isazofos (1231)+TX, ivermectin (alternative name) [CCN]+TX, kinetin (alternative name) (210)+TX, mecarphon (1258)+TX, metam (519)+TX, metam-potassium (alternative name) (519)+TX, metam-sodium (519)+TX, methyl bromide (537)+TX, methyl isothiocyanate (543)+TX, milbemycin oxime (alternative name) [CCN]+TX, moxidectin (alternative name) [CCN]+TX, Myrothecium verrucaria composition (alternative name) (565)+TX, NC-184 (compound code)+TX, oxamyl (602)+TX, phorate (636)+TX, phosphamidon (639)+TX, phosphocarb [CCN]+TX, sebufos (alternative name)+TX, selamectin (alternative name) [CCN]+TX, spinosad (737)+TX, terbam (alternative name)+TX, terbufos (773)+TX, tetrachlorothiophene (IUPAC/Chemical Abstracts name) (1422)+TX, thiafenox (alternative name)+TX, thionazin (1434)+TX, triazophos (820)+TX, triazuron (alternative name)+TX, xylenols [CCN]+TX, YI-5302 (compound code) and zeatin (alternative name) (210)+TX, fluensulfone [318290-98-1]+TX, fluopyram+TX;
a nitrification inhibitor selected from the group of substances consisting of potassium ethylxanthate [CCN] and nitrapyrin (580)+TX;
a plant activator selected from the group of substances consisting of acibenzolar (6)+TX, acibenzolar-S-methyl (6)+TX, probenazole (658) and Reynoutria sachalinensis extract (alternative name) (720)+TX;
a rodenticide selected from the group of substances consisting of 2-isovalerylindan-1,3-dione (IUPAC name) (1246)+TX, 4-(quinoxalin-2-ylamino)benzenesulfonamide (IUPAC name) (748)+TX, alpha-chlorohydrin [CCN]+TX, aluminium phosphide (640)+TX, antu (880)+TX, arsenous oxide (882)+TX, barium carbonate (891)+TX, bisthiosemi (912)+TX, brodifacoum (89)+TX, bromadiolone (including alpha-bromadiolone)+TX, bromethalin (92)+TX, calcium cyanide (444)+TX, chloralose (127)+TX, chlorophacinone (140)+TX, cholecalciferol (alternative name) (850)+TX, coumachlor (1004)+TX, coumafuryl (1005)+TX, coumatetralyl (175)+TX, crimidine (1009)+TX, difenacoum (246)+TX, difethialone (249)+TX, diphacinone (273)+TX, ergocalciferol (301)+TX, flocoumafen (357)+TX, fluoroacetamide (379)+TX, flupropadine (1183)+TX, flupropadine hydrochloride (1183)+TX, gamma-HCH (430)+TX, HCH (430)+TX, hydrogen cyanide (444)+TX, iodomethane (IUPAC name) (542)+TX, lindane (430)+TX, magnesium phosphide (IUPAC name) (640)+TX, methyl bromide (537)+TX, norbormide (1318)+TX, phosacetim (1336)+TX, phosphine (IUPAC name) (640)+TX, phosphorus [CCN]+TX, pindone (1341)+TX, potassium arsenite [CCN]+TX, pyrinuron (1371)+TX, scilliroside (1390)+TX, sodium arsenite [CCN]+TX, sodium cyanide (444)+TX, sodium fluoroacetate (735)+TX, strychnine (745)+TX, thallium sulfate [CCN]+TX, warfarin (851) and zinc phosphide (640)+TX; a synergist selected from the group of substances consisting of 2-(2-butoxyethoxy)ethyl piperonylate (IUPAC name) (934)+TX, 5-(1,3-benzodioxol-5-yl)-3-hexylcyclohex-2-enone (IUPAC name) (903)+TX, farnesol with nerolidol (alternative name) (324)+TX, MB-599 (development code) (498)+TX, MGK 264 (development code) (296)+TX, piperonyl butoxide (649)+TX, piprotal (1343)+TX, propyl isomer (1358)+TX, S421 (development code) (724)+TX, sesamex (1393)+TX, sesasmolin (1394) and sulfoxide (1406)+TX;
an animal repellent selected from the group of substances consisting of anthraquinone (32)+TX, chloralose (127)+TX, copper naphthenate [CCN]+TX, copper oxychloride (171)+TX, diazinon (227)+TX, dicyclopentadiene (chemical name) (1069)+TX, guazatine (422)+TX, guazatine acetates (422)+TX, methiocarb (530)+TX, pyridin-4-amine (IUPAC name) (23)+TX, thiram (804)+TX, trimethacarb (840)+TX, zinc naphthenate [CCN] and ziram (856)+TX; a virucide selected from the group of substances consisting of imanin (alternative name) [CCN] and ribavirin (alternative name) [CCN]+TX;
a wound protectant selected from the group of substances consisting of mercuric oxide (512)+TX, octhilinone (590) and thiophanate-methyl (802)+TX;
a biologically active substance selected from 1,1-bis(4-chlorophenyl)-2-ethoxyethanol+TX, 2,4-dichlorophenyl benzenesulfonate+TX, 2-fluoro-N-methyl-N-1-naphthylacetamide+TX, 4-chlorophenyl phenyl sulfone+TX, acetoprole+TX, aldoxycarb+TX, amidithion+TX, amidothioate+TX, amiton+TX, amiton hydrogen oxalate+TX, amitraz+TX, aramite+TX, arsenous oxide+TX, azobenzene+TX, azothoate+TX, benomyl+TX, benoxafos+TX, benzyl benzoate+TX, bixafen+TX, brofenvalerate+TX, bromocyclen+TX, bromophos+TX, bromopropylate+TX, buprofezin+TX, butocarboxim+TX, butoxycarboxim+TX, butylpyridaben+TX, calcium polysulfide+TX, camphechlor+TX, carbanolate+TX, carbophenothion+TX, cymiazole+TX, chinomethionat+TX, chlorbenside+TX, chlordimeform+TX, chlordimeform hydrochloride+TX, chlorfenethol+TX, chlorfenson+TX, chlorfensulfide+TX, chlorobenzilate+TX, chloromebuform+TX, chloromethiuron+TX, chloropropylate+TX, chlorthiophos+TX, cinerin I+TX, cinerin II+TX, cinerins+TX, closantel+TX, coumaphos+TX, crotamiton+TX, crotoxyphos+TX, cufraneb+TX, cyanthoate+TX, DCPM+TX, DDT+TX, demephion+TX, demephion-O+TX, demephion-S+TX, demeton-methyl+TX, demeton-O+TX, demeton-O-methyl+TX, demeton-S+TX, demeton-S-methyl+TX, demeton-S-methylsulfon+TX, dichlofluanid+TX, dichlorvos+TX, dicliphos+TX, dienochlor+TX, dimefox+TX, dinex+TX, dinex-diclexine+TX, dinocap-4+TX, dinocap-6+TX, dinocton+TX, dinopenton+TX, dinosulfon+TX, dinoterbon+TX, dioxathion+TX, diphenyl sulfone+TX, disulfiram+TX, DNOC+TX, dofenapyn+TX, doramectin+TX, endothion+TX, eprinomectin+TX, ethoate-methyl+TX, etrimfos+TX, fenazaflor+TX, fenbutatin oxide+TX, fenothiocarb+TX, fenpyrad+TX, fenpyroximate+TX, fenpyrazamine+TX, fenson+TX, fentrifanil+TX, flubenzimine+TX, flucycloxuron+TX, fluenetil+TX, fluorbenside+TX, FMC 1137+TX, formetanate+TX, formetanate hydrochloride+TX, formparanate+TX, gamma-HCH+TX, glyodin+TX, halfenprox+TX, hexadecyl cyclopropanecarboxylate+TX, isocarbophos+TX, jasmolin I+TX, jasmolin II+TX, jodfenphos+TX, lindane+TX, malonoben+TX, mecarbam+TX, mephosfolan+TX, mesulfen+TX, methacrifos+TX, methyl bromide+TX, metolcarb+TX, mexacarbate+TX, milbemycin oxime+TX, mipafox+TX, monocrotophos+TX, morphothion+TX, moxidectin+TX, naled+TX, 4-chloro-2-(2-chloro-2-methyl-propyl)-5-[(6-iodo-3-pyridyl)methoxy]pyridazin-3-one+TX, nifluridide+TX, nikkomycins+TX, nitrilacarb+TX, nitrilacarb 1:1 zinc chloride complex+TX, omethoate+TX, oxydeprofos+TX, oxydisulfoton+TX, pp′-DDT+TX, parathion+TX, permethrin+TX, phenkapton+TX, phosalone+TX, phosfolan+TX, phosphamidon+TX, polychloroterpenes+TX, polynactins+TX, proclonol+TX, promacyl+TX, propoxur+TX, prothidathion+TX, prothoate+TX, pyrethrin I+TX, pyrethrin II+TX, pyrethrins+TX, pyridaphenthion+TX, pyrimitate+TX, quinalphos+TX, quintiofos+TX, R-1492+TX, phosglycin+TX, rotenone+TX, schradan+TX, sebufos+TX, selamectin+TX, sophamide+TX, SSI-121+TX, sulfiram+TX, sulfluramid+TX, sulfotep+TX, sulfur+TX, diflovidazin+TX, tau-fluvalinate+TX, TEPP+TX, terbam+TX, tetradifon+TX, tetrasul+TX, thiafenox+TX, thiocarboxime+TX, thiofanox+TX, thiometon+TX, thioquinox+TX, thuringiensin+TX, triamiphos+TX, triarathene+TX, triazophos+TX, triazuron+TX, trifenofos+TX, trinactin+TX, vamidothion+TX, vaniliprole+TX, bethoxazin+TX, copper dioctanoate+TX, copper sulfate+TX, cybutryne+TX, dichlone+TX, dichlorophen+TX, endothal+TX, fentin+TX, hydrated lime+TX, nabam+TX, quinoclamine+TX, quinonamid+TX, simazine+TX, triphenyltin acetate+TX, triphenyltin hydroxide+TX, crufomate+TX, piperazine+TX, thiophanate+TX, chloralose+TX, fenthion+TX, pyridin-4-amine+TX, strychnine+TX, 1-hydroxy-1H-pyridine-2-thione+TX, 4-(quinoxalin-2-ylamino)benzenesulfonamide+TX, 8-hydroxyquinoline sulfate+TX, bronopol+TX, copper hydroxide+TX, cresol+TX, dipyrithione+TX, dodicin+TX, fenaminosulf+TX, formaldehyde+TX, hydrargaphen+TX, kasugamycin+TX, kasugamycin hydrochloride hydrate+TX, nickel bis(dimethyldithiocarbamate)+TX, nitrapyrin+TX, octhilinone+TX, oxolinic acid+TX, oxytetracycline+TX, potassium hydroxyquinoline sulfate+TX, probenazole+TX, streptomycin+TX, streptomycin sesquisulfate+TX, tecloftalam+TX, thiomersal+TX, Adoxophyes orana GV+TX, Agrobacterium radiobacter+TX, Amblyseius spp.+TX, Anagrapha falcifera NPV+TX, Anagrus atomus+TX, Aphelinus abdominalis+TX, Aphidius colemani+TX, Aphidoletes aphidimyza+TX, Autographa californica NPV+TX, Bacillus sphaericus Neide+TX, Beauveria brongniartii+TX, Chrysoperla carnea+TX, Cryptolaemus montrouzieri+TX, Cydia pomonella GV+TX, Dacnusa sibirica+TX, Diglyphus isaea+TX, Encarsia formosa+TX, Eretmocerus eremicus+TX, Heterorhabditis bacteriophora and H. megidis+TX, Hippodamia convergens+TX, Leptomastix dactylopii+TX, Macrolophus caliginosus+TX, Mamestra brassicae NPV+TX, Metaphycus helvolus+TX, Metarhizium anisopliae var. acridum+TX, Metarhizium anisopliae var. anisopliae+TX, Neodiprion sertifer NPV and N. lecontei NPV+TX, Orius spp.+TX, Paecilomyces fumosoroseus+TX, Phytoseiulus persimilis+TX, Steinernema bibionis+TX, Steinernema carpocapsae+TX, Steinernema feltiae+TX, Steinernema glaseri+TX, Steinernema riobrave+TX, Steinernema riobravis+TX, Steinernema scapterisci+TX, Steinernema spp.+TX, Trichogramma spp.+TX, Typhlodromus occidentalis+TX, Verticillium lecanii+TX, apholate+TX, bisazir+TX, busulfan+TX, dimatif+TX, hemel+TX, hempa+TX, metepa+TX, methiotepa+TX, methyl apholate+TX, morzid+TX, penfluron+TX, tepa+TX, thiohempa+TX, thiotepa+TX, tretamine+TX, uredepa+TX, (E)-dec-5-en-1-yl acetate with (E)-dec-5-en-1-ol+TX, (E)-tridec-4-en-1-yl acetate+TX, (E)-6-methylhept-2-en-4-ol+TX, (E,Z)-tetradeca-4,10-dien-1-yl acetate+TX, (Z)-dodec-7-en-1-yl acetate+TX, (Z)-hexadec-11-enal+TX, (Z)-hexadec-11-en-1-yl acetate+TX, (Z)-hexadec-13-en-11-yn-1-yl acetate+TX, (Z)-icos-13-en-10-one+TX, (Z)-tetradec-7-en-1-al+TX, (Z)-tetradec-9-en-1-ol+TX, (Z)-tetradec-9-en-1-yl acetate+TX, (7E,9Z)-dodeca-7,9-dien-1-yl acetate+TX, (9Z,11E)-tetradeca-9,11-dien-1-yl acetate+TX, (9Z,12E)-tetradeca-9,12-dien-1-yl acetate+TX, 14-methyloctadec-1-ene+TX, 4-methylnonan-5-ol with 4-methylnonan-5-one+TX, alpha-multistriatin+TX, brevicomin+TX, codlelure+TX, codlemone+TX, cuelure+TX, disparlure+TX, dodec-8-en-1-yl acetate+TX, dodec-9-en-1-yl acetate+TX, dodeca-8+TX, 10-dien-1-yl acetate+TX, dominicalure+TX, ethyl 4-methyloctanoate+TX, eugenol+TX, frontalin+TX, grandlure+TX, grandlure I+TX, 5 grandlure II+TX, grandlure IlIl+TX, grandlure IV+TX, hexalure+TX, ipsdienol+TX, ipsenol+TX, japonilure+TX, lineatin+TX, litlure+TX, looplure+TX, medlure+TX, megatomoic acid+TX, methyl eugenol+TX, muscalure+TX, octadeca-2,13-dien-1-yl acetate+TX, octadeca-3,13-dien-1-yl acetate+TX, orfralure+TX, oryctalure+TX, ostramone+TX, siglure+TX, sordidin+TX, sulcatol+TX, tetradec-11-en-1-yl acetate+TX, trimedlure+TX, trimedlure A+TX, trimedlure B₁+TX, trimedlure B2+TX, trimedlure C+TX, trunc-call+TX, 2-(octylthio)ethanol+TX, butopyronoxyl+TX, butoxy(polypropylene glycol)+TX, dibutyl adipate+TX, dibutyl phthalate+TX, dibutyl succinate+TX, diethyltoluamide+TX, dimethyl carbate+TX, dimethyl phthalate+TX, ethyl hexanediol+TX, hexamide+TX, methoquin-butyl+TX, methylneodecanamide+TX, oxamate+TX, picaridin+TX, 1-dichloro-1-nitroethane+TX, 1,1-dichloro-2,2-bis(4-ethylphenyl)ethane+TX, 1,2-dichloropropane with 1,3-dichloropropene+TX, 1-bromo-2-chloroethane+TX, 2,2,2-trichloro-1-(3,4-dichlorophenyl)ethyl acetate+TX, 2,2-dichlorovinyl 2-ethylsulfinylethyl methyl phosphate+TX, 2-(1,3-dithiolan-2-yl)phenyl dimethylcarbamate+TX, 2-(2-butoxyethoxy)ethyl thiocyanate+TX, 2-(4,5-dimethyl-1,3-dioxolan-2-yl)phenyl methylcarbamate+TX, 2-(4-chloro-3,5-xylyloxy)ethanol+TX, 2-chlorovinyl diethyl phosphate+TX, 2-imidazolidone+TX, 2-isovalerylindan-1,3-dione+TX, 2-methyl(prop-2-ynyl)aminophenyl methylcarbamate+TX, 2-thiocyanatoethyl laurate+TX, 3-bromo-1-chloroprop-1-ene+TX, 3-methyl-1-phenylpyrazol-5-yl dimethylcarbamate+TX, 4-methyl(prop-2-ynyl)amino-3,5-xylyl methylcarbamate+TX, 5,5-dimethyl-3-oxocyclohex-1-enyl dimethylcarbamate+TX, acethion+TX, acrylonitrile+TX, aldrin+TX, allosamidin+TX, allyxycarb+TX, alpha-ecdysone+TX, aluminium phosphide+TX, aminocarb+TX, anabasine+TX, athidathion+TX, azamethiphos+TX, Bacillus thuringiensis delta endotoxins+TX, barium hexafluorosilicate+TX, barium polysulfide+TX, barthrin+TX, Bayer 22/190+TX, Bayer 22408+TX, beta-cyfluthrin+TX, beta-cypermethrin+TX, bioethanomethrin+TX, biopermethrin+TX, bis(2-chloroethyl) ether+TX, borax+TX, bromfenvinfos+TX, bromo-DDT+TX, bufencarb+TX, butacarb+TX, butathiofos+TX, butonate+TX, calcium arsenate+TX, calcium cyanide+TX, carbon disulfide+TX, carbon tetrachloride+TX, cartap hydrochloride+TX, cevadine+TX, chlorbicyclen+TX, chlordane+TX, chlordecone+TX, chloroform+TX, chloropicrin+TX, chlorphoxim+TX, chlorprazophos+TX, cis-resmethrin+TX, cismethrin+TX, clocythrin+TX, copper acetoarsenite+TX, copper arsenate+TX, copper oleate+TX, coumithoate+TX, cryolite+TX, CS 708+TX, cyanofenphos+TX, cyanophos+TX, cyclethrin+TX, cythioate+TX, d-tetramethrin+TX, DAEP+TX, dazomet+TX, decarbofuran+TX, diamidafos+TX, dicapthon+TX, dichlofenthion+TX, dicresyl+TX, dicyclanil+TX, dieldrin+TX, diethyl 5-methylpyrazol-3-yl phosphate+TX, dilor+TX, dimefluthrin+TX, dimetan+TX, dimethrin+TX, dimethylvinphos+TX, dimetilan+TX, dinoprop+TX, dinosam+TX, dinoseb+TX, diofenolan+TX, dioxabenzofos+TX, dithicrofos+TX, DSP+TX, ecdysterone+TX, EI 1642+TX, EMPC+TX, EPBP+TX, etaphos+TX, ethiofencarb+TX, ethyl formate+TX, ethylene dibromide+TX, ethylene dichloride+TX, ethylene oxide+TX, EXD+TX, fenchlorphos+TX, fenethacarb+TX, fenitrothion+TX, fenoxacrim+TX, fenpirithrin+TX, fensulfothion+TX, fenthion-ethyl+TX, flucofuron+TX, fosmethilan+TX, fospirate+TX, fosthietan+TX, furathiocarb+TX, furethrin+TX, guazatine+TX, guazatine acetates+TX, sodium tetrathiocarbonate+TX, halfenprox+TX, HCH+TX, HEOD+TX, heptachlor+TX, heterophos+TX, HHDN+TX, hydrogen cyanide+TX, hyquincarb+TX, IPSP+TX, isazofos+TX, isobenzan+TX, isodrin+TX, isofenphos+TX, isolane+TX, isoprothiolane+TX, isoxathion+TX, juvenile hormone I+TX, juvenile hormone II+TX, juvenile hormone IlIl+TX, kelevan+TX, kinoprene+TX, lead arsenate+TX, leptophos+TX, lirimfos+TX, lythidathion+TX, m-cumenyl methylcarbamate+TX, magnesium phosphide+TX, mazidox+TX, mecarphon+TX, menazon+TX, mercurous chloride+TX, mesulfenfos+TX, metam+TX, metam-potassium+TX, metam-sodium+TX, methanesulfonyl fluoride+TX, methocrotophos+TX, methoprene+TX, methothrin+TX, methoxychlor+TX, methyl isothiocyanate+TX, methylchloroform+TX, methylene chloride+TX, metoxadiazone+TX, mirex+TX, naftalofos+TX, naphthalene+TX, NC-170+TX, nicotine+TX, nicotine sulfate+TX, nithiazine+TX, nornicotine+TX, O-5-dichloro-4-iodophenyl O-ethyl ethylphosphonothioate+TX, O,O-diethyl O-4-methyl-2-oxo-2H-chromen-7-yl phosphorothioate+TX, O,O-diethyl O-6-methyl-2-propylpyrimidin-4-yl phosphorothioate+TX, O,O,O′,O′-tetrapropyl dithiopyrophosphate+TX, oleic acid+TX, para-dichlorobenzene+TX, parathion-methyl+TX, pentachlorophenol+TX, pentachlorophenyl laurate+TX, PH 60-38+TX, phenkapton+TX, phosnichlor+TX, phosphine+TX, phoxim-methyl+TX, pirimetaphos+TX, polychlorodicyclopentadiene isomers+TX, potassium arsenite+TX, potassium thiocyanate+TX, precocene I+TX, precocene II+TX, precocene III+TX, primidophos+TX, profluthrin+TX, promecarb+TX, prothiofos+TX, pyrazophos+TX, pyresmethrin+TX, quassia+TX, quinalphos-methyl+TX, quinothion+TX, rafoxanide+TX, resmethrin+TX, rotenone+TX, kadethrin+TX, ryania+TX, ryanodine+TX, sabadilla+TX, schradan+TX, sebufos+TX, SI-0009+TX, thiapronil+TX, sodium arsenite+TX, sodium cyanide+TX, sodium fluoride+TX, sodium hexafluorosilicate+TX, sodium pentachlorophenoxide+TX, sodium selenate+TX, sodium thiocyanate+TX, sulcofuron+TX, sulcofuron-sodium+TX, sulfuryl fluoride+TX, sulprofos+TX, tar oils+TX, tazimcarb+TX, TDE+TX, tebupirimfos+TX, temephos+TX, terallethrin+TX, tetrachloroethane+TX, thicrofos+TX, thiocyclam+TX, thiocyclam hydrogen oxalate+TX, thionazin+TX, thiosultap+TX, thiosultap-sodium+TX, tralomethrin+TX, transpermethrin+TX, triazamate+TX, trichlormetaphos-3+TX, trichloronat+TX, trimethacarb+TX, tolprocarb+TX, triclopyricarb+TX, triprene+TX, veratridine+TX, veratrine+TX, XMC+TX, zetamethrin+TX, zinc phosphide+TX, zolaprofos+TX, meperfluthrin+TX, tetramethylfluthrin+TX, bis(tributyltin) oxide+TX, bromoacetamide+TX, ferric phosphate+TX, niclosamide-olamine+TX, tributyltin oxide+TX, pyrimorph+TX, trifenmorph+TX, 1,2-dibromo-3-chloropropane+TX, 1,3-dichloropropene+TX, 3,4-dichlorotetrahydrothiophene 1,1-dioxide+TX, 3-(4-chlorophenyl)-5-methylrhodanine+TX, 5-methyl-6-thioxo-1,3,5-thiadiazinan-3-ylacetic acid+TX, 6-isopentenylaminopurine+TX, anisiflupurin+TX, benclothiaz+TX, cytokinins+TX, DCIP+TX, furfural+TX, isamidofos+TX, kinetin+TX, Myrothecium verrucaria composition+TX, tetrachlorothiophene+TX, xylenols+TX, zeatin+TX, potassium ethylxanthate+TX,acibenzolar+TX, acibenzolar-S-methyl+TX, Reynoutria sachalinensis extract+TX, alpha-chlorohydrin+TX, antu+TX, barium carbonate+TX, bisthiosemi+TX, brodifacoum+TX, bromadiolone+TX, bromethalin+TX, chlorophacinone+TX, cholecalciferol+TX, coumachlor+TX, coumafuryl+TX, coumatetralyl+TX, crimidine+TX, difenacoum+TX, difethialone+TX, diphacinone+TX, ergocalciferol+TX, flocoumafen+TX, fluoroacetamide+TX, flupropadine+TX, flupropadine hydrochloride+TX, norbormide+TX, phosacetim+TX, phosphorus+TX, pindone+TX, pyrinuron+TX, scilliroside+TX, sodium fluoroacetate+TX, thallium sulfate+TX, warfarin+TX, 2-(2-butoxyethoxy)ethyl piperonylate+TX, 5-(1,3-benzodioxol-5-yl)-3-hexylcyclohex-2-enone+TX, farnesol with nerolidol+TX, verbutin+TX, MGK 264+TX, piperonyl butoxide+TX, piprotal+TX, propyl isomer+TX, S421+TX, sesamex+TX, sesasmolin+TX, sulfoxide+TX, anthraquinone+TX, copper naphthenate+TX, copper oxychloride+TX, dicyclopentadiene+TX, thiram+TX, zinc naphthenate+TX, ziram+TX, imanin+TX, ribavirin+TX, chloroinconazide+TX, mercuric oxide+TX, thiophanate-methyl+TX, azaconazole+TX, bitertanol+TX, bromuconazole+TX, cyproconazole+TX, difenoconazole+TX, diniconazole+TX, epoxiconazole+TX, fenbuconazole+TX, fluquinconazole+TX, flusilazole+TX, flutriafol+TX, furametpyr+TX, hexaconazole+TX, imazalil+TX, imibenconazole+TX, ipconazole+TX, metconazole+TX, myclobutanil+TX, paclobutrazole+TX, pefurazoate+TX, penconazole+TX, prothioconazole+TX, pyrifenox+TX, prochloraz+TX, propiconazole+TX, pyrisoxazole+TX, simeconazole+TX, tebuconazole+TX, tetraconazole+TX, triadimefon+TX, triadimenol+TX, triflumizole+TX, triticonazole+TX, ancymidol+TX, fenarimol+TX, nuarimol+TX, bupirimate+TX, dimethirimol+TX, ethirimol+TX, dodemorph+TX, fenpropidin+TX, fenpropimorph+TX, spiroxamine+TX, tridemorph+TX, cyprodinil+TX, mepanipyrim+TX, pyrimethanil+TX, fenpiclonil+TX, fludioxonil+TX, benalaxyl+TX, furalaxyl+TX, metalaxyl−+TX, Rmetalaxyl+TX, ofurace+TX, oxadixyl+TX, carbendazim+TX, debacarb+TX, fuberidazole+TX, thiabendazole+TX, chlozolinate+TX, dichlozoline+TX, myclozoline+TX, procymidone+TX, vinclozoline+TX, boscalid+TX, carboxin+TX, fenfuram+TX, flutolanil+TX, mepronil+TX, oxycarboxin+TX, penthiopyrad+TX, thifluzamide+TX, dodine+TX, iminoctadine+TX, azoxystrobin+TX, dimoxystrobin+TX, enestroburin+TX, fenaminstrobin+TX, flufenoxystrobin+TX, fluoxastrobin+TX, kresoxim-methyl+TX, metominostrobin+TX, trifloxystrobin+TX, orysastrobin+TX, picoxystrobin+TX, pyraclostrobin+TX, pyrametostrobin+TX, pyraoxystrobin+TX, ferbam+TX, mancozeb+TX, maneb+TX, metiram+TX, propineb+TX, zineb+TX, captafol+TX, captan+TX, fluoroimide+TX, folpet+TX, tolylfluanid+TX, bordeaux mixture+TX, copper oxide+TX, mancopper+TX, oxine-copper+TX, nitrothal-isopropyl+TX, edifenphos+TX, iprobenphos+TX, phosdiphen+TX, tolclofos-methyl+TX, anilazine+TX, benthiavalicarb+TX, blasticidin-S+TX, chloroneb+TX, chlorothalonil+TX, cyflufenamid+TX, cymoxanil+TX, cyclobutrifluram+TX, diclocymet+TX, diclomezine+TX, dicloran+TX, diethofencarb+TX, dimethomorph+TX, flumorph+TX, dithianon+TX, ethaboxam+TX, etridiazole+TX, famoxadone+TX, fenamidone+TX, fenoxanil+TX, ferimzone+TX, fluazinam+TX, flumetylsulforim+TX, fluopicolide+TX, fluoxytioconazole+TX, flusulfamide+TX, fluxapyroxad+TX, fenhexamid+TX, fosetyl-aluminium+TX, hymexazol+TX, iprovalicarb+TX, cyazofamid+TX, methasulfocarb+TX, metrafenone+TX, pencycuron+TX, phthalide+TX, polyoxins+TX, propamocarb+TX, pyribencarb+TX, proquinazid+TX, pyroquilon+TX, pyriofenone+TX, quinoxyfen+TX, quintozene+TX, tiadinil+TX, triazoxide+TX, tricyclazole+TX, triforine+TX, validamycin+TX, valifenalate+TX, zoxamide+TX, mandipropamid+TX, flubeneteram+TX, isopyrazam+TX, sedaxane+TX, benzovindiflupyr+TX, pydiflumetofen+TX, 3-difluoromethyl-1-methyl-1H-pyrazole-4-carboxylic acid (3′,4′,5′-trifluoro-biphenyl-2-yl)-amide+TX, isoflucypram+TX, isotianil+TX, dipymetitrone+TX, 6-ethyl-5,7-dioxo-pyrrolo[4,5][1,4]dithiino[1,2-c]isothiazole-3-carbonitrile+TX, 2-(difluoromethyl)-N-[3-ethyl-1,1-dimethyl-indan-4-yl]pyridine-3-carboxamide+TX, 4-(2,6-difluorophenyl)-6-methyl-5-phenyl-pyridazine-3-carbonitrile+TX, (R)-3-(difluoromethyl)-1-methyl-N-[1,1,3-trimethylindan-4-yl]pyrazole-4-carboxamide+TX, 4-(2-bromo-4-fluoro-phenyl)-N-(2-chloro-6-fluoro-phenyl)-2,5-dimethyl-pyrazol-3-amine+TX, 4-(2-bromo-4-fluorophenyl) -N-(2-chloro-6-fluorophenyl)-1, 3-dimethyl-1H-pyrazol-5-amine+TX, fluindapyr+TX, coumethoxystrobin (jiaxiangjunzhi)+TX, Ivbenmixianan+TX, dichlobentiazox+TX, mandestrobin+TX, 3-(4,4-difluoro-3,4-dihydro-3,3-dimethylisoquinolin-1-yl)quinolone+TX, 2-[2-fluoro-6-[(8-fluoro-2-methyl-3-quinolyl)oxy]phenyl]propan-2-ol+TX, oxathiapiprolin+TX, tert-butyl N-[6-[[[(1-methyltetrazol-5-yl)-phenyl-methylene]amino]oxymethyl]-2-pyridyl]carbamate+TX, pyraziflumid+TX, inpyrfluxam+TX, trolprocarb+TX, mefentrifluconazole+TX, ipfentrifluconazole+TX, 2-(difluoromethyl)-N-[(3R)-3-ethyl-1,1-dimethyl-indan-4-yl]pyridine-3-carboxamide+TX, N′-(2,5-dimethyl-4-phenoxy-phenyl)-N-ethyl-N-methyl-formamidine+TX, N′-[4-(4,5-dichlorothiazol-2-yl)oxy-2,5-dimethyl-phenyl]-N-ethyl-N-methyl-formamidine+TX, [2-[3-[2-[1-[2-[3,5-bis(difluoromethyl)pyrazol-1-yl]acetyl]-4-piperidyl]thiazol-4-yl]-4,5-dihydroisoxazol-5-yl]-3-chloro-phenyl]methanesulfonate+TX, but-3-ynyl N-[6-[[(Z)-[(1-methyltetrazol-5-yl)-phenyl-methylene]amino]oxymethyl]-2-pyridyl]carbamate+TX, methyl N-[[5-[4-(2,4-dimethylphenyl)triazol-2-yl]-2-methyl-phenyl]methyl]carbamate+TX, 3-chloro-6-methyl-5-phenyl-4-(2,4,6-trifluorophenyl)pyridazine+TX, pyridachlometyl+TX, 3-(difluoromethyl)-1-methyl-N-[1,1,3-trimethylindan-4-yl]pyrazole-4-carboxamide+TX, 1-[2-[[1-(4-chlorophenyl)pyrazol-3-yl]oxymethyl]-3-methyl-phenyl]-4-methyl-tetrazol-5-one+TX, 1-methyl-4-[3-methyl-2-[[2-methyl-4-(3,4,5-trimethylpyrazol-1-yl)phenoxy]methyl]phenyl]tetrazol-5-one+TX, aminopyrifen+TX, ametoctradin+TX, amisulbrom+TX, penflufen+TX, (Z,2E)-5-[1-(4-chlorophenyl)pyrazol-3-yl]oxy-2-methoxyimino-N,3-dimethyl-pent-3-enamide+TX, florylpicoxamid+TX, fenpicoxamid+TX, metarylpicoxamid+TX, tebufloquin+TX, ipflufenoquin+TX, quinofumelin+TX, isofetamid+TX, ethyl 1-[[4-[[2-(trifluoromethyl)-1,3-dioxolan-2-yl]methoxy]phenyl]methyl]pyrazole-3-carboxylate+TX (may be prepared from the methods described in WO 2020/056090), ethyl 1-[[4-[(Z)-2-ethoxy-3,3,3-trifluoro-prop-1-enoxy]phenyl]methyl]pyrazole-3-carboxylate+TX (may be prepared from the methods described in WO 2020/056090), methyl N-[[4-[1-(4-cyclopropyl-2,6-difluoro-phenyl)pyrazol-4-yl]-2-methyl-phenyl]methyl]carbamate+TX (may be prepared from the methods described in WO 2020/097012), methyl N-[[4-[1-(2,6-difluoro-4-isopropyl-phenyl)pyrazol-4-yl]-2-methyl-phenyl]methyl]carbamate+TX (may be prepared from the methods described in WO 2020/097012), 6-chloro-3-(3-cyclopropyl-2-fluoro-phenoxy)-N-[2-(2,4-dimethylphenyl)-2,2-difluoro-ethyl]-5-methyl-pyridazine-4-carboxamide+TX (may be prepared from the methods described in WO 2020/109391), 6-chloro-N-[2-(2-chloro-4-methyl-phenyl)-2,2-difluoro-ethyl]-3-(3-cyclopropyl-2-fluoro-phenoxy)-5-methyl-pyridazine-4-carboxamide+TX (may be prepared from the methods described in WO 2020/109391), 6-chloro-3-(3-cyclopropyl-2-fluoro-phenoxy)-N-[2-(3,4-dimethylphenyl)-2,2-difluoro-ethyl]-5-methyl-pyridazine-4-carboxamide+TX (may be prepared from the methods described in WO 2020/109391), N-[2-[2,4-dichloro-phenoxy]phenyl]-3-(difluoromethyl)-1-methyl-pyrazole-4-carboxamide+TX, N-[2-[2-chloro-4-(trifluoromethyl)phenoxy]phenyl]-3-(difluoromethyl)-1-methyl-pyrazole-4-carboxamide+TX, benzothiostrobin+TX, phenamacril+TX, 5-amino-1,3,4-thiadiazole-2-thiol zinc salt (2:1)+TX, fluopyram+TX, flufenoxadiazam+TX, flutianil+TX, fluopimomide+TX, pyrapropoyne+TX, picarbutrazox+TX, 2-(difluoromethyl)-N-(3-ethyl-1,1-dimethyl-indan-4-yl)pyridine-3-carboxamide+TX, 2-(difluoromethyl) -N-((3R)-1, 1, 3-trimethylindan-4-yl) pyridine-3-carboxamide+TX, 4-[[6-[2-(2,4-difluorophenyl)-1,1-difluoro-2-hydroxy-3-(1,2,4-triazol-1-yl)propyl]-3-pyridyl]oxy]benzonitrile+TX, metyltetraprole+TX, 2-(difluoromethyl) -N-((3R)-1, 1, 3-trimethylindan-4-yl) pyridine-3-carboxamide+TX, α-(1, 1-dimethylethyl)-α-[4′-(trifluoromethoxy) [1, 1′-biphenyl]-4-yl]-5-pyrimidinemethanol+TX, fluoxapiprolin+TX, enoxastrobin+TX, methyl (Z)-3-methoxy-2-[2-methyl-5-[4-(trifluoromethyl)triazol-2-yl]phenoxy]prop-2-enoate+TX, methyl (Z)-3-methoxy-2-[2-methyl-5-(4-propyltriazol-2-yl)phenoxy]prop-2-enoate+TX, methyl (Z)-2-[5-(3-isopropylpyrazol-1-yl)-2-methyl-phenoxy]-3-methoxy-prop-2-enoate+TX, methyl (Z)-3-methoxy-2-[2-methyl-5-(3-propylpyrazol-1-yl)phenoxy]prop-2-enoate+TX, methyl (Z)-3-methoxy-2-[2-methyl-5-[3-(trifluoromethyl)pyrazol-1-yl]phenoxy]prop-2-enoate+TX (these compounds may be prepared from the methods described in WO2020/079111), methyl (Z)-2-(5-cyclohexyl-2-methyl-phenoxy)-3-methoxy-prop-2-enoate+TX, methyl (Z)-2-(5-cyclopentyl-2-methyl-phenoxy)-3-methoxy-prop-2-enoate+TX (these compounds may be prepared from the methods described in WO2020/193387), 4-[[6-[2-(2,4-difluorophenyl)-1,1-difluoro-2-hydroxy-3-(1,2,4-triazol-1-yl)propyl]-3-pyridyl]oxy]benzonitrile+TX, 4-[[6-[2-(2,4-difluorophenyl)-1,1-difluoro-2-hydroxy-3-(5-sulfanyl-1,2,4-triazol-1-yl)propyl]-3-pyridyl]oxy]benzonitrile+TX, 4-[[6-[2-(2,4-difluorophenyl)-1,1-difluoro-2-hydroxy-3-(5-thioxo-4H-1,2,4-triazol-1-yl)propyl]-3-pyridyl]oxy]benzonitrile+TX, trinexapac+TX, coumoxystrobin+TX, zhongshengmycin+TX, thiodiazole copper+TX, zinc thiazole+TX, amectotractin+TX, iprodione+TX, seboctylamine+TX; N′-[5-bromo-2-methyl-6-[(1S)-1-methyl-2-propoxy-ethoxy]-3-pyridyl]-N-ethyl-N-methyl-formamidine+TX, N′-[5-bromo-2-methyl-6-[(1R)-1-methyl-2-propoxy-ethoxy]-3-pyridyl]-N-ethyl-N-methyl-formamidine+TX, N′-[5-bromo-2-methyl-6-(1-methyl-2-propoxy-ethoxy)-3-pyridyl]-N-ethyl-N-methyl-formamidine+TX, N′-[5-chloro-2-methyl-6-(1-methyl-2-propoxy-ethoxy)-3-pyridyl]-N-ethyl-N-methyl-formamidine+TX, N′-[5-bromo-2-methyl-6-(1-methyl-2-propoxy-ethoxy)-3-pyridyl]-N-isopropyl-N-methyl-formamidine+TX (these compounds may be prepared from the methods described in WO2015/155075); N′-[5-bromo-2-methyl-6-(2-propoxypropoxy)-3-pyridyl]-N-ethyl-N-methyl-formamidine+TX (this compound may be prepared from the methods described in IPCOM000249876D); N-isopropyl-N′-[5-methoxy-2-methyl-4-(2,2,2-trifluoro-1-hydroxy-1-phenyl-ethyl)phenyl]-N-methyl-formamidine+TX, N′-[4-(1-cyclopropyl-2,2,2-trifluoro-1-hydroxy-ethyl)-5-methoxy-2-methyl-phenyl]-N-isopropyl-N-methyl-formamidine+TX (these compounds may be prepared from the methods described in WO2018/228896); N-ethyl-N′-[5-methoxy-2-methyl-4-[(2-trifluoromethyl)oxetan-2-yl]phenyl]-N-methyl-formamidine+TX, N-ethyl-N′-[5-methoxy-2-methyl-4-[(2-trifuoromethyl)tetrahydrofuran-2-yl]phenyl]-N-methyl-formamidine+TX (these compounds may be prepared from the methods described in WO2019/110427); N-[(1R)-1-benzyl-3-chloro-1-methyl-but-3-enyl]-8-fluoro-quinoline-3-carboxamide+TX, N-[(1S)-1-benzyl-3-chloro-1-methyl-but-3-enyl]-8-fluoro-quinoline-3-carboxamide+TX, N-[(1R)-1-benzyl-3,3,3-trifluoro-1-methyl-propyl]-8-fluoro-quinoline-3-carboxamide+TX, N-[(1S)-1-benzyl-3,3,3-trifluoro-1-methyl-propyl]-8-fluoro-quinoline-3-carboxamide+TX, N-[(1R)-1-benzyl-1,3-dimethyl-butyl]-7,8-difluoro-quinoline-3-carboxamide+TX, N-[(1S)-1-benzyl-1,3-dimethyl-butyl]-7,8-difluoro-quinoline-3-carboxamide+TX, 8-fluoro-N-[(1R)-1-[(3-fluorophenyl)methyl]-1,3-dimethyl-butyl]quinoline-3-carboxamide+TX, 8-fluoro-N-[(1S)-1-[(3-fluorophenyl)methyl]-1,3-dimethyl-butyl]quinoline-3-carboxamide+TX, N-[(1R)-1-benzyl-1,3-dimethyl-butyl]-8-fluoro-quinoline-3-carboxamide+TX, N-[(1S)-1-benzyl-1,3-dimethyl-butyl]-8-fluoro-quinoline-3-carboxamide+TX, N-((1R)-1-benzyl-3-chloro-1-methyl-but-3-enyl)-8-fluoro-quinoline-3-carboxamide+TX, N-((1S)-1-benzyl-3-chloro-1-methyl-but-3-enyl)-8-fluoro-quinoline-3-carboxamide+TX (these compounds may be prepared from the methods described in WO2017/153380); 1-(6,7-dimethylpyrazolo[1,5-a]pyridin-3-yl)-4,4,5-trifluoro-3,3-dimethyl-isoquinoline+TX, 1-(6,7-dimethylpyrazolo[1,5-a]pyridin-3-yl)-4,4,6-trifluoro-3,3-dimethyl-isoquinoline+TX, 4,4-difluoro-3,3-dimethyl-1-(6-methylpyrazolo[1,5-a]pyridin-3-yl)isoquinoline+TX, 4,4-difluoro-3,3-dimethyl-1-(7-methylpyrazolo[1,5-a]pyridin-3-yl)isoquinoline+TX, 1-(6-chloro-7-methyl-pyrazolo[1,5-a]pyridin-3-yl)-4,4-difluoro-3,3-dimethyl-isoquinoline+TX (these compounds may be prepared from the methods described in WO2017/025510); 1-(4,5-dimethylbenzimidazol-1-yl)-4,4,5-trifluoro-3,3-dimethyl-isoquinoline+TX, 1-(4,5-dimethylbenzimidazol-1-yl)-4,4-difluoro-3,3-dimethyl-isoquinoline+TX, 6-chloro-4,4-difluoro-3,3-dimethyl-1-(4-methylbenzimidazol-1-yl)isoquinoline+TX, 4,4-difluoro-1-(5-fluoro-4-methyl-benzimidazol-1-yl)-3,3-dimethyl-isoquinoline+TX, 3-(4,4-difluoro-3,3-dimethyl-1-isoquinolyl)-7,8-dihydro-6H-cyclopenta[e]benzimidazole+TX (these compounds may be prepared from the methods described in WO2016/156085); N-methoxy-N-[[4-[5-(trifluoromethyl)-1,2,4-oxadiazol-3-yl]phenyl]methyl]cyclopropanecarboxamide+TX, N,2-dimethoxy-N-[[4-[5-(trifluoromethyl)-1,2,4-oxadiazol-3-yl]phenyl]methyl]propanamide+TX, N-ethyl-2-methyl-N-[[4-[5-(trifluoromethyl)-1,2,4-oxadiazol-3-yl]phenyl]methyl]propanamide+TX, 1-methoxy-3-methyl-1-[[4-[5-(trifluoromethyl)-1,2,4-oxadiazol-3-yl]phenyl]methyl]urea+TX, 1,3-dimethoxy-1-[[4-[5-(trifluoromethyl)-1,2,4-oxadiazol-3-yl]phenyl]methyl]urea+TX, 3-ethyl-1-methoxy-1-[[4-[5-(trifluoromethyl)-1,2,4-oxadiazol-3-yl]phenyl]methyl]urea+TX, N-[[4-[5-(trifluoromethyl)-1,2,4-oxadiazol-3-yl]phenyl]methyl]propanamide+TX, 4,4-dimethyl-2-[[4-[5-(trifluoromethyl)-1,2,4-oxadiazol-3-yl]phenyl]methyl]isoxazolidin-3-one+TX, 5,5-dimethyl-2-[[4-[5-(trifluoromethyl)-1,2,4-oxadiazol-3-yl]phenyl]methyl]isoxazolidin-3-one+TX, ethyl 1-[[4-[5-(trifluoromethyl)-1,2,4-oxadiazol-3-yl]phenyl]methyl]pyrazole-4-carboxylate+TX, N,N-dimethyl-1-[[4-[5-(trifluoromethyl)-1,2,4-oxadiazol-3-yl]phenyl]methyl]-1,2,4-triazol-3-amine+TX. The compounds in this paragraph may be prepared from the methods described in WO 2017/055473, WO 2017/055469, WO 2017/093348 and WO 2017/118689; 2-[6-(4-chlorophenoxy)-2-(trifluoromethyl)-3-pyridyl]-1-(1,2,4-triazol-1-yl)propan-2-ol+TX (this compound may be prepared from the methods described in WO 2017/029179); 2-[6-(4-bromophenoxy)-2-(trifluoromethyl)-3-pyridyl]-1-(1,2,4-triazol-1-yl)propan-2-ol+TX (this compound may be prepared from the methods described in WO 2017/029179); 3-[2-(1-chlorocyclopropyl)-3-(2-fluorophenyl)-2-hydroxy-propyl]imidazole-4-carbonitrile+TX (this compound may be prepared from the methods described in WO 2016/156290); 3-[2-(1-chlorocyclopropyl)-3-(3-chloro-2-fluoro-phenyl)-2-hydroxy-propyl]imidazole-4-carbonitrile+TX (this compound may be prepared from the methods described in WO 2016/156290); (4-phenoxyphenyl)methyl 2-amino-6-methyl-pyridine-3-carboxylate+TX (this compound may be prepared from the methods described in WO 2014/006945); 2,6-Dimethyl-1H,5H-[1,4]dithiino[2,3-c:5,6-c′]dipyrrole-1,3,5,7(2H,6H)-tetrone+TX (this compound may be prepared from the methods described in WO 2011/138281); N-methyl-4-[5-(trifluoromethyl)-1,2,4-oxadiazol-3-yl]benzenecarbothioamide+TX; N-methyl-4-[5-(trifluoromethyl)-1,2,4-oxadiazol-3-yl]benzamide+TX; (Z,2E)-5-[1-(2,4-dichlorophenyl)pyrazol-3-yl]oxy-2-methoxyimino-N,3-dimethyl-pent-3-enamide+TX (this compound may be prepared from the methods described in WO 2018/153707); N′-(2-chloro-5-methyl-4-phenoxy-phenyl)-N-ethyl-N-methyl-formamidine+TX; N′-[2-chloro-4-(2-fluorophenoxy)-5-methyl-phenyl]-N-ethyl-N-methyl-formamidine+TX (this compound may be prepared from the methods described in WO 2016/202742); 2-(difluoromethyl)-N-[(3S)-3-ethyl-1,1-dimethyl-indan-4-yl]pyridine-3-carboxamide+TX (this compound may be prepared from the methods described in WO 2014/095675); (5-methyl-2-pyridyl)-[4-[5-(trifluoromethyl)-1,2,4-oxadiazol-3-yl]phenyl]methanone+TX, (3-methylisoxazol-5-yl)-[4-[5-(trifluoromethyl)-1,2,4-oxadiazol-3-yl]phenyl]methanone+TX (these compounds may be prepared from the methods described in WO 2017/220485); 2-oxo-N-propyl-2-[4-[5-(trifluoromethyl)-1,2,4-oxadiazol-3-yl]phenyl]acetamide+TX (this compound may be prepared from the methods described in WO 2018/065414); ethyl 1-[[5-[5-(trifluoromethyl)-1,2,4-oxadiazol-3-yl]-2-thienyl]methyl]pyrazole-4-carboxylate+TX (this compound may be prepared from the methods described in WO 2018/158365); 2,2-difluoro-N-methyl-2-[4-[5-(trifluoromethyl)-1,2,4-oxadiazol-3-yl]phenyl]acetamide+TX, N-[(E)-methoxyiminomethyl]-4-[5-(trifluoromethyl)-1,2,4-oxadiazol-3-yl]benzamide+TX, N—[(Z)-methoxyiminomethyl]-4-[5-(trifluoromethyl)-1,2,4-oxadiazol-3-yl]benzamide+TX, N—[N-methoxy-C-methyl-carbonimidoyl]-4-[5-(trifluoromethyl)-1,2,4-oxadiazol-3-yl]benzamide+TX (these compounds may be prepared from the methods described in WO 2018/202428);
microbials including: Acinetobacter lwoffii+TX, Acremonium alternatum+TX+TX, Acremonium cephalosporium+TX+TX, Acremonium diospyri+TX, Acremonium obclavatum+TX, Adoxophyes orana granulovirus (AdoxGV) (Capex®)+TX, Agrobacterium radiobacter strain K84 (Galltrol-A®)+TX, Alternaria alternate+TX, Alternaria cassia+TX, Alternaria destruens (Smolder®)+TX, Ampelomyces quisqualis (AQ10®)+TX, Aspergillus flavus AF36 (AF36®)+TX, Aspergillus flavus NRRL 21882 (Aflaguard®)+TX, Aspergillus spp.+TX, Aureobasidium pullulans+TX, Azospirillum+TX, (MicroAZ®+TX, TAZO B®)+TX, Azotobacter+TX, Azotobacter chroocuccum (Azotomeal®)+TX, Azotobacter cysts (Bionatural Blooming Blossoms®)+TX, Bacillus amyloliquefaciens+TX, Bacillus cereus+TX, Bacillus chitinosporus strain CM-1+TX, Bacillus chitinosporus strain AQ746+TX, Bacillus licheniformis strain HB-2 (Biostart™ Rhizoboost®)+TX, Bacillus licheniformis strain 3086 (EcoGuard®+TX, Green Releaf®)+TX, Bacillus circulans+TX, Bacillus firmus (BioSafe®+TX, BioNem-WP®+TX, VOTiVO®)+TX, Bacillus firmus strain 1-1582+TX, Bacillus macerans+TX, Bacillus marismortui+TX, Bacillus megaterium+TX, Bacillus mycoides strain AQ726+TX, Bacillus papillae (Milky Spore Powder®)+TX, Bacillus pumilus spp.+TX, Bacillus pumilus strain GB34 (Yield Shield®)+TX, Bacillus pumilus strain AQ717+TX, Bacillus pumilus strain QST 2808 (Sonata®+TX, Ballad Plus®)+TX, Bacillus spahericus (VectoLex®)+TX, Bacillus spp.+TX, Bacillus spp. strain AQ175+TX, Bacillus spp. strain AQ177+TX, Bacillus spp. strain AQ178+TX, Bacillus subtilis strain QST 713 (CEASE®+TX, Serenade®+TX, Rhapsody®)+TX, Bacillus subtilis strain QST 714 (JAZZ®)+TX, Bacillus subtilis strain AQ153+TX, Bacillus subtilis strain AQ743+TX, Bacillus subtilis strain QST3002+TX, Bacillus subtilis strain QST3004+TX, Bacillus subtilis var. amyloliquefaciens strain FZB24 (Taegro®+TX, Rhizopro®)+TX, Bacillus thuringiensis Cry 2Ae+TX, Bacillus thuringiensis Cry1Ab+TX, Bacillus thuringiensis aizawai GC 91 (Agree®)+TX, Bacillus thuringiensis israelensis (BMP123®+TX, Aquabac®+TX, VectoBac®)+TX, Bacillus thuringiensis kurstaki (Javelin®+TX, Deliver®+TX, CryMax®+TX, Bonide®+TX, Scutella WP®+TX, Turilav WP®+TX, Astuto®+TX, Dipel WP®+TX, Biobit®+TX, Foray®)+TX, Bacillus thuringiensis kurstaki BMP 123 (Baritone®)+TX, Bacillus thuringiensis kurstaki HD-1 (Bioprotec-CAF/3P®)+TX, Bacillus thuringiensis strain BD #32+TX, Bacillus thuringiensis strain AQ52+TX, Bacillus thuringiensis var. aizawai (XenTari®+TX, DiPel®)+TX, bacteria spp. (GROWMEND®+TX, GROWSWEET®+TX, Shootup®)+TX, bacteriophage of Clavipacter michiganensis (AgriPhage®)+TX, Bakflor®+TX, Beauveria bassiana (Beaugenic®+TX, Brocaril WP®)+TX, Beauveria bassiana GHA (Mycotrol ES®+TX, Mycotrol O®+TX, BotaniGuard®)+TX, Beauveria brongniartii (Engerlingspilz®+TX, Schweizer Beauveria®+TX, Melocont®)+TX, Beauveria spp.+TX, Botrytis cineria+TX, Bradyrhizobium japonicum (TerraMax®)+TX, Brevibacillus brevis+TX, Bacillus thuringiensis tenebrionis (Novodor®)+TX, BtBooster+TX, Burkholderia cepacia (Deny®+TX, Intercept®+TX, Blue Circle®)+TX, Burkholderia gladii+TX, Burkholderia gladioli+TX, Burkholderia spp.+TX, Canadian thistle fungus (CBH Canadian Bioherbicide®)+TX, Candida butyri+TX, Candida famata+TX, Candida fructus+TX, Candida glabrata+TX, Candida guilliermondii+TX, Candida melibiosica+TX, Candida oleophila strain O+TX, Candida parapsilosis+TX, Candida pelliculosa+TX, Candida pulcherrima+TX, Candida reukaufii+TX, Candida saitoana (Bio-Coat®+TX, Biocure®)+TX, Candida sake+TX, Candida spp.+TX, Candida tenius+TX, Cedecea dravisae+TX, Cellulomonas flavigena+TX, Chaetomium cochliodes (Nova-Cide®)+TX, Chaetomium globosum (Nova-Cide®)+TX, Chromobacterium subtsugae strain PRAA4-1T (Grandevo®)+TX, Cladosporium cladosporioides+TX, Cladosporium oxysporum+TX, Cladosporium chlorocephalum+TX, Cladosporium spp.+TX, Cladosporium tenuissimum+TX, Clonostachys rosea (EndoFine®)+TX, Colletotrichum acutatum+TX, Coniothyrium minitans (Cotans WG®)+TX, Coniothyrium spp.+TX, Cryptococcus albidus (YIELDPLUS®)+TX, Cryptococcus humicola+TX, Cryptococcus infirmominiatus+TX, Cryptococcus laurentii+TX, Cryptophlebia leucotreta granulovirus (Cryptex®)+TX, Cupriavidus campinensis+TX, Cydia pomonella granulovirus (CYD-X®)+TX, Cydia pomonella granulovirus (Madex®+TX, Madex Plus®+TX, Madex Max/Carpovirusine®)+TX, Cylindrobasidium laeve (Stumpout®)+TX, Cylindrocladium+TX, Debaryomyces hansenii+TX, Drechslera hawaiinensis+TX, Enterobacter cloacae+TX, Enterobacteriaceae+TX, Entomophtora virulenta (Vektor®)+TX, Epicoccum nigrum+TX, Epicoccum purpurascens+TX, Epicoccum spp.+TX, Filobasidium floriforme+TX, Fusarium acuminatum+TX, Fusarium chlamydosporum+TX, Fusarium oxysporum (Fusaclean®/Biofox C®)+TX, Fusarium proliferatum+TX, Fusarium spp.+TX, Galactomyces geotrichum+TX, Gliocladium catenulatum (Primastop®+TX, Prestop®)+TX, Gliocladium roseum+TX, Gliocladium spp. (SoilGard®)+TX, Gliocladium virens (Soilgard®)+TX, Granulovirus (Granupom®)+TX, Halobacillus halophilus+TX, Halobacillus litoralis+TX, Halobacillus trueperi+TX, Halomonas spp.+TX, Halomonas subglaciescola+TX, Halovibrio variabilis+TX, Hanseniaspora uvarum+TX, Helicoverpa armigera nucleopolyhedrovirus (Helicovex®)+TX, Helicoverpa zea nuclear polyhedrosis virus (Gemstar®)+TX, Isoflavone-formononetin (Myconate®)+TX, Kloeckera apiculata+TX, Kloeckera spp.+TX, Lagenidium giganteum (Laginex®)+TX, Lecanicillium longisporum (Vertiblast®)+TX, Lecanicillium muscarium (Vertikil®)+TX, Lymantria Dispar nucleopolyhedrosis virus (Disparvirus®)+TX, Marinococcus halophilus+TX, Meira geulakonigli+TX, Metarhizium anisopliae (Met52®)+TX, Metarhizium anisopliae (Destruxin WP®)+TX, Metschnikowia fruticola (Shemer®)+TX, Metschnikowia pulcherrima+TX, Microdochium dimerum (Antibot®)+TX, Micromonospora coerulea+TX, Microsphaeropsis ochracea+TX, Muscodor albus 620 (Muscudor®)+TX, Muscodor roseus strain A3-5+TX, Mycorrhizae spp. (AMykor®+TX, Root Maximizer®)+TX, Myrothecium verrucaria strain AARC-0255 (DiTera®)+TX, BROS PLUS®+TX, Ophiostoma piliferum strain D97 (Sylvanex®)+TX, Paecilomyces farinosus+TX, Paecilomyces fumosoroseus (PFR-97®+TX, PreFeRal®)+TX, Paecilomyces linacinus (Biostat WP®)+TX, Paecilomyces lilacinus strain 251 (MeloCon WG®)+TX, Paenibacillus polymyxa+TX, Pantoea agglomerans (BlightBan C9-1®)+TX, Pantoea spp.+TX, Pasteuria spp. (Econem®)+TX, Pasteuria nishizawae+TX, Penicillium aurantiogriseum+TX, Penicillium billai (Jumpstart®+TX, TagTeam®)+TX, Penicillium brevicompactum+TX, Penicillium frequentans+TX, Penicillium griseofulvum+TX, Penicillium purpurogenum+TX, Penicillium spp.+TX, Penicillium viridicatum+TX, Phlebiopsis gigantean (Rotstop®)+TX, phosphate solubilizing bacteria (Phosphomeal®)+TX, Phytophthora cryptogea+TX, Phytophthora palmivora (Devine®)+TX, Pichia anomala+TX, Pichia guilermondii+TX, Pichia membranaefaciens+TX, Pichia onychis+TX, Pichia stipites+TX, Pseudomonas aeruginosa+TX, Pseudomonas aureofasciens (Spot-Less Biofungicide®)+TX, Pseudomonas cepacia+TX, Pseudomonas chlororaphis (AtEze®)+TX, Pseudomonas corrugate+TX, Pseudomonas fluorescens strain A506 (BlightBan A506®)+TX, Pseudomonas putida+TX, Pseudomonas reactans+TX, Pseudomonas spp.+TX, Pseudomonas syringae (Bio-Save®)+TX, Pseudomonas viridiflava+TX, Pseudomons fluorescens (Zequanox®)+TX, Pseudozyma flocculosa strain PF-A22 UL (Sporodex L®)+TX, Puccinia canaliculata+TX, Puccinia thlaspeos (Wood Warrior®)+TX, Pythium paroecandrum+TX, Pythium oligandrum (Polygandron®+TX, Polyversum®)+TX, Pythium periplocum+TX, Rhanella aquatilis+TX, Rhanella spp.+TX, Rhizobia (Dormal®+TX, Vault®)+TX, Rhizoctonia+TX, Rhodococcus globerulus strain AQ719+TX, Rhodosporidium diobovatum+TX, Rhodosporidium toruloides+TX, Rhodotorula spp.+TX, Rhodotorula glutinis+TX, Rhodotorula graminis+TX, Rhodotorula mucilagnosa+TX, Rhodotorula rubra+TX, Saccharomyces cerevisiae+TX, Salinococcus roseus+TX, Sclerotinia minor+TX, Sclerotinia minor (SARRITOR®)+TX, Scytalidium spp.+TX, Scytalidium uredinicola+TX, Spodoptera exigua nuclear polyhedrosis virus (Spod-X®+TX, Spexit®)+TX, Serratia marcescens+TX, Serratia plymuthica+TX, Serratia spp.+TX, Sordaria fimicola+TX, Spodoptera littoralis nucleopolyhedrovirus (Littovir®)+TX, Sporobolomyces roseus+TX, Stenotrophomonas maltophilia+TX, Streptomyces ahygroscopicus+TX, Streptomyces albaduncus+TX, Streptomyces exfoliates+TX, Streptomyces galbus+TX, Streptomyces griseoplanus+TX, Streptomyces griseoviridis (Mycostop®)+TX, Streptomyces lydicus (Actinovate®)+TX, Streptomyces lydicus WYEC-108 (ActinoGrow®)+TX, Streptomyces violaceus+TX, Tilletiopsis minor+TX, Tilletiopsis spp.+TX, Trichoderma asperellum (T34 Biocontrol®)+TX, Trichoderma gamsii (Tenet®)+TX, Trichoderma atroviride (Plantmate®)+TX, Trichoderma hamatum TH 382+TX, Trichoderma harzianum rifai (Mycostar®)+TX, Trichoderma harzianum T-22 (Trianum-P®+TX, PlantShield HC®+TX, RootShield®+TX, Trianum-G®)+TX, Trichoderma harzianum T-39 (Trichodex®)+TX, Trichoderma inhamatum+TX, Trichoderma koningii+TX, Trichoderma spp. LC 52 (Sentinel®)+TX, Trichoderma lignorum+TX, Trichoderma longibrachiatum+TX, Trichoderma polysporum (Binab T®)+TX, Trichoderma taxi+TX, Trichoderma virens+TX, Trichoderma virens (formerly Gliocladium virens GL-21) (SoilGuard®)+TX, Trichoderma viride+TX, Trichoderma viride strain ICC 080 (Remedier®)+TX, Trichosporon pullulans+TX, Trichosporon spp.+TX, Trichothecium spp.+TX, Trichothecium roseum+TX, Typhula phacorrhiza strain 94670+TX, Typhula phacorrhiza strain 94671+TX, Ulocladium atrum+TX, Ulocladium oudemansii (Botry-Zen®)+TX, Ustilago maydis+TX, various bacteria and supplementary micronutrients (Natural 11®)+TX, various fungi (Millennium Microbes®)+TX, Verticillium chlamydosporium+TX, Verticillium lecanii (Mycotal®+TX, Vertalec®)+TX, Vip3Aa20 (VIPtera®)+TX, Virgibaclillus marismortui+TX, Xanthomonas campestris pv. Poae (Camperico®)+TX, Xenorhabdus bovienii+TX, Xenorhabdus nematophilus;
Plant extracts including: pine oil (Retenol®)+TX, azadirachtin (Plasma Neem Oil®+TX, AzaGuard®+TX, MeemAzal®+TX, Molt-X®+TX, Botanical IGR (Neemazad®+TX, Neemix®)+TX, canola oil (Lilly Miller Vegol®)+TX, Chenopodium ambrosioides near ambrosioides (Requiem®)+TX, Chrysanthemum extract (Crisant®)+TX, extract of neem oil (Trilogy®)+TX, essentials oils of Labiatae (Botania®)+TX, extracts of clove rosemary peppermint and thyme oil (Garden Insect Killer®)+TX, Glycinebetaine (Greenstim®)+TX, garlic+TX, lemongrass oil (GreenMatch®)+TX, neem oil+TX, Nepeta cataria (Catnip oil)+TX, Nepeta catarina+TX, nicotine+TX, oregano oil (MossBuster®)+TX, Pedaliaceae oil (Nematon®)+TX, pyrethrum+TX, Quillaja saponaria (NemaQ®)+TX, Reynoutria sachalinensis (Regalia®+TX, Sakalia®)+TX, rotenone (Eco Roten®)+TX, Rutaceae plant extract (Soleo®)+TX, soybean oil (Ortho Ecosense®)+TX, tea tree oil (Timorex Gold®)+TX, thymus oil+TX, AGNIQUE® MMF+TX, BugOil®+TX, mixture of rosemary sesame peppermint thyme and cinnamon extracts (EF 300®)+TX, mixture of clove rosemary and peppermint extract (EF 400®)+TX, mixture of clove peppermint garlic oil and mint (Soil Shot®)+TX, kaolin (Screen®)+TX, storage glucam of brown algae (Laminarin®);
pheromones including: blackheaded fireworm pheromone (3M Sprayable Blackheaded Fireworm Pheromone®)+TX, Codling Moth Pheromone (Paramount dispenser-(CM)/Isomate C-Plus®)+TX, Grape Berry Moth Pheromone (3M MEC-GBM Sprayable Pheromone®)+TX, Leafroller pheromone (3M MEC-LR Sprayable Pheromone®)+TX, Muscamone (Snip7 Fly Bait®+TX, Starbar Premium Fly Bait®)+TX, Oriental Fruit Moth Pheromone (3M oriental fruit moth sprayable Pheromone®)+TX, Peachtree Borer Pheromone (Isomate-P®)+TX, Tomato Pinworm Pheromone (3M Sprayable Pheromone®)+TX, Entostat powder (extract from palm tree) (Exosex CM®)+TX, (E+TX,Z+TX,Z)-3+TX,8+TX, 11 Tetradecatrienyl acetate+TX, (Z+TX,Z+TX,E)-7+TX, 11+TX,13-Hexadecatrienal+TX, (E+TX,Z)-7+TX,9-Dodecadien-1-yl acetate+TX, 2-Methyl-1-butanol+TX, Calcium acetate+TX, Scenturion®+TX, Biolure®+TX, Check-Mate®+TX, Lavandulyl senecioate; Macrobials including: Aphelinus abdominalis+TX, Aphidius ervi (Aphelinus-System®)+TX, Acerophagus papaya+TX, Adalia bipunctata (Adalia-System®)+TX, Adalia bipunctata (Adaline®)+TX, Adalia bipunctata (Aphidalia®)+TX, Ageniaspis citricola+TX, Ageniaspis fuscicollis+TX, Amblyseius andersoni (Anderline®+TX, Andersoni-System®)+TX, Amblyseius californicus (Amblyline®+TX, Spical®)+TX, Amblyseius cucumeris (Thripex®+TX, Bugline cucumeris®)+TX, Amblyseius fallacis (Fallacis®)+TX, Amblyseius swirskii (Bugline swirskii®+TX, Swirskii-Mite®)+TX, Amblyseius womersleyi (WomerMite®)+TX, Amitus hesperidum+TX, Anagrus atomus+TX, Anagyrus fusciventris+TX, Anagyrus kamali+TX, Anagyrus loecki+TX, Anagyrus pseudococci (Citripar®)+TX, Anicetus benefices+TX, Anisopteromalus calandrae+TX, Anthocoris nemoralis (Anthocoris-System®)+TX, Aphelinus abdominalis (Apheline®+TX, Aphiline®)+TX, Aphelinus asychis+TX, Aphidius colemani (Aphipar®)+TX, Aphidius ervi (Ervipar®)+TX, Aphidius gifuensis+TX, Aphidius matricariae (Aphipar-M®)+TX, Aphidoletes aphidimyza (Aphidend®)+TX, Aphidoletes aphidimyza (Aphidoline®)+TX, Aphytis lingnanensis+TX, Aphytis melinus+TX, Aprostocetus hagenowii+TX, Atheta coriaria (Staphyline®)+TX, Bombus spp.+TX, Bombus terrestris (Natupol Beehive®)+TX, Bombus terrestris (Beeline®+TX, Tripol®)+TX, Cephalonomia stephanoderis+TX, Chilocorus nigritus+TX, Chrysoperla carnea (Chrysoline®)+TX, Chrysoperla carnea (Chrysopa®)+TX, Chrysoperla rufilabris+TX, Cirrospilus ingenuus+TX, Cirrospilus quadristriatus+TX, Citrostichus phyllocnistoides+TX, Closterocerus chamaeleon+TX, Closterocerus spp.+TX, Coccidoxenoides perminutus (Planopar®)+TX, Coccophagus cowperi+TX, Coccophagus lycimnia+TX, Cotesia flavipes+TX, Cotesia plutellae+TX, Cryptolaemus montrouzieri (Cryptobug®+TX, Cryptoline®)+TX, Cybocephalus nipponicus+TX, Dacnusa sibirica+TX, Dacnusa sibirica (Minusa®)+TX, Diglyphus isaea (Diminex®)+TX, Delphastus catalinae (Delphastus®)+TX, Delphastus pusillus+TX, Diachasmimorpha krausii+TX, Diachasmimorpha longicaudata+TX, Diaparsis jucunda+TX, Diaphorencyrtus aligarhensis+TX, Diglyphus isaea+TX, Diglyphus isaea (Miglyphus®+TX, Digline®)+TX, Dacnusa sibirica (DacDigline®+TX, Minex®)+TX, Diversinervus spp.+TX, Encarsia citrina+TX, Encarsia formosa (Encarsia Max®+TX, Encarline®+TX, En-Strip®)+TX, Eretmocerus eremicus (Enermix®)+TX, Encarsia guadeloupae+TX, Encarsia haitiensis+TX, Episyrphus balteatus (Syrphidend®)+TX, Eretmoceris siphonini+TX, Eretmocerus californicus+TX, Eretmocerus eremicus (Ercal®+TX, Eretline E®)+TX, Eretmocerus eremicus (Bemimix®)+TX, Eretmocerus hayati+TX, Eretmocerus mundus (Bemipar®+TX, Eretline M®)+TX, Eretmocerus siphonini+TX, Exochomus quadripustulatus+TX, Feltiella acarisuga (Spidend®)+TX, Feltiella acarisuga (Feltiline®)+TX, Fopius arisanus+TX, Fopius ceratitivorus+TX, Formononetin (Wirless Beehome®)+TX, Franklinothrips vespiformis (Vespop®)+TX, Galendromus occidentalis+TX, Goniozus legneri+TX, Habrobracon hebetor+TX, Harmonia axyridis (HarmoBeetle®)+TX, Heterorhabditis spp. (Lawn Patrol®)+TX, Heterorhabditis bacteriophora (NemaShield HB®+TX, Nemaseek®+TX, Terranem-Nam®+TX, Terranem®+TX, Larvanem®+TX, B-Green®+TX, NemAttack®+TX, Nematop®)+TX, Heterorhabditis megidis (Nemasys H®+TX, BioNem H®+TX, Exhibitline hm®+TX, Larvanem-M®)+TX, Hippodamia convergens+TX, Hypoaspis aculeifer (Aculeifer-System®+TX, Entomite-A®)+TX, Hypoaspis miles (Hypoline m®+TX, Entomite-M®)+TX, Lbalia leucospoides+TX, Lecanoideus floccissimus+TX, Lemophagus errabundus+TX, Leptomastidea abnormis+TX, Leptomastix dactylopii (Leptopar®)+TX, Leptomastix epona+TX, Lindorus lophanthae+TX, Lipolexis oregmae+TX, Lucilia caesar (Natufly®)+TX, Lysiphlebus testaceipes+TX, Macrolophus caliginosus (Mirical-N®+TX, Macroline c®+TX, Mirical®)+TX, Mesoseiulus longipes+TX, Metaphycus flavus+TX, Metaphycus lounsburyi+TX, Micromus angulatus (Milacewing®)+TX, Microterys flavus+TX, Muscidifurax raptorellus and Spalangia cameroni (Biopar®)+TX, Neodryinus typhlocybae+TX, Neoseiulus californicus+TX, Neoseiulus cucumeris (THRYPEX®)+TX, Neoseiulus fallacis+TX, Nesideocoris tenuis (NesidioBug®+TX, Nesibug®)+TX, Ophyra aenescens (Biofly®)+TX, Orius insidiosus (Thripor-I®+TX, Oriline I®)+TX, Orius laevigatus (Thripor-L®+TX, Oriline I®)+TX, Orius majusculus (Oriline m®)+TX, Orius strigicollis (Thripor-S®)+TX, Pauesia juniperorum+TX, Pediobius foveolatus+TX, Phasmarhabditis hermaphrodita (Nemaslug®)+TX, Phymastichus coffea+TX, Phytoseiulus macropilus+TX, Phytoseiulus persimilis (Spidex®+TX, Phytoline p®)+TX, Podisus maculiventris (Podisus®)+TX, Pseudacteon curvatus+TX, Pseudacteon obtusus+TX, Pseudacteon tricuspis+TX, Pseudaphycus maculipennis+TX, Pseudleptomastix mexicana+TX, Psyllaephagus pilosus+TX, Psyttalia concolor (complex)+TX, Quadrastichus spp.+TX, Rhyzobius lophanthae+TX, Rodolia cardinalis+TX, Rumina decollate+TX, Semielacher petiolatus+TX, Sitobion avenae (Ervibank®)+TX, Steinernema carpocapsae (Nematac C®+TX, Millenium®+TX, BioNem C®+TX, NemAttack®+TX, Nemastar®+TX, Capsanem®)+TX, Steinernema feltiae (NemaShield®+TX, Nemasys F®+TX, BioNem F®+TX, Steinernema-System®+TX, NemAttack®+TX, Nemaplus®+TX, Exhibitline sf®+TX, Scia-Rid®+TX, Entonem®)+TX, Steinernema kraussei (Nemasys L®+TX, BioNem L®+TX, Exhibitline srb®)+TX, Steinernema riobrave (BioVector®+TX, BioVektor®)+TX, Steinernema scapterisci (Nematac S®)+TX, Steinernema spp.+TX, Steinernematid spp. (Guardian Nematodes®)+TX, Stethorus punctillum (Stethorus®)+TX, Tamarixia radiate+TX, Tetrastichus setifer+TX, Thripobius semiluteus+TX, Torymus sinensis+TX, Trichogramma brassicae (Tricholine b®)+TX, Trichogramma brassicae (Tricho-Strip®)+TX, Trichogramma evanescens+TX, Trichogramma minutum+TX, Trichogramma ostriniae+TX, Trichogramma platneri+TX, Trichogramma pretiosum+TX, Xanthopimpla stemmator; other biologicals including: abscisic acid+TX, bioSea®+TX, Chondrostereum purpureum (Chontrol Paste®)+TX, Colletotrichum gloeosporioides (Collego®)+TX, Copper Octanoate (Cueva®)+TX, Delta traps (Trapline d®)+TX, Erwinia amylovora (Harpin) (ProAct®+TX, Ni-HIBIT Gold CST®)+TX, fatty acids derived from a natural by-product of extra virgin olive oil (FLIPPER®)+TX, Ferri-phosphate (Ferramol®)+TX, Funnel traps (Trapline y®)+TX, Gallex®+TX, Grower's Secret®+TX, Homo-brassonolide+TX, Iron Phosphate (Lilly Miller Worry Free Ferramol Slug & Snail Bait®)+TX, MCP hail trap (Trapline f®)+TX, Microctonus hyperodae+TX, Mycoleptodiscus terrestris (Des-X®)+TX, BioGain®+TX, Aminomite®+TX, Zenox®+TX, Pheromone trap (Thripline ams®)+TX, potassium bicarbonate (MilStop®)+TX, potassium salts of fatty acids (Sanova®)+TX, potassium silicate solution (Sil-Matrix®)+TX, potassium iodide+potassiumthiocyanate (Enzicur®)+TX, SuffOil-X®+TX, Spider venom+TX, Nosema locustae (Semaspore Organic Grasshopper Control®)+TX, Sticky traps (Trapline YF®+TX, Rebell Amarillo®)+TX and Traps (Takitrapline y+b®)+TX;
(1) antibacterial agents selected from the group of:
(1.1) bacteria, examples of which are Bacillus mojavensis strain R3B (Accession No. NCAIM (P) B001389) (WO 2013/034938) from Certis USA LLC, a subsidiary of Mitsui & Co.+TX; Bacillus pumilus, in particular strain BU F-33, having NRRL Accession No. 50185 (available as part of the CARTISSA® product from BASF, EPA Reg. No. 71840-19)+TX; Bacillus subtilis, in particular strain QST713/AQ713 (available as SERENADE OPTI or SERENADE ASO from Bayer CropScience LP, US, having NRRL Accession No. B21661, U.S. Pat. No. 6,060,051)+TX; Bacillus subtilis strain BU1814, (available as VELONDIS® PLUS, VELONDIS® FLEX and VELONDIS® EXTRA from BASF SE)+TX; Bacillus subtilis var. amyloliquefaciens strain FZB24 having Accession No. DSM 10271 (available from Novozymes as TAEGRO® or TAEGRO® ECO (EPA Registration No. 70127-5))+TX; Bacillus subtilis CX-9060 from Certis USA LLC, a subsidiary of Mitsui & Co.+TX; Bacillus sp., in particular strain D747 (available as DOUBLE NICKEL® from Kumiai Chemical Industry Co., Ltd.), having Accession No. FERM BP-8234, U.S. Pat. No. 7,094,592+TX; Paenibacillus sp. strain having Accession No. NRRL B-50972 or Accession No. NRRL B-67129, WO 2016/154297+TX; Paenibacillus polymyxa, in particular strain AC-1 (e.g. TOPSEED® from Green Biotech Company Ltd.)+TX; Pantoea agglomerans, in particular strain E325 (Accession No. NRRL B-21856) (available as BLOOMTIME BIOLOGICAL™ FD BIOPESTICIDE from Northwest Agri Products)+TX; Pseudomonas proradix (e.g. PRORADIX® from Sourcon Padena)+TX; and
(1.2) fungi, examples of which are Aureobasidium pullulans, in particular blastospores of strain DSM14940, blastospores of strain DSM 14941 or mixtures of blastospores of strains DSM14940 and DSM14941 (e.g., BOTECTOR® and BLOSSOM PROTECT® from bio-ferm, CH)+TX; Pseudozyma aphidis (as disclosed in WO2011/151819 by Yissum Research Development Company of the Hebrew University of Jerusalem)+TX; Saccharomyces cerevisiae, in particular strains CNCM No. 1-3936, CNCM No. 1-3937, CNCM No. 1-3938 or CNCM No. 1-3939 (WO 2010/086790) from Lesaffre et Compagnie, FR;
(2) biological fungicides selected from the group of:
(2.1) bacteria, examples of which are Agrobacterium radiobacter strain K84 (e.g. GALLTROL-A® from AgBioChem, CA)+TX; Agrobacterium radiobacter strain K1026 (e.g. NOGALL™ from BASF SE)+TX; Bacillus subtilis var. amyloliquefaciens strain FZB24 having Accession No. DSM 10271 (available from Novozymes as TAEGRO® or TAEGRO® ECO (EPA Registration No. 70127-5))+TX; Bacillus amyloliquefaciens, in particular strain D747 (available as Double Nickel™ from Kumiai Chemical Industry Co., Ltd., having accession number FERM BP-8234, U.S. Pat. No. 7,094,592)+TX; Bacillus amyloliquefaciens strain F727 (also known as strain MB1110) (NRRL Accession No. B-50768, WO 2014/028521) (STARGUS® from Marrone Bio Innovations)+TX; Bacillus amyloliquefaciens strain FZB42, Accession No. DSM 23117 (available as RHIZOVITAL® from ABiTEP, DE)+TX; Bacillus amyloliquefaciens isolate B246 (e.g. AVOGREEN™ from University of Pretoria)+TX; Bacillus licheniformis, in particular strain SB3086, having Accession No. ATCC 55406, WO 2003/000051 (available as ECOGUARD® Biofungicide and GREEN RELEAF™ from Novozymes)+TX+TX; Bacillus licheniformis FMCH001 and Bacillus subtilis FMCH002 (QUARTZO® (WG) and PRESENCE® (WP) from FMC Corporation)+TX; Bacillus methylotrophicus strain BAC-9912 (from Chinese Academy of Sciences' Institute of Applied Ecology)+TX; Bacillus mojavensis strain R3B (Accession No. NCAIM (P) B001389) (WO 2013/034938) from Certis USA LLC, a subsidiary of Mitsui & Co.+TX; Bacillus mycoides, isolate, having Accession No. B-30890 (available as BMJ TGAI® or WG and LifeGard™ from Certis USA LLC, a subsidiary of Mitsui & Co.)+TX; Bacillus pumilus, in particular strain QST2808 (available as SONATA® from Bayer CropScience LP, US, having Accession No. NRRL B-30087 and described in U.S. Pat. No. 6,245,551)+TX; Bacillus pumilus, in particular strain GB34 (available as Yield Shield® from Bayer AG, DE)+TX; Bacillus pumilus, in particular strain BU F-33, having NRRL Accession No. 50185 (available as part of the CARTISSA product from BASF, EPA Reg. No. 71840-19)+TX; Bacillus subtilis, in particular strain QST713/AQ713 (available as SERENADE OPTI or SERENADE ASO from Bayer CropScience LP, US, having NRRL Accession No. B21661 and described in U.S. Pat. No. 6,060,051)+TX; Bacillus subtilis Y1336 (available as BIOBAC® WP from Bion-Tech, Taiwan, registered as a biological fungicide in Taiwan under Registration Nos. 4764, 5454, 5096 and 5277)+TX; Bacillus subtilis strain MBI 600 (available as SUBTILEX from BASF SE), having Accession Number NRRL B-50595, U.S. Pat. No. 5,061,495+TX; Bacillus subtilis strain GB03 (available as Kodiak® from Bayer AG, DE)+TX; Bacillus subtilis strain BU1814, (available as VELONDIS® PLUS, VELONDIS® FLEX and VELONDIS® EXTRA from BASF SE)+TX; Bacillus subtilis CX-9060 from Certis USA LLC, a subsidiary of Mitsui & Co.+TX; Bacillus subtilis KTSB strain (FOLIACTIVE® from Donaghys)+TX; Bacillus subtilis IAB/BS03 (AVIV™ from STK Bio-Ag Technologies, PORTENTO® from Idai Nature)+TX; Bacillus subtilis strain Y1336 (available as BIOBAC® WP from Bion-Tech, Taiwan, registered as a biological fungicide in Taiwan under Registration Nos. 4764, 5454, 5096 and 5277)+TX; Paenibacillus epiphyticus (WO 2016/020371) from BASF SE+TX; Paenibacillus polymyxa ssp. plantarum (WO 2016/020371) from BASF SE+TX; Paenibacillus sp. strain having Accession No. NRRL B-50972 or Accession No. NRRL B-67129, WO 2016/154297+TX; Pseudomonas chlororaphis strain AFS009, having Accession No.

NRRL B-50897, WO 2017/019448 (e.g., HOWLER™ and ZIO® from AgBiome Innovations, US)+TX; Pseudomonas chlororaphis, in particular strain MA342 (e.g. CEDOMON®, CERALL®, and CEDRESS® by Bioagri and Koppert)+TX; Pseudomonas fluorescens strain A506 (e.g. BLIGHTBAN® A506 by NuFarm)+TX; Pseudomonas proradix (e.g. PRORADIX® from Sourcon Padena)+TX; Streptomyces griseoviridis strain K61 (also known as Streptomyces galbus strain K61) (Accession No. DSM 7206) (MYCOSTOP® from Verdera, PREFENCE® from BioWorks, cf. Crop Protection 2006, 25, 468-475)+TX; Streptomyces lydicus strain WYEC108 (also known as Streptomyces lydicus strain WYCD108US) (ACTINO-IRON® and ACTINOVATE® from Novozymes)+TX; and (2.2) fungi, examples of which are Ampelomyces quisqualis, in particular strain AQ 10 (e.g. AQ 10® by IntrachemBio Italia)+TX; Ampelomyces quisqualis strain AQ10, having Accession No. CNCM 1-807 (e.g., AQ 10® by IntrachemBio Italia)+TX; Aspergillus flavus strain NRRL 21882 (products known as AFLA-GUARD® from Syngenta/ChemChina)+TX; Aureobasidium pullulans, in particular blastospores of strain DSM14940+TX; Aureobasidium pullulans, in particular blastospores of strain DSM 14941+TX; Aureobasidium pullulans, in particular mixtures of blastospores of strains DSM14940 and DSM 14941 (e.g. Botector® by bio-ferm, CH)+TX; Chaetomium cupreum (Accession No. CABI 353812) (e.g. BIOKUPRUM™ by AgriLife)+TX; Chaetomium globosum (available as RIVADIOM® by Rivale)+TX; Cladosporium cladosporioides, strain H39, having Accession No. CBS122244, US 2010/0291039 (by Stichting Dienst Landbouwkundig Onderzoek)+TX; Coniothyrium minitans, in particular strain CON/M/91-8 (Accession No. DSM9660, e.g. Contans® from Bayer CropScience Biologics GmbH)+TX; Cryptococcus flavescens, strain 3C (NRRL Y-50378), (B2.2.99)+TX; Dactylaria candida+TX; Dilophosphora alopecuri (available as TWIST FUNGUS®)+TX; Fusarium oxysporum, strain Fo47 (available as FUSACLEAN® by Natural Plant Protection)+TX; Gliocladium catenulatum (Synonym: Clonostachys rosea f. catenulate) strain J1446 (e.g. Prestop® by Lallemand)+TX; Gliocladium roseum (also known as Clonostachys rosea f rosea), in particular strain 321U from Adjuvants Plus, strain ACM941 as disclosed in Xue (Efficacy of Clonostachys rosea strain ACM941 and fungicide seed treatments for controlling the root tot complex of field pea, Can Jour Plant Sci 83(3): 519-524), or strain IK726 (Jensen D F, et al. Development of a biocontrol agent for plant disease control with special emphasis on the near commercial fungal antagonist Clonostachys rosea strain ‘IK726’, Australas Plant Pathol. 2007,36:95-101)+TX; Lecanicillium lecanii (formerly known as Verticillium lecanii) conidia of strain KV01 (e.g. Vertalec® by Koppert/Arysta)+TX; Metschnikowia fructicola, in particular strain NRRL Y-30752, (B2.2.3)+TX; Microsphaeropsis ochracea+TX; Muscodor roseus, in particular strain A3-5 (Accession No. NRRL 30548)+TX; Penicillium steckii (DSM 27859, WO 2015/067800) from BASF SE+TX; Penicillium vermiculatum+TX; Phlebiopsis gigantea strain VRA 1992 (ROTSTOP® C from Danstar Ferment)+TX; Pichia anomala, strain WRL-076 (NRRL Y-30842), U.S. Pat. No. 7,579,183+TX; Pseudozyma flocculosa, strain PF-A22 UL (available as SPORODEX® L by Plant Products Co., CA)+TX; Saccharomyces cerevisiae, in particular strain LASO2 (from Agro-Levures et Derives), strain LAS117 cell walls (CEREVISANE® from Lesaffre, ROMEO® from BASF SE), strains CNCM No. 1-3936, CNCM No. 1-3937, CNCM No. 1-3938, CNCM No. 1-3939 (WO 2010/086790) from Lesaffre et Compagnie, FR+TX; Simplicillium lanosoniveum+TX; Talaromyces flavus, strain V117b+TX; Trichoderma asperelloides JM41R (Accession No. NRRL B-50759) (TRICHO PLUS® from BASF SE)+TX; Trichoderma asperellum, in particular, strain kd (e.g. T-Gro from Andermatt Biocontrol)+TX; Trichoderma asperellum, in particular strain SKT-1, having Accession No. FERM P-16510 (e.g. ECO-HOPE® from Kumiai Chemical Industry), strain T34 (e.g. T34 Biocontrol by Biocontrol Technologies S.L., ES) or strain ICC 012 from Isagro+TX; Trichoderma atroviride, in particular strain SC1 (having Accession No. CBS 122089, WO 2009/116106 and U.S. Pat. No. 8,431,120 (from Bi-PA)), strain 77B (T77 from Andermatt Biocontrol) or strain LU132 (e.g. Sentinel from Agrimm Technologies Limited)+TX; Trichoderma atroviride, strain CNCM 1-1237 (e.g. Esquive® WP from Agrauxine, FR)+TX; Trichoderma atroviride, strain no. V08/002387+TX; Trichoderma atroviride, strain NMI no. V08/002388+TX; Trichoderma atroviride, strain NMI no. V08/002389+TX; Trichoderma atroviride, strain NMI no. V08/002390+TX; Trichoderma atroviride, strain LC52 (e.g. Tenet by Agrimm Technologies Limited)+TX; Trichoderma atroviride, strain ATCC 20476 (IMI 206040)+TX; Trichoderma atroviride, strain T11 (IM1352941/CECT20498)+TX; Trichoderma atroviride, strain SKT-1 (FERM P-16510), JP Patent Publication (Kokai) 11-253151 A+TX; Trichoderma atroviride, strain SKT-2 (FERM P-16511), JP Patent Publication (Kokai) 11-253151 A+TX; Trichoderma atroviride, strain SKT-3 (FERM P-17021), JP Patent Publication (Kokai) 11-253151 A+TX; Trichoderma fertile (e.g. product TrichoPlus from BASF)+TX; Trichoderma gamsii (formerly T. viride), strain ICC080 (IMI CC 392151 CABI, e.g. BioDerma by AGROBIOSOL DE MEXICO, S.A. DE C.V.)+TX; Trichoderma gamsii (formerly T. viride), strain ICC 080 (IMI CC 392151 CABI) (available as BIODERMA® by AGROBIOSOL DE MEXICO, S.A. DE C.V.)+TX; Trichoderma harmatum+TX; Trichoderma harmatum, having Accession No. ATCC 28012+TX; Trichoderma harzianum strain T-22 (e.g. Trianum-P from Andermatt Biocontrol or Koppert) or strain Cepa SimbT5 (from Simbiose Agro)+TX; Trichoderma harzianum+TX; Trichoderma harzianum rifai T39 (e.g. Trichodex® from Makhteshim, US)+TX; Trichoderma harzianum, strain ITEM 908 (e.g. Trianum-P from Koppert)+TX; Trichoderma harzianum, strain TH35 (e.g. Root-Pro by Mycontrol)+TX; Trichoderma harzianum, strain DB 103 (available as T-GRO® 7456 by Dagutat Biolab)+TX; Trichoderma polysporum, strain IMI 206039 (e.g. Binab TF WP by BINAB Bio-Innovation AB, Sweden)+TX; Trichoderma stromaticum, having Accession No. Ts3550 (e.g. Tricovab by CEPLAC, Brazil)+TX; Trichoderma virens (also known as Gliocladium virens), in particular strain GL-21 (e.g. SoilGard by Certis, US)+TX; Trichoderma virens strain G-41, formerly known as Gliocladium virens (Accession No. ATCC 20906) (e.g., ROOTSHIELD® PLUS WP and TURFSHIELD® PLUS WP from BioWorks, US)+TX; Trichoderma viride, strain TV1(e.g. Trianum-P by Koppert)+TX; Trichoderma viride, in particular strain B35 (Pietr et al., 1993, Zesz. Nauk. A R w Szczecinie 161: 125-137)+TX; mixtures of Trichoderma asperellum strain ICC 012 (also known as Trichoderma harzianum I00012), having Accession No. CABI CC IMI 392716 and Trichoderma gamsii (formerly T. viride) strain ICC 080, having Accession No. IMI 392151 (e.g., BIO-TAM™ from Isagro USA, Inc. and BIODERMA® by Agrobiosol de Mexico, S.A. de C.V.)+TX; Ulocladium oudemansii strain U3, having Accession No. NM 99/06216 (e.g., BOTRY-ZEN® by Botry-Zen Ltd, New Zealand and BOTRYSTOP® from BioWorks, Inc.)+TX; Verticillium albo-atrum (formerly V. dahliae), strain WCS850 having Accession No. WCS850, deposited at the Central Bureau for Fungi Cultures (e.g., DUTCH TRIG® by Tree Care Innovations)+TX; Verticillium chlamydosporium+TX;
(3) biological control agents having an effect for improving plant growth and/or plant health selected from the group of:
(3.1) bacteria, examples of which are Azospirillum brasilense (e.g., VIGOR® from KALO, Inc.)+TX; Azospirillum lipoferum (e.g., VERTEX-IF™ from TerraMax, Inc.)+TX; Azorhizobium caulinodans, in particular strain ZB-SK-5+TX; Azotobacter chroococcum, in particular strain H23+TX; Azotobacter vinelandii, in particular strain ATCC 12837+TX; a mixture of Azotobacter vinelandii and Clostridium pasteurianum (available as INVIGORATE® from Agrinos)+TX; Bacillus amyloliquefaciens pm414 (LOLI-PEPTA® from Biofilm Crop Protection)+TX; Bacillus amyloliquefaciens SB3281 (ATCC #PTA-7542, WO 2017/205258)+TX; Bacillus amyloliquefaciens TJ1000 (available as QUIKROOTS® from Novozymes)+TX; Bacillus amyloliquefaciens, in particular strain IN937a+TX; Bacillus amyloliquefaciens, in particular strain FZB42 (e.g. RHIZOVITAL® from ABiTEP, DE)+TX; Bacillus amyloliquefaciens BS27 (Accession No. NRRL B-5015)+TX; Bacillus cereus family member EE128 (NRRL No. B-50917)+TX; Bacillus cereus family member EE349 (NRRL No. B-50928)+TX; Bacillus cereus, in particular strain BP01 (ATCC 55675, e.g. MEPICHLOR® from Arysta Lifescience, US)+TX; Bacillus firmus, in particular strain CNMC 1-1582 (e.g. VOTIVO® from BASF SE)+TX; Bacillus mycoides BT155 (NRRL No. B-50921)+TX; Bacillus mycoides EE118 (NRRL No. B-50918)+TX; Bacillus mycoides EE141 (NRRL No. B-50916)+TX; Bacillus mycoides BT46-3 (NRRL No. B-50922)+TX; Bacillus pumilus, in particular strain QST2808 (having Accession No. NRRL No. B-30087)+TX; Bacillus pumilus, in particular strain GB34 (e.g. YIELD SHIELD® from Bayer Crop Science, DE)+TX; Bacillus siamensis, in particular strain KCTC 13613T+TX; Bacillus subtilis, in particular strain QST713/AQ713 (having NRRL Accession No. B-21661 and described in U.S. Pat. No. 6,060,051, available as SERENADE® OPTI or SERENADE® ASO from Bayer CropScience LP, US)+TX; Bacillus subtilis, in particular strain AQ30002 (having Accession Nos. NRRL B-50421 and described in U.S. patent application Ser. No. 13/330,576)+TX; Bacillus subtilis, in particular strain AQ30004 (and NRRL B-50455 and described in U.S. patent application Ser. No. 13/330,576)+TX; Bacillus subtilis strain BU1814, (available as TEQUALIS® from BASF SE), Bacillus subtilis rm303 (RHIZOMAX® from Biofilm Crop Protection)+TX; Bacillus thuringiensis BT013A (NRRL No. B-50924) also known as Bacillus thuringiensis 4Q7+TX; a mixture of Bacillus licheniformis FMCH001 and Bacillus subtilis FMCH002 (available as QUARTZO® (WG), PRESENCE® (WP) from FMC Corporation)+TX; Bacillus subtilis, in particular strain MBI 600 (e.g. SUBTILEX® from BASF SE)+TX; Bacillus tequilensis, in particular strain NII-0943+TX; Bradyrhizobium japonicum (e.g. OPTIMIZE® from Novozymes)+TX; Delftia acidovorans, in particular strain RAY209 (e.g. BIOBOOST® from Brett Young Seeds)+TX; Mesorhizobium cicer (e.g., NODULATOR from BASF SE)+TX; Lactobacillus sp. (e.g. LACTOPLANT® from LactoPAFI)+TX; Rhizobium leguminosarium biovar viciae (e.g., NODULATOR from BASF SE)+TX; Pseudomonas proradix (e.g. PRORADIX® from Sourcon Padena)+TX; Pseudomonas aeruginosa, in particular strain PN1+TX; Rhizobium leguminosarum, in particular bv. viceae strain Z25 (Accession No. CECT 4585)+TX; Paenibacillus polymyxa, in particular strain AC-1 (e.g. TOPSEED® from Green Biotech Company Ltd.)+TX; Serratia marcescens, in particular strain SRM (Accession No. MTCC 8708)+TX; Sinorhizobium meliloti strain NRG-185-1 (NITRAGIN® GOLD from Bayer CropScience)+TX; Thiobacillus sp. (e.g. CROPAID® from Cropaid Ltd UK)+TX; and (3.2) fungi, examples of which are Purpureocillium lilacinum (previously known as Paecilomyces lilacinus) strain 251 (AGAL 89/030550, e.g. BioAct from Bayer CropScience Biologics GmbH)+TX; Penicillium bilaii, strain ATCC 22348 (e.g. JumpStart® from Acceleron BioAg), Talaromyces flavus, strain V117b+TX; Trichoderma atroviride strain CNCM 1-1237 (e.g. Esquive® WP from Agrauxine, FR), Trichoderma viride, e.g. strain B35 (Pietr et al., 1993, Zesz. Nauk. A R w Szczecinie 161: 125-137)+TX; Trichoderma atroviride strain LC52 (also known as Trichoderma atroviride strain LU132, e.g. Sentinel from Agrimm Technologies Limited)+TX; Trichoderma atroviride strain SC1 described in International Application No. PCT/IT2008/000196)+TX;Trichoderma asperellum strain kd (e.g. T-Gro from Andermatt Biocontrol)+TX; Trichoderma asperellum strain Eco-T (Plant Health Products, ZA), Trichoderma harzianum strain T-22 (e.g. Trianum-P from Andermatt Biocontrol or Koppert)+TX; Myrothecium verrucaria strain AARC-0255 (e.g. DiTera™ from Valent Biosciences)+TX; Penicillium bilaii strain ATCC ATCC20851+TX; Pythium oligandrum strain M1 (ATCC 38472, e.g. Polyversum from Bioprepraty, CZ)+TX; Trichoderma virens strain GL-21 (e.g. SoilGard® from Certis, USA)+TX; Verticillium albo-atrum (formerly V. dahliae) strain WCS850 (CBS 276.92, e.g. Dutch Trig from Tree Care Innovations)+TX; Trichoderma atroviride, in particular strain no. V08/002387, strain no. NMI No. V08/002388, strain no. NMI No. V08/002389, strain no. NMI No. V08/002390+TX; Trichoderma harzianum strain ITEM 908, Trichoderma harzianum, strain TSTh20+TX; Trichoderma harzianum strain 1295-22+TX; Pythium oligandrum strain DV74+TX; Rhizopogon amylopogon (e.g. comprised in Myco-Sol from Helena Chemical Company)+TX; Rhizopogon fulvigleba (e.g. comprised in Myco-Sol from Helena Chemical Company)+TX;Trichoderma virens strain GI-3+TX; (4) insecticidally active biological control agents selected from (4.1) bacteria, examples of which are Agrobacterium radiobacter strain K84 (Galltrol from AgBiochem Inc.)+TX; Bacillus amyloliquefaciens, in particular strain PTS-4838 (e.g. AVEO from Valent Biosciences, US)+TX; Bacillus firmus, in particular strain CNMC 1-1582 (e.g. VOTIVO® from BASF SE)+TX; Bacillus mycoides, isolate J. (e.g. BmJ from Certis USA LLC, a subsidiary of Mitsui & Co.)+TX; Bacillus sphaericus, in particular Serotype H5a5b strain 2362 (strain ABTS-1743) (e.g.

VECTOLEX® from Valent BioSciences, US)+TX; Bacillus thuringiensis subsp. aizawai, in particular strain ABTS-1857 (SD-1372, e.g. XENTARI® from Valent BioSciences)+TX; Bacillus thuringiensis subsp. aizawai, in particular serotype H-7 (e.g. FLORBAC® WG from Valent BioSciences, US)+TX; Bacillus thuringiensis israelensis strain BMP 144 (e.g. AQUABAC® by Becker Microbial Products IL)+TX; Bacillus thuringiensis subsp. israelensis (serotype H-14) strain AM65-52 (Accession No. ATCC 1276) (e.g. VECTOBAC® by Valent BioSciences, US)+TX; Bacillus thuringiensis subsp. aizawai strain GC-91+TX; Bacillus thuringiensis var. colmeri (e.g. TIANBAOBTC by Changzhou Jianghai Chemical Factory)+TX; Bacillus thuringiensis var. japonensis strain Buibui+TX; Bacillus thuringiensis subsp. kurstaki strain BMP 123 from Becker Microbial Products, IL+TX; Bacillus thuringiensis subsp. kurstaki strain BMP 123 by Becker Microbial Products, IL, e.g. BARITONE from Bayer CropScience+TX; Bacillus thuringiensis subsp. kurstaki strain HD-1 (e.g. DIPEL® ES from Valent BioSciences, US)+TX; Bacillus thuringiensis var. kurstaki strain EVB-113-19 (e.g., BIOPROTEC® from AEF Global)+TX; Bacillus thuringiensis subsp. kurstaki strain ABTS 351+TX; Bacillus thuringiensis subsp. kurstaki strain PB 54+TX; Bacillus thuringiensis subsp. kurstaki strain SA 11, (JAVELIN from Certis, US)+TX; Bacillus thuringiensis subsp. kurstaki strain SA 12 (THURICIDE from Certis, US)+TX; Bacillus thuringiensis subsp. kurstaki strain EG 2348 (LEPINOX from Certis, US)+TX; Bacillus thuringiensis subsp. kurstaki strain EG 7841 (CRYMAX from Certis, US)+TX; Bacillus thuringiensis subsp. tenebrionis strain NB 176 (SD-5428, e.g. NOVODOR® FC from BioFa DE)+TX; Brevibacillus laterosporus (LATERAL from Ecolibrium Biologicals)+TX; Burkholderia spp., in particular Burkholderia rinojensis strain A396 (also known as Burkholderia rinojensis strain MBI 305) (Accession No. NRRL B-50319+TX; WO 2011/106491 and WO 2013/032693+TX; e.g. MB1206 TGAI and ZELTO® from Marrone Bio Innovations)+TX; Chromobacterium subtsugae, in particular strain PRAA4-1T (MBI-203+TX; e.g. GRANDEVO® from Marrone Bio Innovations)+TX; Lecanicillium muscarium Ve6 (MYCOTAL from Koppert)+TX; Paenibacillus popilliae (formerly Bacillus popilliae+TX; e.g. MILKY SPORE POWDER™ and MILKY SPORE GRANULAR™ from St. Gabriel Laboratories)+TX; Pasteuria nishizawae strain Pn1 (CLARIVA from Syngenta/ChemChina)+TX; Serratia entomophila (e.g. INVADE® by Wrightson Seeds)+TX; Serratia marcescens, in particular strain SRM (Accession No. MTCC 8708)+TX;Trichoderma asperellum (TRICHODERMAX from Novozymes)+TX; Wolbachia pipientis ZAP strain (e.g., ZAP MALES® from MosquitoMate)+TX; and
(4.2) fungi, examples of which are Beauveria bassiana strain ATCC 74040 (e.g. NATURALIS® from Intrachem Bio Italia)+TX; Beauveria bassiana strain GHA (Accession No. ATCC74250, e.g. BOTANIGUARD® ES and MYCONTROL-O® from Laverlam International Corporation)+TX; Beauveria bassiana strain ATP02 (Accession No. DSM 24665)+TX; Isaria fumosorosea (previously known as Paecilomyces fumosoroseus) strain Apopka 97) PREFERAL from SePRO+TX; Metarhizium anisopliae 3213-1 (deposited under NRRL accession number 67074) (WO 2017/066094+TX; Pioneer Hi-Bred International)+TX; Metarhizium robertsii 15013-1 (deposited under NRRL accession number 67073)+TX; Metarhizium robertsii 23013-3 (deposited under NRRL accession number 67075)+TX; Paecilomyces lilacinus strain 251 (MELOCON from Certis, US)+TX; Zoophtora radicans+TX;
(5) Viruses selected from the group consisting of Adoxophyes orana (summer fruit tortrix) granulosis virus (GV)+TX; Cydia pomonella (codling moth) granulosis virus (GV)+TX; Helicoverpa armigera (cotton bollworm) nuclear polyhedrosis virus (NPV)+TX; Spodoptera exigua (beet armyworm) mNPV+TX; Spodoptera frugiperda (fall armyworm) mNPV+TX; Spodoptera littoralis (African cotton leafworm) NPV+TX;
(6) Bacteria and fungi which can be added as ‘inoculant’ to plants or plant parts or plant organs and which, by virtue of their particular properties, promote plant growth and plant health selected from Agrobacterium spp.+TX; Azorhizobium caulinodans+TX; Azospirillum spp.+TX; Azotobacter spp.+TX; Bradyrhizobium spp.+TX; Burkholderia spp., in particular Burkholderia cepacia (formerly known as Pseudomonas cepacia)+TX; Gigaspora spp., or Gigaspora monosporum+TX; Glomus spp.+TX; Laccaria spp.+TX; LactoBacillus buchneri+TX; Paraglomus spp.+TX; Pisolithus tinctorus+TX; Pseudomonas spp.+TX; Rhizobium spp., in particular Rhizobium trifolii+TX; Rhizopogon spp.+TX; Scleroderma spp.+TX; Suillus spp.+TX; Streptomyces spp.+TX;
(7) Plant extracts and products formed by microorganisms including proteins and secondary metabolites which can be used as biological control agents, selected from Allium sativum (NEMGUARD from Eco-Spray+TX; BRALIC from ADAMA)+TX; Armour-Zen+TX; Artemisia absinthium+TX; Azadirachtin (e.g. AZATIN XL from Certis, US)+TX; Biokeeper WP+TX; Brassicaceae extract, in particular oilseed rape powder or mustard powder+TX; Cassia nigricans+TX; Celastrus angulatus+TX; Chenopodium anthelminticum+TX; Chitin+TX; Dryopteris filix-mas+TX; Equisetum arvense+TX; Fortune Aza+TX; Fungastop+TX; Heads Up (Chenopodium quinoa saponin extract)+TX; PROBLAD (naturally occurring Blad polypeptide from Lupin seeds), Certis EU+TX; FRACTURE (naturally occurring Blad polypeptide from Lupin seeds), FMC+TX; Pyrethrum/Pyrethrins+TX; Quassia amara+TX; Quercus+TX; Quillaja extract (QL AGRI 35 from BASF)+TX; Reynoutria sachalinensis extract (REGALLIA/REGALIA MAXX from Marrone Bio)+TX; “Requiem™ Insecticide”+TX; Rotenone+TX; ryania/ryanodine+TX; Symphytum officinale+TX; Tanacetum vulgare+TX; Thymol+TX; Thymol mixed with Geraniol (CEDROZ from Eden Research)+TX; Thymol mixed with Geraniol and Eugenol (MEVALONE from Eden Research)+TX; Triact 70+TX; TriCon+TX; Tropaeulum majus+TX; Melaleuca alternifolia extract (TIMOREX GOLD from STK)+TX; Urtica dioica+TX; Veratrin+TX; and Viscum album+TX; and a safener, such as benoxacor+TX, cloquintocet (including cloquintocet-mexyl)+TX, cyprosulfamide+TX, dichlormid+TX, fenchlorazole (including fenchlorazole-ethyl)+TX, fenclorim+TX, fluxofenim+TX, furilazole+TX, isoxadifen (including isoxadifen-ethyl)+TX, mefenpyr (including mefenpyr-diethyl)+TX, metcamifen+TX and oxabetrinil+TX.

The references in brackets behind the active ingredients, e.g. [3878-19-1]refer to the Chemical Abstracts Registry number. The above described mixing partners are known. Where the active ingredients are included in “The Pesticide Manual” [The Pesticide Manual—A World Compendium; Thirteenth Edition; Editor: C. D. S. TomLin; The British Crop Protection Council], they are described therein under the entry number given in round brackets hereinabove for the particular compound; for example, the compound “abamectin” is described under entry number (1). Where “[CCN]” is added hereinabove to the particular compound, the compound in question is included in the “Compendium of Pesticide Common Names”, which is accessible on the internet [A. Wood; Compendium of Pesticide Common Names, Copyright © 1995-2004]; for example, the compound “acetoprole” is described under the internet address http://www.alanwood.net/pesticides/acetoprole.html.

Most of the active ingredients described above are referred to hereinabove by a so-called “common name”, the relevant “ISO common name” or another “common name” being used in individual cases. If the designation is not a “common name”, the nature of the designation used instead is given in round brackets for the particular compound; in that case, the IUPAC name, the IUPAC/Chemical Abstracts name, a “chemical name”, a “traditional name”, a “compound name” or a “development code” is used or, if neither one of those designations nor a “common name” is used, an “alternative name” is employed. “CAS Reg. No” means the Chemical Abstracts Registry Number.

The active ingredient mixture of the compounds of formula I selected from the compounds defined in the Tables P with active ingredients described above comprises a compound selected from one compound defined in the Table P and an active ingredient as described above preferably in a mixing ratio of from 100:1 to 1:6000, especially from 50:1 to 1:50, more especially in a ratio of from 20:1 to 1:20, even more especially from 10:1 to 1:10, very especially from 5:1 to 1:5, special preference being given to a ratio of from 2:1 to 1:2, and a ratio of from 4:1 to 2:1 being likewise preferred, above all in a ratio of 1:1, or 5:1, or 5:2, or 5:3, or 5:4, or 4:1, or 4:2, or 4:3, or 3:1, or 3:2, or 2:1, or 1:5, or 2:5, or 3:5, or 4:5, or 1:4, or 2:4, or 3:4, or 1:3, or 2:3, or 1:2, or 1:600, or 1:300, or 1:150, or 1:35, or 2:35, or 4:35, or 1:75, or 2:75, or 4:75, or 1:6000, or 1:3000, or 1:1500, or 1:350, or 2:350, or 4:350, or 1:750, or 2:750, or 4:750. Those mixing ratios are by weight.

The compounds and mixtures as described above can be used in a method for controlling pests, which comprises applying a composition comprising a compound or mixture respectively as described above to the pests or their environment, with the exception of a method for treatment of the human or animal body by surgery or therapy and diagnostic methods practised on the human or animal body.

The mixtures comprising a compound of formula I selected from the compounds defined in the Tables X & P and one or more active ingredients as described above can be applied, for example, in a single “ready-mix” form, in a combined spray mixture composed from separate formulations of the single active ingredient components, such as a “tank-mix”, and in a combined use of the single active ingredients when applied in a sequential manner, i.e. one after the other with a reasonably short period, such as a few hours or days. The order of applying the compounds of formula I and the active ingredients as described above is not essential for working the present invention.

The compositions according to the invention can also comprise further solid or liquid auxiliaries, such as stabilizers, for example unepoxidized or epoxidized vegetable oils (for example epoxidized coconut oil, rapeseed oil or soya oil), antifoams, for example silicone oil, preservatives, viscosity regulators, binders and/or tackifiers, fertilizers or other active ingredients for achieving specific effects, for example bactericides, fungicides, nematocides, plant activators, molluscicides or herbicides.

The compositions according to the invention are prepared in a manner known per se, in the absence of auxiliaries for example by grinding, screening and/or compressing a solid active ingredient and in the presence of at least one auxiliary for example by intimately mixing and/or grinding the active ingredient with the auxiliary (auxiliaries). These processes for the preparation of the compositions and the use of the compounds I for the preparation of these compositions are also a subject of the invention.

The application methods for the compositions, that is the methods of controlling pests of the abovementioned type, such as spraying, atomizing, dusting, brushing on, dressing, scattering or pouring—which are to be selected to suit the intended aims of the prevailing circumstances—and the use of the compositions for controlling pests of the abovementioned type are other subjects of the invention. Typical rates of concentration are between 0.1 and 1000 ppm, preferably between 0.1 and 500 ppm, of active ingredient. The rate of application per hectare is generally 1 to 2000 g of active ingredient per hectare, in particular 10 to 1000 g/ha, preferably 10 to 600 g/ha, especially 10 to 200 g/ha.

A preferred method of application in the field of crop protection is application to the foliage of the plants (foliar application), it being possible to select frequency and rate of application to match the danger of infestation with the pest in question. Alternatively, the active ingredient can reach the plants via the root system (systemic action), by drenching the locus of the plants with a liquid composition or by incorporating the active ingredient in solid form into the locus of the plants, for example into the soil, for example in the form of granules (soil application). In the case of paddy rice crops, such granules can be metered into the flooded paddy-field.

The compounds of formula I of the invention and compositions thereof are also be suitable for the protection of plant propagation material, for example seeds, such as fruit, tubers or kernels, or nursery plants, against pests of the abovementioned type. The propagation material can be treated with the compound prior to planting, for example seed can be treated prior to sowing. Alternatively, the compound can be applied to seed kernels (coating), either by soaking the kernels in a liquid composition or by applying a layer of a solid composition. It is also possible to apply the compositions when the propagation material is planted to the site of application, for example into the seed furrow during drilling. These treatment methods for plant propagation material and the plant propagation material thus treated are further subjects of the invention. Typical treatment rates would depend on the plant and pest/fungi to be controlled and are generally between 1 to 200 grams per 100 kg of seeds, preferably between 5 to 150 grams per 100 kg of seeds, such as between 10 to 100 grams per 100 kg of seeds.

The term seed embraces seeds and plant propagules of all kinds including but not limited to true seeds, seed pieces, suckers, corns, bulbs, fruit, tubers, grains, rhizomes, cuttings, cut shoots and the like and means in a preferred embodiment true seeds.

The present invention also comprises seeds coated or treated with or containing a compound of formula I. The term “coated or treated with and/or containing” generally signifies that the active ingredient is for the most part on the surface of the seed at the time of application, although a greater or lesser part of the ingredient may penetrate into the seed material, depending on the method of application. When the said seed product is (re)planted, it may absorb the active ingredient. In an embodiment, the present invention makes available a plant propagation material adhered thereto with a compound of formula I. Further, it is hereby made available, a composition comprising a plant propagation material treated with a compound of formula I.

Seed treatment comprises all suitable seed treatment techniques known in the art, such as seed dressing, seed coating, seed dusting, seed soaking and seed pelleting. The seed treatment application of the compound formula I can be carried out by any known methods, such as spraying or by dusting the seeds before sowing or during the sowing/planting of the seeds.

The compounds of the invention can be distinguished from other similar compounds by virtue of greater efficacy at low application rates and/or different pest control, which can be verified by the person skilled in the art using the experimental procedures, using lower concentrations if necessary, for example 10 ppm, 5 ppm, 2 ppm, 1 ppm or 0.2 ppm; or lower application rates, such as 300, 200 or 100, mg of Al per m². The greater efficacy can be observed by an increased safety profile (against non-target organisms above and below ground (such as fish, birds and bees), improved physico-chemical properties, or increased biodegradability).

In each aspect and embodiment of the invention, “consisting essentially” and inflections thereof are a preferred embodiment of “comprising” and its inflections, and “consisting of” and inflections thereof are a preferred embodiment of “consisting essentially of” and its inflections.

The disclosure in the present application makes available each and every combination of embodiments disclosed herein.

It should be noted that the disclosure herein in respect of a compound of formula I applies equally in respect of a compound of each of formulae I-1, la, Ib, Ic, and Id, and Tables A-1 to A-4 & P.

The compounds of the invention can be distinguished from other similar compounds by virtue of greater efficacy at low application rates and/or different pest control, which can be verified by the person skilled in the art using the experimental procedures, using lower concentrations if necessary, for example 10 ppm, 5 ppm, 2 ppm, 1 ppm or 0.2 ppm; or lower application rates, such as 300, 200 or 100, mg of Al per m². The greater efficacy can be observed by an increased safety profile (against non-target organisms above and below ground (such as fish, birds and bees), improved physico-chemical properties, or increased biodegradability).

### Biological Examples

The Examples which follow serve to illustrate the invention. Certain compounds of the invention can be distinguished from known compounds by virtue of greater efficacy at low application rates, which can be verified by the person skilled in the art using the experimental procedures outlined in the Examples, using lower application rates if necessary, for example 50 ppm, 24 ppm, 12.5 ppm, δ ppm, 3 ppm, 1.5 ppm, 0.8 ppm or 0.2 ppm.

Resistant Plutella xylostella R1 (Diamond back moth) larvicide L3, feeding/contact Chinese cabbage plants were sprayed with diluted test solutions in an application chamber. Cut off leaves were placed into petri dishes with wetted filter paper and infested 1 day after application with 10 L3 multi-resistant Plutella xylostella larvae having the G4946E resistance mutation.

Samples were assessed 4 days after infestation for mortality and growth regulation.

Chlorantraniliprole was used as standard and a resistance factor of 146 was obtained for this strain.

### Origin:

Plutella xylostella resistant strain R₁ originally collected from Taiwan in 2012 that carries the RyR mutation G4946E conferring resistance to diamides. The strain is reared on cabbage plants (Brassica aleracea) and selected approximately every two weeks with chlorantraniliprole.

The following compounds, according to the present invention, gave at least 80% control of the resistant strain of Plutella xylostella R1 at 50 ppm or below: P2, P6, P8, P9, P10, P11, P15, P18, P19, P20, P21, P22, P23, P24, P25, P26, P27, P28, P30, P31, P32, P33, P34, P36, P38, P43, P44, P49, P50, P57, P58, P62, P63, P67, P76.

Resistant Plutella xylostella R4 (Diamond back moth) larvicide L1, feeding/contact 24-well microtiter plates with artificial diet were treated with aqueous test solutions prepared from 10′000 ppm DMSO stock solutions by pipetting. After drying, around 30 Plutella eggs were pipetted through a plastic stencil onto a gel blotting paper and the plate was closed with it. The samples were assessed for mortality in comparison to untreated samples 8 days after infestation.

Chlorantraniliprole was used as standard and a resistance factor of 242 was obtained for this strain

### Origin:

Plutella xylostella resistant strain R4 originated in the lab in 2021 from crossing the R1 strain with a lab-reared susceptible P. xylostella strain (SUS). R4 can be reared and tested on artificial diet and also carries the RyR mutation G4946E conferring resistance to diamides. The strain is selected approximately every two weeks with chlorantraniliprole. The following compounds, according to the present invention, gave at least 80% control of the resistant strain of Plutella xylostella R₄ at 50 ppm or below: P11, P18, P26, P31, P33, P34, P36, P38, P42, P43, P46, P49, P50, P52, P56, P58, P60, P61, P63, P64, P65, P66, P67, P73, P76, P84, P97, P99, P102, P105.

### In Vitro Assay Method Description

Human embryonic kidney cells expressing the Plutella xylostella (Diamond back moth) ryanodine receptor, containing the G4946E resistance mutation, are loaded with Fluo-8 No Wash (NW) calcium-sensitive dye which responds by fluorescence to a change in intracellular calcium (e.g. stimulated by activation of ryanodine receptor). Test compounds are added in 10 rates to a 384-well plate containing dye-loaded cells and the fluorescent signal is measured using the Hamamatsu FDSS (Functional Drug Screening system). Dose-response curves are plotted to estimate the EC₅₀. EC₅₀ are normalized against an in-assay reference standard (cyantraniliprole) to address inter-assay variability. The ratio for a given compound is then obtained by the following formula:

R_{EC50}=EC₅₀ (compound)/EC₅₀ (cyantraniliprole). Compounds for which the ratio R_{EC50} is inferior or equal to 1 are equally or more active than cyantraniliprole.

The following compounds, according to the present invention, obtained a ratio R_{EC50}≤1: P2, P3, P4, P5, P6, P7, P8, P9, P10, P11, P12, P14, P16, P17, P18, P19, P20, P21, P22, P23, P24, P25, P26, P27, P28, P30, P31, P32, P33, P34, P35, P36, P37, P38, P39, P41, P42, P45, P46; P47, P50, P52, P53, P55, P59, P60, P61, P63, P64, P65, P66, P67, P70, P71, P72, P73, P75, P76, P79, P83, P84, P85, P86, P87, P88, P89, P90, P91, P92, P93, P96, P98, P105.

Table below provides the R_{EC50} for the compounds

| Compound of the invention | | Comparative compound | |
|---|---|---|---|
| | R_{EC50} | | R_{EC50} |
| P5 | 0.31 | N-[4-chloro-2-(isopropylcarbamoyl)-6-methyl- | 1.07 |
| | | phenyl]-5-[(4-chlorophenyl)methoxymethyl]-2-(3- | |
| | | chloro-2-pyridyl)pyrazole-3-carboxamide | |
| P8 | 0.14 | N-[4-chloro-2-methyl-6-(methylcarbamoyl)phenyl]- | 1.85 |
| | | 2-(3-chloro-2-pyridyl)-5-(2,2,3,3,3- | |
| | | pentafluoropropoxy)pyrazole-3-carboxamide | |
| P9 | 0.37 | N-[4-chloro-2-(isopropylcarbamoyl)-6-methyl- | 2.57 |
| | | phenyl]-2-(3-chloro-2-pyridyl)-5-methoxy-pyrazole- | |
| | | 3-carboxamide | |
| P21 | 0.25 | N-[4-chloro-2-methyl-6-(methylcarbamoyl)phenyl]- | 0.58 |
| | | 2-(3-chloro-2-pyridyl)-5-(difluoromethoxy)pyrazole- | |
| | | 3-carboxamide | |
| P23 | 0.17 | N-[4-chloro-2-methyl-6-(methylcarbamoyl)phenyl]- | 1.29 |
| | | 2-(3-chloro-2-pyridyl)-5-(2,2,2- | |
| | | trifluoroethoxy)pyrazole-3-carboxamide | |
| P24 | 0.18 | 5-bromo-N-[4-chloro-2-methyl-6- | 0.35 |
| | | (methylcarbamoyl)phenyl]-2-(3-chloro-2- | |
| | | pyridyl)pyrazole-3-carboxamide | |
| | | (Chlorantraniliprole) | |
| P25 | 0.18 | N-[4-chloro-2-methyl-6-(methylcarbamoyl)phenyl]- | 1.48 |
| | | 2-(3-chloro-2-pyridyl)-5-(trifluoromethyl)pyrazole-3- | |
| | | carboxamide | |
| P26 | 0.19 | N-[4-bromo-2-(isopropylcarbamoyl)-6-methyl- | 8.84 |
| | | phenyl]-2-(3-chloro-2-pyridyl)-5- | |
| | | (trifluoromethyl)pyrazole-3-carboxamide | |
| P27 | 0.18 | 2-(3-chloro-2-pyridyl)-N-[4-iodo-2- | 3.13 |
| | | (isopropylcarbamoyl)-6-methyl-phenyl]-5- | |
| | | (trifluoromethyl)pyrazole-3-carboxamide | |
| P30 | 0.39 | 5-bromo-2-(3-chloro-2-pyridyl)-N-[4-cyano-2- | 1.00 |
| | | methyl-6-(methylcarbamoyl)phenyl]pyrazole-3- | |
| | | carboxamide | |
| | | (Cyantraniliprole) | |
| P31 | 0.28 | 2-(3-chloro-2-pyridyl)-N-[4-cyano-2-methyl-6- | 3.65 |
| | | (methylcarbamoyl)phenyl]-5- | |
| | | (trifluoromethyl)pyrazole-3-carboxamide | |
| P33 | 0.31 | 5-bromo-N-[2,4-dichloro-6- | 0.47 |
| | | (methylcarbamoyl)phenyl]-2-(3,5-dichloro-2- | |
| | | pyridyl)pyrazole-3-carboxamide | |
| P34 | 0.18 | 5-bromo-N-[2-bromo-4-chloro-6-(1- | 4.70 |
| | | cyclopropylethylcarbamoyl)phenyl]-2-(3-chloro-2- | |
| | | pyridyl)pyrazole-3-carboxamide | |
| | | (Cyclaniliprole) | |
| P45 | 0.24 | N-[4-bromo-2-methyl-6-(methylcarbamoyl)phenyl]- | 1.39 |
| | | 2-(3-chloro-2-pyridyl)-5-methoxy-pyrazole-3- | |
| | | carboxamide | |
| P46 | 0.39 | 2-(3-chloro-2-pyridyl)-N-[2,4-dibromo-6- | 0.98 |
| | | (methylcarbamoyl)phenyl]-5- | |
| | | (trifluoromethyl)pyrazole-3-carboxamide | |
| P47 | 0.61 | N-[4-bromo-2-chloro-6-(methylcarbamoyl)phenyl]- | 1.32 |
| | | 2-(3-chloro-2-pyridyl)-5-methoxy-pyrazole-3- | |
| | | carboxamide | |
| P59 | 0.62 | 2-(3-chloro-2-pyridyl)-N-[2,4-dichloro-6- | 1.20 |
| | | (methylcarbamoyl)phenyl]-5-methoxy-pyrazole-3- | |
| | | carboxamide | |
| P63 | 0.19 | 5-bromo-2-(3-chloro-2-pyridyl)-N-[4-iodo-2-methyl- | 0.76 |
| | | 6-(methylcarbamoyl)phenyl]pyrazole-3- | |
| | | carboxamide | |
| P64 | 0.69 | 2-(3-chloro-2-pyridyl)-N-[2-fluoro-4-iodo-6- | 3.99 |
| | | (methylcarbamoyl)phenyl]-5- | |
| | | (trifluoromethyl)pyrazole-3-carboxamide | |
| P67 | 0.63 | 2-(3-chloro-2-pyridyl)-N-[4-cyano-2-methyl-6- | 2.41 |
| | | (methylcarbamoyl)phenyl]-5-[[5-[4- | |
| | | (trifluoromethyl)phenyl]tetrazol-2- | |
| | | yl]methyl]pyrazole-3-carboxamide | |
| P75 | 0.69 | N-[4-chloro-2-methyl-6-(methylcarbamoyl)phenyl]- | 1.41 |
| | | 2-(3-chloro-2-pyridyl)-5-ethyl-pyrazole-3- | |
| | | carboxamide | |
| P76 | 0.42 | N-[4-chloro-2-methyl-6-(methylcarbamoyl)phenyl]- | 1.15 |
| | | 2-(3-chloro-2-pyridyl)-5-cyclopropyl-pyrazole-3- | |
| | | carboxamide | |

1. A method for combating and controlling diamide-resistant insects to
(i) reduce damage on a plant, which comprises applying to the insect, to a locus of the insect, or to a plant susceptible to attack by the insect, an effective amount of a compound of formula I; or
(ii) protect plant propagation material, which comprises treating the propagation material or the site, where the propagation material is planted, with an effective amount of a compound of formula I;
wherein the compound of formula I is 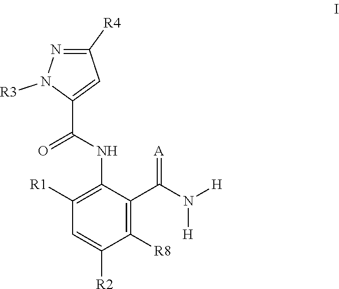
wherein
A is O or S;
R₁ is hydrogen, halogen, C₁-C₆alkyl, C₂-C₆alkenyl, C₂-C₆alkynyl, C₁-C₆haloalkyl, or C₃-C₆cycloalkyl;
R₂ is halogen or cyano;
R₃ is phenyl, or a 6-membered heteroaromatic ring, each of which is unsubstituted or substituted with one to three substituents independently selected from R₆;
R₄ is hydrogen, halogen, C₁-C₆alkyl, C₂-C₆alkenyl, C₂-C₆alkynyl, C₁-C₆haloalkyl, C₃-C₆cycloalkyl, C₃-C₆halocycloalkyl, C₃-C₆cyanocycloalkyl, C₁-C₆alkoxy, C₁-C₆haloalkoxy, or X2-Y, where X2 is C₁-C₆alkanediyl or C₁-C₆haloalkanediyl, and Y is cyano, C₂-C₆alkenyl, C₂-C₆alkynyl, C₁-C₆alkoxy, C₁-C₆haloalkoxy, C₃-C₆cycloalkyl, C₃-C₆halocycloalkyl, C₁-C₄alkylsulfanyl, C₁-C₄alkylsulfinyl, C₁-C₄alkylsulfonyl, C₁-C₄haloalkylsulfanyl, C₁-C₄haloalkylsulfinyl, C₁-C₄haloalkylsulfonyl, benzyloxy, halobenzyloxy, 5- or 6-membered heteroaromatic ring, which is unsubstituted or substituted with one to three independently selected R₇, or a 9- or 10-membered heteroaromatic bicyclic system, which is unsubstituted or substituted with one to three independently selected R₇;
R₆ is independently selected from halogen, cyano, C₁-C₃alkyl, C₁-C₃haloalkyl, C₁-C₃haloalkylthio, C₁-C₃alkoxy, C₁-C₃haloalkoxy, (C₁-C₃alkyl)NHC(═O), (C₁-C₃alkyl)₂NC(═O), (C₃-C₆cycloalkyl)NHC(═O), and (C₃-C₆cycloalkyl)(C₁-C₃alkyl)NC(═O);
R₇ is independently selected from halogen, cyano, C₁-C₃alkyl, C₁-C₃haloalkyl, C₃-C₆cycloalkyl, (C₁-C₃alkyl)NHC(═O), (C₁-C₃alkyl)₂NC(═O), (C₃-C₆cycloalkyl)NHC(═O), and (C₃-C₆cycloalkyl)(C₁-C₃alkyl)NC(═O), phenyl which may be substituted with one to three substituents independently selected from halogen, cyano, C₁-C₄alkyl, C₁-C₃alkoxy and C₁-C₃haloalkyl, and 6-membered heteroaromatic ring which may be substituted with one to three substituents independently selected from halogen, cyano, C₁-C₄alkyl, C₁-C₃alkoxy and C₁-C₃haloalkyl; and
R₈ is hydrogen, halogen, cyano, C₁-C₆alkyl, C₂-C₆alkenyl, C₂-C₆alkynyl, C₁-C₆haloalkyl, C₃-C₆cycloalkyl, C₁-C₆alkoxy, or C₁-C₆haloalkoxy; or an agronomically acceptable salt, isomer, enantiomer, tautomer and/or N-oxide of the compound of formula I.

2. The method according to claim 1, wherein the diamide-resistant insect is from the order Lepidoptera.

3. The method according to claim 1, wherein the diamide-resistant insect is resistant to least one compound selected from chlorantraniliprole, cyantraniliprole, cyclantraniliprole, fluchlordiniliprole, tetrachlorantraniliprole, tetraniliprole, flubendiamide and cyhalodiamide.

4. The method according to claim 1, wherein compound of formula I is represented by formula Ia 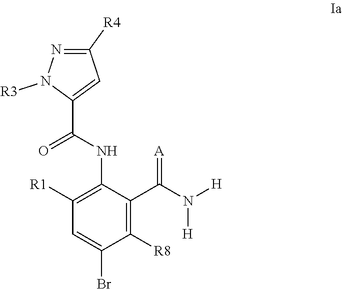
wherein bromine is R₂ from formula I; A is oxygen or sulfur; R₁ is hydrogen, halogen, C₁-C₆alkyl, C₂-C₆alkenyl, C₂-C₆alkynyl, C₁-C₆haloalkyl, or C₃-C₆cycloalkyl; R₄ is as defined in the first aspect; R₃ is 6-membered heteroaromatic ring, which is unsubstituted or substituted with one to three substituents independently selected from R₆; R₆ is independently selected from: halogen, C₁-C₃alkyl, and C₁-C₃haloalkyl; R₈ is hydrogen, halogen, cyano, C₁-C₆alkyl, C₂-C₆alkenyl, C₂-C₆alkynyl, C₁-C₆haloalkyl, C₃-C₆cycloalkyl, C₁-C₆alkoxy, or C₁-C₆haloalkoxy;
in the instance R₁ is chlorine, R₄ is hydrogen, fluorine, iodine, C₂-C₆alkyl, C₂-C₆alkenyl, C₂-C₆alkynyl, C₃-C₆cycloalkyl, C₃-C₆halocycloalkyl, C₃-C₆cyanocycloalkyl, C₁-C₆alkoxy, or X2-Y1;
in the instance R₁ is fluorine or bromine, R₄ is hydrogen, fluorine, iodine, C₁-C₆alkyl, C₂-C₆alkenyl, C₂-C₆alkynyl, C₁-C₆haloalkyl, C₃-C₆cycloalkyl, C₃-C₆halocycloalkyl, C₃-C₆cyanocycloalkyl, C₁-C₆alkoxy, C₁-C₆haloalkoxy, or X2-Y2; or
in the instance R₁ is methyl, R₄ is hydrogen, iodine, C₂-C₆alkyl, C₂-C₆alkenyl, C₂-C₆alkynyl, C₃-C₆cycloalkyl, C₃-C₆halocycloalkyl, C₃-C₆cyanocycloalkyl, C₁-C₆alkoxy, or X2-Y3;
or in the instance R₁ is hydrogen, iodine, C₂-C₆alkyl, C₂-C₆alkenyl, C₂-C₆alkynyl, C₁-C₆haloalkyl, or C₃-C₆cycloalkyl, R₄ is hydrogen, halogen, C₁-C₆alkyl, C₂-C₆alkenyl, C₂-C₆alkynyl, C₁-C₆haloalkyl, C₃-C₆cycloalkyl, C₃-C₆halocycloalkyl, C₃-C₆cyanocycloalkyl, C₁-C₆alkoxy, C₁-C₆haloalkoxy, or X2-Y4; where X2 is C₁-C₆alkanediyl or C₁-C₆haloalkanediyl;
Y1 is cyano, C₁-C₆haloalkoxy, C₃-C₆halocycloalkyl, C₁-C₄alkylsulfanyl, C₁-C₄alkylsulfinyl, C₁-C₄alkylsulfonyl, C₁-C₄haloalkylsulfanyl, C₁-C₄haloalkylsulfinyl, C₁-C₄haloalkylsulfonyl, benzyloxy, halobenzyloxy, or Y1.1-Y1.8; 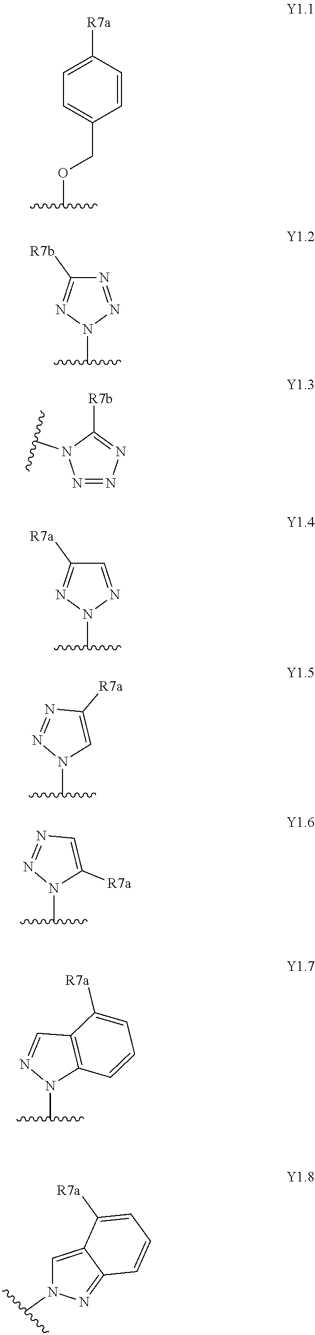
Y2 is cyano, C₁-C₆alkoxy, C₁-C₆haloalkoxy, C₃-C₆halocycloalkyl, C₁-C₄alkylsulfanyl, C₁-C₄alkylsulfinyl, C₁-C₄alkylsulfonyl, C₁-C₄haloalkylsulfanyl, C₁-C₄haloalkylsulfinyl, C₁-C₄haloalkylsulfonyl, benzyloxy, or Y2.1-Y2.8; 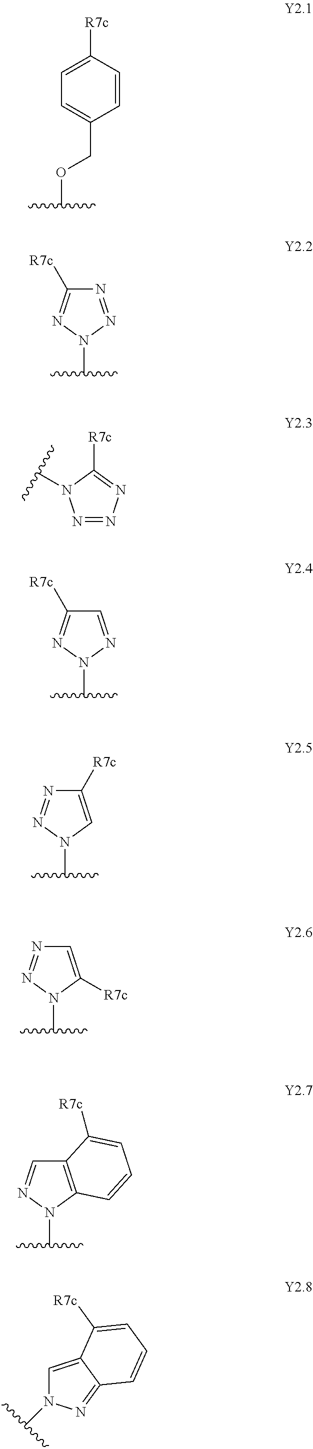
Y3 is cyano, C₁-C₆haloalkoxyC₃-C₆halocycloalkyl, C₁-C₄alkylsulfanyl, C₁-C₄alkylsulfinyl, C₁-C₄alkylsulfonyl, C₁-C₄haloalkylsulfanyl, C₁-C₄haloalkylsulfinyl, C₁-C₄haloalkylsulfonyl, benzyloxy, halobenzyloxy, or Y3.1-Y3.8; 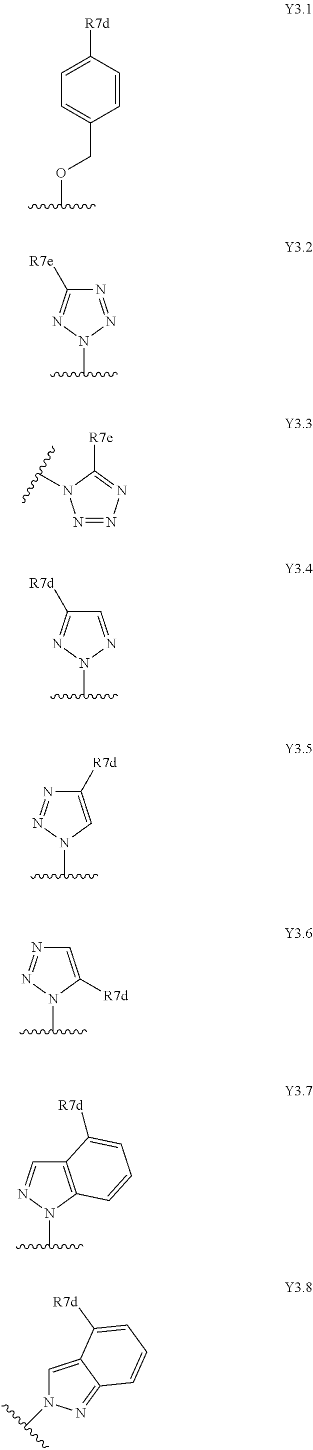
Y4 is cyano, C₁-C₆alkoxy, C₁-C₆haloalkoxy, C₃-C₆halocycloalkyl, C₁-C₄alkylsulfanyl, C₁-C₄alkylsulfinyl, C₁-C₄alkylsulfonyl, C₁-C₄haloalkylsulfanyl, C₁-C₄haloalkylsulfinyl, C₁-C₄haloalkylsulfonyl, benzyloxy, halobenzyloxy, or Y4.1-Y4.8; 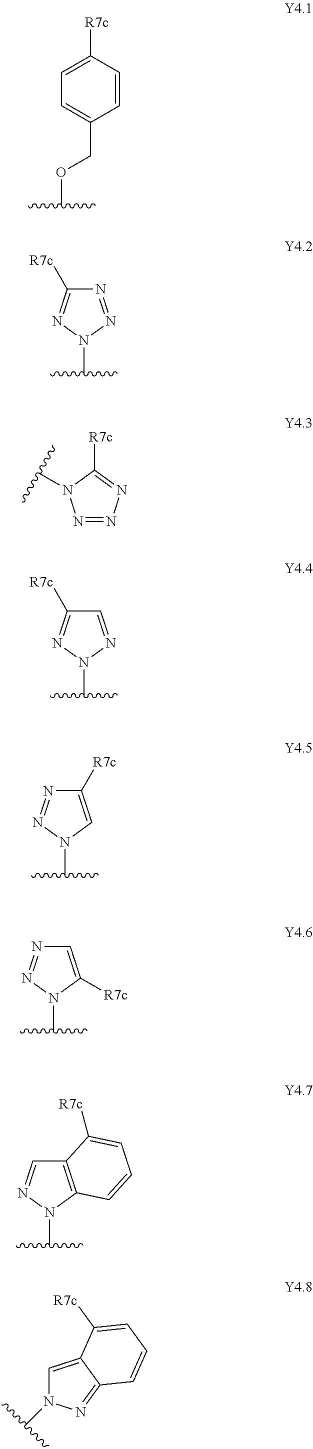
R₇ₐ is independently selected from hydrogen, halogen, cyano, C₁-C₃alkyl, C₁-C₃haloalkyl, C₃-C₆cycloalkyl, (C₁-C₃alkyl)NHC(O), (C₁-C₃alkyl)₂NC(O), (C₁-C₃cycloalkyl)NHC(O), and (C₁-C₃cycloalkyl)(C₁-C₃alkyl)NC(O), phenyl, and 6-membered heteroaromatic ring wherein the phenyl or the 6-membered ring is optionally substituted with one to three substituents independently selected from halogen, cyano, C₁-C₄alkyl, C₁-C₃alkoxy and C₁-C₃haloalkyl;
R_{7b} is independently selected from halogen, cyano, C₁-C₃alkyl, C₂-C₃haloalkyl, CHF₂, C₃-C₆cycloalkyl, (C₁-C₃alkyl)NHC(O), (C₁-C₃alkyl)₂NC(O), (C₁-C₃cycloalkyl)NHC(O), (C₁-C₃cycloalkyl)(C₁-C₃alkyl)NC(O), phenyl, and 6-membered heteroaromatic ring wherein the phenyl or the 6-membered ring is optionally substituted with one to three substituents independently selected from halogen, cyano, C₁-C₄alkyl, C₁-C₃alkoxy and C₁-C₃haloalkyl;
R_{7c} is independently selected from halogen, cyano, C₁-C₃alkyl, C₁-C₃haloalkyl, C₃-C₆cycloalkyl, (C₁-C₃alkyl)NHC(O), (C₁-C₃alkyl)₂NC(O), (C₁-C₃cycloalkyl)NHC(O), (C₁-C₃cycloalkyl)(C₁-C₃alkyl)NC(O),
phenyl, and 6-membered heteroaromatic ring wherein the phenyl or the 6-membered ring is optionally substituted with one to three substituents independently selected from halogen, cyano, C₁-C₄alkyl, C₁-C₃alkoxy and C₁-C₃haloalkyl;
R_{7d} is independently selected from hydrogen, halogen, cyano, C₁-C₃alkyl, C₁-C₃haloalkyl, C₃-C₆cycloalkyl, (C₁-C₃alkyl)NHC(O), (C₁-C₃alkyl)₂NC(O), (C₁-C₃cycloalkyl)NHC(O), (C₁-C₃cycloalkyl)(C₁-C₃alkyl)NC(O), and phenyl, wherein the phenyl is unsubstituted or substituted by one to three substituents selected from halogen, cyano, C₁-C₄alkyl, C₁-C₃alkoxy and C₁-C₃haloalkyl;
R₇ₑ is independently selected from: halogen, cyano, C₁-C₃alkyl, C₂-C₃haloalkyl, CHF₂, C₃-C₆cycloalkyl, (C₁-C₃alkyl)NHC(O), (C₁-C₃alkyl)₂NC(O), (C₁-C₃cycloalkyl)NHC(O), (C₁-C₃cycloalkyl)(C₁-C₃alkyl)NC(O), phenyl, and 6-membered heteroaromatic ring wherein the phenyl or the 6-membered ring is optionally substituted with one to three substituents independently selected from halogen, cyano, C₁-C₄alkyl, C₁-C₃alkoxy and C₁-C₃haloalkyl;
or an agronomically acceptable salt, isomer, enantiomer, tautomer and/or N-oxide of the compound of formula Ia.

5. The method according to claim 1, wherein compound of formula I is represented by formula Ib 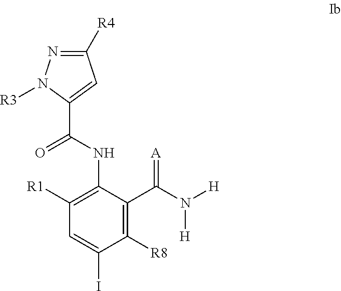
wherein iodine is R₂ from formula I; A is oxygen or sulfur; R₁ is hydrogen, halogen, C₁-C₆alkyl, C₂-C₆alkenyl, C₂-C₆alkynyl, C₁-C₆haloalkyl, or C₃-C₆cycloalkyl; R₄ is as defined in the first aspect; R₃ is 6-membered heteroaromatic ring, which is unsubstituted or substituted with one to three substituents independently selected from R₆; R₆ is independently selected from: halogen, C₁-C₃alkyl, and C₁-C₃haloalkyl; R₈ is hydrogen, halogen, cyano, C₁-C₆alkyl, C₂-C₆alkenyl, C₂-C₆alkynyl, C₁-C₆haloalkyl, C₃-C₆cycloalkyl, C₁-C₆alkoxy, or C₁-C₆haloalkoxy;
in the instance R₁ is hydrogen, halogen, C₂-C₆alkyl, C₂-C₆alkenyl, C₂-C₆alkynyl, C₁-C₆haloalkyl, or C₃-C₆cycloalkyl, R₄ is hydrogen, halogen, C₁-C₆alkyl, C₂-C₆alkenyl, C₂-C₆alkynyl, C₁-C₆haloalkyl, C₃-C₆cycloalkyl, C₃-C₆halocycloalkyl, C₃-C₆cyanocycloalkyl, C₁-C₆alkoxy, C₁-C₆haloalkoxy, or X2-Y5;
or in the instance R₁ is methyl, R₄ is hydrogen, halogen, C₁-C₆alkyl, C₂-C₆alkenyl, C₂-C₆alkynyl, CHF₂, C₂-C₆haloalkyl, C₃-C₆cycloalkyl, C₃-C₆halocycloalkyl, C₃-C₆cyanocycloalkyl, C₁-C₆alkoxy, C₁-C₆haloalkoxy, or X2-Y6;
where X2 is C₁-C₆alkanediyl or C₁-C₆haloalkanediyl;
Y5 is cyano, C₁-C₆alkoxy, C₁-C₆haloalkoxy, C₃-C₆halocycloalkyl, C₁-C₄alkylsulfanyl, C₁-C₄alkylsulfinyl, C₁-C₄alkylsulfonyl, C₁-C₄haloalkylsulfanyl, C₁-C₄haloalkylsulfinyl, C₁-C₄haloalkylsulfonyl, benzyloxy, halobenzyloxy, or Y5.1-Y5.8; 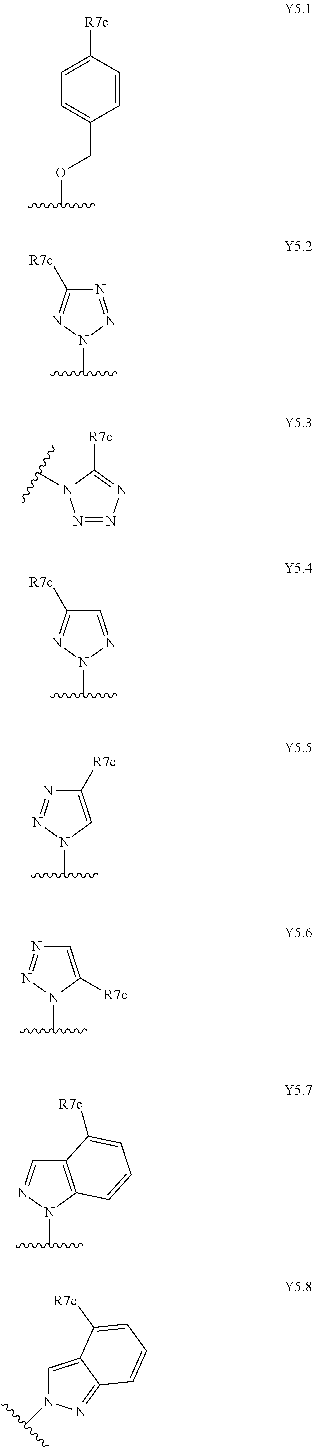
Y6 is cyano, C₁-C₆haloalkoxy, C₃-C₆halocycloalkyl, C₁-C₄alkylsulfanyl, C₁-C₄alkylsulfinyl, C₁-C₄alkylsulfonyl, C₁-C₄haloalkylsulfanyl, C₁-C₄haloalkylsulfinyl, C₁-C₄haloalkylsulfonyl, benzyloxy, halobenzyloxy, or Y6.1-Y6.8; 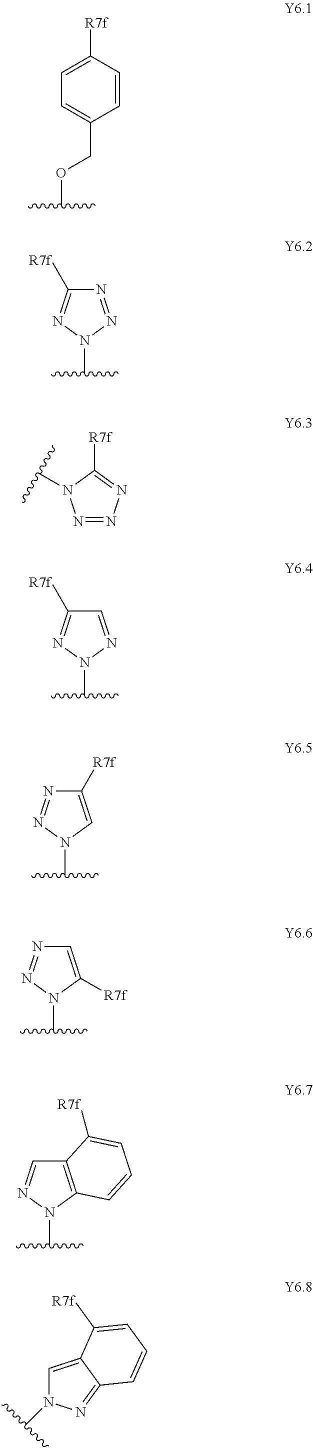
R_{7c} is independently selected from halogen, cyano, C₁-C₃alkyl, C₁-C₃haloalkyl, C₃-C₆cycloalkyl, (C₁-C₃alkyl)NHC(O), (C₁-C₃alkyl)₂NC(O), (C₁-C₃cycloalkyl)NHC(O), (C₁-C₃cycloalkyl)(C₁-C₃alkyl)NC(O),
phenyl, and 6-membered heteroaromatic ring wherein the phenyl or the 6-membered ring is optionally substituted with one to three substituents independently selected from halogen, cyano, C₁-C₄alkyl, C₁-C₃alkoxy and C₁-C₃haloalkyl;
R_{7f} is independently selected from hydrogen, halogen, cyano, C₁-C₃alkyl, C₃-C₆cycloalkyl, (C₁-C₃alkyl)NHC(O), (C₁-C₃alkyl)₂NC(O), (C₁-C₃cycloalkyl)NHC(O), (C₁-C₃cycloalkyl)(C₁-C₃alkyl)NC(O),
phenyl, and 6-membered heteroaromatic ring wherein the phenyl or the 6-membered ring is optionally substituted with one to three substituents independently selected from halogen, cyano, C₁-C₄alkyl, C₁-C₃alkoxy and C₁-C₃haloalkyl;
or an agronomically acceptable salt, isomer, enantiomer, tautomer and/or N-oxide of the compound of formula Ib.

6. The method according to claim 1, wherein compound of formula I is represented by formula Ic 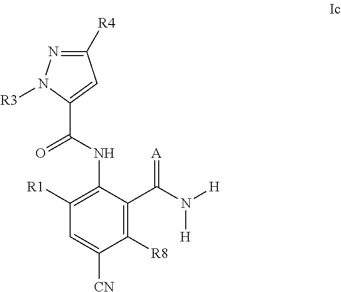
wherein cyano is R₂ from formula I; A is oxygen or sulfur; R₁ is hydrogen, halogen, C₁-C₆alkyl, C₂-C₆alkenyl, C₂-C₆alkynyl, C₁-C₆haloalkyl, or C₃-C₆cycloalkyl; R₄ is as defined in the first aspect; R₃ is 6-membered heteroaromatic ring, which is unsubstituted or substituted with one to three substituents independently selected from R₆; R₆ is independently selected from halogen, C₁-C₃alkyl, and C₁-C₃haloalkyl; R₈ is hydrogen, halogen, cyano, C₁-C₆alkyl, C₂-C₆alkenyl, C₂-C₆alkynyl, C₁-C₆haloalkyl, C₃-C₆cycloalkyl, C₁-C₆alkoxy, or C₁-C₆haloalkoxy;
in the instance R₁ is hydrogen, bromine, iodine, C₂-C₆alkyl, C₂-C₆alkenyl, C₂-C₆alkynyl, C₁-C₆haloalkyl, or C₃-C₆cycloalkyl, R₄ is hydrogen, halogen, C₁-C₆alkyl, C₂-C₆alkenyl, C₂-C₆alkynyl, C₁-C₆haloalkyl, C₃-C₆cycloalkyl, C₃-C₆halocycloalkyl, C₃-C₆cyanocycloalkyl, C₁-C₆alkoxy, C₁-C₆haloalkoxy, X2-Y7;
in the instance R₁ is methyl, R₄ is hydrogen, fluorine, iodine, C₂-C₆alkyl, C₂-C₆alkenyl, C₃-C₆cycloalkyl, C₃-C₆halocycloalkyl, C₃-C₆cyanocycloalkyl, C₁-C₆alkoxy, or X2-Y8;
in the instance R₁ is chlorine or fluorine, R₄ is hydrogen, fluorine, iodine, C₂-C₆alkyl, C₂-C₆alkenyl, C₂-C₆alkynyl, C₃-C₆cycloalkyl, C₃-C₆halocycloalkyl, C₃-C₆cyanocycloalkyl, or X2-Y9;
where X2 is C₁-C₆alkanediyl or C₁-C₆haloalkanediyl;
Y7 is cyano, C₁-C₆alkoxy, C₁-C₆haloalkoxy, C₃-C₆halocycloalkyl, C₁-C₄alkylsulfanyl, C₁-C₄alkylsulfinyl, C₁-C₄alkylsulfonyl, C₁-C₄haloalkylsulfanyl, C₁-C₄haloalkylsulfinyl, C₁-C₄haloalkylsulfonyl, benzyloxy, halobenzyloxy, or Y7.1-Y7.8; 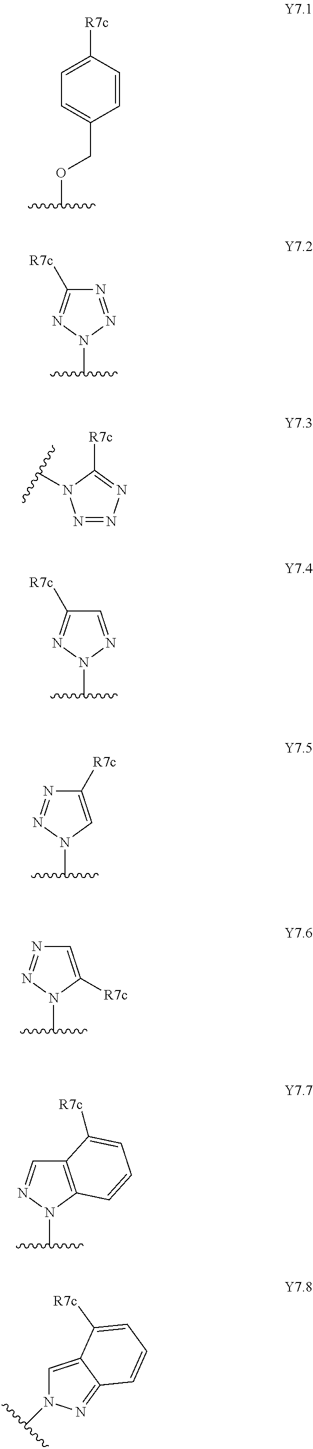
Y8 is cyano, C₁-C₆haloalkoxy, C₃-C₆halocycloalkyl, C₁-C₄alkylsulfanyl, C₁-C₄alkylsulfinyl, C₁-C₄alkylsulfonyl, C₁-C₄haloalkylsulfanyl, C₁-C₄haloalkylsulfinyl, C₁-C₄haloalkylsulfonyl, benzyloxy, halobenzyloxy, or Y8.1-Y8.8; 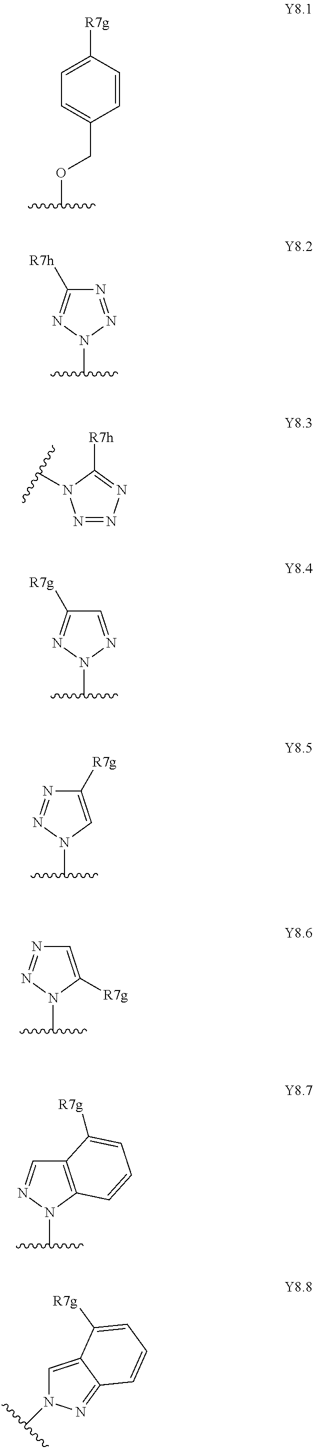
Y9 is cyano, C₁-C₆alkoxy, C₁-C₆haloalkoxy, C₃-C₆halocycloalkyl, C₁-C₄alkylsulfanyl, C₁-C₄alkylsulfinyl, C₁-C₄alkylsulfonyl, C₁-C₄haloalkylsulfanyl, C₁-C₄haloalkylsulfinyl, C₁-C₄haloalkylsulfonyl, benzyloxy, halobenzyloxy, or Y9.1-Y9.8; 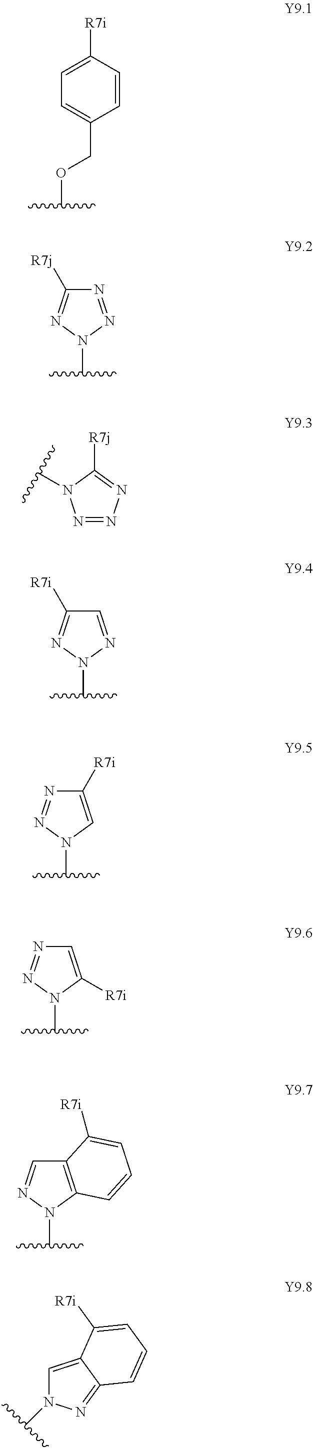
R_{7c} is independently selected from halogen, cyano, C₁-C₃alkyl, C₁-C₃haloalkyl, C₃-C₆cycloalkyl, (C₁-C₃alkyl)NHC(O), (C₁-C₃alkyl)₂NC(O), (C₁-C₃cycloalkyl)NHC(O), (C₁-C₃cycloalkyl)(C₁-C₃alkyl)NC(O), phenyl, and 6-membered heteroaromatic ring wherein the phenyl or the 6-membered ring is optionally substituted with one to three substituents independently selected from halogen, cyano, C₁-C₄alkyl, C₁-C₃alkoxy and C₁-C₃haloalkyl;
R_{7g} is independently selected from: hydrogen, halogen, cyano, C₁-C₃alkyl, C₃-C₆cycloalkyl, (C₁-C₃alkyl)NHC(O), (C₁-C₃alkyl)₂NC(O), (C₁-C₃cycloalkyl)NHC(O), (C₁-C₃cycloalkyl)(C₁-C₃alkyl)NC(O),
phenyl, and 6-membered heteroaromatic ring wherein the phenyl or the 6-membered ring is optionally substituted with one to three substituents independently selected from halogen, cyano, C₁-C₄alkyl, C₁-C₃alkoxy and C₁-C₃haloalkyl;
R₇ₕ is independently selected from: halogen, cyano, C₁-C₃alkyl, C₃-C₆cycloalkyl, (C₁-C₃alkyl)NHC(O), (C₁-C₃alkyl)₂NC(O), (C₁-C₃cycloalkyl)NHC(O), (C₁-C₃cycloalkyl)(C₁-C₃alkyl)NC(O),
phenyl, and 6-membered heteroaromatic ring wherein the phenyl or the 6-membered ring is optionally substituted with one to three substituents independently selected from halogen, cyano, C₁-C₄alkyl, C₁-C₃alkoxy and C₁-C₃haloalkyl;
R₇ᵢ is independently selected from hydrogen, halogen, cyano, C₁-C₃alkyl, C₁-C₃haloalkyl, C₃-C₆cycloalkyl, (C₁-C₃alkyl)NHC(O), (C₁-C₃alkyl)₂NC(O), (C₁-C₃cycloalkyl)NHC(O), (C₁-C₃cycloalkyl)(C₁-C₃alkyl)NC(O), phenyl, and 6-membered heteroaromatic ring wherein the phenyl or the 6-membered ring is optionally substituted with one to three substituents independently selected from halogen, cyano, C₁-C₄alkyl, C₁-C₃alkoxy and C₁-C₃haloalkyl;
R₇ⱼ is independently selected from halogen, cyano, C₁-C₃alkyl, C₂-C₃haloalkyl, CHF₂, C₃-C₆cycloalkyl, (C₁-C₃alkyl)NHC(O), (C₁-C₃alkyl)₂NC(O), (C₁-C₃cycloalkyl)NHC(O), (C₁-C₃cycloalkyl)(C₁-C₃alkyl)NC(O), phenyl, and 6-membered heteroaromatic ring wherein the phenyl or the 6-membered ring is optionally substituted with one to three substituents independently selected from halogen, cyano, C₁-C₄alkyl, C₁-C₃alkoxy and C₁-C₃haloalkyl;
or an agronomically acceptable salt, isomer, enantiomer, tautomer and/or N-oxide of the compound of formula Ic.

7. The method according to claim 1, wherein compound of formula I is represented by formula Id 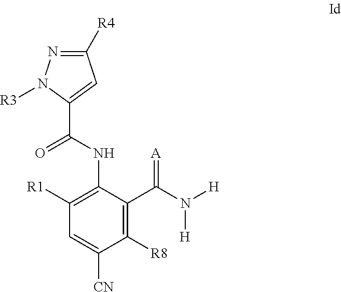
wherein chlorine is R₂ from formula I; A is oxygen or sulfur; R₁ is hydrogen, halogen, C₁-C₆alkyl, C₂-C₆alkenyl, C₂-C₆alkynyl, C₁-C₆haloalkyl, or C₃-C₆cycloalkyl; R₄ is as defined in the first aspect; R₃ is 6-membered heteroaromatic ring, which is unsubstituted or substituted with one to three substituents independently selected from R₆; R₆ is independently selected from halogen, C₁-C₃alkyl, and C₁-C₃haloalkyl; R₈ is hydrogen, halogen, cyano, C₁-C₆alkyl, C₂-C₆alkenyl, C₂-C₆alkynyl, C₁-C₆haloalkyl, C₃-C₆cycloalkyl, C₁-C₆alkoxy, or C₁-C₆haloalkoxy;
in the instance R₁ is chlorine, R₄ is hydrogen, fluorine, iodine, C₂-C₆alkyl, C₃-C₆cycloalkyl, C₃-C₆halocycloalkyl, C₃-C₆cyanocycloalkyl, C₁-C₆alkoxy or X2-Y10;
in the instance R₁ is bromine, R₄ is hydrogen, fluorine, iodine, C₂-C₆alkyl, C₂-C₆alkenyl, C₂-C₆alkynyl, C₆cycloalkyl, C₃-C₆halocycloalkyl, C₃-C₆cyanocycloalkyl, C₁-C₆alkoxy, OCF₃, or X2-Y11;
in the instance R₁ is methyl, R₄ is hydrogen, CH₂Cl, C₂-C₆alkyl, C₂-C₆alkenyl, C₃-C₆cycloalkyl, C₃-C₆cyanocycloalkyl, C₁-C₆alkoxy, OCH₂CHF₂, C₃-C₆haloalkoxy, or X2-Y12;
in the instance R₁ is hydrogen, C₂-C₆alkyl, C₂-C₆alkenyl, C₂-C₆alkynyl, C₁-C₆haloalkyl, or C₃-C₆cycloalkyl, R₄ is hydrogen, halogen, C₁-C₆alkyl, C₂-C₆alkenyl, C₂-C₆alkynyl, C₁-C₆haloalkyl, C₃-C₆cycloalkyl, C₃-C₆halocycloalkyl, C₃-C₆cyanocycloalkyl, C₁-C₆alkoxy, C₁-C₆haloalkoxy, or X2-Y13;
where X2 is C₁-C₆alkanediyl or C₁-C₆haloalkanediyl;
Y10 is cyano, C₃-C₆halocycloalkyl, C₁-C₄alkylsulfanyl, C₁-C₄alkylsulfinyl, C₁-C₄alkylsulfonyl, benzyloxy, halobenzyloxy, or Y10.1-Y10.8; 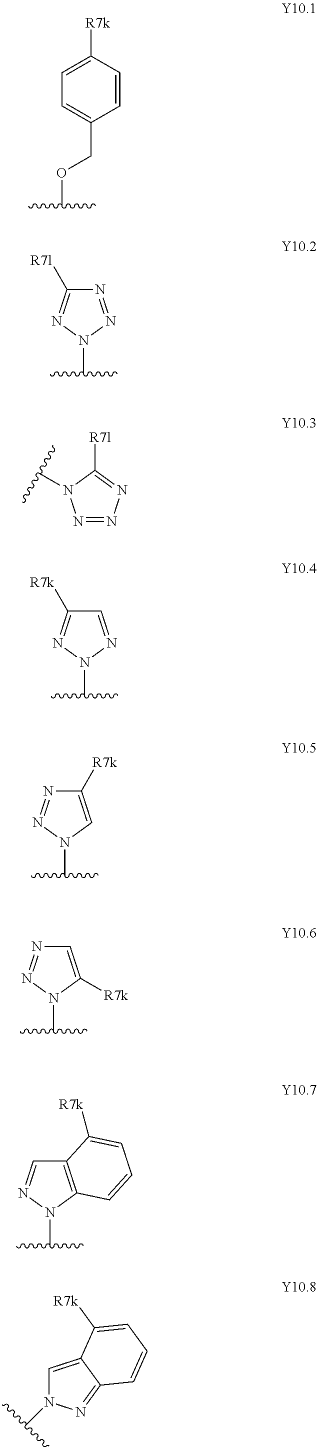
Y11 is cyano, C₁-C₆haloalkoxy, C₃-C₆halocycloalkyl, C₁-C₄alkylsulfanyl, C₁-C₄alkylsulfinyl, C₁-C₄alkylsulfonyl, C₁-C₄haloalkylsulfanyl, C₁-C₄haloalkylsulfinyl, C₁-C₄haloalkylsulfonyl, benzyloxy, halobenzyloxy, or Y11.1-Y11.8 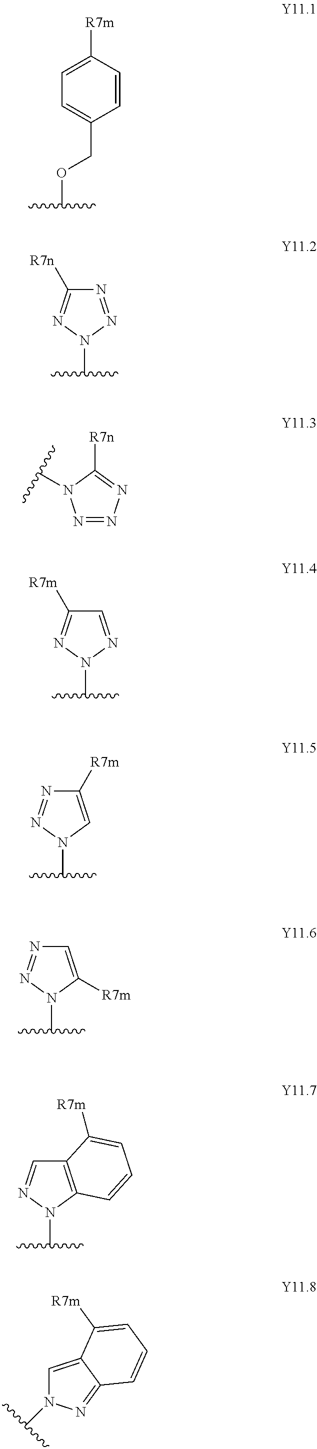
Y12 is cyano, C₂-C₆haloalkoxy, benzyloxy, halobenzyloxy, or Y12.1-Y12.8; 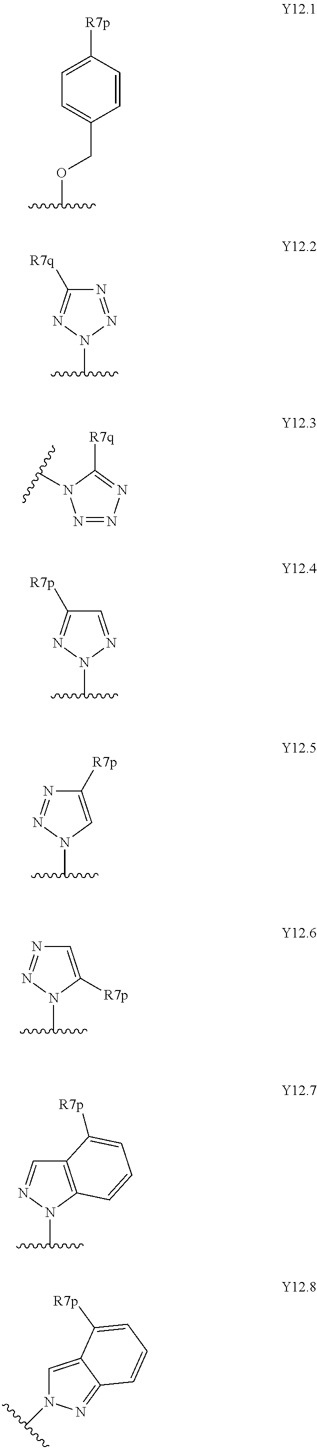
Y13 is cyano, C₁-C₆alkoxy, C₁-C₆haloalkoxy, C₃-C₆halocycloalkyl, C₁-C₄alkylsulfanyl, C₁-C₄alkylsulfinyl, C₁-C₄alkylsulfonyl, C₁-C₄haloalkylsulfanyl, C₁-C₄haloalkylsulfinyl, C₁-C₄haloalkylsulfonyl, benzyloxy, halobenzyloxy, or Y13.1-Y13.8; 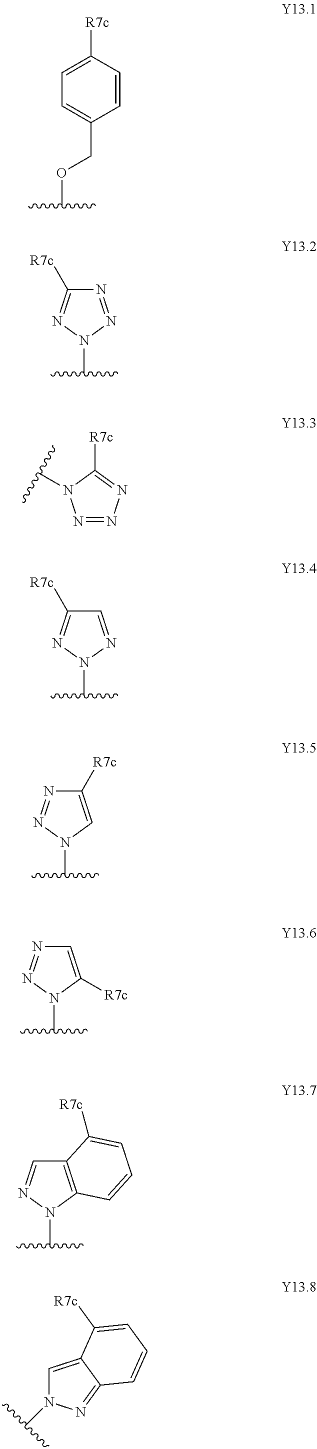
R_{7c} is independently selected from halogen, cyano, C₁-C₃alkyl, C₁-C₃haloalkyl, C₃-C₆cycloalkyl, (C₁-C₃alkyl)NHC(O), (C₁-C₃alkyl)₂NC(O), (C₁-C₃cycloalkyl)NHC(O), (C₁-C₃cycloalkyl)(C₁-C₃alkyl)NC(O),
phenyl, and 6-membered heteroaromatic ring wherein the phenyl or the 6-membered ring is optionally substituted with one to three substituents independently selected from halogen, cyano, C₁-C₄alkyl, C₁-C₃alkoxy and C₁-C₃haloalkyl;
R₇ₖ is independently selected from hydrogen, halogen, cyano, C₁-C₃alkyl, (C₁-C₃alkyl)NHC(O), (C₁-C₃alkyl)₂NC(O), (C₁-C₃cycloalkyl)NHC(O), (C₁-C₃cycloalkyl)(C₁-C₃alkyl)NC(O), phenyl, and 6-membered heteroaromatic ring wherein the phenyl or the 6-membered ring is optionally substituted with one to three substituents independently selected from halogen, cyano, C₁-C₄alkyl, C₁-C₃alkoxy and C₁-C₃haloalkyl;
R₇ₗ is independently selected from: halogen, cyano, C₁-C₃alkyl, C₁-C₃alkyl)NHC(O), (C₁-C₃alkyl)₂NC(O), (C₁-C₃cycloalkyl)NHC(O), (C₁-C₃cycloalkyl)(C₁-C₃alkyl)NC(O), phenyl, and 6-membered heteroaromatic ring wherein the phenyl or the 6-membered ring is optionally substituted with one to three substituents independently selected from halogen, cyano, C₁-C₄alkyl, C₁-C₃alkoxy and C₁-C₃haloalkyl;
R₇ₘ is independently selected from: hydrogen, halogen, cyano, C₁-C₃alkyl, C₁-C₃haloalkyl, C₃-C₆cycloalkyl, (C₁-C₃alkyl)NHC(O), (C₁-C₃alkyl)₂NC(O), (C₁-C₃cycloalkyl)NHC(O), (C₁-C₃cycloalkyl)(C₁-C₃alkyl)NC(O), phenyl, and 6-membered heteroaromatic ring wherein the phenyl or the 6-membered ring is optionally substituted with one to three substituents independently selected from halogen, cyano, C₁-C₄alkyl, C₁-C₃alkoxy and C₁-C₃haloalkyl;
R₇ₙ is independently selected from halogen, cyano, C₁-C₃alkyl, C₂-C₃haloalkyl, CHF₂, C₃-C₆cycloalkyl, (C₁-C₃alkyl)NHC(O), (C₁-C₃alkyl)₂NC(O), (C₁-C₃cycloalkyl)NHC(O), (C₁-C₃cycloalkyl)(C₁-C₃alkyl)NC(O), phenyl, and 6-membered heteroaromatic ring wherein the phenyl or the 6-membered ring is optionally substituted with one to three substituents independently selected from halogen, cyano, C₁-C₄alkyl, C₁-C₃alkoxy and C₁-C₃haloalkyl;
R₇ₚ is independently selected from hydrogen, halogen, cyano, C₁-C₃alkyl, (C₁-C₃alkyl)NHC(O), (C₁-C₃alkyl)₂NC(O), (C₁-C₃cycloalkyl)NHC(O), (C₁-C₃cycloalkyl)(C₁-C₃alkyl)NC(O), phenyl, and 6-membered heteroaromatic ring wherein the phenyl or the 6-membered ring is optionally substituted with one to three substituents independently selected from halogen, cyano, C₁-C₄alkyl, C₁-C₃alkoxy and C₁-C₃haloalkyl;
R_{7q} is independently selected from halogen, cyano, C₁-C₃alkyl, C₃-C₆cycloalkyl, (C₁-C₃alkyl)NHC(O), (C₁-C₃alkyl)₂NC(O), (C₁-C₃cycloalkyl)NHC(O), (C₁-C₃cycloalkyl)(C₁-C₃alkyl)NC(O), phenyl, and 6-membered heteroaromatic ring wherein the phenyl or the 6-membered ring is optionally substituted with one to three substituents independently selected from halogen, cyano, C₁-C₄alkyl, C₁-C₃alkoxy and C₁-C₃haloalkyl;
or an agronomically acceptable salt, isomer, enantiomer, tautomer and/or N-oxide of the compound of formula Id.

8. The method according to claim 1, wherein the diamide-resistant insect is Plutella xylostella (Troczka et al. 2012; Steinbach et al. 2015; Guo et al. 2014), Tuta absoluta (Roditakis et al. 2017; Zimmer et al. 2019), Spodoptera frugiperda (Bolzan et al. 2019) Spodoptera exigua (Zuo et al. 2020, 2017), or Chilo suppressalis (Yao et al. 2017; Yang et al. 2017).

9. The method according to claim 1, wherein the diamide-resistant insect is in a defined area/field of plants where the ratio of diamide-resistant insects to their corresponding sensitive strains is greater than 1:20 (based on number of insects).

10. The method according to claim 1, wherein the compound of formula I (and formulae Ia to Id) controls the diamide-resistant insect better compared to the secondary amide analog of the compound of formula I.

11. A compound of formula I as defined in claim 1, wherein the compound is further defined as formula Ia, Ib, Ic, or Id.

12. A composition comprising a compound of formula Ia, Ib, Ic or Id, as defined in claim 11, one or more auxiliaries and diluent, and optionally one or more other active ingredient.

13. A method of combating and controlling insects, acarines, nematodes or molluscs which comprises applying to a pest, to a locus of a pest, or to a plant susceptible to attack by a pest an insecticidally, acaricidally, nematicidally or molluscicidally effective amount of a compound of formula Ia, Ib, Ic or Id, as defined in claim 11.

14. A method for the protection of plant propagation material from the attack by insects, acarines, nematodes or molluscs, which comprises treating the propagation material or the site, where the propagation material is planted, with an effective amount of a compound of formula Ia, Ib, Ic or Id, as defined in claim 11.

15. A plant propagation material, such as a seed, comprising, or treated with or adhered thereto, a compound of formula Ia, Ib, Ic or Id, as defined in claim 11.
